(19) United States
(12) Patent Application Publication
(10) Pub. No.: US 2025/0101375 A1
(43) Pub. Date: Mar. 27, 2025

(54) METHODS OF GENERATING A SYNTHETIC EMBRYO

(71) Applicant: Yeda Research and Development Co. Ltd., Rehovot (IL)

(72) Inventors: Yaqub HANNA, Tel Aviv-Yafo (IL); Sergey VIUKOV, Rehovot (IL); Emilie WILDSCHUTZ, Rehovot (IL); Noa NOVERSHTERN, Rehovot (IL); Carine JOUBRAN, Rehovot (IL); Segev NAVEH TASSA, Rehovot (IL); Alejandro CASTREJON AGUILERA, Rehovot (IL); Bernardo OLDAK, Rehovot (IL); Shadi TARAZI, Rehovot (IL); Francesco RONCATO, Rehovot (IL)

(73) Assignee: Yeda Research and Development Co. Ltd., Rehovot (IL)

(21) Appl. No.: 18/825,336

(22) Filed: Sep. 5, 2024

Related U.S. Application Data
(63) Continuation of application No. PCT/IL2023/050235, filed on Mar. 6, 2023.
(60) Provisional application No. 63/317,036, filed on Mar. 6, 2022.
(60) Provisional application No. 63/390,695, filed on Jul. 20, 2022.

(51) Int. Cl. C12N 5/073; C12N 5/071

(52) U.S. Cl. CPC C12N 5/0604; C12N 5/0608; C12N 2501/115; C12N 2501/119; C12N 2501/135; C12N 2501/235; C12N 2501/33; C12N 2501/40; C12N 2501/415; C12N 2506/45

(57) ABSTRACT

Methods of generating a synthetic embryo are provided. Accordingly, there is provided a method of generating a synthetic embryo comprising inducing expression of a factor that induces differentiation to trophectoderm cells in a subpopulation of naïve pluripotent stem cells (PSCs) to obtain a trophectoderm primed cells; inducing expression of a factor that induces differentiation to extra embryonic primitive endodermal cells in a second subpopulation of naïve PSCs to obtain extra embryonic primitive endodermal primed cells; and mixing said trophectoderm primed cells and said extra embryonic primitive endodermal primed cells with naïve PSCs under conditions that allow formation of aggregated cells. Also provided are articles of manufactures, mixtures and aggregates of cells and methods of using same.

### RELATED APPLICATIONS

This application is a Continuation of PCT Patent Application No. PCT/IL2023/050235 having International filing date of Mar. 6, 2023, which claims the benefit of priority under 35 USC § 119 (e) of U.S. Provisional Patent Applications Nos. 63/317,036 filed on Mar. 6, 2022 and 63/390,695 filed on Jul. 20, 2022. The contents of the above applications are all incorporated by reference as if fully set forth herein in their entirety.

### SEQUENCE LISTING STATEMENT

The XML file, entitled 97313ReplacementSequenceListing.xml, created on Oct. 21, 2024, comprising 319,728 bytes, is incorporated herein by reference.

### FIELD AND BACKGROUND OF THE INVENTION

The present invention, in some embodiments thereof, relates to methods of generating a synthetic embryo.

Advanced artificial embryogenesis utilizing in vitro established embryonic stem cell lines and induced pluripotent stem cell lines (iPSCs) while bypassing the use of oocytes and zygotes or blastocysts has been a desired goal in the fields of developmental biology and reproductive medicine. The establishment of methods and systems for producing such synthetic embryos (abbreviated as sEmbryo or sEmbryoids; also could be referred to as Stem Cell-Derived Embryo or Embryoid Model (SEMs) or Stem Cell-Derived Whole Embryoid Model (SWEM)) ex utero may constitute a powerful tool in basic research e.g. as a framework to investigate the emergence of cellular diversity, cell fate decisions and how tissues and organs emerge from a single pluripotent cell; as well as a source of cells, tissue, embryos and organs for transplantation, testing the effect of drugs on embryonic development and possibly the bioengineering of embryos.

The blastocyst (the early mammalian embryo) forms all embryonic and extraembryonic tissues, including the placenta. This structure occurs at the 64-cell stage, which is shortly after the embryo loses its “ball of cells” shape and forms a cavity. In this structure, the inner cell mass (ICM) will develop into the embryo proper, and is placed above the blastocoel cavity which is the hallmark of the blastocyst stage. Cells enveloped around the ICM are trophectoderm (TE) cells, which will later contribute to trophoblast extraembryonic placental tissue (fetal placenta). Part of the ICM further differentiates into the primitive endoderm (PRE) which gives rise later on to the extraembryonic yolk sac and membranes, while preparing for implantation [Zhang et al. (2020) Front. Bioeng. Biotechnol. 8:781].

Different pluripotent states of embryonic stem cell lines (ESCs) and iPSCs can be expanded in vitro and can contribute to embryonic and/or extraembryonic tissues when injected into host embryos. For example, upon microinjection into mouse blastocysts, mouse naive ESCs or iPSCs (Bayerl et al. Cell Stem Cell (2021) 28 (9): 1549-1565.e12; Weinberger et al. Nat Rev Mol Cell Biol (2016) 17 (3): 155-69; Manor et al. Curr Opin Genet Dev (2015) 34:35-45; Shakiba et al. Nat Commun (2015) 16 6:7329; Gafni et al. Nature (2013) 504 (7479): 282-6; Geula et al. Science (2015) 347 (6225): 1002-6) can function like the resident pluripotent cells and equally contribute to the embryos being formed (Nicols and Smith Cell Stem Cell (2009) 4 (6): 487-92). To the contrary, mouse primed pluripotent stem cells (also known as Epiblast stem cells (EpiSCs)), expanded in N2B27 FGF2/ACTIVIN conditions are extremely inefficient at making chimeric embryos and not able to significantly contribute to such embryos when injected into host blastocysts or E7.5 embryos (Alejandro-Castrejon et al. Nature (2021) 593 (7857): 119-124).

Mouse trophoblast stem cells (TSCs) can be derived from early mouse embryos (E5.5-E7.5 post-implantation mouse embryos) or directly from mouse ESCs in vitro (through a process that takes few passages of growth and selection to establish stable mouse TSC lines and typically alongside overexpression of transgene like Cdx2). Upon microinjection of conventional stabilized mouse TSC lines into blastocysts they can mildly contribute to advanced embryonic placental development, but have never been shown to be able to reconstitute the entire placental and trophectoderm progeny (Posfai et al. Nat Cell Biol (2021) 23 (1): 49-60). Injecting mouse naïve ESCs or iPSCs in the tetraploid embryo complementation microinjection approach can generate unique “all ESC/iPSC” chimeras in which all the embryonic cells are derived only from the injected mouse stem cells, and the extraembryonic cells are derived only from the electrofused cells of the host embryo (Choi et al. Nature (2017) 548 (7666): 219-223).

However, the goal of artificial synthetic embryos is to establish embryo-like structures without any donated germ cells, zygotes or blastocysts. Several studies attempted to make synthetic embryos by co-aggregations of two or three different cell types [see e.g. Zhang et al. (2020) Front. Bioeng. Biotechnol. 8:781]. For example, Harrison et al. [Science (2017) DOI:10.1126/science.aa1810] used in vitro expanded mouse PSCs, non-pluripotent conventional trophoblast stem cells (TSCs), Sozen et al. (Nature Cell Biology volume 20, pages 979-989 (2018)) used in vitro expanded mouse PSCs, non-pluripotent conventional trophoblast stem cells (TSCs) and non-pluripotent extra embryonic endodermal cells (referred to as XEN cells); while Amadei et al. [Developmental Cell (2021) 56, 366-382] used naïve iPSCs, non-pluripotent conventional TSC lines and mouse ES cells that transiently overexpress Gata4 transcription factor that helps promote primitive endoderm fate (instead of using stable mouse XEN lines). However, the embryos obtained were heavily aberrant and could not proceed through or complete gastrulation or initiate organogenesis and had aberrant morphology and missing key embryo defining features (DOI: 10.1126/sciadv.add2913).

### SUMMARY OF THE INVENTION

According to an aspect of some embodiments of the present invention there is provided a method of generating a synthetic embryo, the method comprising:
(a) inducing expression of a factor that induces differentiation to trophectoderm cells in a subpopulation of naïve pluripotent stem cells (PSCs) to obtain trophectoderm primed cells; and
(b) mixing the trophectoderm primed cells with naïve PSCs under conditions that allow formation of aggregated cells,
wherein when the factor that induces differentiation to trophectoderm cells is an exogenous factor the inducing the expression comprises inducing transient expression starting within 14 days-0 hours prior to the mixing and ending no later than 120 hours following the mixing; and
wherein the factor that induces differentiation to trophectoderm cells is an endogenous factor the inducing the expression comprises culturing the subpopulation of naïve PSCs under conditions enabling expression of the factor starting within 14 days-6 hours prior to the mixing,
thereby generating the synthetic embryo.

According to some embodiments of the invention, the method further comprising inducing expression of a factor that induces differentiation to extra embryonic primitive endodermal cells in a second subpopulation of naïve PSCs to obtain extra embryonic primitive endodermal primed cells in the (a); and mixing the extra embryonic primitive endodermal primed cells with the cells in the (b).

According to some embodiments of the invention, the wherein when the factor that induces differentiation to extra embryonic primitive endodermal cells is an exogenous factor the inducing the expression comprises inducing transient expression starting within 14 days-0 hours prior to the mixing and ending no later than 120 hours following the mixing; and
wherein the factor that induces differentiation to extra embryonic primitive endodermal cells is an endogenous factor the inducing the expression comprises culturing the subpopulation of naïve PSCs under conditions enabling expression of the factor starting within 14 days-6 hours prior to the mixing.

According to an aspect of some embodiments of the present invention there is provided a method of generating a synthetic embryo, the method comprising:
(a) inducing transient expression of an exogenous factor that induces differentiation to trophectoderm cells in a subpopulation of naïve pluripotent stem cells (PSCs) to obtain trophectoderm primed cells; and
(b) mixing the trophectoderm primed cells with naïve PSCs under conditions that allow formation of aggregated cells,
wherein the inducing the transient expression starts within 14 days-0 hours prior to the mixing and ends no later than 120 hours following the mixing,
thereby generating the synthetic embryo.

According to some embodiments of the invention, the method further comprising inducing transient expression of an exogenous factor that induces differentiation to extra embryonic primitive endodermal cells in a second subpopulation of naïve PSCs to obtain extra embryonic primitive endodermal primed cells in the (a); and mixing the extra embryonic primitive endodermal primed cells with the cells in the (b).

According to some embodiments of the invention, mixing the naïve PSCs, the trophectoderm primed cells and the extra embryonic primitive endodermal primed cells is effected concomitantly.

According to some embodiments of the invention, mixing the naïve PSCs and the extra embryonic primitive endodermal primed cells is effected prior to the mixing with the trophectoderm primed cells.

According to some embodiments of the invention, the trophectoderm cells and/or the extra embryonic primitive endodermal primed cells express a pluripotency marker at the 0 hours.

According to some embodiments of the invention, the trophectoderm cells and/or the extra embryonic primitive endodermal primed cells express a pluripotency marker at the mixing.

According to some embodiments of the invention, the trophectoderm primed cells express a trophectoderm marker at the 0 hours.

According to some embodiments of the invention, the trophectoderm primed cells express a trophectoderm marker at the mixing.

According to some embodiments of the invention, the extra embryonic endodermal primed cells express an extra embryonic primitive endoderm marker at the 0 hours.

According to some embodiments of the invention, the extra embryonic endodermal primed cells express an extra embryonic primitive endoderm marker at the mixing. According to an aspect of some embodiments of the present invention there is provided an article of manufacture comprising:
(i) trophectoderm primed cells, wherein the trophectoderm primed cells comprise naïve pluripotent stem cell (PSCs) comprising an exogenous polynucleotide suitable for transient expression of a factor that induces differentiation to trophectoderm cells; and
(ii) naïve PSCs.

According to some embodiments of the invention, the article of manufacture further comprising:
(iii) extra embryonic primitive endodermal primed cells, wherein the extra embryonic primitive endodermal primed cells comprise naïve pluripotent stem cells (PSCs) comprising an exogenous polynucleotide suitable for transient expression of factor that induces differentiation to extra embryonic primitive endodermal cells.

According to an aspect of some embodiments of the present invention there is provided an article of manufacture comprising:
(i) trophectoderm primed cells, wherein the trophectoderm primed cells comprise naïve pluripotent stem cells (PSCs) comprising an exogenous polynucleotide suitable for transient expression of a factor that induces differentiation to trophectoderm cells; and
(ii) extra embryonic primitive endodermal primed cells, wherein the extra embryonic primitive endodermal primed cells comprise naïve pluripotent stem cells (PSCs) comprising an exogenous polynucleotide suitable for transient expression of factor that induces differentiation to extra embryonic primitive endodermal cells.

According to some embodiments of the invention, the (i) the (ii) and the (iii) are in a single container.

According to some embodiments of the invention, the (i) the (ii) and the (iii) are in separate containers.

According to an aspect of some embodiments of the present invention there is provided a method of ex-utero culturing a synthetic embryo, the method comprising mixing the cells of the article of manufacture under conditions the allow formation of aggregated cells, wherein the method comprises inducing transient expression of the factor starting within 14 days-0 hours prior to the mixing and ending no later than 120 hours following the mixing, thereby generating the synthetic embryo.

According to an aspect of some embodiments of the present invention there is provided a mixture or aggregate, obtainable by the method.

According to an aspect of some embodiments of the present invention there is provided a mixture or aggregate comprising:
(i) naïve pluripotent stem cell (PSCs);
(ii) trophectoderm primed cells, wherein the trophectoderm primed cells comprise stem cells expressing an endogenous trophectoderm marker and an endogenous pluripotent marker; and
(iii) extra embryonic primitive endodermal primed cells, wherein the extra embryonic primitive endodermal primed cells comprise stem cells expressing an endogenous extra embryonic primitive endoderm marker and an endogenous pluripotency marker,
wherein the mixture or aggregate is not structurally organized as a native embryo at any developmental stage; and
wherein the mixture or aggregate is capable of forming a synthetic embryo structurally organized as a native embryo of at least a post-implantation egg cylinder or a bilaminar disc developmental stage.

According to an aspect of some embodiments of the present invention there is provided a mixture or aggregate, comprising:
(i) trophectoderm primed cells, wherein the trophectoderm primed cells comprise stem cells expressing an exogenous factor that induces differentiation to trophectoderm cells, an endogenous trophectoderm marker and an endogenous pluripotent marker; and
(ii) naïve pluripotent stem cell (PSCs).

According to some embodiments of the invention, the mixture or aggregate further comprising:
(iii) extra embryonic primitive endodermal primed cells, wherein the extra embryonic primitive endodermal primed cells comprise stem cells expressing an exogenous factor that induces differentiation to extra embryonic primitive endodermal cells, an endogenous extra embryonic primitive endoderm marker and an endogenous pluripotency marker.

According to some embodiments of the invention, the factor that induces differentiation to extra embryonic primitive endodermal cells comprises Gata4, Gata6 and/or SOX17.

According to some embodiments of the invention, the factor that induces differentiation to extra embryonic primitive endodermal cells comprises Gata4 and/or Gata6.

According to some embodiments of the invention, inducing expression of a factor that induces differentiation to extra embryonic primitive endodermal cells comprises culturing the second subpopulation of naïve PSCs with a factor that induces differentiation to extra embryonic primitive endodermal cells starting within 14 days-6 hours prior to the mixing.

According to some embodiments of the invention, the method further comprises contacting the second subpopulation of naïve PSCs with a factor that induces differentiation to extra embryonic primitive endodermal cells within 14 days-0 hours prior to the mixing.

According to some embodiments of the invention, the factor that induces differentiation to extra embryonic primitive endodermal cells is selected from the group consisting of a GSK-3 inhibitor, WNT ligand, heparin, FGF2, FGF4, PDGF, leukemia inhibitory factor (LIF) and insulin.

According to some embodiments of the invention, the factor that induces differentiation to extra embryonic primitive endodermal cells is selected from the group consisting of a GSK-3 inhibitor, WNT ligand, heparin, FGF2 and FGF4.

According to some embodiments of the invention, the factor that induces differentiation to trophectoderm cells is selected from the group consisting of Cdx2, Gata3 and Gata2.

According to some embodiments of the invention, inducing expression of a factor that induces differentiation to trophectoderm cells comprises culturing the subpopulation of naïve PSCs with a factor that induces differentiation to trophectoderm cells starting within 14 days-6 hours prior to the mixing.

According to some embodiments of the invention, the method further comprises contacting the subpopulation of naïve PSCs with a factor that induces differentiation to trophectoderm cells within 14 days-0 hours prior to the mixing.

According to some embodiments of the invention, the factor that induces differentiation to trophectoderm cells is selected from the group consisting of TGFR inhibitor, FGFR inhibitor, MEK/ERK inhibitor, BMP4, JAK inhibitor, a ROCK pathway inhibitor, FGF4, FGF2, heparin, a SUMOylation inhibitor, a Histone Deacetylase inhibitor, a HIPPO signaling pathway inhibitor and a factor that induces YAP nuclear translocation.

According to some embodiments of the invention, the factor that induces differentiation to trophectoderm cells is selected from the group consisting of FGF4, FGF2, heparin, a SUMOylation inhibitor, a Histone Deacetylase inhibitor, a HIPPO signaling pathway inhibitor and a factor that induces YAP nuclear translocation.

According to some embodiments of the invention, the transient expression is effected by introducing a polynucleotide encoding the factor.

According to some embodiments of the invention, the polynucleotide is a DNA.

According to some embodiments of the invention, the expression of the polynucleotide is regulated by an inducible promoter.

According to some embodiments of the invention, the introducing is by transient transfection.

According to some embodiments of the invention, the polynucleotide is a RNA.

According to some embodiments of the invention, the inducing the transient expression starts within 120-0 hours prior to the mixing

According to some embodiments of the invention, the inducing the transient expression starts within 48-0 hours prior to the mixing.

According to some embodiments of the invention, the inducing the transient expression starts within 24-0 hours prior to the mixing.

According to some embodiments of the invention, the inducing the transient expression starts within 48-12 hours prior to the mixing.

According to some embodiments of the invention, the inducing the transient expression ends no later than 48 hours following the mixing.

According to some embodiments of the invention, the inducing the transient expression is effected for no more than 96 hours.

According to some embodiments of the invention, the inducing the expression of the endogenous factor starts within 10-1 days prior to the mixing.

According to some embodiments of the invention, a ratio between the naïve PSCs and the trophectoderm primed cells is between 1:1 and 1:5.

According to some embodiments of the invention, a ratio between the naïve PSCs and the trophectoderm primed cells is 1:3.

According to some embodiments of the invention, a ratio between the naïve PSCs and the primitive endodermal primed cells is between 1:1 and 2:1.

According to some embodiments of the invention, a ratio between the naïve PSCs and the primitive endodermal primed cells is 1:1.

According to some embodiments of the invention, the method further comprising ex-utero culturing the aggregated cells following the (b).

According to an aspect of some embodiments of the present invention there is provided a method of ex-utero culturing a synthetic embryo, the method comprising ex-utero culturing the mixture or aggregate.

According to some embodiments of the invention, the culturing is effected in a static culture followed by a dynamic culture.

According to some embodiments of the invention, the static culture is effected until the embryo reaches at least an early post-implantation egg-cylinder or bilaminar discstage.

According to some embodiments of the invention, the dynamic culture starts the latest when the embryo reaches an early somite stage.

According to an aspect of some embodiments of the present invention there is provided a method of ex-utero culturing a synthetic embryo, the method comprising ex-utero culturing a synthetic embryo at an aggregate stage in a static culture followed by a dynamic culture, wherein the static culture is effected until the embryo reaches at least an early post-implantation egg-cylinder or bilaminar disc stage; and the dynamic culture starts the latest when the embryo reaches an early somite stage.

According to some embodiments of the invention, the dynamic culture comprises a shaker culture, a roller culture or a sequential combination thereof.

According to some embodiments of the invention, the dynamic culture is effected under a hyperbaric pressure of more than 0.1 and less than 10.2 pounds per square inch (psi).

According to some embodiments of the invention, the dynamic culture is effected under a hyperbaric pressure of more than 5 and less than 10.2 pounds per square inch (psi).

According to some embodiments of the invention, the culturing is effected in an atmosphere comprising 10-40% oxygen.

According to some embodiments of the invention, the culturing is effected with a medium comprising increasing concentrations of serum or serum replacement throughout the culturing.

According to some embodiments of the invention, the culturing is effected with a medium comprising at least 15% serum or serum replacement from the beginning until the embryo reaches an early post-implantation egg-cylinder or bilaminar disc stage and at least 30% serum or serum replacement thereafter.

According to some embodiments of the invention, the culturing is effected with a medium comprising a human serum for at least part of the culturing.

According to some embodiments of the invention, the method further comprising transferring the synthetic embryo in-utero.

According to some embodiments of the invention, the cells are mammalian cells.

According to some embodiments of the invention, the mammalian cells are mouse cells.

According to some embodiments of the invention, the mammalian cells are human cells.

According to some embodiments of the invention, the mammalian cells are non-human primate cells.

According to some embodiments of the invention, the mammalian cells are live stock cells.

According to an aspect of some embodiments of the present invention there is provided a synthetic embryo obtainable by the method.

According to an aspect of some embodiments of the present invention there is provided a method of obtaining extra embryonic primitive endodermal primed cells, the method comprising culturing naïve PSCs in a base medium devoid of Activin A under conditions that allow differentiation of the naïve PSCs to GATA4⁺GATA6⁺OCT4⁺Nanog⁻ extra embryonic primitive endodermal primed cells, thereby obtaining the extra embryonic primitive endodermal primed cells.

According to some embodiments of the invention, the culturing is effected for at least 1 day.

According to some embodiments of the invention, the culturing is effected for up to 10 days.

According to some embodiments of the invention, the cells can be expanded in culture for at least five passages while maintaining the GATA4⁺GATA6⁺OCT4⁺Nanog⁻ phenotype.

According to some embodiments of the invention, the base medium is supplemented with at least one of a GSK-3 inhibitor, leukemia inhibitory factor (LIF) and insulin.

According to some embodiments of the invention, the base medium is supplemented with at least one of GSK-3 inhibitor, heparin, FGF4, PDGF and BSA.

According to some embodiments of the invention, the cells are human cells.

According to some embodiments of the invention, the cells are mouse cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows immunostaining images of E6.5 mouse embryo derived mouse TSC line #1 at passage 3 (day 14 since derivation), demonstrating expression of the TSC markers Cdx2 and Gata3. DAPI was used for counterstaining.

FIG. 2 shows immunostaining images of mouse TSC line #1 at passage 5 (day 25 since derivation), demonstrating expression of the TSC markers Cdx2, TBR2, Gata3 and TFAP2C and no expression of Oct4 and Nanog. DAPI was used for counterstaining.

FIGS. 3A-B show immunostaining images of parental ESC line (FIG. 3A) and mouse TSC line #2 at passage 3 (approximately day 12 since protocol initiation) (FIG. 3B) which was derived after transient Oct4 knockdown to the parental ES lines, demonstrating expression of the TSC marker Cdx2 and Gata3 and no expression of Oct4 in the established TSC line. DAPI was used for counterstaining.

FIG. 4 shows immunostaining images of mouse TSC line #2 at passage 5 (day 30 since protocol initiation), demonstrating expression of the TSC markers Cdx2, TBR2, Gata3 and TFAP2C and no expression of Oct4 and Nanog. DAPI was used for counterstaining.

FIG. 5 shows immunostaining images of mouse TSC line #3 at passage 4 (approximately day 24 since protocol initiation and transfection), demonstrating expression of the TSC markers Cdx2 and Gata3 and TFAP2C and no expression of Oct4. DAPI was used for counterstaining.

FIG. 6A shows immunostaining images of mouse XEN line #7 at passage 5, demonstrating expression of the XEN markers Foxa2 and Gata4 and no expression of Oct4 and Nanog. DAPI was used for counterstaining.

FIG. 6B shows RNA expression levels of the XEN markers Gata6, PDGFRA, Sox 17 and Gata4 in the mouse XEN lines XEN-ICR clone #7 and XEN-Prep1, as determined by real-time PCR analysis. Mouse V6.5 naïve ESCs expanded in 2i-LIF conditions were used as a reference control (value set as 1).

FIGS. 7A-C demonstrate that co-aggregation of naïve mouse PSCs (V6.5-GFP), mouse XEN cells (mouse XEN line #7 cells) and mouse TSCs (mouse TSC line #1 cells) followed by ex-utero culturing does not lead to generation of an organized embryo. FIG. 7A is a schematic representation of the protocol. Aggregation medium (AM)—1:1 DMEM-F12 (Thermo 11320033) & Neurobasal (Thermo 21103049), Glutamax 1×, Sodium pyruvate 1 mM, Pen/Strep 1×, NEAA 1×, N2 supplement 1× (Thermo 17502048), B27 supplement 1× (Thermo 17504044), beta-mercaptoethanol 0.1 mM (Thermo 31350010) and 0.45% BSA (Sigma Aldrich A7979) (For 500 ml Total). IVC1 medium—Advanced DMEM/F12 (Gibco 12634010) Glutamax 1×, Pen/Strep 1×, ITS-X 1× (Gibco 51500056), beta-Estradiol 8 nM, Progesterone 20 ng/ml, N-acetyl-L-cysteine 25 μM and serum as indicated in the scheme [For 500 ml total]. EUCM—25% DMEM (GIBCO 11880), 25% HAS (Human Adult Serum or Human cord serum), 50% Rat Serum (RAS), Glutamax 1× (GIBCO 35050038), HEPES 2 mM (GIBCO 15630056), Pen/Strep 1×. FIG. 7B shows images of representative embryoid aggregates at Day 4 of the protocol immunostained for TBR2 (red) which marks trophectoderm lineage, Gata4 which marks primitive endoderm lineage, and GFP signals marked the labeled ESCs contributing to the epiblast. FIG. 7C shows images or representative structures at Day 7 of the protocol demonstrating no organized embryos in the yolk sac with no signs of emergence of neural folds and cardiac regions.

FIGS. 7D-F demonstrate that co-aggregation of naïve mouse PSCs (V6.5-GFP), mouse KH2-Gata4 Dox inducible ES cells and mouse TSCs (mouse TSC line #1 cells) followed by ex-utero culturing does not lead to generation of an organized embryo. FIG. 7D is a schematic representation of the protocol. Aggregation medium (AM)—1:1 DMEM-F12 (Thermo 11320033) & Neurobasal (Thermo 21103049), Glutamax 1×, Sodium pyruvate 1 mM, Pen/Strep 1×, NEAA 1×, N2 supplement 1× (Thermo 17502048), B27 supplement 1× (Thermo 17504044), Beta-mercaptoethanol 0.1 mM (Thermo 31350010), 0.45% BSA (SigmaAldrich A7979) (For 500 ml Total). IVC1 medium—Advanced DMEM/F12 (Gibco 12634010) Glutamax 1×, Pen/Strep 1×, ITS-X 1× (Gibco 51500056), beta-Estradiol 8 nM, Progesterone 20 ng/ml, N-acetyl-L-cysteine 25 μM and serum or KSR as indicated in the scheme (For 500 ml total). EUCM—25% DMEM (GIBCO 11880), 25% HAS (Human Adult Serum or Human cord serum), 50% Rat Serum (RAS), Glutamax 1× (GIBCO 35050038), HEPES 2 mM (GIBCO 15630056), Pen/Strep 1×. FIG. 7E shows images of representative embryoid aggregates at Days 5 and 6 of the protocol immunostained for TBR2 (red) which marks trophectoderm lineage, Gata4 which marks primitive endoderm lineage, and GFP signals marked the labeled ESCs contributing to the epiblast. FIG. 7F shows images or representative structures at Days 7 and 8 of the protocol demonstrating no organized embryos in the yolk sac with no signs of emergence of neural folds and cardiac regions.

FIG. 8 shows a schematic representation of the generation and validation of KH2-Gata4 Dox inducible naïve ES cells.

FIG. 9 shows Real-Time PCR analysis of exogenous GATA4 expression in different KH2-Gata4 mouse ES clones targeted with TetOn-Gata4 in the collagen locus after treatment with DOX. KH2-WT and mouse irradiated fibroblast cells (MEFs) were used as negative control.

FIG. 10 shows immunostaining images of different KH2 mouse naïve ES clones targeted with TetOn-Gata4 (KH2-Gata4) in the collagen locus before and after treatment with DOX in naïve 2i/LIF conditions. Gata4 was robustly expressed in the tested clones only after DOX treatment. DAPI was used for counterstaining.

FIG. 11 shows a schematic representation of the generation and validation of KH2-Cdx2 Dox inducible cells.

FIG. 12 shows Real-Time PCR analysis of exogenous Cdx2 expression in different KH2-Cdx2 mouse ES clones targeted with TetOn-Cdx2 in the collagen locus after treatment with DOX. KH2-WT cells were used as negative control.

FIG. 13 shows immunostaining KH2 mouse naïve ES clone #3 targeted with TetOn-Cdx2 (KH2-Cdx2) in the collagen locus before and after treatment with DOX in naïve 2i/LIF conditions. Cdx2 was robustly expressed in the tested clone only after DOX treatment. DAPI was used for counterstaining.

FIG. 14 shows immunostaining KH2 mouse naïve ES clone #7 targeted with TetOn-Cdx2 (KH2-Cdx2) in the collagen locus before and after treatment with DOX in naïve 2i/LIF conditions. Cdx2 was robustly expressed in the tested clone only after DOX treatment. DAPI was used for counterstaining.

FIGS. 15A-F demonstrate that co-aggregation of naïve KH2-WT ES cells with DOX pre-treated KH2-Gata4 naïve ES cells and DOX pre-treated KH2-Cdx2 naïve ES cells, followed by ex-utero culturing leads to generation of an organized embryo. FIG. 15A is a schematic representation of the protocol. Aggregation medium (AM)—1:1 DMEM-F12 (Thermo 21331-020) & Neurobasal (Thermo 21103049), Glutamax 1×, Sodium pyruvate 1 mM, Pen/Strep 1×, NEAA 1×, N2 supplement 1× (Thermo 17502048), B27 supplement 1× (Thermo 17504044), beta-mercaptoethanol 0.1 mM (Thermo 31350010), 0.45% BSA (SigmaAldrich A7979) (For 500 ml Total), EUCM2-CMRL—CMRL (Gibco 11530037), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 20% FBS (For 500 ml total). EUCM3-CMRL—CMRL (Gibco 11530037), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 30% FBS (For 500 ml total). EUCM4-CMRL—CMRL (Gibco 11530037), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 30% HAS (For 500 ml total). EUCM—25% DMEM (GIBCO 11880), 25% HAS, 50% Rat Serum, Glutamax 1× (GIBCO 35050038), HEPES 2 mM (GIBCO 15630056), Pen/Strep 1×. FIG. 15B shows phase contrast images of the synthetic embryos obtained at the indicated time points. At Days 4 and 5, embryos with clear egg cylinder shape are evident, with extra embryonic ectoderm (EXE) appearing on the opposite side. At Day 6.5, Yolk sac and ectoplacental cone are evident with embryo-like structure engulfed inside). At Day 7, an embryo that completed gastrulation with evident neural folds and somite formation and beating heart was evident. FIGS. 15C-F show immunostaining images of the synthetic embryos obtained. The embryo obtained at Day 7 of the protocol corresponds to an E8.5 stage embryo (FIGS. 15C-D). In FIG. 15C, the embryo demonstrates neural folds staining positive for Sox2 marker (red) and brachyury (green) staining for mesoderm. In FIG. 15D, the embryo demonstrates neural folds staining positive for Pax6 marker (green), and Sox 17 marker (red) that marks the developing endodermal gut. The embryo obtained at Day 8 of the protocol corresponds to an E9.5 stage embryo (FIGS. 15E-F). In FIG. 15E, the embryo demonstrates neural tube closure and somite formation (positively stained for Sox9 in Red). Otx2 (magenta) marks forebrain region. In FIG. 15F, the embryo demonstrates advanced organ formation and normal morphology with adequate distribution of neural brain cells positively stained with Tuj1 marker (green).

FIGS. 16A-B demonstrate that co-aggregation of KH2-WT naïve ES cells with naïve KH2-Gata4 and KH2-Cdx2 naïve cells in the presence of Dox, followed by ex-utero culturing leads to generation of an organized embryo. FIG. 16A is a schematic representation of the protocol. Aggregation media (AM)—1:1 DMEM-F12 (Thermo 11320033) & Neurobasal (Thermo 21103049), Glutamax 1×, Sodium pyruvate 1 mM, Pen/Strep 1×, NEAA 1×, N2 supplement 1× (Thermo 17502048), B27 supplement 1× (Thermo 17504044), beta-mercaptoethanol 0.1 mM (Thermo 31350010), 0.45% BSA (SigmaAldrich A7979) (For 500 ml Total). EUCM2-CMRL—CMRL (Gibco 11530037), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 20% FBS (For 500 ml total). EUCM3-CMRL-CMRL (Gibco 11530037), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 30% FBS (For 500 ml total). EUCM4-CMRL-CMRL (Gibco 11530037), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 30% HAS (For 500 ml total). EUCM-25% DMEM (GIBCO 11880), 25% HAS, 50% Rat Serum (RAS), Glutamax 1× (GIBCO 35050038), HEPES 2 mM (GIBCO 15630056), Pen/Strep 1×. FIG. 16B shows phase contrast images of the synthetic embryos obtained at the indicated time points. At Days 4 and 6, embryos with clear egg cylinder shape were evident, and with extra embryonic ectoderm (EXE) appearing on the opposite side. At Day 6.5, Yolk sac and ectoplacental cone were evident with embryo-like structure engulfed in side. At Day 7, an embryo that completed gastrulation with evident neural folds and early somite formation and beating heart was evident.

FIGS. 17A-B demonstrate that co-aggregation of KH2-WT naïve ES cells with Dox pre-treated naïve KH2-Gata4 ES cells and Dox pre-treated KH2-Cdx2 naïve ES cells, followed by ex-utero culturing leads to generation of an organized embryo. FIG. 17A is a schematic representation of the protocol. Aggregation media (AM)—1:1 DMEM-F12 (Thermo 11320033) & Neurobasal (Thermo 21103049), Glutamax 1×, Sodium pyruvate 1 mM, Pen/Strep 1×, NEAA 1×. N2 supplement 1× (Thermo 17502048), B27 supplement 1× (Thermo 17504044), beta-mercaptoethanol 0.1 mM (Thermo 31350010), 0.45% BSA (SigmaAldrich A7979) (For 500 ml Total). EUCM2-CMRL—CMRL (Gibco 11530037), Glutamax 1×, Sodium pyruvate 1 mM. Pen/Strep 1×+NEAA 1×, D-Glucose 4 mg/mL, ITS-X 1× (Gibco 51500056), beta-Estradiol 8 nM, Progesterone 20 ng/ml, 20% FBS (For 500 ml total). EUCM3-CMRL—CMRL (Gibco 11530037), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 30% FBS (For 500 ml total). EUCM4-CMRL—CMRL (Gibco 11530037), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 30% HAS (For 500 ml total). EUCM—25% DMEM (GIBCO 11880), 25% HAS, 50% RAS, Glutamax 1× (GIBCO 35050038), HEPES 10 mM (GIBCO 15630056), Pen/Strep 1×. FIG. 17B shows phase contrast images of the synthetic embryos obtained at the indicated time points. At Day 6, embryos with clear egg cylinder shape were evident, and with extra embryonic ectoderm (EXE) appearing on the opposite side. At Day 6.5. Yolk sac and ectoplacental cone were evident with embryo-like structure engulfed inside. At Day 7, an embryo that completed gastrulation with evident neural folds and early somite formation and beating heart was evident equivalent to E8.5.

FIGS. 18A-B demonstrate that co-aggregation of KH2-WT naïve ES, cells with KH2-Gata4 naïve ES cells and KH2-Cdx2 naïve ES cells in the presence of Dox, followed by ex-utero culturing leads to generation of an organized embryo. FIG. 18A is a schematic representation of the protocol. Aggregation media (AM)—1:1 DMEM-F12 (Thermo 11320033) & Neurobasal (Thermo 21103049), Glutamax 1×, Sodium pyruvate 1 mM, Pen/Strep 1×, NEAA 1×, N2 supplement 1× (Thermo 17502048), B27 supplement 1× (Thermo 17504044), beta-mercaptoethanol 0.1 mM (Thermo 31350010), 0.45% BSA (SigmaAldrich A7979) (For 500 ml Total). EUCM2-CMRL-advDMEM/F12—1:1 mix of CMRL (Gibco 11530037) & Advanced DMEM/F12 (Thermo 12634010), Glutamax 1×, Sodium pyruvate 1 mM, Pen/Strep 1×+NEAA 1×, D-Glucose 1 mg/mL, ITS-X 1× (Gibco 51500056), beta-Estradiol 8 nM, Progesterone 20 ng/ml, 20% FBS (For 500 ml total). EUCM3-CMRL-advDMEM/F12—1:1 mix of CMRL (Gibco 11530037) & Advanced DMEM/F12 (Thermo 12634010), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 30% FBS (For 500 ml total). EUCM4-CMRL-advDMEM/F12—1:1 mix of CMRL (Gibco 11530037) & Advanced DMEM/F12 (Thermo 12634010), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 30% HAS (For 500 ml total). EUCM—25% DMEM (GIBCO 11880), 25% HAS, 50% RAS, Glutamax 1× (GIBCO 35050038), HEPES 10 mM (GIBCO 15630056), Pen/Strep 1×. FIG. 18B shows phase contrast images of the synthetic embryos obtained at the indicated time points. At Days 4, 5 and 6, embryos with clear egg cylinder shape were evident, and with extra embryonic ectoderm (EXE) appearing on the opposite side. At Day 6.5, Yolk sac, ectoplacental cone and umbilical cord invagination were evident with embryo-like structure engulfed inside. At Day 7, an embryo that completed gastrulation with evident neural folds and early somite formation and beating heart was evident.

FIGS. 19A-B demonstrate that co-aggregation of KH2-WT naïve ES cells with Dox pre-treated KH2-Gata4 naïve ES and Dox pre-treated KH2-Cdx2 naïve ES cells, followed by ex-utero culturing leads to generation of an organized embryo. FIG. 19A is a schematic representation of the protocol. Aggregation media (AM)—1:1 DMEM-F12 (Thermo 11320033) & Neurobasal (Thermo 21103049), Glutamax 1×, Sodium pyruvate 1 mM, Pen/Strep 1×, NEAA 1×, N2 supplement 1× (Thermo 17502048), B27 supplement 1× (Thermo 17504044), beta-mercaptoethanol 0.1 mM (Thermo 31350010), 0.45% BSA (SigmaAldrich A7979) (For 500 ml Total). IVC1—Advanced DMEM/F12 (Gibco 12634010) Glutamax 1×, Pen/Strep 1×, ITS-X 1× (Gibco 51500056), beta-Estradiol 8 nM, Progesterone 20 ng/ml, N-acetyl-L-cysteine 25 μM and FBS or HAS as indicated (For 500 ml total). EUCM—25% DMEM (GIBCO 11880), 25% HAS, 50% RAS, Glutamax 1× (GIBCO 35050038), HEPES 2 mM (GIBCO 15630056), Pen/Strep 1×. FIG. 19B shows phase contrast images of the synthetic embryos obtained at the indicated time points. At Day 6, embryos with clear egg cylinder shape were evident, and with extra embryonic ectoderm (EXE) appearing on the opposite side. At Day 6.5, Yolk sac and ectoplacental cone were evident with embryo-like structure engulfed in side.

FIGS. 20A-B demonstrate that co-aggregation of KH2-WT naïve ES cells with Dox pre-treated KH2-Gata4 and Dox+FGF4+LPA pre-treated KH2-Cdx2 naïve ES cells, followed by ex-utero culturing leads to generation of an organized embryo. FIG. 20A is a schematic representation of the protocol. Aggregation media (AM)—1:1 DMEM-F12 (Thermo 11320033) & Neurobasal (Thermo 21103049), Glutamax 1×, Sodium pyruvate 1 mM, Pen/Strep 1×, NEAA 1×, N2 supplement 1× (Thermo 17502048), B27 supplement 1× (Thermo 17504044), beta-mercaptoethanol 0.1 mM (Thermo 31350010), 0.45% BSA (SigmaAldrich A7979) (For 500 ml Total). EUCM2-CMRL-advDMEM/F12—1:1 mix of CMRL (Gibco 11530037) & Advanced DMEM/F12 (Thermo 12634010), Glutamax 1×, Sodium pyruvate 0.5 mM, Pen/Strep 1×+NEAA 0.5×, D-Glucose 4 mg/mL, ITS-X 1× (Gibco 51500056), beta-Estradiol 8 nM, Progesterone 20 ng/ml, 20% FBS (For 500 ml total). EUCM3-CMRL-advDMEM/F12—1:1 mix of CMRL (Gibco 11530037) & Advanced DMEM/F12 (Thermo 12634010), Glutamax 1×, Sodium pyruvate 0.5 mM, Pen/Strep 1×+NEAA 0.5×, D-Glucose 4 mg/mL, ITS-X 1× (Gibco 51500056), beta-Estradiol 8 nM, Progesterone 20 ng/ml, 30% FBS (For 500 ml total). EUCM4-CMRL-advDMEM/F12—1:1 mix of CMRL (Gibco 11530037) & Advanced DMEM/F12 (Thermo 12634010), Glutamax 1×, Sodium pyruvate 0.5 mM, Pen/Strep 1×+NEAA 0.5×, D-Glucose 4 mg/mL, ITS-X 1× (Gibco 51500056), beta-Estradiol 8 nM, Progesterone 20 ng/ml, 30% HAS (For 500 ml total). EUCM—25% DMEM (GIBCO 11880), 25% HAS, 50% RAS, Glutamax 1× (GIBCO 35050038), HEPES 2 mM (GIBCO 15630056), Pen/Strep 1×. FIG. 20B shows phase contrast images of the synthetic embryos obtained at the indicated time points. At Day 6, embryos with clear egg cylinder shape and amnion were evident, and with extra embryonic ectoderm (EXE) appearing on the opposite side. At Day 6.5, Yolk sac and ectoplacental cone and umbilical cord invagination were evident with embryo-like structure engulfed inside. At Day 7, an embryo that completed gastrulation with evident neural folds and early somite formation and beating heart was evident.

FIG. 21 shows a schematic protocol for co-aggregation of KH2-WT naïve cells with Dox+CHIR99021 pre-treated KH2-Gata4 naïve cells and Dox+LPA pre-treated KH2-Cdx2 naïve cells, followed by ex-utero culturing that leads to generation of an organized embryo. Aggregation media (AM)—1:1 DMEM-F12 (Thermo 11320033) & Neurobasal (Thermo 21103049), Glutamax 1×, Sodium pyruvate 1 mM, Pen/Strep 1×, NEAA 1×, N2 supplement 1× (Thermo 17502048), B27 supplement 1× (Thermo 17504044), beta-mercaptoethanol 0.1 mM (Thermo 31350010), 0.45% BSA (SigmaAldrich A7979) (For 500 ml Total). EUCM2-CMRL-advDMEM/F12—1:4 mix of CMRL (Gibco 11530037) & Advanced DMEM/F12 (Thermo 12634010), Glutamax 1×, Sodium pyruvate 0.5 mM, Pen/Strep 1×+NEAA 0.5×, extra D-Glucose 2 mg/mL, ITS-X 1× (Gibco 51500056), beta-Estradiol 8 nM, Progesterone 20 ng/ml, 20% FBS (For 500 ml total). EUCM3-CMRL-advDMEM/F12—4:1 mix of CMRL (Gibco 11530037) & Advanced DMEM/F12 (Thermo 12634010), Glutamax 1×, Sodium pyruvate 0.5 mM, Pen/Strep 1×+NEAA 0.5×, +Extra D-Glucose 2 mg/mL, ITS-X 1× (Gibco 51500056), beta-Estradiol 8 nM, Progesterone 20 ng/ml, 30% FBS (For 500 ml total). EUCM4*-CMRL-advDMEM/F12—4:1 mix of CMRL (Gibco 11530037) & Advanced DMEM/F12 (Thermo 12634010), Glutamax 1×, Sodium pyruvate 0.5 mM, Pen/Strep 1×+NEAA 0.5×, extra D-Glucose 3 mg/mL, ITS-X 1× (Gibco 51500056), beta-Estradiol 8 nM, Progesterone 20 ng/ml, 20% RAS, 10% HAS, 10% KSR, HEPES 2 mM (GIBCO 15630056) (For 500 ml total). EUCM5-CMRL-advDMEM/F12—4:1 mix of CMRL (Gibco 11530037) & Advanced DMEM/F12 (Thermo 12634010), Glutamax 1×, Sodium pyruvate 0.5 mM, Pen/Strep 1×+NEAA 0.5×, +extra D-Glucose 3 mg/mL, ITS-X 1× (Gibco 51500056), beta-Estradiol 8 nM, Progesterone 20 ng/ml, 30% RAS, 15% HAS, 5% KSR, HEPES 2 mM (GIBCO 15630056) (For 500 ml total). EUCM—25% Advanced DMEM/F12 (Thermo 12634010), 25% HAS, 50% RAS, Glutamax 1× (GIBCO 35050038), HEPES 2 mM (GIBCO 15630056), Pen/Strep 1×.

FIG. 22 shows a schematic protocol for co-aggregation of KH2-WT naive ES cells with Dox+CHIR99021 pre-treated KH2-Gata4 naïve ES cells and Dox+LPA pre-treated KH2-Cdx2 naïve cells, followed by ex-utero culturing that leads to generation of an organized embryo. Aggregation media (AM)—1:1 DMEM-F12 (Thermo 11320033) & Neurobasal (Thermo 21103049), Glutamax 1×, Sodium pyruvate 1 mM, Pen/Strep 1×, NEAA 1×, N2 supplement 1× (Thermo 17502048), B27 supplement 1× (Thermo 17504044), beta-mercaptoethanol 0.1 mM (Thermo 31350010), 0.45% BSA (SigmaAldrich A7979) (For 500 ml Total). EUCM2-CMRL-advDMEM/F12:1:4 mix of CMRL (Gibco 11530037) & Advanced DMEM/F12 (Thermo 12634010), Glutamax 1×, Sodium pyruvate 0.5 mM, Pen/Strep 1×+NEAA 0.5×, extra D-Glucose 2 mg/mL, ITS-X 1× (Gibco 51500056), beta-Estradiol 8 nM, Progesterone 20 ng/ml, 20% FBS (For 500 ml total). EUCM3-CMRL-advDMEM/F12:4:1 mix of CMRL (Gibco 11530037) & Advanced DMEM/F12 (Thermo 12634010), Glutamax 1×, Sodium pyruvate 0.5 mM, Pen/Strep 1×+NEAA 0.5×, +Extra D-Glucose 2 mg/mL, ITS-X 1× (Gibco 51500056), beta-Estradiol 8 nM, Progesterone 20 ng/ml, 30% FBS (For 500 ml total). EUCM4*-CMRL-advDMEM/F12—4:1 mix of CMRL (Gibco 11530037) & Advanced DMEM/F12 (Thermo 12634010), Glutamax 1×, Sodium pyruvate 0.5 mM, Pen/Strep 1×+NEAA 0.5×, extra D-Glucose 3 mg/mL, ITS-X 1× (Gibco 51500056), beta-Estradiol 8 nM, Progesterone 20 ng/ml, 20% RAS, 10% HAS+HEPES 2 mM (GIBCO 15630056) (For 500 ml total). EUCM5-CMRL-advDMEM/F12—4:1 mix of CMRL (Gibco 11530037) & Advanced DMEM/F12 (Thermo 12634010), Glutamax 1×, Sodium pyruvate 0.5 mM, Pen/Strep 1×+NEAA 0.5×, +extra D-Glucose 3 mg/mL, ITS-X 1× (Gibco 51500056), beta-Estradiol 8 nM, Progesterone 20 ng/ml, 30% RAS, 15% HAS, HEPES 2 mM (GIBCO 15630056) (For 50 0 ml total). EUCM—25% Advanced DMEM/F12 (Thermo 12634010), 25% HAS, 50% RAS, Glutamax 1× (GIBCO 35050038), HEPES 2 mM (GIBCO 15630056), Pen/Strep 1×.

FIGS. 23A-D shows immunostaining images of representative synthetic embryos obtained according to the protocols described in Example 2 demonstrating generation of organized synthetic embryos. FIG. 23A shows immunostaining of representative day 4 mouse synthetic embryos from the indicated regimens. OTX2 (grey) was used to stain the primed pluripotent epiblast, Gata4 (red) was used to stain extraembryonic primitive endoderm progeny, and TFAP2C (green) to mark trophoblast lineage. Images show normal embryo structure with egg cylinder morphology, in which the trophoblast EXE and pluripotent epiblast self-organize on opposite sides pf the embryo as expected, and that Gata4 primitive endoderm cells surround the embryo. FIG. 23B shows immunostaining of representative day 4 mouse synthetic embryos from the indicated regimens. Oct4 (grey) was used to stain the primed pluripotent epiblast in the egg cylinder shaped epiblast, Sox 17 (red) was used to stain extraembryonic primitive endoderm progeny, and Cdx2 to mark trophoblast lineage. Images show normal embryo structure with egg cylinder morphology, in which the trophoblast EXE and pluripotent epiblast (Oct4+ region) self-organize to opposite sides of the Cdx2+ region, and that Sox17+primitive endoderm cells surround the embryos. FIG. 23C shows immunostaining of representative day 4 or day 5 mouse synthetic embryos from the indicated regimens. Oct4 (grey) was used to stain the primed pluripotent epiblast in the egg cylinder shaped epiblast, Sox17 (red) was used to stain extraembryonic primitive endoderm progeny, and Cdx2 to mark trophoblast lineage. Images show normal embryo structure with egg cylinder morphology, in which the trophoblast EXE and pluripotent epiblast (Oct4+ region) self-organize to opposite sides of the Cdx2+ region, and that Sox 17+primitive endoderm cells surround the embryos. FIG. 23D shows immunostaining of representative mouse synthetic embryos from the regimen shown in FIG. 21). Oct4 (Magenta) was used to stain the primed pluripotent epiblast in the egg cylinder shaped epiblast. T/Brachyury which marks the emergence of the primitive streak that is stained in Green. DAPI is in blue for counterstain. Day 6 egg cylinder shaped mouse embryoids show primitive streak formation and imitation of gastrulation at day 7 of the protocol as evident by specific immunostaining.

FIG. 24 demonstrates endogenous expression of the indicated markers in KH2-Gata4 naïve ES cells prior to and following Dox administration and in 4 Days synthetic embryos obtained according to the protocol described in FIG. 17A, as determined by Real-time PCR.

FIG. 25 demonstrates endogenous expression of the indicated markers in KH2-Cdx2 naïve ES cells prior to and following Dox administration and in 4 Days synthetic embryos obtained according to the protocol described in FIG. 17A, as determined by Real-time PCR.

FIG. 26 demonstrates endogenous expression of the indicated markers in KH2-Cdx2 naïve cells prior to and following Dox and LPA administration and in 4 Days synthetic embryos obtained according to the protocol described in FIG. 19A), as determined by Real-time PCR.

FIG. 27 demonstrates endogenous expression of the indicated markers in KH2-Cdx2 naïve ES cells prior to and following treatment with Dox and the indicated media and factors, as determined by Real-time PCR.

FIG. 28 shows schematic representations of targeting vectors designed to generate human TetON inducible Gata4, or Cdx2 or Gata3 of Gata3-2A-Cdx2 overexpression inserts.

FIG. 29 demonstrates human naïve PSC clones correctly targeted for either GATA4 or CDX2 overexpression. Shown the targeting schemes and western blot analysis before and after DOX treatment which showed specific overexpression of the respective TF following DOX.

FIG. 30 shows plasmid design for generating a lentivirus allowing TetOn inducible Gata4 overexpression after Dox treatment of lentiviral infected mouse or human PSCs. Green reporter is also DOX responsive. This lentivirus can be used with PSCs already expressing M2RTTA or by co-infecting with FUW-CAGGS-M2Rtta encoding lentivirus.

FIG. 31 shows plasmid design for generating a lentivirus allowing TetOn inducible CDX2 overexpression after Dox treatment of lentiviral infected mouse or human PSCs. Green reporter is also DOX responsive. This lentivirus can be used with PSCs already expressing M2RTTA or by co-infecting with FUW-CAGGS-M2Rtta encoding lentivirus.

FIG. 32 shows plasmid design of pcDNA3 plasmid that is used for transient transfection of mouse or human naïve PSCs. It encodes GATA4-IRES-Green fluorescence under a CMV promoter.

FIG. 33 shows plasmid design that is used for transient transfection of human naïve PSCs. It encodes human CDX2-IRES-Green fluorescence under a CAGGS promoter.

FIG. 34 shows plasmid design for generating a human lentivirus allowing TetOn inducible YAP*mut (carries a mutant that it enforces YAP nuclear localization) overexpression after Dox treatment of lentiviral infected mouse or human PSCs. Green GFP reporter is also DOX responsive. This lentivirus can be used with PSCs already expressing M2RTTA or by co-infecting with FUW-CAGGS-M2Rtta encoding lentivirus. For this construct the constitutively active (mut) human YAP was used-Serines at positions 61, 109, 127, 128, 131, 163, 164, and 381 changed to Alanine (phosphorylation sites), see addgene entry #27371 from which the insert was taken (Yap-mut).

FIG. 35 shows plasmid design for generating a lentivirus allowing TetOn inducible Gata3 overexpression after Dox treatment of lentiviral infected mouse or human PSCs. CFP reporter is also DOX responsive. This lentivirus can be used with PSCs already expressing M2RTTA or by co-infecting with FUW-CAGGS-M2Rtta encoding lentivirus.

FIG. 36 shows plasmid design of plasmid that is used for transient transfection of mouse or human naïve PSCs. It encodes GATA3-IRES-CFP fluorescence under a CAGGS promoter.

FIG. 37 shows plasmid design of pcDNA3 plasmid that is used for transient transfection of mouse or human naïve PSCs. It encodes YAP*mut-IRES-CFP fluorescence under a CMV promoter. Yap-mut: carries a mutant that it enforces YAP nuclear localization. For this construct the constitutively active (mut) human YAP was used-Serines at positions 61, 109, 127, 128, 131, 163, 164, and 381 changed to Alanine (phosphorylation sites), see addgene entry #27371 from which the insert was taken (Yap-mut).

FIGS. 38A-B shows images of representative synthetic embryoids obtained according to the protocols described in Example 3 demonstrating generation of human day 4.5 synthetic embryos. FIG. 38A shows a phase contrast image of day 4.5 synthetic human embryoid. FIG. 38B shows images of a representative day 4.5 synthetic human embryoid immunostained for Oct4 pluripotency marker, Sox17 primitive endoderm (hypoblast) marker and CDX2 Trophectoderm marker. The images demonstrate a typic human embryo with an aggregate of OCT4+pluripotent cells surrounded by a sphere of trophectoderm CDX2+ cells, and SOX17+primitive endoderm—hypoblast region lying just below the OCT4+ cell mass.

FIGS. 39A-G demonstrate that the naïve ESC-derived synthetic embryos complete gastrulation and reach organogenesis stages within extraembryonic membranes. FIG. 39A shows a schematic depiction of the synthetic embryo generation and culture protocol. DOX Pre-induction (−1 day for Gata4 in 2iLIF and −14 up to −1 days for Cdx2 in TSC-LPA media) and aggregation of the 3 types of naïve ESCs derived populations followed by culture in AM (with DOX in first 2 days), EUCM2 (20-30% FBS), EUCM2 (30% FBS), and EUCM for 8 days generates self-organized synthetic embryo [“sEmbryos (iCdx2)] that grow to early organogenesis. FIG. 39B shows bright field images of representative embryos at each day of the culture protocol compared to stage-matched natural embryos. FIG. 39C shows the electronically controlled roller bottle ex utero culture platform set-up used for sEmbryo propagation. FIG. 39D shows images of day 7 and day 8 sEmbryos cultured ex utero inside the roller culture bottles. FIG. 39E shows bright-field images of day 8 sEmbryos growing ex utero within extraembryonic membranes (yolk sac and amnion). FIG. 39F shows images of day 8 sEmbryo (iCdx2) and E8.5 natural embryos after dissection and removal of extraembryonic membranes. Insets are enlargements of the dashed boxes. FIG. 39G is a representative image of empty yolk sacs obtained after continuous culture in IVC-static culture based protocol. All images represent a minimum of three biological replicates. Scale bars, 100 μM in FIGS. 39B-C, 500 μm in FIG. 39G. A-anterior; AC-amniotic cavity; Am—amnion; A—allantois; AB—allantoic bud; BI—blood islands; Epi—epiblast; EC—exocoelomic cavity; EPC—ectoplacental cone; ExE—extraembryonic ectoderm; Fg—foregut pocket; H—heart; NF—neural folds; NT—neural tube; P—posterior; PAC—pro—amniotic cavity; PS—primitive streak; S—somites; VE—visceral endoderm; YS—yolk sac.

FIGS. 40A-G demonstrate that expression patterns of lineage markers in egg-cylinder shaped sEmbryos (iCdx2) resemble those of natural embryos. FIG. 40A shows middle section confocal images of day 4 egg cylinder sEmbryos (iCdx2) and natural E5.5 embryos immunostained for extraembryonic ectoderm, visceral endoderm and epiblast markers. FIG. 40B shows migration of the Anterior Visceral Endoderm (AVE) from the distal to the future anterior part revealed by CER1 staining (magenta) in day 4 sEmbryo (iCdx2). FIG. 40C shows representative immunofluorescence images (middle section) of visceral endoderm, trophoblast, epiblast and gastrulation markers (Brachyury, yellow arrows) in day 5 sEmbryos (iCdx2) compared to matched E6.5 natural embryos. FIGS. 40D-E demonstrate migration of the primitive streak and establishment of the definitive endoderm in sEmbryos (iCdx2) at day 6 and control E7.5 natural embryos. FIG. 40D shows immunofluorescence images showing migration of BRACHYURY⁺ cells and presence of Brachyury/Foxa2 double positive cells. FIG. 40E shows SOX17/FOXA2 immunostaining exposing invaginating definitive endoderm cells; FOXA2⁺/SOX17⁻ cells along the epiblast reveal a node-like organizer in sEmbryos (iCdx2). Insets are enlargements of the dashed boxes. FIG. 40F shows middle section images of Blimp1-mVenus fluorescence at day 5 sEmbryos (iCdx2) detected by live imaging marking PGC specification (upper panel). Sox2+ PGCs show proper allocation to the anterior ventral side of the sEmbryos (iCdx2) at day 8 (lower panel). FIG. 40G shows flow cytometry plots for Blimp1-Venus/Stella-CFP in sEmbryos (iCdx2) at day 4 and day 5, marking their emergence at day 5 as expected (equivalent to E6.5). Scale bars are indicated on each panel.

FIGS. 41A-E demonstrate that day 8 post-gastrulation sEmbryos (iCdx2) properly recapitulate spatial expression patterns of tissues derived from all three germ-layers. FIG. 41A shows representative whole-mount immunofluorescence images of day 8 sEmbryos (iCdx2) showing proper expression of neural (Sox2), heart (MHC-II) and notochord (Brachyury) lineage markers. Scale bars, 200 μM. FIG. 41B shows day 8 sEmbryo (iCdx2) maximum intensity projection confocal images for Sox2 and Brachyury compared to natural in utero E8.5 embryo. Scale bars, 200 μM. FIG. 41C shows day 8 sEmbryo (iCdx2) maximum intensity projection confocal images for OTX2 and FOXA2 compared to natural in utero grown E8.5 embryo. Scale bars, 200 μM. FIG. 41D shows schematic representation of the cutting planes for neural tube section (upper panel). Representative immunosection confocal images (mid-section, transversal plane) of the closed neural tube in day 8 sEmbryos (iCdx2) compared to natural in utero E8.5 embryos (lower panel). Scale bars, 20 μM. FIG. 41E shows schematic representation of the cutting plane for heart sections (upper panel). Representative immunohistochemistry images (mid-section, transversal plane) of the heart tube in day 8 sEmbryos (iCdx2) (lower panel). Scale bars, 50 μM. Images are representative of a minimum of 3 biological replicates. FB—forebrain; FP—floor plate; H—heart; LV—left ventricle; Nc—notochord; NF—neural folds; NT—neural tube; RV—right ventricle; TB—tail bud.

FIGS. 42A-F demonstrate that sEmbryos (iCdx2) grow within the extraembryonic membranes and develop allantois, blood islands and ectoplacental cone. FIG. 42A shows representative images of day 7 and day 8 sEmbryos (iCdx2) showing the presence of amnion, yolk sac, allantois, ectoplacental cone and blood islands. Scale bars, 200 μM. FIG. 42B is a schematic illustration of the extraembryonic compartments present in day 8 sEmbryos (iCdx2). FIG. 42C shows representative whole-mount immunofluorescence images of canonical markers in yolk sacs isolated from sEmbryos (iCdx2) at day 8 and E8.5 in utero natural embryos. Scale bars, 500 M. FIG. 42D shows representative images of RUNX1 immunostaining in yolk sacs obtained from sEmbryos (iCdx2) at day 8 and equivalent to E8.5 natural embryos. Scale bars, 50 μM. FIG. 42E shows representative flow cytometry contour plots for CD45 and CD34 expression among Kit⁺ CD41⁺ progenitor cells, and Kit and CD41 expression among lineage negative population from E8.5 in the yolk sac (Upper panel) and sEmbryo (iCdx2) (bottom panel). Gates were set based on the fluorescence minus one (FMO) of each cell surface marker antibody. FIG. 42F shows methylcellulose based in vitro culture, colony forming potential and morphology of erythroid progenitors derived from day 8 sEmbryo (iCDX2) along with natural in utero control E8.5 derived ones. Data represents a minimum of 3 biological replicates. Scale bars are indicated on each panel.

FIGS. 43A-D demonstrate scRNA-seq analysis of natural embryos versus synthetic embryos. FIG. 43A is a UMAP plot displaying individual cells (n=15,284 Natural E8.5 embryos: n=31,151 Synthetic day 8 embryos). Points are colored according to their assigned cell cluster. Cell lineage annotation of clusters based on marker genes of the major cell types identified in E8.5 mouse embryos. EN=total Embryo Number; CN=total Cell Number. FIG. 43B is a UMAP plot displaying individual cells of the different samples as indicated. Colors as in FIG. 43A. FIG. 43C shows natural embryo cells (black) and synthetic embryo cells (red) projected on the same UMAP plot. FIG. 43D shows pie charts depicting the proportional abundance of each cell cluster in both natural and synthetic embryos. Asterisks denote clusters with statistically significant differences between the two groups. *T-test p<0.05.

FIGS. 44A-M demonstrate an optimized gas and pressure regulating controller for roller culture incubators. FIG. 44A is a schematic representation of a fetal incubation system. FIG. 44B is an image of an exemplary fetal incubation system. FIG. 44C is a diagram depicting the configuration of the electronic module for gas and pressure regulation. O₂, N₂ and CO₂ enter into the system at a pressure of 0.1-0.5 psi and are mixed into by a centrifugal blower. Gases are then injected into a water bottle inside the incubator by a pump that allows control of the gas pressure (hyperbaric conditions). Lph=liters per hour. FIG. 44D is a schematic representation of an exemplary gas and pressure controller. FIG. 44E shows a perspective view of an exemplary gas and pressure controller configured to monitor and manipulate CO₂ and O₂ levels by providing CO₂ and/or N₂. FIG. 44F shows a top view of the gas and pressure controller open and showing internal components. FIG. 44G is a front view of the gas and pressure controller. FIG. 44H is a schematic representation of an exemplary gas mixing box. FIG. 44I is an image of an exemplary gas mixing box. FIG. 44J is a schematic representation of an exemplary incubator. FIG. 44K is an image of the interior of the precision incubator system (B.T.C. Engineering) showing the direction of the gas flow (indicated by the white arrowheads). FIG. 44L is an image of embryos cultured in rotating bottles (yellow arrowheads). FIG. 44M is a flowchart of an exemplary method related to the fetal incubation system.

FIGS. 45A-E demonstrate that co-aggregation of human naïve PSCs cells with DOX pre-treated iGata4 human naïve PSCs and DOX pre-treated iCdx2 human naïve PSCs, followed by ex-utero culturing leads to generation of an organized embryo. FIG. 45A is a schematic representation of the protocol. FIGS. 45B-E show phase contrast and immunostaining images of the synthetic embryos obtained at the indicated time points after aggregation. FIG. 45B demonstrates appearance of the bilaminar disc on day +4. FIG. 45C demonstrates the formation of the primary yolk sac on day +5. FIG. 45D demonstrates the formation of the Secondary Yolk sac day +6 after co-aggregation. FIG. 45E demonstrates the consolidation of the bilaminar disc on day +7 human synthetic-embryo.

FIG. 45F shows a schematic representation of an inducible plasmid for transient expression of human CDX2, GATA3 GATA4 or GATA6. Shown is a PiggyBac plasmid expressing a cDNA insert of choice under the control of a doxycycline-inducible promotor. The Piggybac vector used (www (dot) addgene (dot) org/104454/), carries M2Rtta and a cite for cDNA insert of transcription factor of interest. This vector was used to generate 4 different plasmids: human CDX2 or Gata3 (to promote human PSC differentiation towards trophectoderm) and human GATA4 or GATA6 (to promote human primitive endoderm priming from human PSCs.

FIGS. 46A-C show immunostaining images of human PSC lines expressing the inducible plasmid demonstrated in FIG. 45F. FIG. 46A shows JH33 iPSs (a genetically unmodified human iPSC lines made from a healthy adult Caucasian male), and WIBR3 WT human female ESCs used to generate CDX2 inducible lines (iCDX2) using a Piggybac plasmid with CDX2 insert. Shown is immunostaining analysis for CDX2 protein expression of the human primed PSCs grown in mTESR conditions with and without DOX addition (2 μg/ml Dox). CDX2 expression was specifically detected only after DOX addition. FIG. 46B shows WIBR3 WT human female ESCs used to generate GATA4 or GATA6 DOX inducible lines (iGATA4 or iGATA6) using a Piggybac plasmid with human GATA4 or GATA6 as indicated. Shown is immunostaining analysis for GATA6 or GATA4 protein expression of the human primed PSCs (iGATA6 or iGATA4, respectively) grown in mTESR conditions with and without DOX addition (2 μg/ml final concentration of Dox). GATA4 or GATA6 expression was specifically detected only after DOX addition in the corresponding lines as shown. FIG. 46C shows WIBR3 WT human female ESCs used to generate GATA3 inducible lines (iGATA3) using a Piggybac plasmid with a GATA3 insert. Shown is immunostaining analysis for GATA3 protein expression of the human primed PSCs grown in mTESR conditions with and without DOX addition (2 μg/ml final Dox concentration in growth media). GATA3 expression was specifically detected only after DOX addition.

FIGS. 47A-C demonstrate the ability of alternative conditions to generate naïve PSCs as the starting population for generating organized embryos. FIG. 47A shows a schematic representation of the protocol. FIG. 47B demonstrates egg-cylinder shaped sEmbryos obtained at day 6 of the protocol from BVSC, KH2-iGata4 and Kh2-iCDX2 cells expanded in 2i/LIF and a2i/LIF (alternative 2i/Lif) conditions (blue arrows highlight normal day 6 sEmbryo morphology). FIG. 47C is a graph demonstrating efficiency of egg-cylinder shaped sEmbryos obtained at day 5 of the protocol from BVSC, KH2-iGata4 and Kh2-iCDX2 cells expanded in 2i/LIF and a2i/LIF (alternative 2i/LIF) conditions, EPS (LCDM naïve conditions) and serum-LIF conditions. Error bars indicate SEM from average.

FIG. 48 demonstrates that a two steps aggregation protocol enhances sEmbryo formation efficiency. Shown is a schematic representation of the protocol and different regimens and a graph demonstrating efficiency of egg-cylinder shaped sEmbryos obtained at day 5 of the protocol.

FIGS. 49A-D demonstrate generation of human trophectoderm (TE) primed cells by inducing expression of endogenous expression of differentiation factors. FIG. 49A is a schematic representation of the culture conditions. FIG. 49B shows immunostaining characterization of the human trophectoderm (TE) primed cells. Staining was conducted in day 3 of the trophectoderm induction regimen (TE). FIG. 49C shows immunostaining characterization of the human trophectoderm primed cells with or without transgenic iGATA3 expression. Staining was conducted in day 3 of the trophectoderm induction regimen (TE). FIG. 49D demonstrates RT PCR expression of different trophectoderm markers in the WT and iGATA3 WIBR3 cells.

FIGS. 50A-D demonstrate generation of extra embryonic primitive endodermal primed cells (PRE) by inducing expression of endogenous expression of differentiation factors. FIG. 50A shows FACS analysis of PDGFRa expression after 6 days of induction in the indicated media. FIG. 50B shows FACS analysis of PDGFRa expression in iGATA6 cells after 6 days of induction in the indicated media. FIG. 50C demonstrates RT PCR expression of PRE markers (PDGFRa, GATA4, GATA6, Nid2, SOX17) and definitive endoderm markers (Gsc and Foxa2) in the WT and iGATA6 WIBR3 cells. FIG. 50D shows immunostaining characterization of the human PRE cells with or without transgenic iGATA3 expression. Staining was conducted in day 6 of the PRE induction regimen.

FIGS. 51A-L demonstrate that co-aggregation of human naïve PSCs cells with trophectoderm (TE) primed cells described in FIGS. 49A-D and the extra embryonic primitive endodermal primed cells described in FIGS. 50A-D, followed by ex-utero culturing leads to generation of an organized embryo. FIG. 51A shows a schematic representation of the protocol. FIG. 51B demonstrates progression of human sEmbryos from D4 to D8 of the protocol (top panel) compared to 9-12 dpf of human embryos (schematically shown at the bottom). The top panel shows images of human days 4-8 sEmbryos immunostained for OCT4 (Cyan), SOX17 (Yellow) and CK7 (Magenta), which are markers for epiblast, hypoblast and trophoblast, respectively. Scale bar: 50 μm. Abbreviations: TB. Trophoblast; EB, Epiblast: HB, Hypoblast; L. Lacuna: AC. Amniotic cavity; PYS, Primary Yolk Sac; ChC, Chorionic Cavity; ExEM, Extraembryonic Mesoderm; SK, Stalk; dpf, days post fertilization. FIG. 51C shows characterization of epiblast pluripotency in human sEmbryos. Shown are image of D4 sEmbryos immunostained for OCT4, SOX2, KLf17 and DNA (DAPI). Scale bar: 25 μm. Top panel—2× zoom into the epiblast region. FIGS. 51D-E shows apical cell polarity and lumenogenesis in human sEmbryos. FIG. 51D shows images of D6 sEmbryos immunostained for OCT4 (cyan), F-actin (red), and DNA (DAPI, grey). Scale bar: 50 μm. Asterisk indicates putative chorionic cavity. Right top, 2× zoom into the amniotic lumen. Right bottom, 2× zoom into the yolk sac lumen. Arrows mark apical cell surfaces with aPKC (green). Scale bar: 12.5 μm. FIG. 51E shows images of D6 sEmbryos immunostained for SOX17 (yellow), F-actin (red), and DNA (DAPI, grey). Scale bar: 50 μm. Right, zoom into the yolk sac lumen showing yolk sac layers with cylindrical (top) and a squamous cell (bottom). Cell perimeters are outlined with dashed lines. Scale bar: 5 μm. OCT4 and SOX17 mark epiblast and hypoblast, respectively. FIGS. 51F-G show Anterior-posterior symmetry breaking in the human sEmbryos. FIG. 51F shows images of D6 sEmbryos immunostained for OCT4 (cyan), CER1 (red), and DNA (DAPI, grey). Scale bar: 50 μm. Bottom, 4× zoom into the hypoblast region enriched for apical vesicles with CER1 (marked with arrows) which makes the AVE (anterior visceral endoderm). FIG. 51G shows images of D6 sEmbryos immunostained for OCT4 (cyan), Brachyury (BRA, red), and DNA (DAPI, grey). Scale bar: 50 μm. Right, 4× zoom into the BRA-positive epiblast cells (marked with arrows). OCT4 marks pluripotent epiblast whereas BRA and CER1 are early markers of epiblast and hypoblast differentiation, respectively, associated with anterior-posterior symmetry breaking in the early post-implantation human embryo. FIGS. 51H-J show trophoblast compartment characterization of the sEmbryos. FIG. 51H shows images of D6 sEmbryos immunostained for epiblast (OCT4, Cyan), hypoblast (SOX17, Yellow) and three different markers for trophoblast (CK7, SDC1, GATA3, Magenta). All three trophoblast are present in and form a surrounding layer covering the structure. FIG. 51I top panel shows images of D6 sEmbryos immunostained for F-ACTIN (Red) and HCGB (Green), demonstrating the membranes and syncytiotrophoblast. Zoom in shows multinucleated cell expressing HCGB, characteristic of syncytiotrophoblast. FIG. 51I bottom panel shows images of D6 sEmbryos immunostained for CK7 (Magenta) and HCGB (Green) demonstrating partial, but not complete overlap of the two markers, indicating the presence of cyto-(CK7+ only) and syncytiotrophoblast (CK7+ and HCGB+). FIG. 51J top panel shows images of D6 sEmbryos immunostained for F-ACTIN and HCGB. Single stack of membrane marker F-actin and HCGB show the formation of lacuna (dotted line) by syncytiotrophoblast. FIG. 51J bottom panel shows maximum projection demonstrating that syncytiotrophoblast surround the whole structure. Scale bar: 50 μm. FIGS. 51K-L demonstrate presence and organization of Extraembryonic mesoderm (ExEm) in the sEmbryos. FIG. 51K shows images of D6 sEmbryos immunostained for epiblast (OCT4, Cyan), PYS (SOX17, Yellow) and ExEm markers (BST2, GATA6, Red). The top panel demonstrates that BST2+ cells invade the space between the yolk sac (SOX17+) and the outer trophoblast layer (DAPI, SOX17−, OCT4−, BST2−) showing the presence of ExEM cells. The middle panel demonstrates that the ExEM are also express GATA6, a shared marker for HB/PYS, but in a lower level then in the PYS. The bottom panel demonstrates that the ExEM cells organize and form a cavity between PYS and trophoblast cells, indicating the formation of chorionic cavity (asterix). FIG. 51L shows a zoom in of the middle-section of immunofluorescent image stained for OCT4 (Cyan), SOX17 (Green) and BST2 (Red). BST2 is expressed in between the yolk sac (SOX17+) and the outer trophoblast cells (DAPI, SOX17−, OCT4−, BST2−) and in the epiblast (OCT4+). ExEM cells are characterized here by expression of BST2 and lack of expression of SOX17 and OCT4. Scale bar: 50 μm. Abbreviations: Max. Proj., Maximum intensity projection of z-stacks.

FIGS. 52A-B demonstrate generation of naïve Rhesus Macaque (RM) PSCs. FIG. 52A shows phase images demonstrating typical domed like morphology of RM-1 naïve hESCs expanded in NENSM conditions at the indicated passages. FIG. 52B shows images of the monkey naïve RM1-ESCs immunostained for several naïve pluripotency markers: DPPA3, TFCP2L1, KLF17 and NANOG. DAPI was used for counterstain.

FIG. 53 is a schematic representation of the protocol for the generation of RM synthetic embryos.

FIG. 54 demonstrates that co-aggregation of RM naïve PSCs cells with human trophectoderm (TE) primed cells and the human extra embryonic primitive endodermal primed cells described in Example 12, followed by ex-utero culturing leads to generation of an organized embryo. The Figure demonstrates progression of human sEmbryos from D6 of the protocol (left panel) compared to 9-12 dpf of RM embryos (schematically shown on the right). The sEmbryo was immunostained for OCT4 (Green), SOX17 (Red) and Gata3 (Magenta), which are markers for epiblast, hypoblast and trophoblast, respectively. Scale bar: 50 μm.

FIG. 55 demonstrates formation of a stable human TSC line. Shown are images of cells immunostained for CDX2, Cytokeratin7 (CK7), GATA3 and TFAP2C. DAPI was used for counterstain.

FIG. 56 demonstrates that human stable derived TSC lines are not capable of forming normal sEmbryos. Three starting cell types were co-aggregated, naïve WT hESCs cultured in HENSM medium; for the primitive endoderm compartment Naïve WT cells were plated on mouse embryonic fibroblast conditions on HENSM supplemented with ROCKi 10 μM (Axon Medchem 1683), next day medium was replaced by RCL medium, RCL was kept for 72 h with 24-hour medium exchanges; and for the Trophoblast compartment, a human TSC line derived from WIBR3 naïve ESCs at (Passage 5) was used. The latter was generated by transferring human naïve WIBR3 expanded in HENSM conditions into TSC media on MEF coated plates and serially passaged in TSC media and validated as TSC line. The TSC line was constitutively labeled with RFP coding lentivirus (labeling it as RED). Human TSCs were introduced in day 0, day 1 and day 2 of the sEmbryo protocol. In all cases it could be appreciated that the cells co-localize in one side of the aggregate (as appreciated by their RED label) without surrounding the entire sEmbryo at Day 5, which does not correspond with the expected morphology and does not yield normally developed embryos that are surrounded by trophoblast lineage only on the outside periphery.

FIGS. 57A-C demonstrate derivation of novel stable mouse extra embryonic primitive endodermal primed cells (mouse PRE) from WT mouse naïve ESCs/iPSCs or following transient overexpression of Gata4 in mouse ESCs. FIG. 57A shows three strategies used to derive the mouse PRE cells that can be permanently expanded in mouse PRE indicated media. Images were taken at time 0 (naive ESC in 2i/LIF stage) and after differentiation start. DOX was used for 72 hours in the first regimen as indicated. FIG. 57B shows FACS analysis of the cells using an anti-PDGFRa antibody. The parental ESC lines are shown as controls. Unstained samples are shown as negative controls. It is noted that MEFs are known to express PDGFRA as well, but at lower levels than mouse PRE cells. FIG. 57C shows representative images of the cells immunostained for the indicated markers. Immunostaining was effected on genetically unmodified CD1 iPSC derived mouse PRE cells expanded in PRE media. DAPI was used as counterstain.

FIG. 58 demonstrates that co-aggregation of naïve mouse PSCs with the mouse PRE cells described in FIGS. 57A-C and mouse iCDX2 trophectoderm primed cells, followed by ex-utero culturing enabled generation of organized embryos. The Figure shows images of Day 8 mouse sEmbryos generated according to protocol shown in the Figures.

FIGS. 59A-B demonstrate that co-aggregation of human naïve PSCs cells with trophectoderm (TE) primed cells and extra embryonic primitive endodermal primed cells as described in Example 15 hereinbelow, followed by ex-utero culturing leads to generation of an organized embryo. FIG. 59A shows a schematic representation of the protocol. FIG. 59B is a representative phase image demonstrating bilaminar disc formation, amniotic cavity and yolk sac cavity, all surrounded by trophoblast compartment.

FIGS. 60A-B demonstrate that co-aggregation of human naïve PSCs cells with trophectoderm (TE) primed cells as described in Example 16 hereinbelow, followed by ex-utero culturing leads to generation of an organized embryo. FIG. 60A shows a schematic representation of the protocol. FIG. 60B is a representative phase image demonstrating bilaminar disc formation, amniotic cavity and yolk sac cavity, all surrounded by trophoblast compartment.

### DESCRIPTION OF SPECIFIC EMBODIMENTS OF THE INVENTION

The present invention, in some embodiments thereof, relates to methods of generating a synthetic embryo.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Advanced artificial embryogenesis utilizing in vitro established embryonic stem cell lines (ESCs) and induced pluripotent stem cell lines (iPSCs) while bypassing the use of oocytes and zygotes or blastocysts has been a desired goal in the fields of developmental biology and reproductive medicine. While different pluripotent states of stem cells can be expanded in vitro and can contribute to embryonic and/or extraembryonic tissues when injected into host embryos, obtaining advanced gastrulating whole embryo-like structures with both embryonic and extra-embryonic compartments without relying on a micro-injected host embryo, has failed to date (see e.g., Zhang et al. (2020) Front. Bioeng. Biotechnol. 8:781; Harrison et al. Science (2017) DOI:10.1126/science.aa1810; Amadei et al. Developmental Cell (2021) 56, 366-382; as also exemplified by Example 1 of the Examples section which follows).

Whilst reducing specific embodiments of the present invention to practice, the present inventors have now developed a method for generation of synthetic embryos, with both embryonic and extra-embryonic compartments, by starting solely from naïve ESCs followed by culturing under a controlled ex-utero embryo culture platform.

As is illustrated hereinunder and in the examples section, which follows, the present inventors show that co-aggregating three populations of mouse stem cells, namely non-transduced naïve PSCs, naïve PSCs that transiently overexpress a master regulatory transcription factor for trophectoderm cells (Cdx2) and naïve ESCs that transiently overexpress a master regulatory transcription factor for an extra embryonic primitive endodermal cells (Gata4) followed by ex-utero culturing the aggregate in a recently described controlled platform and growth conditions (Aguilera-Castrejon et al., 2021a), resulted in assembly into complete synthetic mouse embryos that accomplish gastrulation, develop brain, neural folds, neural tube, beating heart, somites, and progenitors of other organs (Examples 2-3 and 7-8 of the Examples section which follows). The synthetic embryos developed within extraembryonic membranes as natural embryos. Further, thorough analysis of both embryonic and extra embryonic compartments showed that these synthetic embryos achieved equivalent level of lineage complexity seen in natural in-utero embryos, where all annotated cell types were represented in synthetic embryos at E8.5 equivalent developmental stage. Furthermore, for each tissue a very high level of correlation in gene expression was seen between natural in-utero embryos and the synthetic mouse embryos. In a similar manner, the present inventors show that co-aggregating the same three populations of stem cells but of human origin, followed by ex-utero culturing the aggregate, resulted in assembly into complete synthetic human embryos with both embryonic and extra embryonic compartments (Examples 4-6 of the Examples section which follows). Following, the present inventors show that various culturing conditions can also support differentiation of naïve PSCs (of mouse, human or Rhesus Macaque origin) into trophectoderm (TE)-primed or extra embryonic primitive endodermal (PRE)-primed cells; and the co-aggregation of these stem cell populations followed by ex-utero culturing the aggregate resulted in assembly into complete synthetic embryos (Examples 9-12 and 14-16 of the Examples section which follows).

The establishment of methods and systems for producing such synthetic embryos ex utero may constitute a powerful tool in basic research e.g. as a framework to investigate the emergence of cellular diversity, cell fate decisions and how tissues and organs emerge from a single pluripotent cell; as well as a source of cells, tissues, embryos and organs for transplantation, testing the effect of drugs on embryonic development and possibly the bioengineering of embryos etc.

Thus, according to an aspect of the present invention, there is provided a method of generating a synthetic embryo, the method comprising:
(a) inducing expression of a factor that induces differentiation to trophectoderm cells in a subpopulation of naïve pluripotent stem cells (PSCs) to obtain trophectoderm primed cells; and
(b) mixing said trophectoderm primed cells with naïve PSCs under conditions that allow formation of aggregated cells,
wherein when said factor that induces differentiation to trophectoderm cells is an exogenous factor said inducing said expression comprises inducing transient expression starting within 14 days—0 hours prior to said mixing and ending no later than 120 hours following said mixing; and
wherein said factor that induces differentiation to trophectoderm cells is an endogenous factor said inducing said expression comprises culturing said subpopulation of naïve PSCs under conditions enabling expression of said factor starting within 14 days-6 hours prior to said mixing,
thereby generating the synthetic embryo.

According to an additional or an alternative aspect of the present invention, there is provided a method of generating a synthetic embryo, the method comprising:
(a) inducing transient expression of an exogenous factor that induces differentiation to trophectoderm cells in a subpopulation of naïve pluripotent stem cells (PSCs) to obtain a trophectoderm primed cells; and
(b) mixing said trophectoderm primed cells with naïve PSCs under conditions that allow formation of aggregated cells,
wherein said inducing said transient expression starts within 14 days-0 hours prior to said mixing and ends no later than 120 hours following said mixing, thereby generating the synthetic embryo.

According to specific embodiments, the method further comprising inducing expression of a factor that induces differentiation to extra embryonic primitive endodermal cells in a second subpopulation of naïve PSCs to obtain extra embryonic primitive endodermal primed cells in said (a); and mixing said extra embryonic primitive endodermal primed cells with said cells in said (b).

According to specific embodiments, the method further comprising inducing transient expression of an exogenous factor that induces differentiation to extra embryonic primitive endodermal cells in a second subpopulation of naïve PSCs to obtain an extra embryonic primitive endodermal primed cells in said (a); and mixing said extra embryonic primitive endodermal primed cells with said cells in said (b).

According to an additional or an alternative aspect of the present invention there is provided a mixture or aggregate of cells obtainable by the methods disclosed herein, as further described infra.

According to an additional or an alternative aspect of the present invention there is provided a synthetic embryo obtainable by the methods disclosed herein.

As used herein, the term “synthetic embryo” refers to at least a collection of distinct cells generated ex-vivo using pluripotent stem cells without any donated germ cells, zygotes or blastocysts. When cultured, the synthetic embryo undergoes self-organization and differentiation that give rise to multiple tissue types which are organized as a native embryo or exhibit embryonic-like properties.

According to specific embodiments, the synthetic embryo structure mimics the developmental stages of a native or naturally occurring embryo, at least in the early stages of embryonic development post the blastocyst developmental stage.

According to specific embodiments, the synthetic embryo is not a blastocyst (e.g. a structure having characteristic of E3.5/4-5 of an in-utero natural mouse embryo or PFD6/PFD7 of an in-utero natural human embryo).

According to specific embodiments, the synthetic embryo mimics at least the Carnegie 5, Carnegie 6, Carnegie 8 or later developmental stages (relates to humans), embryo with primary and secondary yolk sac, bilaminar disc stage surrounded by trophoblast surround and formation of amnion, yolk and chorionic cavity including the formation of extra-embryonic mesoderm cells.

According to specific embodiments, the mouse synthetic embryo mimics at least the formation of egg-cylinder stage, completion of gastrulation (E7.5) and beginning of organogenesis (E8.0 in mouse embryos or later).

According to specific embodiments, the synthetic embryo is generated solely ex-utero.

As used herein the phrase “pluripotent stem cell (PSC)” refers to an undifferentiated cell (e.g., a mammalian cell, a primate cell) capable of differentiating into all three embryonic germ cell layers, i.e., to the mesoderm, ectoderm and endoderm embryonic germ layers; or remaining in an undifferentiated state.

Methods of determining ability of stem cells to differentiate into the endodermal, mesodermal and ectodermal embryonic germ layers are known in the art and disclosed e.g. in International Patent Application Publication No. WO2014/174470, the contents of which is fully incorporated herein by reference in its entirety, and include for example generation of embryoid bodies (in vitro) or teratomas (in vivo).

According to some embodiments of the invention, the pluripotent stem cell is selected from the group consisting of embryonic stem cell (ESC), induced pluripotent stem cells (iPSCs), and embryonic germ cell (EGC).

According to specific embodiments, the cell is a mammalian cell e.g., rodent (e.g. mouse, rat), human (Homo sapiens), monkey (e.g. chimpanzee, Gorillas, Rhesus and/or Baboon), livestock animal (e.g. cattle, sheep, pigs, goats, horses, donkeys, mules).

According to specific embodiments, the cell is a mouse cell.

According to specific embodiments, the cell is a human cell.

According to other specific embodiments, the cell is of a non-human primate (e.g. Macaques).

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763) and embryonic germ (EG) cells which are obtained from the genital tissue of a fetus any time during gestation, preferably before 10 weeks of gestation.

The embryonic stem cells of some embodiments of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting.

The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., [U.S. Pat. No. 5,843,780; Science 282:1145, 1998; Curr. Top. Dev. Biol. 38:133, 1998; Proc. Natl. Acad. Sci. USA 92:7844, 1995]; Bongso et al., [Hum Reprod 4:706, 1989]; and Gardner et al., [Fertil. Steril. 69:84, 1998].

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

It will be appreciated that commercially available stem cells can also be used according to some embodiments of the invention. Human ES cells can be purchased from the NIH human embryonic stem cells registry [Hypertext Transfer Protocol://grants(dot)nih(dot)gov/stem_cells/registry/current(dot)htm]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES1, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), HUES 62, HUES 63, HUES 64, CT1, CT2, CT3, CT4, MA135, Eneavour-2, WIBR1, WIBR2, WIBR3, WIBR4, WIBR5, WIBR6, HUES 45, Shef 3, Shef 6, BJNhem19, BJNhem20, SA001, SA001.

In addition, ES cells can be obtained from other species as well, including mouse (Mills and Bradley, 2001), golden hamster [Doetschman et al., 1988, Dev Biol. 127:224-7], rat [Iannaccone et al., 1994, Dev Biol. 163:288-92] rabbit [Giles et al. 1993, Mol Reprod Dev. 36:130-8; Graves & Moreadith, 1993, Mol Reprod Dev. 1993, 36:424-33], several domestic animal species [Notarianni et al., 1991, J Reprod Fertil Suppl. 43:255-60; Wheeler 1994, Reprod Fertil Dev. 6:563-8; Mitalipova et al., 2001, Cloning. 3:59-67] and non-human primate species (Rhesus monkey and marmoset) [Thomson et al., 1995, Proc Natl Acad Sci USA. 92:7844-8; Thomson et al., 1996, Biol Reprod. 55:254-9].

Non-limiting examples of commercially available mouse embryonic stem cell lines are v6.5, KH2, BVSC ES line, NGFP1 iPSC line, NGFP2 iPSC lines.

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, MO, USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

EG cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95:13726, 1998] and U.S. Pat. No. 6,090,622.

Induced pluripotent stem cells (iPS: embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kf14 and c-Myc in a somatic stem cell.

Induced pluripotent stem cells (iPS) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1 (1): 39-49; Aoi T, et al., Generation of Pluripotent Stem Cells from Adult Mouse Liver and Stomach Cells. Science. 2008 February 14. (Epub ahead of print); I H Park, Zhao R, West J A, et al. Reprogramming of human somatic cells to pluripotency with defined factors. Nature 2008:451:141-146; K Takahashi, Tanabe K. Ohnuki M, et al. Induction of pluripotent stem cells from adult human fibroblasts by defined factors. Cell 2007; 131:861-872]. Other combinations of pluripotency factor overexpression generated iPSCs are included as well (Buganim et al. Cell (2012) 150 (6): 1209-1222 www(dot)doi(dot)org/10.1016/j.cell.2012.08.023) Other embryonic-like stem cells can be generated by nuclear transfer to oocytes, fusion with embryonic stem cells or nuclear transfer into zygotes if the recipient cells are arrested in mitosis.

The methods of some embodiments of the invention utilize PSCs in their naïve state.

The phrase “naïve pluripotent stem cells (PSCs)” refers to PSCs that express pluripotent markers (such as Oct4, Nanog and Klf4), lack of extraembryonic endoderm, trophectoderm and somatic early lineage specific markers. The cells retain a non-restricted developmental potential as they can robustly differentiate into all cell types in vitro and, upon injection into the blastocyst, they efficiently contribute to the three germ layers and to the germ-line of chimeric animals.

### Human and Non-Human Primate Naïve PSCs Also a Pre X-Inactivation State.

Chromosome X inactivation is an early developmental process in mammalian females that transcriptionally silences one of the pair of X chromosomes, thus providing dosage equivalence between males and females. The process is regulated by several factors, including a region of chromosome X called the X inactivation center (XIC). The XIC comprises several non-coding and protein-coding genes, and XIST gene was the first non-coding gene identified within the XIC.

When XIST bodies are present, then XIST is exclusively expressed from the XIC of the inactive X chromosome, and is essential for the spread of X-inactivation.

The pre-X-inactivation state according to some embodiments of the invention is characterized by presence of two unmethylated alleles of the promoter of the X-inactive specific transcript (XIST) gene in the female cell, and presence an unmethylated allele of the promoter of the XIST gene in a male cell.

This is contrary to human and non-human primate “primed PSC” or “conventional PSC” which are characterized by one methylated allele of XIST and one unmethylated allele of XIST in the female cell, and by one methylated allele in the male cell.

The XIST gene is located on human Xq13.2 chromosome and has the sequence depicted in clone NC_000023.10 (73040486.73072588, complement, based on GenBank version GRCh37. p10). The XIST gene has a non-coding RNA which is provided in GenBank Accession NO. NR_001564.2.

Methods of determining the methylation status of the XIST gene are known in the art and disclosed e.g. in Lengner Cell 141, 872-883 (2010); Hanna et al., Cell 143, 508-525; (2010); TAKASHI SADO et al., DEVELOPMENTAL DYNAMICS 205:421-434 (1996); and International Patent Application Publication No. WO2016/016894 and WO2014/174470, each of which is fully incorporated herein by reference in its entirety, and include e.g. bisulfite sequencing and Southern blot analysis.

According to some embodiments of the invention, presence of two unmethylated alleles of XIST gene in a female cell refers to having below about 20% of CpG methylated reads sequenced in the XIST promoter, e.g., below about 19%, below about 18%, below about 17%, below about 16%, below about 15%, below about 14%, below about 13%, below about 12%, below about 11%, below about 10%, below about 9%, below about 8%, below about 7%, below about 6%, below about 5%, below about 4%, below about 3%, below about 2%, below about 1%, e.g., 0% (e.g., complete absence) of CpG methylated reads sequenced in the XIST promoter.

According to some embodiments of the invention, presence of one unmethylated allele of XIST gene in a male cell refers to having below about 20% of CpG methylated reads sequenced in the XIST promoter, e.g., below about 19%, below about 18%, below about 17%, below about 16%, below about 15%, below about 14%, below about 13%, below about 12%, below about 11%, below about 10%, below about 9%, below about 8%, below about 7%, below about 6%, below about 5%, below about 4%, below about 3%, below about 2%, below about 1%, e.g., 0% of CpG methylated reads sequenced in the XIST promoter.

A non-limited example of the XIST promoter which includes CpG islands which can be either methylated or unmethylated is provided in the XIST promoter amplicon set forth by SEQ ID NO: 65.

According to some embodiments of the invention, the naive PSC exhibits a pre-X inactivation status similar to the pre-X inactivation status of a human Inner cell mass (ICM).

According to some embodiments of the invention, the naive PSC is characterized by a reduced methylation of CpG islands as compared to a level of methylation of the CpG islands in a primed PSC.

According to some embodiments of the invention, naive PSCs are characterized by significantly low levels of total methylated cytosine out of the total guanine nucleotides in each cell (e.g., 1-2%) as determined by Liquid Chromatography-Mass Spectrometry (LC-MS) quantitative analysis.

According to some embodiments of the invention, the naive PSC is characterized by 0-3% of total methylated cytosine out of the total Guanine nucleotides in the naive PSC cell. For comparison, the primed PSC or a somatic cell has between 3.5%-5% of total methylated cytosine out of the total Guanine nucleotides in the primed PSC cell.

According to some embodiments of the invention, the human naive PSC expresses XIST.

Methods of detecting XIST expression are known in the art and include for example reverse transcriptase—polymerase chain reaction (RT-PCR) analysis using XIST specific PCR primers, e.g., the forward primer: 5′-AGGGAGCAGTTTGCCCTACT (SEQ ID NO: 66), and the reverse primer: 5′-CACATGCAGCGTGGTATCTT (SEQ ID NO: 67).

According to some embodiments of the invention, the naive PSC is devoid of XIST bodies.

As used herein the phrase “XIST bodies” refers to a XIST-coated inactive X chromosome.

Methods of detecting XIST bodies are known in the art and include for example RNA fluorescent in situ hybridization. RNA fluorescence in situ hybridization (FISH) is carried out as previously described [Hanna J., et al., Cell 143, 508-525]. Briefly, human pluripotent stem cells are harvested, MEF-depleted, and cytospun onto glass slides before fixation. cDNA probes are generated to XIST exon 1 (GenBank Accession No. U80460: 61251-69449, SEQ ID NO: 26) and exon 6 (GenBank Accession No. U80460: 75081-78658, SEQ ID NO: 27) and labeled by nick translation (Roche) with Cy3-dUTP (Amersham), and Cot-1 DNA is labeled with fluorescein-12-dUTP using the Prime-It Fluor Labeling Kit (Stratagene).

According to some embodiments of the invention, the naive PSC is devoid of an H3K27me3/polycomb focus.

As used herein the phrase “H3K27me3/polycomb focus” refers to nuclear focus obtained following immuno-staining that corresponds to condensed inactive X chromosome.

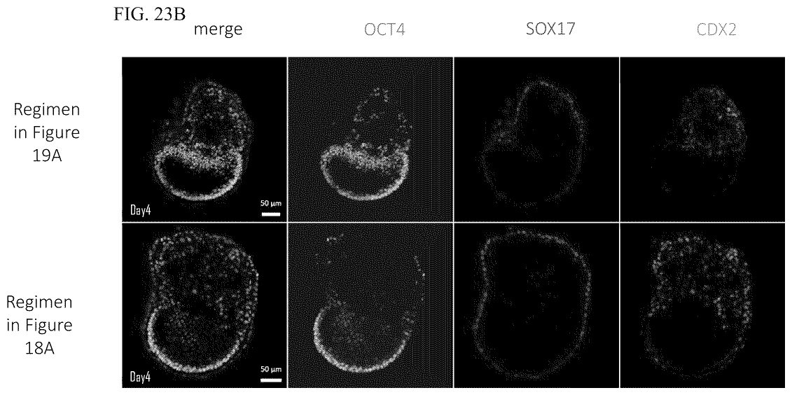

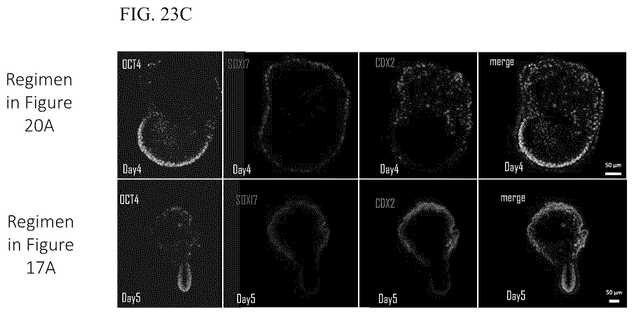

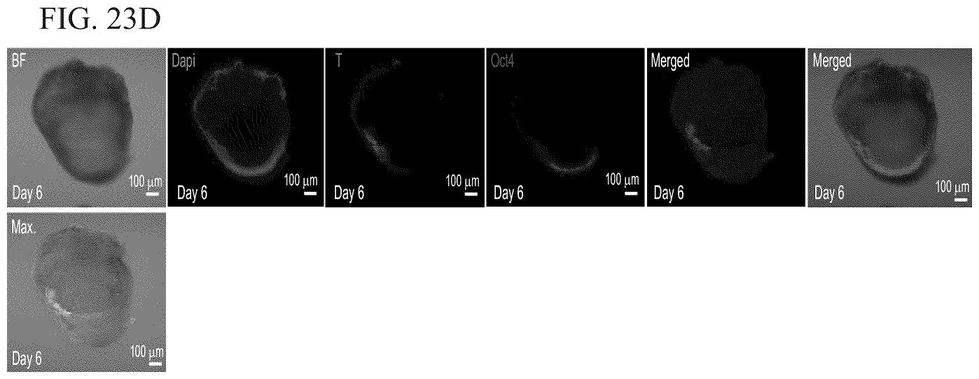

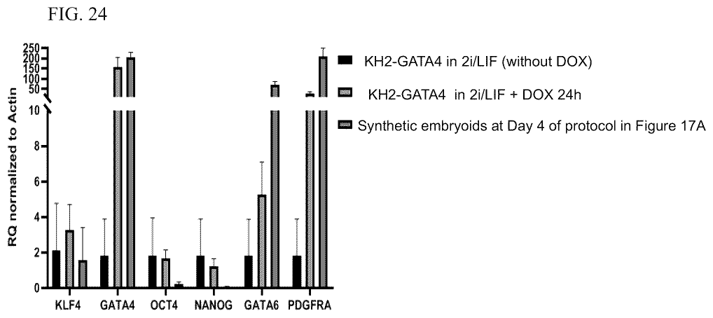

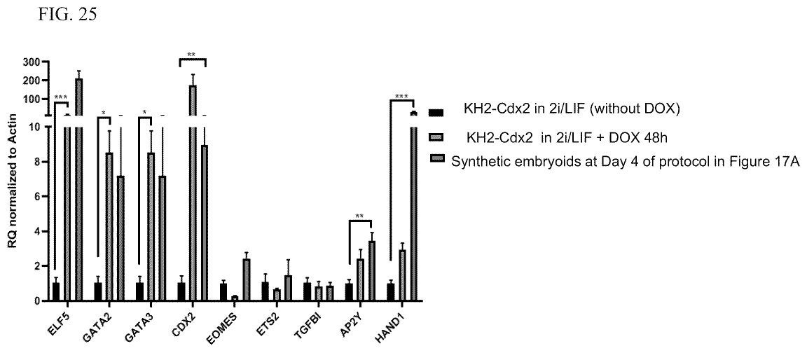

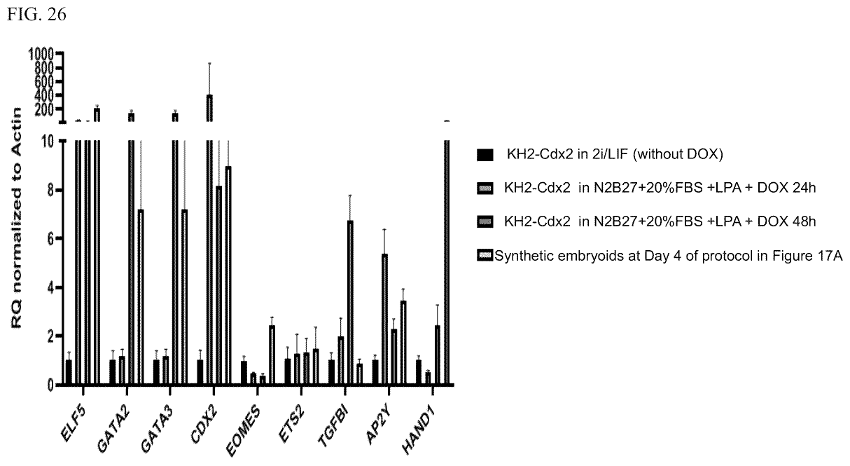

Methods of detecting H3K27me3/polycomb focus are known in the art and include for example, the use of immuno-fluorescence analysis using anti H3K27me3 antibodies (e.g., Rabbit anti H3K27me3, Millipore, CA, USA Catalogue number 07-449), as shown for example in FIGS. 23C-D.

According to some embodiments of the invention, the naive PSC has a low XIST expression level while being in the naive state, without inactivation of any of the X chromosomes and without presence of XIST bodies.

It should be noted that the naive PSCs of some embodiments of the invention (which are in a pre-X inactivation and a naive state) can upon differentiation inactivate one of the X chromosome alleles and methylate one of the XIST genes.

According to specific embodiments, expression level of the transcription factor E3 (TFE3) in the naive PSC is characterized by a nucleus to cytoplasm expression ratio which is equal to or higher than 1, as determined by e.g. an immunostaining assay.

According to specific embodiments, the naïve cells express c-Kit on their cell surface, as determined by e.g. flow cytometry or immunostaining assay.

Contrary to the known primed PSC, the naive PSC of some embodiments of the invention is “resistant” to induction of differentiation by FGFR/MEK/ERK inhibitor, JNK inhibitor, and/or P38 inhibitor. Thus, according to some embodiments of the invention, when the isolated naive PSC is incubated in the presence of an agent selected from the group consisting of FGFR/MEK/ERK inhibitor, JNK inhibitor, and P38 inhibitor, the naive PSC remains in the pluripotent state.

According to some embodiments of the invention, the naive PSC has an inhibited p38 pathway as compared to a primed PSC. For example, p38 activity is inhibited in the naive PSC.

According to some embodiments of the invention, the level of p38 RNA and/or phosphorylated p38 protein in the naive PSC is less than about 30%, e.g., less than about 20%, e.g., less than about 5%, e.g., less than about 0.5%, e.g., less than about 0.1% as compared to the level of p38 RNA and/or phosphorylated p38 protein, respectively, in a non-naive PSC incubated and/or cultured under the same conditions, yet without being subject to p38 inhibition.

According to some embodiments of the invention, the naive PSC has an inhibited JNK pathway as compared to a primed PSC. For example, JNK activity is inhibited in the naive PSC.

According to some embodiments of the invention, the level of JNK RNA and/or phosphorylated JNK protein in the naive PSC is less than about 30%, e.g., less than about 20%, e.g., less than about 5%, e.g., less than about 0.5%, e.g., less than about 0.1% as compared to the level of JNK RNA and/or phosphorylated JNK protein, respectively, in a non-naive PSC incubated and/or cultured under the same conditions, yet without being subject to JNK inhibition.

According to some embodiments of the invention, the naive PSC has an inhibited ROCK pathway as compared to a primed PSC. For example, ROCK activity is inhibited in the naive PSC.

According to some embodiments of the invention, the level of ROCK RNA and/or phosphorylated ROCK protein in the naive PSC is less than about 30%, e.g., less than about 20%, e.g., less than about 5%, e.g., less than about 0.5%, e.g., less than about 0.1% as compared to the level of ROCK RNA and/or phosphorylated ROCK protein, respectively, in a non-naive PSC incubated and/or cultured under the same conditions, yet without being subject to ROCK inhibition.

According to some embodiments of the invention, the naive PSC which is maintained in the undifferentiated, pluripotent and naive state (as defined above), expresses significantly lower levels SOX1 as compared to the level of expression present in primed PSC (e.g., primed ESC) under identical SOX1 assay conditions, and wherein the primed PSC exhibits one methylated and one unmethylated allele of XIST (in a female cell) or one methylated allele of XIST (in a male cell); expresses XIST; exhibits XIST bodies; and exhibits a H3K27me3/polycomb focus.

According to some embodiments of the invention, the naive PSC expresses a lower level of MHC class I as compared to a primed PSC under identical detection assay conditions, and wherein the primed PSC exhibits one methylated and one unmethylated allele of XIST (in a female cell) or one methylated allele of XIST (in a male cell); expresses XIST; exhibits XIST bodies; and exhibits a H3K27me3/polycomb focus.

The level of MHC class I can be determined by various methods known in the art such as FACS analysis using specific antibodies to detect the surface expression of the MHC class I molecules, and using fluorescently labeled anti HLA-A,B,C antibody (BD Biosciences).

According to some embodiments of the invention, the naive PSC is characterized by at least about 10%, at least about 20%, at least about 30%, at least about 40%, at least about 50%, at least about 60%, at least about 70%, at least about 80%, at least about 90% more RNA polymerase II pausing on chromosomes as compared to a primed PSC under identical assay conditions, and wherein the primed PSC exhibits one methylated and one unmethylated allele of XIST (in a female cell) or one methylated allele of XIST (in a male cell); expresses XIST; exhibits XIST bodies; and exhibits a H3K27me3/polycomb focus.

According to specific embodiments, the naïve PSCs express a pluripotency marker, i.e., positive for one or more pluripotency markers.

According to specific embodiments, the expression is of an endogenous protein.

Positive is also abbreviated by (+). Positive for a marker means that at least about 70%, 80%, 85%, 90%, 95%, or 100% of the cells in the population present detectable levels of the marker assayed by a method known to those of skill in the art. Developmental markers can be detected using immunological techniques well known in the art [described e.g. in Thomson J A et al., (1998). Science 282:1145-7]. Examples include, but are not limited to, immunostaining, microscopy, flow cytometry, western blot, and enzymatic immunoassays. Other non-limiting methods include PCR analysis, RNA fluorescence in situ hybridization (FISH), northern blot, single cell RNA sequencing.

Pluripotency markers are well known in the art and include, but not limited to Oct4, NANOG, KLF4, KLF17, SUSD2, TFCP2L1.

The naïve PSC of some embodiments of the invention is characterized by a positive expression of the C-KIT protein (also known as CD117) on the cell surface.

According to specific embodiments, the naïve PSCs can be obtained in culture in the naive state without the need of exogenous expression of Oct4, Sox2, Klf4 and/or c-Myc factors.

According to specific embodiments, the naïve PSCs are negative for a differentiation marker.

Negative is also abbreviated by (−). Negative for a marker means that no more than about 30%, no more than about 25%, no more than about 20%, no more than about 15%, no more than about 10%, no more than about 5%, no more than about 4%, no more than about 3%, no more than about 2%, no more than about 1%, of the cells in the population present detectable levels of the marker assayed by a method known to those of skill in the art such as FACS, immunofluorescence, immunohistochemistry, Western blot analysis, RT-PCR. Non-limiting examples of differentiation markers include Gata3, Gata2, tbr2, tfap2c, Cdx2, Elf5, Hand1, EOMES, ETS2, Foxa2, Gata4, Gata6, PDGFRA, Sox17.

Determination of PSC differentiation can also be effected via measurements of alkaline phosphatase activity. Undifferentiated human ES cells have alkaline phosphatase activity which can be detected by fixing the cells with 4% paraformaldehyde and developing with the Vector Red substrate kit according to manufacturer's instructions (Vector Laboratories, Burlingame, California, USA).

According to specific embodiments, the naïve PSCs can be obtained in culture in the naive state under feeder layer free conditions [e.g., on a Matrigel, Laminin, and/or fibronectin-coated surface and not on feeder cell e.g., fibroblast feeder cells, e.g., mouse embryonic fibroblasts (MEFs)].

According to specific embodiments, the naïve PSCs can be obtained in culture in the naïve pluripotent state in the absence of exogenous L-glutamine in the culture medium.

According to specific embodiments, the naïve PSCs can be obtained in culture in the naïve pluripotent state in the absence of expression of DNA methyltransferase 1 (DNMT1).

Methods of obtaining naïve PSCs and maintaining them in a naïve and pluripotent state are known in the art and are further disclosed in the Examples section which follows which serve as an integral part of the specification and in e.g. Choi, J., Huebner, A., Clement, K. et al. Nature 548, 219-223 (2017), www(dot)doi(dot)org/10.1038/nature23274; Yang et al. Cell (2017) 169 (2): 243-257, www(dot)cell(dot)com/fulltext/S0092-8674% 2817%2930183-6; Bayerl et al. 2021, Cell Stem Cell 28, 1549-1565 and International Patent Application Publication No. WO2016/016894 and WO2014/174470, the contents or which are fully incorporated herein by reference.

Multiple naïve PSCs cell lines are also known, for example, V6.5 mouse ESC line, BVSc ES line, NGFP1 iPSC line, WIBR3 line, WIS1 ES line and WIS ES line, and are commercially available from public cell line repositories.

As used herein, the term “trophectoderm (TE) primed cell” refers to a stem cell originating from a naïve PSC that comprises an exogenous nucleic acid sequence encoding a factor that induces differentiation to a trophectoderm cell and/or a cell originating from a naïve PSC that is committed to the trophectoderm lineage (e.g. trophectoderm cell). It will be appreciated that prior to induction of expression of the factor (i.e. the protein product expressed from the exogenous nucleic acid sequence or the endogenous factor) the cell is in a naïve and undifferentiated state (i.e. naive PSC) and following induction of expression of the factor the cell acquires characteristics of a trophectoderm cell.

According to specific embodiments, the trophectoderm primed cell comprises an exogenous polynucleotide suitable for transient expression of the factor that induces differentiation to trophectoderm cells, as further described hereinbelow.

According to specific embodiments, the trophectoderm primed cell comprises the protein product expressed from the exogenous nucleic acid sequence encoding the factor.

According to other specific embodiments, the trophectoderm primed cell does not comprise the protein product expressed from the exogenous nucleic acid sequence encoding the factor.

As used herein, “trophectoderm cell” refers to a cell having a restricted potential to generate all placental lineages but not other cell lineages.

According to some embodiment, the trophectoderm cells gives rise to the ecto-placental cone, and/or extra-embryonic ectoderm (ExE), and/or the umbilical cord and/or the chorion.

Non-limiting examples of trophectoderm cell markers include Gata3, Gata2, tbr2, tfap2c, Cdx2, Elf5, Hand1, EOMES, KLF5 and ETS2.

According to specific embodiments, the trophectoderm cell expresses Cdx2, i.e. positive for Cdx2.

According to specific embodiments, the trophectoderm primed cell expresses a trophectoderm marker. According to specific embodiments, the trophectoderm primed cell expresses at least one trophectoderm marker selected from the group consisting of Gata3, Gata2, Cdx2 and Elf5.

According to specific embodiments, the trophectoderm primed cell expresses pluripotency marker. Such markers are known in the art and are further described hereinabove and below. According to specific embodiments, the trophectoderm primed cell expresses (positive for) at least one pluripotency marker selected from the group consisting of Oct4, Tfap2c. Susd2, Sox2, SSEA1, SSEA3, SSEA4, TRA1-60. According to specific embodiments, the trophectoderm primed cell expresses Oct4. According to specific embodiments, the trophectoderm primed cell is negative for a pluripotency marker selected from the group consisting of Klf17, Klf4 and Nanog.

According to specific embodiments, the trophectoderm primed cell expresses a trophectoderm marker and a pluripotency marker.

According to specific embodiments, the marker is endogenous to the cell.

Thus, according to specific embodiments, the trophectoderm primed cell expresses an exogenous factor that induces differentiation to trophectoderm cells, an endogenous trophectoderm marker and an endogenous pluripotency marker.

According to specific embodiments, the trophectoderm primed cell expresses a trophectoderm marker upon mixing (i.e., at time 0 hours), in the mixture or in the aggregate.

According to specific embodiments, the trophectoderm primed cell expresses a pluripotency marker upon mixing (i.e., at time 0 hours), in the mixture or in the aggregate.

According to specific embodiments, the trophectoderm primed cell does not express Oct4 and Nanog upon mixing (i.e., at time 0 hours), in the mixture or in the aggregate.

According to specific embodiments, the trophectoderm primed cell (e.g., upon mixing, in the mixture, in the aggregate) is not in a differentiation stage of a trophoblast stem cell (TSC). In other words, it does not have a phenotypic signature of a TSC (derived from the trophectoderm or an induced TSC or obtained by de-differentiation or re-programming of a cell).

According to specific embodiments, the TSC (e.g., human TSC) has a CDX2⁻ TFAP2C⁺GATA3⁺SIGLE6⁺ phenotype.

In comparison, the human trophectoderm primed cell or some embodiments of the invention has a CDX2+TFAP2c⁺GATA3⁺SIGLEC6⁻ phenotype.

According to specific embodiments, the TSC (e.g., mouse TSC) has a CDX2⁺TFAP2C⁺MHC class 1⁺OCT4⁻ phenotype.

In comparison, the mouse trophectoderm primed cell or some embodiments of the invention has a CDX2⁺TFAP2C⁺MHC class 1⁻Oct4^{+(low)} phenotype.

The factor which expression induces differentiation of the naïve PSCs of some embodiments of the invention may be endogenous and/or exogenous to the cell.

According to specific embodiments, the method comprises inducing expression of an exogenous factor that induces differentiation to (towards) trophectoderm cells.

As used herein, the term “exogenous” refers to a heterologous polynucleotide or polypeptide which is not naturally expressed within the cell or which overexpression in the cell is desired. It should be noted that the exogenous polynucleotide and/or polypeptide may comprise a nucleic acid sequence and/or an amino acid sequence, respectively, which is identical or partially homologous to an endogenous nucleic acid sequence and/or an endogenous amino acid sequence of the cell.

According to additional or alternative embodiments, the method comprises inducing expression of an endogenous factor that induces differentiation to (toward) trophectoderm cells. Hence, according to specific embodiments, the cell is cultured under conditions enabling expression of an endogenous factor that induces differentiation to the trophectoderm primed cell. Such conditions include for example an appropriate temperature (e.g., 37° C.), atmosphere (e.g., % O₂, % CO₂), pressure, pH, light, medium, supplements (e.g. a differentiation factor as disclosed herein) and the like. Further description on culturing conditions are provided hereinbelow. Hence, according to specific embodiments, the naïve PSC cell or the trophectoderm primed cell is contacted with the factor. Under this culturing scenario the cells may further comprise or be devoid of an exogenous polynucleotide suitable for expression of a factor that induces differentiation to trophectoderm cells. According to a specific embodiment, the cell does not comprise an exogenous polynucleotide suitable for expression of a factor that induces differentiation to trophectoderm cells. In this case inducing expression of the endogenous factor, the culturing or the contacting is typically effected prior to mixing the subpopulations of cells (e.g. starting within 14 days-0 hours prior to the mixing, within 13 days-0 hours prior to the mixing, within 12 days-0 hours prior to the mixing, within 11 days-0 hours prior to the mixing, within 10 days-0 hours prior to the mixing, within 9 days-0 hours prior to the mixing, within 8 days-0 hours prior to the mixing, within 7 days-0 hours prior to the mixing, within 6 days-0 hours prior to the mixing within 5 days-0 hours prior to the mixing, within 120 hours-0 hours prior to the mixing, within 100 hours-0 hours prior to the mixing, within 80 hours-0 hours prior to the mixing, within 60 hours-0 hours prior to the mixing, within 48 hours-0 hours prior to the mixing, within 36 hours-0 hours prior to the mixing, within 24 hours-0 hours prior to the mixing, within 12 hours-0 hours prior to the mixing, within 14-1 days prior to the mixing, within 13-1 days prior to the mixing, within 12-1 days prior to the mixing, within 11-1 days prior to the mixing, within 10-1 days prior to the mixing, within 9-1 days prior to the mixing, within 8-1 days prior to the mixing, within 7-1 days prior to the mixing, within 6-1 days prior to the mixing within 5-1 days prior to the mixing, within 120-20 hours prior to the mixing, within 100-20 hours prior to the mixing, within 80-20 hours prior to the mixing, within 60-20 hours prior to the mixing, within 48-20 hours prior to the mixing, within 14 days-12 hours prior to the mixing, within 13 days-12 hours prior to the mixing, within 12 days-12 hours prior to the mixing, within 11 days-12 hours prior to the mixing, within 10 days-12 hours prior to the mixing, within 9 days-12 hours prior to the mixing, within 8 days-12 hours prior to the mixing, within 7 days-12 hours prior to the mixing, within 6 days-12 hours prior to the mixing within 5 days-12 hours prior to the mixing, within 120-12 hours prior to the mixing, within 100-12 hours prior to the mixing, within 80-12 hours prior to the mixing, within 60-12 hours prior to the mixing, within 48-12 hours prior to the mixing, within 14 days-6 hours prior to the mixing, within 13 days-6 hours prior to the mixing, within 12 days-6 hours prior to the mixing, within 11 days-6 hours prior to the mixing, within 10 days-6 hours prior to the mixing, within 9 days-6 hours prior to the mixing, within 8 days-6 hours prior to the mixing, within 7 days-6 hours prior to the mixing, within 6 days-6 hours prior to the mixing within 5 days-6 hours prior to the mixing, within 120 hours-6 hours prior to the mixing, within 100 hours-6 hours prior to the mixing, within 80 hours-6 hours prior to the mixing, within 60 hours-6 hours prior to the mixing, within 48 hours-6 hours prior to the mixing, within 36 hours-6 hours prior to the mixing, within 24 hours-6 hours prior to the mixing, or within 12 hours-6 hours prior to the mixing, each possibility represents a separate embodiment of the invention).

According to specific embodiments, the culturing or the contacting starts at least 6 hours, at least 10 hours, at least 12 hours, at least 16 hours, at least 24 hours, at least 36 hours, at least 48 hours, at least 72 hours, or at least 96 hours prior to the mixing.

According to specific embodiments, the culturing or the contacting starts no more than 3 days, no more than 4 days, no more than 5 days, no more than 6 days, no more that 7 days, no more than 8 days, no more than 9 days, no more than 10 days, not more that 14 days, no more than 16 days, or no more than 20 days prior to the mixing.

According to specific embodiments, the culturing or the contacting starts within about 6-3 days prior to the mixing.

According to specific embodiments, the culturing or the contacting starts about 3 days prior to the mixing.

Factors that induce differentiation towards trophectoderm cells are well known in the art, and include factor expressed in the cells and factor that are contacted with the cells. Non-limiting examples of such factors include Cdx2, Gata3, Gata2, TGFR inhibitor, FGFR inhibitor, MEK/ERK inhibitor, BMP4, JAK inhibitor, FGF4, FGF2, heparin, a SUMOylation inhibitor, a Histone Deacetylase inhibitor, a HIPPO signaling pathway inhibitor and a factor that induces YAP nuclear translocation.

According to specific embodiments, the factor that induces differentiation to trophectoderm cells is Cdx2.

As used herein, the term “Cdx2”, refers to the polynucleotide and expression product e.g., polypeptide of the CDX2 gene (corresponding to human gene ID NO: 1045) or homolog or ortholog of same. According to specific embodiments the Cdx2 refers to the human Cdx2, such as provided in the following GeneBank Numbers NP_001256, NP_001341629, NM_001265, NM_001354700 (SEQ ID NO: 68-70). According to specific embodiments the Cdx2 refers to the mouse Cdx2, such as provided in the following GeneBank Numbers NP_031699 and NM_007673 and (SEQ ID NO: 72-73). A homolog (as further defined hereinbelow) refers to a functional expression product of Cdx2 capable of inducing the differentiation of naïve PSCs to trophectoderm cells.

Thus, as mentioned any of the polynucleotides and expression products described herein (e.g., Cdx2, Gata4 etc.), also refer to homologues which exhibit the desired activity as defined herein. Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polypeptide or polynucleotide sequences provided herein.

The homolog may also refer to an ortholog, a deletion, insertion, or substitution variant, including an amino acid substitution, as long as it retains the activity.

Sequence identity or homology can be determined using any protein or nucleic acid sequence alignment algorithm such as Blast, ClustalW, and MUSCLE.

According to specific embodiments, the factor that induces differentiation to trophectoderm cells is Gata3.

As used herein, the term “Gata3”, also known as GATA Binding Protein 3 and HDRS, refers to the polynucleotide and expression product e.g., polypeptide of the GATA3 gene (corresponding to human gene ID NO: 2625) or homolog or ortholog of same. According to specific embodiments the Gata3 refers to the human Gata3, such as provided in the following GeneBank Numbers NP_001002295 and NM_001002295 (SEQ ID NO: 74). According to specific embodiments the Gata3 refers to the mouse Gata3, such as provided in the following GeneBank Numbers NP_032117, NP_001342039, NP_001342040, NP_001342041, NM_008091. NM_001355110, NM_001355111 and NM_001355112 (SEQ ID NO: 76-83). A homolog (as further defined hereinabove) refers to a functional expression product of Gata3 capable of inducing the differentiation of naïve PSCs to trophectoderm cells.

According to specific embodiments, the factor that induces differentiation to trophectoderm cells is Gata2.

As used herein, the term “Gata2”, also known as GATA Binding Protein 2, refers to the polynucleotide and expression product e.g., polypeptide of the GATA2 gene (corresponding to human gene ID NO: 2624) or homolog or ortholog of same. According to specific embodiments the Gata2 refers to the human Gata2, such as provided in the following GeneBank Numbers NP_001139133, NP_001139134, NP_116027, NM_032638, NM_001145661 and NM_001145662 (SEQ ID NO: 84-89). According to specific embodiments the Gata2 refers to the mouse Gata2, such as provided in the following GeneBank Numbers NP_032116, NP_001342182, NM_008090 and NM_001355253 (SEQ ID NO: 90-93). A homolog (as further defined hereinabove) refer to a functional expression product of Gata2 capable of inducing the differentiation of naïve PSCs to trophectoderm cells.

According to specific embodiments, the factor that induces differentiation to trophectoderm cells is a TGFR inhibitor.

As used herein the term “transforming growth factor receptor (TGFR)” (also known as “TGFβR”) refers to TGF-β type I receptor ALK5, type I activin/nodal receptor ALK4 and type I nodal receptor ALK7.

As used herein the term “TGFR inhibitor (or TGFRi)” refers to a molecule capable of inhibiting TGFR expression and/or activity level as determined by phosphorylated ALK4, 5 and 7. Non-limiting examples of TGFR inhibitors include SB431542 and A 83-01 small molecule compound.

According to specific embodiments, the factor that induces differentiation to trophectoderm cells is a FGFR inhibitor.

As used herein the term “fibroblast growth factor receptor (FGFR)” refers to FGFR1, FGFR2 and FGFR3.

As used herein the term “FGFR inhibitor (or FGFRi)” refers to a molecule capable of inhibiting FGFR expression and/or activity level as determined by levels of phosphorylated FGFR1, 2, and 3. Non-limiting examples of FGFR inhibitors include PD173074, SU5401 and PD0325901.

According to specific embodiments, the factor that induces differentiation to trophectoderm cells is ROCK inhibitor.

As used herein the term “ROCK”, also known as Rho-associated protein kinase and includes ROCK1 and ROCK2 having a serine/threonine kinase activity.

As used herein the term “ROCK inhibitor” refers to any molecule capable of inhibiting the activity of ROCK as determined by inhibition of ROCK phosphorylation levels (e.g. detected by western blot analysis).

Non-limiting examples of ROCK inhibitors include Y27632 (TOCRIS, Catalogue number 1254), Blebbistatin (TOCRIS Catalogue number 1760) and Thiazovivin (Axon Medchem—Axon 1535). Blebbistatin is a selective inhibitor of myosin II ATpase, a downstream effector of ROCK pathway, and thus effectively leads to ROCK pathway inhibition (Chen G1, Hou Z et al. Actin-myosin contractility is responsible for the reduced viability of dissociated human embryonic stem cells. Cell Stem Cell. 2010; 7 (2): 240-8, which is fully incorporated herein by reference in its entirety).

According to specific embodiments, the factor that induces differentiation to trophectoderm cells is a MEK/ERK inhibitor (as further defined infra).

According to specific embodiments, the factor that induces differentiation to trophectoderm cells is BMP4.

As used herein, the term “BMP4”, also known as Bone morphogenic protein 4, refers to the polynucleotide and expression product e.g., polypeptide of the BMP4 gene (corresponding to human gene ID NO: 652) or a homolog or ortholog of same. According to specific embodiments the BMP4 refers to the human BMP4, such as provided in the following UniProt Number P12644 (SEQ ID NO: 195). According to specific embodiments the BMP4 refers to the mouse BMP4, such as provided in the following UniProt Number P21275 (SEQ ID NO: 196). A homolog (as further defined hereinabove) refers to a functional expression product of BMP4 capable of inducing the differentiation of naïve PSCs to trophectoderm cells.

According to specific embodiments, the factor that induces differentiation to trophectoderm cells is a JAK inhibitor.

As used herein the term “JAK” refers to a Janus kinase and includes JAK1, JAK2, JAK3 and TYK2.

As used herein the term “JAK inhibitor (or JAKi)” refers to a molecule capable of inhibiting JAK expression and/or activity level as determined by levels of phosphorylated STAT (signal transducer and activator of transcription). Non-limiting examples of JAK inhibitors include Calbiochem 420099, Tofacitinib, Baricitinib, Ruxolitinib, Fedratinib, Upadacitinib, Cedulatinib, Gandotinib, Lesraurtinib, Momelotinib, Pacritinib, Curuebitacin I, CHZ868.

According to specific embodiments, the factor that induces differentiation to trophectoderm cells is FGF4.

As used herein, the term “FGF4”, also known as Fibroblast growth factor 4, refers to the polynucleotide and expression product e.g., polypeptide of the FGF4 gene (corresponding to human gene ID NO: 2249) or homolog or ortholog of same. According to specific embodiments the FGF4 refers to the human FGF4, such as provided in the following GeneBank Numbers NP_001998 and NM_002007 (SEQ ID NO: 94-95). According to specific embodiments the FGF4 refers to the mouse FGF4, such as provided in the following GeneBank Numbers NP_034332 and NM_010202 (SEQ ID NO: 96-97). A homolog (as further defined hereinabove) refers to a functional expression product of FGF4 capable of inducing the differentiation of naïve PSCs to trophectoderm cells and/or extra embryonic primitive endodermal cells.

According to specific embodiments, the factor that induces differentiation to trophectoderm cells is FGF2.

As used herein, the term “FGF2”, also known as basic fibroblast growth factor (bFGF) and FGF-β, refers to the polynucleotide and expression product e.g., polypeptide of the FGF2 gene (corresponding to human gene ID NO: 2247) or homolog or ortholog of same. According to specific embodiments the FGF2 refers to the human FGF2, such as provided in the following GeneBank Numbers NP_001997, NP_001348594, NM_002006 and NM_001361665 (SEQ ID NO: 98-101). According to specific embodiments the FGF2 refers to the mouse FGF2, such as provided in the following GeneBank Numbers NP_032032 and NM_008006 (SEQ ID NO: 102-103). A homolog (as further defined hereinabove) refers to a functional expression product of FGF2 capable of inducing the differentiation of naïve PSCs to trophectoderm cells and/or extra embryonic primitive endodermal cells.

According to specific embodiments, the factor that induces differentiation to trophectoderm cells is a SUMOylation inhibitor, such as but not limited to DC 2-D08 (2′,3′,4′-trihydroxy flavone).

According to specific embodiments, the factor that induces differentiation to trophectoderm cells is a Histone Deacetylase inhibitor (HDACi), such as but not limited to VPA (valproic acid), Trichostatin A, sodium butyrate or UF010 small molecule inhibitor.

According to specific embodiments, the factor that induces differentiation to trophectoderm cells is a HIPPO signaling pathway inhibitor or a factor that induces YAP nuclear translocation.

The HIPPO signaling pathway, also known as the Salvador/Warts/Hippo (SWH) pathway, comprise a complex cascade of serine/threonine-protein kinases that eventually regulates phosphorylation of Yap/Taz. Specifically, when the Hippo pathway is ‘off’, the phosphorylated YAP/TAZ is retained in the cytoplasm and may also undergo proteolytic degradation. When the Hippo pathway is ‘on’, the unphosphorylated YAP/TAZ moves into the nucleus and binds to transcription factors called TEA DNA-binding proteins (TEAD1-4). Non-limiting examples of components of the HIPPO signaling pathway include Hpo, Serine threonine kinase 3 (STK3) and STK4 (also known as MST1 and MST2), salvador homolog 1 (SAV1), large tumor suppressor 1/2 (LATS1/2). MAP4K, TAOK, MOB kinase activator 1A/B (MOB1A/B), yes-associated protein (YAP1), transcriptional co-activator with PDZ-binding motif (TAZ or WWTR1) and Neurofibromin 2 (NF2).

The HIPPO signaling pathway inhibitor disclosed herein binds a component of the HIPPO signaling pathway and inhibits expression and/or activity of same. Such inhibitors are known in the art and include, but not limited to LPA and XMU-MP-1.

As used herein, the term “extra embryonic primitive endodermal (PRE) primed cell” refers to a stem cell originating from a naïve PSC that comprises an exogenous nucleic acid sequence encoding a factor that induces differentiation to an extra embryonic primitive endodermal cell and/or a cell originating from a naïve PSC that is committed to the extra embryonic primitive endoderm lineage (e.g. extra embryonic primitive endodermal cell).

It will be appreciated that prior to induction of expression of the exogenous factor (i.e. the protein product expressed from the exogenous nucleic acid sequence or the endogenous factor) the cell is in a naïve and undifferentiated state (i.e. naive PSC) and following induction of expression of the exogenous factor the cell acquires characteristics of an extra embryonic primitive endodermal cell.

According to specific embodiments, the extra embryonic primitive endodermal primed cell comprises an exogenous polynucleotide suitable for transient expression of the factor that induces differentiation to extra embryonic primitive endodermal cells, as further described hereinbelow.

According to specific embodiments, the extra embryonic primitive endodermal primed cell comprises the protein product expressed from the exogenous nucleic acid sequence encoding the factor.

According to other specific embodiments, the extra embryonic primitive endodermal primed cell does not comprise the protein product expressed from the exogenous nucleic acid sequence encoding the factor.

As used herein, “extra embryonic primitive endodermal cell” refers to a cell having a restricted potential to generate all primitive endoderm lineages but not other cell lineages.

Non-limiting examples of extra embryonic primitive endodermal cell markers include Foxa2, Gata4, Gata6, PDGFRA and Sox17.

According to specific embodiments, the extra embryonic primitive endodermal cell expresses Gata4, i.e. positive for Gata4.

According to specific embodiments, the extra embryonic primitive endodermal primed cell expresses an extra embryonic primitive endoderm marker. According to specific embodiments, the extra embryonic primitive endodermal primed cell expresses at least one extra embryonic primitive endoderm marker selected from the group consisting of PDGFRA, GATA4, SOX17, NID2 and GATA6.

According to specific embodiments, the extra embryonic primitive endodermal primed cell expresses a pluripotency marker. Such markers are known in the art and are further described hereinabove and below. According to specific embodiments, the extra embryonic primitive endodermal primed cell expresses (positive for) at least one pluripotency marker selected from the group consisting of OCT4, SSEA1 and SSEA4. According to specific embodiments, the extra embryonic primitive endodermal primed cell is negative for a pluripotency marker selected from the group consisting of NANOG, KLF17, NANOG-NB and, TFCP2L1.

According to specific embodiments, the extra embryonic primitive endodermal primed cell expresses an extra embryonic primitive endoderm marker and a pluripotency marker.

According to specific embodiments, the marker is endogenous to the cell.

Thus, according to specific embodiments, the extra embryonic primitive endodermal primed cell expresses an exogenous factor that induces differentiation to extra embryonic primitive endodermal cells, an endogenous extra embryonic primitive endoderm marker and an endogenous pluripotency marker.

According to specific embodiments, the extra embryonic primitive endodermal primed cell expresses an extra embryonic primitive endoderm marker upon mixing (i.e. at time 0 hours), in the mixture or in the aggregate.

According to specific embodiments, the extra embryonic primitive endodermal primed cell expresses a pluripotency marker upon mixing (i.e. at time 0 hours), in the mixture or in the aggregate.

According to specific embodiments, the extra embryonic primitive endodermal primed cell expresses Oct4 upon mixing (i.e. at time 0 hours), in the mixture or in the aggregate

According to other specific embodiments, the extra embryonic primitive endodermal primed cell does not express Oct4 upon mixing (i.e. at time 0 hours), in the mixture or in the aggregate.

According to specific embodiments, the extra embryonic primitive endodermal primed cell (e.g. upon mixing, in the mixture, in the aggregate) is not in a differentiation stage of a XEN cell. In other words, it does not have a phenotypic signature of a XEN (which can be obtained from blastocysts, early post-implantation embryo or an induced XEN obtained by de-differentiation or re-programming of a cell).

According to specific embodiments, the XEN has an OCT4⁻ GATA6⁺GATA4⁺SOX17⁺PDGFRa⁺ phenotype.

In comparison the extra embryonic primitive endodermal primed cell of some embodiments of the invention has an OCT4⁺GATA6⁺GATA4⁺SOX17⁺PDGFRa⁺ phenotype.

As mentioned, the factor which expression induces differentiation of the naïve PSCs of some embodiments of the invention may be endogenous and/or exogenous to the cell.

According to specific embodiments, the method comprises inducing expression of an exogenous factor that induces differentiation to (towards) extra embryonic primitive endodermal cells.

According to additional or alternative embodiments, the method comprises inducing expression of an endogenous factor that induces differentiation to (towards) extra embryonic primitive endodermal cells. Hence, according to specific embodiments, the cell is cultured under conditions enabling expression of an endogenous factor that induces differentiation to the extra embryonic primitive endodermal primed cell. Such conditions include for example an appropriate temperature (e.g., 37° C.), atmosphere (e.g., % O₂, % CO₂), pressure, pH, light, medium, supplements (e.g. a differentiation factor as disclosed herein) and the like. Further description on culturing conditions are provided hereinbelow. Hence, according to specific embodiments, the naïve PSC cell or the extra embryonic primitive endodermal primed cell is contacted with the factor. Under this culturing scenario the cells may further comprise or be devoid of an exogenous polynucleotide suitable for expression of a factor that induces differentiation to extra embryonic primitive endodermal cells. According to a specific embodiment, the cell does not comprise an exogenous polynucleotide suitable for expression of a factor that induces differentiation to extra embryonic primitive endodermal. In this case inducing expression of the endogenous factor, the culturing or the contacting is typically effected prior to mixing the subpopulations of cells (e.g. starting within 14 days-0 hours prior to the mixing, within 13 days-0 hours prior to the mixing, within 12 days-0 hours prior to the mixing, within 11 days-0 hours prior to the mixing, within 10 days-0 hours prior to the mixing, within 9 days-0 hours prior to the mixing, within 8 days-0 hours prior to the mixing, within 7 days-0 hours prior to the mixing, within 6 days-0 hours prior to the mixing within 5 days-0 hours prior to the mixing, within 120 hours-0 hours prior to the mixing, within 100 hours-0 hours prior to the mixing, within 80 hours-0 hours prior to the mixing, within 60 hours-0 hours prior to the mixing, within 48 hours-0 hours prior to the mixing, within 36 hours-0 hours prior to the mixing, within 24 hours-0 hours prior to the mixing, within 12 hours-0 hours prior to the mixing, within 14-1 days prior to the mixing, within 13-1 days prior to the mixing, within 12-1 days prior to the mixing, within 11-1 days prior to the mixing, within 10-1 days prior to the mixing, within 9-1 days prior to the mixing, within 8-1 days prior to the mixing, within 7-1 days prior to the mixing, within 6-1 days prior to the mixing within 5-1 days prior to the mixing, within 120-20 hours prior to the mixing, within 100-20 hours prior to the mixing, within 80-20 hours prior to the mixing, within 60-20 hours prior to the mixing, within 48-20 hours prior to the mixing, within 14 days-12 hours prior to the mixing, within 13 days-12 hours prior to the mixing, within 12 days-12 hours prior to the mixing, within 11 days-12 hours prior to the mixing, within 10 days-12 hours prior to the mixing, within 9 days-12 hours prior to the mixing, within 8 days-12 hours prior to the mixing, within 7 days-12 hours prior to the mixing, within 6 days-12 hours prior to the mixing within 5 days-12 hours prior to the mixing, within 120-12 hours prior to the mixing, within 100-12 hours prior to the mixing, within 80-12 hours prior to the mixing, within 60-12 hours prior to the mixing, or within 48-12 hours prior to the mixing, within 14 days-6 hours prior to the mixing, within 13 days-6 hours prior to the mixing, within 12 days-6 hours prior to the mixing, within 11 days-6 hours prior to the mixing, within 10 days-6 hours prior to the mixing, within 9 days-6 hours prior to the mixing, within 8 days-6 hours prior to the mixing, within 7 days-6 hours prior to the mixing, within 6 days-6 hours prior to the mixing within 5 days-6 hours prior to the mixing, within 120 hours-6 hours prior to the mixing, within 100 hours-6 hours prior to the mixing, within 80 hours-6 hours prior to the mixing, within 60 hours-6 hours prior to the mixing, within 48 hours-6 hours prior to the mixing, within 36 hours-6 hours prior to the mixing, within 24 hours-6 hours prior to the mixing, or within 12 hours-6 hours prior to the mixing, each possibility represents a separate embodiment of the invention).

According to specific embodiments, the culturing or the contacting starts at least 6 hours, at least 10 hours, at least 12 hours, at least 16 hours, at least 24 hours, at least 36 hours, at least 48 hours, at least 72 hours, or at least 96 hours prior to the mixing.

According to specific embodiments, the culturing or the contacting starts no more than 3 days, no more than 4 days, no more than 5 days, no more than 6 days, no more that 7 days, no more than 8 days, no more than 9 days, no more than 10 days, not more that 14 days, no more than 16 days, or no more than 20 days prior to the mixing.

According to specific embodiments, the culturing or the contacting starts within about 6-3 days prior to the mixing.

According to specific embodiments, the culturing or the contacting starts about 3 days prior to the mixing.

Factors that induce differentiation to extra embryonic primitive endodermal cells are well known in the art, and include factor expressed in the cells and factor that are contacted with the cells. Non-limiting examples of such factors include Gata4, Gata6, a GSK-3 inhibitor, WNT ligand, heparin, FGF2, FGF4, PDGFRA, leukemia inhibitory factor (LIF) and insulin.

According to specific embodiments, the factor that induces differentiation to extra embryonic primitive endodermal cells is Gata4.

As used herein, the term “Gata4”, also known as GATA Binding Protein 4 and Transcription factor Gata4, refers to the polynucleotide and expression product e.g., polypeptide of the GATA4 gene (corresponding to human gene ID NO: 2626) or homolog or ortholog of same. According to specific embodiments the Gata4 refers to the human Gata4, such as provided in the following GeneBank Numbers NP_001295022, NP_001295023, NP_002043, NP_001361202, NP_001361203, NM_001308093, NM_001308094, NM_002052, NM_001374273 and NM_001374274 (SEQ ID NO: 104-113). According to specific embodiments the Gata4 refers to the mouse Gata4, such as provided in the following GeneBank Numbers NP_001297539, NP_032118, NM_008092 and NM_001310610 (SEQ ID NO: 114-117). A homolog (as further defined hereinabove) refers to a functional expression product of Gata4 capable of inducing the differentiation of naïve PSCs to extra embryonic primitive endodermal cells.

According to specific embodiments, the factor that induces differentiation to extra embryonic primitive endodermal cells is Gata6.

As used herein, the term “Gata6”, also known as GATA Binding Protein 6 and Transcription factor Gata6, refers to the polynucleotide and expression product e.g., polypeptide of the GATA6 gene (corresponding to human gene ID NO: 2627) or homolog or ortholog of same. According to specific embodiments the Gata6 refers to the human Gata6, such as provided in the following GeneBank Numbers NP_005248 and NP_005257 (SEQ ID NO: 118-119). According to specific embodiments the Gata6 refers to the mouse Gata6, such as provided in the following GeneBank Numbers NP_034388 and NM_010258 (SEQ ID NO: 120-121). A homolog (as further defined hereinabove) refers to a functional expression product of Gata6 capable of inducing the differentiation of naïve PSCs to extra embryonic primitive endodermal cells.

According to specific embodiments, the factor that induces differentiation to extra embryonic primitive endodermal cells is SOX17.

As used herein, the term “SOX17”, also known as SRY-box 17, refers to the polynucleotide and expression product e.g., polypeptide of the SOX17 gene (corresponding to human gene ID NO: 64321) or homolog or ortholog of same. According to specific embodiments the SOX17 refers to the human SOX17, such as provided in the following GeneBank Numbers NP_071899 and NM_022454 (SEQ ID NO: 197-198). According to specific embodiments the SOX17 refers to the mouse SOX17, such as provided in the following UniProt No. Q61473 (SEQ ID NO: 199). A homolog (as further defined hereinabove) refers to a functional expression product of SOX17 capable of inducing the differentiation of naïve PSCs to extra embryonic primitive endodermal cells.

According to specific embodiments, the factor that induces differentiation to extra embryonic primitive endodermal cells is a GSK-3 inhibitor.

As used herein the term “GSK-3” refers to the glycogen synthase kinase 3 beta protein set forth by GenBank Accession Nos. NP_002084 (SEQ ID NO: 122) and/or NP_001139628 (SEQ ID NO: 123) having the WNT signaling regulatory activity via its kinase activity.

As used herein the term “GSK-3 inhibitor” refers to any molecule capable of inhibiting the activity of GSK-3 as determined by specifically inhibiting levels of phosphorylated GSK3b (out of total GSK3b present in a cell).

Non-limiting examples of GSK-3 inhibitors include CHIR99021 (AXON MEDCHEM-AXON 1386), BIO (AXONMEDCHEM—Axon 1693), and Kenpaullone (TOCRIS-cat no. 1398).

According to specific embodiments, the factor that induces differentiation to extra embryonic primitive endodermal cells is a WNT ligand.

Wnt ligands are a large family of secreted glycoproteins that are cysteine-rich and highly hydrophobic. Non-limiting examples of Wnt ligands that can be used with specific embodiments of the invention include Wnt-1, Wnt-2, Wnt-2b, Wnt-3a, Wnt-4, Wnt-5a, Wnt-5b, Wnt-6, Wnt-7a. Wnt-7b, Wnt8a, Wnt-8b, Wnt-9a, Wnt-9b, Wnt-10a, Wnt-10b, Wnt-11 and Wnt16b.

According to specific embodiments, the factor that induces differentiation to extra embryonic primitive endodermal cells is FGF2.

According to specific embodiments, the factor that induces differentiation to extra embryonic primitive endodermal cells is FGF4.

According to specific embodiments, the factor that induces differentiation to extra embryonic primitive endodermal cells is PDGF.

As used herein, the term “PDGF”, refers to a platelet-derived growth factor, and includes the various subtypes of PDGF including PDGF-B (e.g. GenBank Accession Nos. X02811 and CAA26579), PDGF-A (e.g. GenBank Accession nos. X06374 and CAA29677), PDGF-C(e.g. GenBank Accession Nos. NM 016205 and NP 057289), PDGF-D, variants 1 and 2 (e.g. GenBank Accession Nos. NM 025208, NP 079484, NM 033135, NP 149126), and dimerized forms thereof, including PDGF-AA, PDGF-AB, PDGF-BB, PDGF-CC, and PDGF-DD. Typically, platelet-derived growth factors includes homo- or heterodimers of A-chain and B-chain that exert their action via binding to and dimerization of two related receptor tyrosine kinase platelet-derived growth factor cell surface receptors PDGFR-α (also known as PDGFRA, e.g. GenBank Accession Nos. NM 006206 and NP 006197) and PDGFR-β (e.g. GenBank Accession Nos. NM 002609 and NP 002600). See also PCT Application Publication No. WO2010/127029 and US Application Publication No. US20150182623, which are incorporated herein in its entirety, for PDGF sequences. According to specific embodiments, the PDGF is capable of inducing the differentiation of naïve PSCs to extra embryonic primitive endodermal cells.

According to specific embodiments, the factor that induces differentiation to extra embryonic primitive endodermal cells is LIF (as further defined infra).

According to specific embodiments, the factor that induces differentiation to extra embryonic primitive endodermal cells is insulin.

As the present inventors developed culture conditions which enabled the generation of extra embryonic primitive endodermal primed cells from naïve PSCs which can be used for example for generating synthetic embryos (Examples 10 and 14 of the Examples section which follows), embodiments of the invention also contemplate methods of obtaining these cells.

Thus, according to an aspect of the present invention, there is provided a method of obtaining extra embryonic primitive endodermal primed cells, the method comprising culturing naïve PSCs in a base medium devoid of Activin A under conditions that allow differentiation of said naïve PSCs to GATA4⁺GATA6⁺OCT4⁺Nanog⁻ extra embryonic primitive endodermal primed cells, thereby obtaining the extra embryonic primitive endodermal primed cells.

According to specific embodiments, the GATA4⁺GATA6⁺OCT4⁺Nanog⁻ cells are also positive for PDGFRA and NID2.

According to specific embodiments of this aspect of the present invention, the method further comprising inducing expression (e.g. transient expression) of an exogenous factor that induces differentiation to extra embryonic primitive endodermal cells.

According to other specific embodiments of this aspect of the present invention, the method does not comprise inducing expression (e.g. transient expression) of an exogenous factor that induces differentiation to extra embryonic primitive endodermal cells.

According to specific embodiments, the culturing is effected for at least 6 hours, at least 12 hours, at least 16 hours, at least 1 day (or at least 24 hours), at least 2 days or at least 3 days.

According to specific embodiments, the culturing is effected for up to 14 days, up to 13 days, up to 12 days, up to 11 days, up to 10 days, up to 9 days, up to 8 days, up to 7 days or up to 6 days.

According to specific embodiments, the culturing is effected for 1-10 days.

According to specific embodiments, the culturing is effected for about 3-6 days.

According to specific embodiments, the culturing is effected from about 3 days.

According to specific embodiments, the obtained cells are a stable line maintaining the extra embryonic primitive endodermal primed cells phenotype (e.g. GATA4⁺GATA6⁺OCT4⁺Nanog⁻ phenotype) over multiple generations/passages. Thus, according to specific embodiments, the cells can be expanded in culture for at least 3, at least 4, at least 5, at least 6, at least 7, at least 8, at least 9, at least 10, at least 20, or at least 30 passages while maintaining the extra embryonic primitive endodermal primed cells phenotype (e.g. GATA4⁺GATA6⁺OCT4⁺Nanog⁻ phenotype).

The exogenous factors of some embodiments of the invention are transiently expressed in the cells.

Methods of expressing an exogenous nucleic acid sequence and/or amino acid sequence are known in the art and include those described for example in the materials and methods of the Examples section which follows and in Mansour et al. 2012; Warren et al. 2010 and Hongyan Zhou al. Cell Stem Cell (2009) 4 (6): 581; Rabinovich and Weissman (2013) Methods Mol Biol. 969:3-28; International Application Publication No. WO 2013049389 and U.S. Pat. No. 8,557,972, which are fully incorporated herein by reference in their entirety.

Further, methods of transient expression are well known in the art and include, but not limited to, expression under the control of an inducible promoter, by introduction of mRNA into the cells (in this way the mRNA will be translated in the cells and degraded after a relatively short time) or by transfecting with the protein, as further described infra.

Distinguishing a cell expressing an exogenous polynucleotide and/or polypeptide from a cell not expressing the exogenous polynucleotide and/or polypeptide can be effected by e.g. determining the level and/or distribution of the RNA and/or protein molecules in the cell, the location of DNA integration in the genome of the cell and/or the number of gene copy number. Methods of determining the presence of an exogenous polynucleotide and/or polypeptide in a cell are well known in the art and include e.g. PCR, DNA and RNA sequencing, Southern blot, Western blot, immunoprecipitation, immunocytochemistry, flow cytometry and imaging.

According to specific embodiments, the expression is affected by introducing to the cells a polynucleotide encoding the factor.

According to specific embodiments, expressing is not in the natural location (i.e., gene locus) and/or expression level (e.g., copy number and/or cellular localization) of the native gene of the factor.

Alternatively or additionally, exogenous expression of a factor may be facilitated by activation of the endogenous locus of these genes such that the factor is overexpressed in the cell. Methods of activating and overexpressing an endogenous gene are well known in the art [see for example Menke D. Genesis (2013) 51:618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include, but not limited to and include targeted homologous recombination (e.g. “Hit and run”, “double-replacement”), site specific recombinases (e.g. the Cre recombinase and the Flp recombinase), PB transposases (e.g. Sleeping Beauty, piggyBac, Tol2 or Frog Prince), genome editing by engineered nucleases (e.g. meganucleases, Zinc finger nucleases (ZFNs), transcription-activator like effector nucleases (TALENs) and CRISPR/Cas system) and genome editing using recombinant adeno-associated virus (rAAV) platform, and small molecules. Agents for introducing nucleic acid alterations to a gene of interest can be designed publically available sources or obtained commercially from Transposagen, Addgene and Sangamo Biosciences.

As used herein the term “polynucleotide” refers to a single or double stranded nucleic acid sequence in the form of an RNA sequence (e.g. mRNA), a complementary polynucleotide sequence (cDNA), a genomic polynucleotide sequence (e.g. sequence isolated from a chromosome), a composite polynucleotide sequences (e.g., a combination of the above) or mimetic or analog thereof. This term includes polynucleotides and/or oligonucleotides derived from naturally occurring nucleic acids molecules (e.g., RNA or DNA), synthetic polynucleotide and/or oligonucleotide molecules composed of naturally occurring bases, sugars, and covalent internucleoside linkages (e.g., backbone), as well as synthetic polynucleotides and/or oligonucleotides having non-naturally occurring portions, which function similarly to the respective naturally occurring portions.

According to specific embodiments, the polynucleotide is a modified polynucleotide e.g. modified RNA.

Such a modified polynucleotide may comprise a modification in either backbone, internucleoside linkages or bases. The modified polynucleotide may comprise naturally modified nucleotides or synthetic nucleoside analogous. Modified polynucleotides may be preferred over native forms according to specific embodiments, because of desirable properties such as, for example, enhanced cellular uptake, enhanced affinity for nucleic acid target, increased stability in the presence of nucleases and decreased immunogenicity.

Such modifications include, but are not limited to 5-methoxyuridine, Pseudouridine, 5-methyl cytidine, N6-methyladenosine, 2′-O-methyl, 2′-O-methyl 3′phosphorothioate, 2′-O-methyl 3′thiophosphonoacetate, Locked nucleic acid (LNA).

Methods of stabilizing mRNA are known in the art and include modulation of the length of the polyadenine tail found at the 3″ end of the mRNA transcript. Alternatively or additionally, the RNA cap found at the molecule's 5′ end can be modified. The naturally occurring cap structure typical in mammalian cells has a tendency to be improperly incorporated into RNAs synthesized in vitro, rendering them less effective. Synthetic “anti-reverse cap analogs” (e.g. those commercially available at Thermo Fisher Scientific) can prevent this misincorporation, which results in more stable RNA with improved translational efficiency. In order to reduce immunogenicity, substitution of particular nucleotides can be exchanged with chemically modified alternatives such as 5-methylcytosine or pseudouridine. Such substitutions can mute the immune response whilst also bolstering the stability of the mRNA and efficiency of translation. Other exemplary chemically modified nucleotides are described herein above.

Alternatively, or additionally, the mRNA may be encapsulated in lipid-based particles to enhance fusion with the lipid cell membrane.

Polynucleotides designed according to the teachings of some embodiments of the invention can be generated according to any polynucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the polynucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The term “polypeptide” or “protein” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH-NNAïve-CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF—CH—), retro amide bonds (—NH—CO—), peptide derivatives Naïve (R)—CH2-CO—), wherein R is the “normal” side chain, naturally present on the carbon atom.

These modifications can occur at any of the bonds along the polypeptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some embodiments of the invention may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The polypeptides of some embodiments of the invention may be synthesized by any techniques known to those skilled in the art of peptide synthesis, for example but not limited to recombinant DNA techniques or solid phase peptide synthesis.

Following is a non-limiting description of expression vectors and modes of administering thereof into cells which can be used to express a polypeptide-of-interest (e.g., any of the factors described hereinabove and below, e.g. Cdx2, Gata4) in a cell.

According to specific embodiments, expressing comprises introducing into the cell a polynucleotide encoding the polypeptide-of-interest (e.g. the factor).

According to specific embodiments, the polynucleotide is a DNA.

According to specific embodiments, the polynucleotide is a RNA. Typically, mRNA introduced into cells exists only in the cytoplasm, does not cause genome perturbations and is essentially transient. Unless expression of the mRNA changes the cell epigenetically, transient transfection is limited by the time of mRNA and cognate protein persistence in the cell, and does not continue after degradation of cognate proteins.

To express an exogenous protein in mammalian cells, a polynucleotide sequence encoding the polypeptide-of-interest is preferably ligated into a nucleic acid construct suitable for mammalian cell expression.

It will be appreciated that over-expression of genes can be effected using knock-in and/or knock-out constructs [see for example, Fukushige, S. and Ikeda, J. E.: Trapping of mammalian promoters by Cre-lox site-specific recombination. DNA Res 3 (1996) 73-50; Bedell, M. A., Jerkins, N. A. and Copeland, N. G.: Mouse models of human disease. Part I: Techniques and resources for genetic analysis in mice. Genes and Development 11 (1997) 1-11; Bermingham, J. J., Scherer, S. S., O'Connell, S., Arroyo, E., Kalla, K. A., Powell, F. L. and Rosenfeld, M. G.: Tst-1/Oct-6/SCIP regulates a unique step in peripheral myelination and is required for normal respiration. Genes Dev 10 (1996) 1751-62].

Such a nucleic acid construct or system includes at least one cis-acting regulatory element for directing expression of the nucleic acid sequence. Cis-acting regulatory sequences include those that direct constitutive expression of a nucleotide sequence as well as those that direct inducible expression of the nucleotide sequence only under certain conditions.

According to specific embodiments, an inducible promoter sequence for directing transcription of the polynucleotide sequence in the cell in an inducible manner is included in the nucleic acid construct. Inducible mammalian promoters are known to those of skill in the art (see, e.g. Bitter et al. (1987) Methods in Enzymology 153:516-544). Inducible promoters can be activated by external signals or agents (i.e. inducer). The inducer may directly activate a promoter or inactivate a repressor of that promoter. For example, inducible systems endogenous to mammalian cells include promoters induced by heavy-metals (Brinster et al. Nature (1982) 296:39-42; Mayo et al. Cell (1982) 29:99-108; and Searle et al. Molecular and Cellular Biology (1985) 5:1480-1489), steroid hormones (Hynes et al. Proc. Natl. Acad. Sci. USA (1981) 78:2038-2042; Lee et al. Nature (1981) 294:228-232; and Klock et al. Nature (1987) 329:734-736), heat shock (Nouer, Heat Shock Response. Boca Raton, FL, Ed. CRC, 1991) (reviewed in Mullick, A. and B. Massie Encyclopedia of Cell Technology pp. 1 140-1 164, 2000)) are well characterized. PCT publication WO2002/088346 discloses a cumate-inducible promoter. Additional inducible promoters are known in the art, and include, but are not limited to inflammation and hypoxia induced promoters. Prokaryotic and insect inducible promoter systems have been adapted for regulated expression in mammalian cells. See, for example, Gossen et al. (1993) TIBS 18:471-475 and No et al. (1996) Proc. Natl. Acad. Sci. USA 93:3346-3351). The insect ecdysone-inducible promoter is tightly regulated with no detectable background expression in the absence of inducer. Ecdysone is suitable for use in vivo because it is a naturally occurring lipophilic steroid that can penetrate tissues, is inert in mammals and exhibits rapid clearance kinetics (No et al). Gupta et al. (PNAS (2004) 101:1927-1932) discloses retroviral delivery of an ecdysone-inducible gene expression system under the control of a modified RNA polymerase Ill-specific U6 promoter.

The prokaryotic repressors from the lac and tet operons have been incorporated in eukaryotic inducible expression systems. Repression of expression is mediated by the repressor bound to operator sites placed downstream of the minimal promoter in the absence of inducer and repression is relieved on the addition of the inducer (Brown et al. (1987) Cell 49:603-612; Hu and Davidson (1987) Cell 48:555-566; Blau and Rossi, Proc. Natl. Acad. Sci. USA (1999) 96:797-799; and Gossen et al. (1995) Science 268:1766-1769).

According to specific embodiments, the inducible promoter is a Tet-on promoter induced by Tetracycline or Doxycycline.

Methods for construction of nucleic acid constructs or systems containing an inducible promoter operatively linked to a coding sequence of any polypeptide are known to those of skill in the art, as are methods for introducing such constructs of systems and vectors containing such expression cassette into cells.

In the case of mRNA, since gene expression from an RNA source does not require transcription, there is no need in a promoter sequence or the additional sequences involved in transcription described hereinbelow.

The nucleic acid construct or system (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vector may also contain a transcription and/or translation initiation sequence, transcription and/or translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding the protein-of-interest can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

According to specific embodiments, the expression construct include labels for imaging in cells, such as fluorescent labels.

Examples for mammalian expression vectors include, but are not limited to, pcDNA3, pcDNA3.1 (+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein.

Various methods can be used to introduce the polynucleotide or polypeptide of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, nucleofection, microinjection, and infection with recombinant viral vectors. In addition, see U.S. Pat. Nos. 5,464,764 and 5,487,992 for positive-negative selection methods. Currently preferred in vivo nucleic acid transfer techniques include transfection with viral or non-viral constructs, such as adenovirus, lentivirus, Herpes simplex I virus, or adeno-associated virus (AAV) and lipid-based systems.

According to specific embodiments, introducing is by transient transfection.

Naked DNA or RNA, cell penetrating peptide or Viral and non-viral vectors (e.g. but not limited to liposomes, nanoparticles, mammalian vectors and the like) may be utilized as delivery vehicles in delivery of the polynucleotide or polypeptide as is known in the art. According to specific embodiments of the invention, the delivery system used is biocompatible and nontoxic.

Following are exemplary embodiments suitable for enhancing penetration of the exogenous polynucleotide or polypeptide to cells.

According to one exemplary embodiment, naked DNA or RNA [e.g., naked plasmid DNA (pDNA)] is non-viral vector, which can be produced in bacteria and manipulated using standard recombinant DNA techniques. It does not induce antibody response against itself (i.e., no anti-DNA or RNA antibodies generated) and enables long-term gene expression even without chromosome integration. Naked DNA or RNA can be introduced by numerous means, for example but not limited to, intravascular and electroporation techniques [Wolff J A, Budker V, 2005, Adv. Genet. 54:3-20], or by jet injection [Walther W, et al., 2004, Mol. Biotechnol. 28: 121-8].

According to another exemplary embodiment, mammalian vectors are used, as further described hereinabove.

According to specific embodiments, the polynucleotide is comprised in a viral vector. Introduction of nucleic acids by viral infection offers several advantages over other methods such as lipofection and electroporation, since higher transfection efficiency can be obtained due to the infectious nature of viruses. The viral vector may be a virus with DNA based genome of a virus with RNA based genome (i.e. positive single stranded and negative single stranded RNA viruses). Examples of viral vectors include, but are not limited to, Lentivirus, Adenovirus and Retrovirus.

A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. Protocols for producing recombinant retroviruses and for infecting cells in-vitro or in-vivo with such viruses can be found in, for example, Ausubel et al., [eds, Current Protocols in Molecular Biology, Greene Publishing Associates, (1989)]. Other suitable expression vectors may be an adenovirus, a lentivirus, a Herpes simplex I virus or adeno-associated virus (AAV).

Regulatory elements that limit expression to particular cell types can also be included. Such features include, for example, promoter and regulatory elements that are specific for the desired cell type.

According to specific embodiments, expressing comprises introducing into the cell the polypeptide-of-interest (e.g. the factor).

According to specific embodiments, the polypeptide is provided in a formulation suitable for cell penetration that enhances intracellular delivery of the polypeptide as further described hereinbelow.

Cell-Penetrating Peptides (CPPs) are short peptides (≤40 amino acids), with the ability to gain access to the interior of almost any cell. They are highly cationic and usually rich in arginine and lysine amino acids. They have the exceptional property of carrying into the cells a wide variety of covalently and noncovalently conjugated cargoes such as proteins, oligonucleotides, and even 200 nm liposomes. Therefore, according to additional exemplary embodiment CPPs can be used to transport the polynucleotide or polypeptide to the interior of cells.

TAT (transcription activator from HIV-1), pAntp (also named penetratin, Drosophila antennapedia homeodomain transcription factor) and VP22 (from Herpes Simplex virus) are examples of CPPs that can enter cells in a non-toxic and efficient manner and may be suitable for use with some embodiments of the invention. Protocols for producing CPPs-cargos conjugates and for infecting cells with such conjugates can be found, for example L Theodore et al. [The Journal of Neuroscience, (1995) 15 (11): 7158-7167], Fawell S, et al. [Proc Natl Acad Sci USA, (1994) 91:664-668], and Jing Bian et al. [Circulation Research. (2007) 100:1626-1633].

The expression level and/or activity level of the exogenous polynucleotide and/or polypeptide expressed in the cells of some embodiments of the invention can be determined using methods known in the arts, e.g. but not limited to Northern blot analysis, PCR analysis, Western blot analysis, Immunohistochemistry, and Fluorescence activated cell sorting (FACS).

As mentioned, according to specific embodiments, expression of the exogenous factor is transient.

According to specific embodiments, inducing the transient expression starts within 14 days-0 hours prior to the mixing.

According to specific embodiments, inducing the transient expression starts within 13 days-0 hours prior to the mixing, within 12 days-0 hours prior to the mixing, within 11 days-0 hours prior to the mixing, within 10 days-0 hours prior to the mixing, within 9 days-0 hours prior to the mixing, within 8 days-0 hours prior to the mixing, within 7 days-0 hours prior to the mixing, within 6 days-0 hours prior to the mixing within 5 days-0 hours prior to the mixing, within 120 hours-0 hours prior to the mixing, within 100 hours-0 hours prior to the mixing, within 80 hours-0 hours prior to the mixing, within 60 hours-0 hours prior to the mixing, within 48 hours-0 hours prior to the mixing, within 36 hours-0 hours prior to the mixing, within 24 hours-0 hours prior to the mixing or within 12 hours-0 hours prior to the mixing, each possibility represents a separate embodiment of the invention.

According to a specific embodiment, inducing the transient expression starts within 120-0 hours prior to the mixing.

According to a specific embodiment, inducing the transient expression starts within 48-0 hours prior to the mixing.

According to a specific embodiment, inducing the transient expression starts within 42-0 hours prior to the mixing.

According to specific embodiments, inducing the transient expression starts no later than 48, 24 or 12 hours prior to the mixing.

According to specific embodiments, inducing the transient expression starts within 14-1 days prior to the mixing, within 13-1 days prior to the mixing, within 12-1 days prior to the mixing, within 11-1 days prior to the mixing, within 10-1 days prior to the mixing, within 9-1 days prior to the mixing, within 8-1 days prior to the mixing, within 7-1 days prior to the mixing, within 6-1 days prior to the mixing within 5-1 days prior to the mixing, within 120-20 hours prior to the mixing, within 100-20 hours prior to the mixing, within 80-20 hours prior to the mixing, within 60-house prior to the mixing, or within 48-20 hours prior to the mixing, each possibility represents a separate embodiment of the invention.

According to specific embodiments, inducing the transient expression starts within 14 days-12 hours prior to the mixing, within 13 days-12 hours prior to the mixing, within 12 days-12 hours prior to the mixing, within 11 days-12 hours prior to the mixing, within 10 days-12 hours prior to the mixing, within 9 days-12 hours prior to the mixing, within 8 days-12 hours prior to the mixing, within 7 days-12 hours prior to the mixing, within 6 days-12 hours prior to the mixing within 5 days-12 hours prior to the mixing, within 120-12 hours prior to the mixing, within 100-12 hours prior to the mixing, within 80-12 hours prior to the mixing, within 60-12 hours prior to the mixing, or within 48-12 hours prior to the mixing, each possibility represents a separate embodiment of the invention.

According to a specific embodiment, inducing the transient expression starts within 48-12 hours prior to the mixing.

According to specific embodiments, inducing the transient expression ends no later than 120 hours following the mixing.

According to specific embodiments, inducing the transient expression ends no later than 110 hours, 100 hours, 90 hours, 80 hours, 70 hours, 60 hours, 50 hours, 48 hours, 36 hours, 24 hours, 20 hours or 12 hours following the mixing, each possibility represents a separate embodiment of the invention.

According to specific embodiments, inducing the transient expression ends no later than 48 hours following the mixing.

According to specific embodiments, inducing the transient expression is effected for no more than 19 days.

According to specific embodiments, inducing the transient expression is effected for no more than 18 days, 17 days, 16 days, 15 days, 14 days, 13 days, 12 days, 11 days, 10 days, 9 days, 8 days, 7 days, 6 days, 5 days.

According to a specific embodiment, inducing the transient expression is effected for no more than 16 days.

According to a specific embodiment, inducing the transient expression is effected for no more than 96 hours.

According to a specific embodiment, inducing the transient expression starts 24 hours prior to the mixing and is effected for 96 hours (i.e. ends 48 hours following the mixing).

It will be appreciated that the culturing conditions suitable for the maintenance of the naïve PSCs and trophectoderm- and extra embryonic primitive endodermal-primed cells prior to the mixing include various tissue culture medium composition, feeder cells, oxygen concentration, culture vessel and the like and it is within the capability of one skilled in the art to determine which conditions should be applied. Non-limiting examples of such are described in the Examples section that follows, which serves as an integral part of the specification and Choi, J., Huebner, A., Clement, K. et al. Nature 548, 219-223 (2017); Yang et al. Cell (2017) 169 (2): 243-257, www(dot)cell(dot)com/fulltext/S0092-8674% 2817%2930183-6; www(dot)doi(dot)org/10.1038/nature23274; www(dot)doi(dot)org/10.1242/dev.180620; DOI: 10.1126/science.aay3325; DOI: 10.1016/j.stem.2017.11.004; Shingo Io et al. (2021) Cell Stem Cell, 28 (6): 1023-1039 and Mitsuyoshi Amita et al. (2013) PNAS E1212-1221; and International Patent Application Publication No. WO2016/016894 and WO2014/174470, the contents of which are fully incorporated herein by reference.

According to specific embodiments, the medium comprises a STAT3 activator.

As used herein the term “STAT3” refers to the signal transducer and activator of transcription 3 gene product (acute-phase response factor) (Gene ID 6774). In response to cytokines and growth factors, STAT family members are phosphorylated by the receptor associated kinases, and then form homo- or heterodimers that translocate to the cell nucleus where they act as transcription activators. Known STAT3 activators include, but are not limited to, interferon (IFN), epidermal growth factor (EGF), interleukin 5 (IL5), interleukin 6 (IL6), hepatocyte growth factor (HGF), leukemia inhibitory factor (LIF) and bone morphogenetic protein 2 (BMP2).

According to some embodiments of the invention, the STAT3 activator, which is used in the medium of some embodiments of the invention, is selected from the group consisting of LIF, IL6 and EGF.

According to some embodiments of the invention, the STAT3 activator, which is used in the medium of some embodiments of the invention, is selected from the group consisting of LIF and IL6.

According to some embodiments of the invention, the STAT3 activator, which is used in the medium of some embodiments of the invention is LIF.

As used herein the term “leukemia inhibitor factor (LIF)” refers to the polynucleotide and expression product e.g. polypeptide of the LIF gene (e.g. human Gene ID NO: 3976). A non-limiting example of a human LIF polypeptide is provided in the following GenBank Accession No. NP_001244064 (SEQ ID NO: 124). Preferably, the LIF used by the method according to some embodiments of the invention is capable of supporting, along with other factors, the undifferentiated growth of naïve PSCs, while maintaining their pluripotent capacity. LIF can be obtained from various manufacturers such as Millipore, Peprotech, and R&D systems.

According to specific embodiments, the medium comprises a GSK-3 inhibitor.

According to specific embodiments, the medium comprises an ERK1/2 inhibitor.

As used herein the term “ERK1” refers to the mitogen-activated protein kinase 3 (MAPK3) isoform 1 e.g. as set forth by GeneBank Accession No. NP_002737 (SEQ ID NO: 125), the MAPK3 isoform 2 e.g., as set forth by GenBank Accession No. NP_001035145 (SEQ ID NO: 126), the MAPK3 isoform 3 e.g. as set forth by GenBank Accession No. NP_001103361 (SEQ ID NO: 127) and/or ERK1 set forth in GenBank Accession No. M84490 (SEQ ID NO: 128) having the MAPK signaling activity.

As used herein the term “ERK2” refers to the mitogen-activated protein kinase 1 (MAPK1) e.g. as set forth by GenBank Accession No. NP_002736 (SEQ ID NO: 129) and/or GenBank Accession No. NP_620407 (SEQ ID NO: 130) having the MAPK signaling activity.

As used herein the term “ERK1/2 inhibitor” refers to any molecule capable of inhibiting the activity of ERK1/2 as determined by Western blot protein detection of phosphorylated ERK1/2 proteins.

Non-limiting examples of ERK1/2 inhibitors (also known as MEK1/2 inhibitors) include PD0325901 (AXONMEDCHEM—AXON 1408), PD98059 (AXONMEDCHEM—Axon 1223), and PD184352 (AXONMEDCHEM—AXON 1368).

According to specific embodiments, the medium comprises at least one of a GSK-3 inhibitor, WNT ligand, heparin, FGF2, FGF4, PDGF, leukemia inhibitory factor (LIF) and insulin.

According to specific embodiments, the medium comprises at least one of a GSK-3 inhibitor, leukemia inhibitory factor (LIF) and insulin.

According to specific embodiments, the medium comprises with at least one of GSK-3 inhibitor, heparin, FGF4, PDGF and BSA.

According to specific embodiments, the medium is devoid of Activin A [also known as Inhibin beta A, a polynucleotide and expression product e.g., polypeptide of the INHBA gene (corresponding to human gene ID: 3624) or homolog or ortholog of same].

According to specific embodiments, the medium is devoid of REPROCELL.

According to specific embodiments, the medium comprises at least one of a TGFR inhibitor, FGFR inhibitor, MEK/ERK inhibitor, BMP4, JAK inhibitor, FGF4, FGF2, heparin, a SUMOylation inhibitor, a Histone Deacetylase inhibitor, a HIPPO signaling pathway inhibitor and a factor that induces YAP nuclear translocation.

According to specific embodiments, for the TSCs primed cell pre-induction the following components may be useful supplements together or each: TGFRi A-83, LIF, HIPPO inhibitor LPA, ROCK inhibitor Y27632, MEK/ERK inhibitor PD0325901, EGF, JAKi.

Additional description of media and supplements that can be used with specific embodiments of the invention is provided infra.

According to specific embodiments, the subpopulation of cells are mixed together under conditions that allow formation of aggregated cells.

These conditions may comprise culturing time, culture vessel, medium composition, oxygen concentration, cells amount and ratio etc. Determining such conditions is well within the capabilities of the skilled artisan. Non-limiting examples of aggregation protocols that can be used with specific embodiments of the invention are described in the Examples section that follows, which serves as an integral part of the specification.

According to specific embodiments, mixing the naïve PSCs, the trophectoderm primed cells and the extra embryonic primitive endodermal primed cells is effected concomitantly.

According to other specific embodiments, mixing the naïve PSCs, the trophectoderm primed cells and the extra embryonic primitive endodermal primed cells is effected sequentially.

Thus, for example, according to specific embodiments, mixing the naïve PSCs and the extra embryonic primitive endodermal primed cells is effected prior to mixing with the trophectoderm primed cells. According to specific embodiments, mixing the naïve PSCs and the trophectoderm primed cells is effected prior to mixing with the extra embryonic primitive endodermal primed cells.

According to specific embodiments, mixing the trophectoderm primed cells with the extra embryonic primitive endodermal primed cells is effected prior to mixing with the naïve PSCs.

According to specific embodiments, this difference in mixing times can be up to 48 hours, up to 40 hours, up to 35 hours, up to 30 hours, up to 24 hours, up to 16 hours or up to 12 hours, each possibility represented a separate embodiment of the invention.

According to specific embodiments, the ratio between the naïve PSCs and the trophectoderm primed cells is between 1:1 and 1:5.

According to specific embodiments, the ratio between the naïve PSCs and the trophectoderm primed cells is between 1:3-1:4.

According to a specific embodiment, the ratio between the naïve PSCs and the trophectoderm primed cells is about 1:3.3.

According to specific embodiments, the ratio between the naïve PSCs and the primitive endodermal primed cells is between 1:1 and 2:1.

According to specific embodiments, the ratio between the naïve PSCs and the primitive endodermal primed cells is about 1:1.

According to specific embodiments, the aggregation is effects in an aggregation plate. Such plates are known to the skilled in the art and can be commercially obtained from e.g. STEMCELL Technologies.

Non-limiting examples of protocols for culturing of naïve PSCs, induction of trophectoderm primed cells and primitive endodermal primed cells and aggregation of the three cell types are provided in the Examples section which follows.

Thus, for example, naïve KH2 ESCs comprising a nucleic acid sequence encoding Gata4 under the control of a TetOn promoter may be cultured in 2iLif media and treated with DOX (e.g. 2 μg/ml—Sigma D9891) in 2i/Lif for 24 hours before starting the experiment. Induction of Gata4 can be similarly effected in N2B27 media, MEF media, TSC media or Aggregation media. Naïve KH2 ESCs comprising a nucleic acid sequence encoding Cdx2 under the control of a TetOn promoter may be cultured in 2i/Lif are treated with DOX (2 μg/ml—Sigma D9891) for different time points (-1 day to −14 days e.g. 24 hours) in TSC media (25 ng/ml FGF4 (Peprotech), 1 μg/ml Heparin (Sigma)) supplemented with or without lysophosphatidic acid (LPA) 0.5-1 μM, which is a Hippo pathway inhibitor. TGFR and/or ERK inhibitors may be optionally added as well in this pre-induction step. The non-inducible ESC fraction does not undergo special pre-treatment and is maintained in 2i/Lif conditions or serum LIF conditions. Other naïve conditions that can be used instead of 2i/LIF conditions include a2i conditions: which use N2B27 supplemented base media with GSK3 inhibitor CHIR99021 (3 μM final concentration), Lif (20 ng/ml final concentration) and SRCI CGP77675 (1 microM final concentration). Following, aggregation is effected in AggreWell 24-well plate 400 (STEMCELL Technologies 34415), or AggreWell 24-well plate 800 (STEMCELL Technologies 34815). Non adherent 96 well shaped wells (NUNC or THERMO) can also be used instead. AggreWell plate preparation may be effected according to manufacturer instructions; for example: 500 μl of anti-adherence rinsing solution (STEMCELL Technologies 07010) is added to each well. Plate is centrifuged at 2,000 g for 5 minutes and incubated 30 minutes at room temperature. After incubation, the rinsing solution is removed and the plate is washed twice with PBS. Following, 500 μl of aggregation media (AM) supplemented with DOX (2 μg/ml final concentration—Sigma D9891) and ROCKi Y27632 (5 nM-10 μM final concentration—Axon Medchem 1683) is added to each well. At the day of aggregation (day 0), the three cell populations may be trypsinized with 0.05% trypsin-EDTA solution (Biological Industries—Sartorius 03-053-1B) for 4-6 minutes at 37° C. Trypsin enzymatic reaction is stopped by adding Aggregation Media (AM). TrypleE or Accutase can be alternatively be used instead of trypsin. Cells are centrifuged at 1200 rpm for 3 minutes and re-suspended in AM with DOX (2 μg/ml—Sigma D9891) and ROCKi Y27632 (5 nM-10 microM final concentration—Axon Medchem 1683). Cells may be plated on gelatinized tissue culture plates for mouse embryonic fibroblast depletion for 20 minutes at 37° C. Following, supernatant is collected, centrifuged and cells are re-suspended. The three cell fractions are counted and re-suspended in AM with DOX (2 μg/ml—Sigma D9891) and ROCKi Y27632 (5 nM—Axon Medchem 1683). An exemplary ratio of (1 Naive ESCs:1 iGata4 ESC:3.33 iCdx2 ESC) is maintained in aggregation experiments with the following exact number of cells depending on the aggregation plate used e.g.: A) AggreWell 800: Number of microwells per well in 24 well plate=300; Number of added cells per each well of a 24 well plate=iCdx2: 5000 cells+iGata4: 1500 cells+Naive ESC 1500 cells; Number of Cells per single microwell=˜27 cells. B) AggreWell 400: Number of microwells per well in 24 well plate=1200; Number of added cells per each well of a 24 well plate=iCdx2: 20000 cells+iGata4: 6000 cells+Naive ESC 6000 cells; Number of Cells per single microwell=˜27 cells.

As another example, naïve PSCs are used to generate the following three population of cells:
1) Naïve WT PSC cells cultured in HENSM medium.
2) For the primitive endoderm compartment—Naïve WT cells plated on mouse embryonic fibroblast conditions on HENSM supplemented with ROCKi followed by replacement with a RCL medium on the next day for 72 hours.
3) For the Trophectoderm lineage—Naïve WT cells plated on feeder free conditions on HENSM supplemented with ROCKi followed by medium replacement on the next day with a (B)AP medium for 24 hours, and then with an with AP(J) for another 48 hours.

All three cell types are supplemented with a ROCKi before harvesting for co-aggregation on the next day. At the day of aggregation (day 0), Aggrewell plates 400, 24 wells (Stemcell technologies) are prepared and prefilled with 500 μL of N2B27 media and kept at 37° C. for medium equilibration. The three cell populations are disaggregated with TrypLE (Thermo Fisher 12604054) for 3 minutes for the WT and Primitive endoderm cells and 5 minutes for the Trophectoderm cells at 37° C., next the enzyme is removed and cells are incubated for two minutes at room temperature, afterwards cells are collected with PBS. Cells are centrifuged at 1300 rpm for 3 minutes and resuspended in N2B27 ROCKi. Following, primitive endoderm cells are plated on gelatinized tissue culture plates for mouse embryonic fibroblast depletion for 30 minutes at 37° C. Supernatant is collected, and all three elements are centrifuged separately and cells resuspended on N2B27 medium. The three cell fractions are counted and resuspended in N2B27 supplemented with ROCKi. A ratio of 1:1:3 (WT:PRE:TE) is maintained in the co-aggregation.

The present invention also contemplates composition and articles of manufactures comprising the cells and aggregates disclosed herein.

Thus, according to an aspect of the present invention there is provided an article of manufacture comprising:
(i) trophectoderm primed cells, wherein said trophectoderm primed cells comprise naïve pluripotent stem cell (PSCs) comprising an exogenous polynucleotide suitable for transient expression of a factor that induces differentiation to trophectoderm cells; and
(ii) naïve PSCs.

According to specific embodiments, the article of manufacture further comprising:
(iii) extra embryonic primitive endodermal primed cells, wherein said extra embryonic primitive endodermal primed cells comprise naïve pluripotent stem cells (PSCs) comprising an exogenous polynucleotide suitable for transient expression of factor that induces differentiation to extra embryonic primitive endodermal cells.

According to an additional or an alternative aspect of the present invention there is provided an article of manufacture comprising:
(i) trophectoderm primed cells, wherein said trophectoderm primed cells comprise naïve pluripotent stem cells (PSCs) comprising an exogenous polynucleotide suitable for transient expression of a factor that induces differentiation to trophectoderm cells; and
(ii) extra embryonic primitive endodermal primed cells, wherein said extra embryonic primitive endodermal primed cells comprise naïve pluripotent stem cells (PSCs) comprising an exogenous polynucleotide suitable for transient expression of factor that induces differentiation to extra embryonic primitive endodermal cells.

According to specific embodiments, the different subpopulations of cells are in a single container.

According to other specific embodiments, the different subpopulations of cells are in separate containers.

According to an additional or an alternative aspect of the present invention there is provided a method of ex-utero culturing a synthetic embryo, the method comprising mixing the cells of the article of manufacture disclosed herein under conditions the allow formation of aggregated cells, wherein the method comprises inducing transient expression of said factor starting within 14 days-0 hours prior to said mixing and ending no later than 120 hours following said mixing, thereby generating the synthetic embryo.

According to an additional or an alternative aspect of the present invention there is provided a mixture or aggregate, generated according to or obtainable by the methods disclosed herein.

According to specific embodiments, the mixture or aggregate is not structurally organized as a native or naturally occurring embryo at any developmental stage. According to specific embodiments, the different cells in the mixture or aggregate are randomly scattered within the mixture of aggregate in an un-organized manner. According to specific embodiments, the mixture or aggregate does not form a blastocyst at any stage. According to specific embodiments, the mixture or aggregate does not comprise a cavity.

According to specific embodiments, the mixture or aggregate comprises at least 12, at least 16, at least 32, at least 40, at least 50 cells, or at least 64 cells.

According to specific embodiments, the mixture or aggregate comprises more than 64 cells.

According to specific embodiments, the mixture or aggregate comprises up to 500 cells, up to 450 cells, up to 400 cells, up to 350 cells, up to 300 cells, up to 250 cells, up to 200 cells, up to 150 cells, or up to 100 cells.

It will be appreciated that during culturing the mixture or aggregate of unorganized cells there is a process of re-organization of the cells to form a synthetic embryo having a structure or organization of a native one.

According to specific embodiments, the mixture or aggregate is capable of forming a synthetic embryo structurally organized as a native embryo of at least a post-implantation egg cylinder or a bilaminar disc developmental stage.

Thus, according to an additional or an alternative aspect of the present invention, there is provided a mixture or aggregate comprising:
(i) naïve pluripotent stem cell (PSCs);
(ii) trophectoderm primed cells, wherein said trophectoderm primed cells comprise stem cells expressing an endogenous trophectoderm marker and an endogenous pluripotent marker; and
(iii) extra embryonic primitive endodermal primed cells, wherein said extra embryonic primitive endodermal primed cells comprise stem cells expressing an endogenous extra embryonic primitive endoderm marker and an endogenous pluripotency marker,
wherein said mixture or aggregate is not structurally organized as a native embryo at any developmental stage; and
wherein said mixture or aggregate is capable of forming a synthetic embryo structurally organized as a native embryo of at least a post-implantation egg cylinder of bilaminar disc developmental stage.

According to an additional or an alternative aspect of the present invention there is provided a mixture or aggregate comprising:
(i) trophectoderm primed cells, wherein said trophectoderm primed cells comprise stem cells expressing an exogenous factor that induces differentiation to trophectoderm cells, an endogenous trophectoderm marker and an endogenous pluripotent marker; and
(ii) naïve pluripotent stem cell (PSCs).

According to specific embodiments, the mixture or aggregate further comprising:
(iii) extra embryonic primitive endodermal primed cells, wherein said extra embryonic primitive endodermal primed cells comprise stem cells expressing an exogenous factor that induces differentiation to extra embryonic primitive endodermal cells, an endogenous extra embryonic primitive endoderm marker and an endogenous pluripotency marker.

It will be appreciated that the mixture or aggregate of some embodiments of the invention comprises distinct populations of stem cells all starting from naïve pluripotent stem cells (PSCs).

According to some embodiments of the invention, the method further comprises allowing the synthetic embryo to grow ex vivo (ex utero) or in vivo (in utero).

Thus, according to an aspect of the present invention, there is provided a method of ex-utero culturing a synthetic embryo, the method comprising ex-utero culturing the mixture or aggregate disclosed herein.

As described in the Examples section which follows the present inventors were able to generate post gastrulation synthetic embryos by utilizing a controlled ex utero embryo culture platform and growth conditions that previously enabled capturing mouse natural embryos gastrulation and organogenesis ex utero (Aguilera-Castrejon et al., 2021a), indicating these platform and growth conditions can be used to culture ex-utero synthetic embryos generated by both the methods disclosed herein as well as by any other method.

Thus, according to an aspect of the present invention, there is provided a method of ex-utero culturing a synthetic embryo, the method comprising ex-utero culturing a synthetic embryo at an aggregate stage in a static culture followed by a dynamic culture, wherein said static culture is effected until said embryo reaches at least an early post-implantation egg-cylinder or bilaminar disc developmental stage; and said dynamic culture starts the latest when said embryo reaches an early somite stage.

According to specific embodiments, the method further comprises determining development of the embryo prior to, during and/or following the culturing. Methods of assessing development are well known in the art and are further described in details herein.

As used herein, the term “culturing a synthetic embryo” refers to at least a synthetic embryo and culture medium in an in-vitro or ex-vivo (ex-utero) environment. The culture is maintained under conditions (or set of conditions) capable of inducing development and growth of the synthetic embryos. Such conditions include for example an appropriate temperature (e.g., 37° C.), atmosphere (e.g., % O₂, % CO₂), pressure, pH, light, medium, supplements and the like.

The culture may be in a glass, plastic or metal vessel that can provide an aseptic environment for culturing. According to specific embodiments, the culture vessel includes dishes, plates, flasks, bottles, vials, bags, bioreactors or any device that can be used to grow cells.

According to specific embodiments, the culture is maintained under sterile conditions.

According to specific embodiments, the culture is maintained at 37-38° C.

According to specific embodiments, the culture is maintained at 38° C.

According to specific embodiments, the culture is maintained at 37° C.

As changes in temperature may affect embryo developments, according to specific embodiments, care should be taken not to keep the embryo in a temperature higher than 38° C. and lower than 35° C. for a long periods of time. Thus, for example, opening the culture incubator or keeping the embryo at room temperature for a long periods of time should be avoided.

According to specific embodiments, the culture is a static culture.

According to other specific embodiments, the culture is a dynamic culture.

According to specific embodiments, the culture is a static culture followed by a dynamic culture.

As used herein, the term “static culture” refers to a cell culture that is carried out without agitation of the culture.

According to specific embodiments, the static culture is effected for 3-4 days.

According to specific embodiments, the static culture is effected for about 3 days.

Embryonic stage and development may be assessed compared to an in-vivo embryo counterpart at the same developmental stage by multiple ways well known in the art, including, but not limited to, morphology, length, weight, expression of developmental marker genes using specific antibodies or primers, transcriptional profiling and the like, as further described in the Examples section which follows which serve as an integral part of the specification.

As used herein, the term “embryonic day (E)”, “post fertilization day (PFD)” or “gestation day (GD)” refers to an embryo having developmental characteristic of an in vivo (in-uterine tube or in utero, depending on the day) embryo counterpart at the specified day following fertilization or mating, wherein E0/PFD0/GD0 is considered as the fertilized egg.

According to specific embodiments, the static culture is effected for 2-5 days.

According to specific embodiments, the static culture is effected for about 4 days.

According to specific embodiments, the static culture is effected for 10-20 days.

According to specific embodiments, the static culture is effected for about 12 days.

According to specific embodiments, the static culture ends the latest when the synthetic embryo reaches the egg cylinder stage (e.g. for mouse) or the bilaminar disc stage (e.g. for human).

As used herein, the term “egg cylinder stage” refers to a synthetic embryo post (and not including) the blastocyst stage and prior to the post-gastrulation stage and is characterized by expansion of yolk-sac surrounding the embryo and completion of gastrulation and emergence of somites.

According to specific embodiments, the egg cylinder stage refers a synthetic embryo having characteristic of E5-7 of an in-utero natural mouse embryo.

As used herein, the term “bilaminar disc stage” refers to a synthetic embryo post (and not including) the blastocyst stage and prior to the day 15 stage (Carnegie stage 6) and is characterized by two, dorsally convex cellular discs, one disc formed by a thick layer of pseudostratified columnar cells called epiblast, and the other by a thin layer of cuboidal cells called hypoblast. The disc-shaped epiblast is slightly convex dorsally, separated from the amnion by the amniotic cavity at the middle of the embryonic disc.

According to specific embodiments, the bilaminar disc stage refers a synthetic embryo having characteristic of PFD8-14 of an in-utero natural human embryo.

According to specific embodiments, to prevent sticking of the embryonic epiblast and yolk sac to the culture vessel during the static culture, the culture is examined to ensure that only the ectoplacental cone remains attached to the surface of the plate. According to specific embodiments, the embryos are carefully pushed away from the plate surface by using e.g. forceps, when needed.

As used herein, the term “dynamic culture” refers to a cell culture that is carried out with agitation (e.g. rolling, shaking, inverting) of the culture. To reiterate, in a dynamic culture the whole culture, including the embryo, is agitated. Non-limiting examples of dynamic cultures include a roller culture (a culture on a rolling device), a shaker culture (a culture on a shaker, e.g. orbital shaker).

According to specific embodiments, the dynamic culture is a roller culture.

According to specific embodiments, the rolling culture is rolled in 30 rpm.

Rotator culture units may be obtained commercially from e.g. B.T.C. Engineering—Cullum Starr Precision Engineering Ltd—UK, but can be further modified and adapted.

According to other specific embodiments, the dynamic culture is a shaker culture.

According to specific embodiments, the shaker rotates at 30-80 rpm, 40-70 rpm, 50-70 rpm or 55-65 rpm.

According to a specific embodiment, the shaker rotates at about 60 rpm.

According to specific embodiments, the dynamic culture comprises a shaker culture, a roller culture or a sequential combination thereof.

According to specific embodiments, the dynamic culture starts when the embryo reaches a pre-gastrulation stage.

As used herein the term “pre-gastrulation” refers to a synthetic embryo following the egg cylinder stage or the bilaminar disc stage and prior to the early gastrulation stage and is characterized by presence of OCT4 and OTX2+epiblast, pro-amniotic cavity formation and extra ectoderm embryo formation (ExE).

According to specific embodiments, the pre-gastrulation stage refers to a synthetic embryo having characteristics of E5-6 of an in-utero natural mouse embryo or PFD8-14 of an in-utero natural a human embryo.

According to specific embodiments, the dynamic culture starts with a shaker culture (for e.g. 1-4 days) followed by a roller culture.

According to specific embodiments, the roller culture starts when the embryo reaches at least the early gastrulation stage.

As used herein the term “early gastrulation” refers to a synthetic embryo following the pre-gastrulation stage and prior to the late gastrulation stage and is characterized e.g. in mice by egg cylinder shape with the primitive streak at the posterior side; in human by bilaminar disc cylinder shape with the primitive streak at the posterior side.

According to specific embodiments, the early gastrulation stage refers a synthetic embryo having characteristic of E6-7 of an in-utero natural mouse embryo or PFD12-18 of an in-utero natural a human embryo.

According to specific embodiments, the roller culture starts the latest when the embryo reaches an early somite stage.

According to specific embodiments, the dynamic culture starts the latest when the embryo reaches an early somite stage.

As used herein, the term “early somite” refers to a synthetic embryo following the late gastrulation stage and prior to the neural tube closure stage and is characterized by the appearance of the somites and formation of the first organs.

According to specific embodiments, the early somite stage refers a synthetic embryo having characteristic of E8-9 of an in-utero natural mouse embryo or PFD18-24 of an in-utero natural a human embryo.

As used herein the phrase “base medium” refers to a liquid substance used to support the growth/survival of stem cells and/or synthetic embryos and optionally induce their development.

The medium (cell or embryo culture medium, aggregation medium etc.) used according to some embodiments of the invention can be a water-based medium which includes a combination of substances such as salts, nutrients, minerals, vitamins, amino acids, nucleic acids, and/or proteins such as cytokines, growth factors and hormones, all of which are needed for cell growth and embryo development.

Preferably, all ingredients included in the culture medium of the present invention are substantially pure, i.e., a tissue culture grade.

For example, the culture medium may comprise a base medium such as a synthetic tissue culture medium, e.g. DMEM, DMEM/F12 or advanced DMEM/F12 (can be commercially obtained from e.g. GIBCOR or Biological Industries), Neurobasal (Invitrogen 21103-049 or ThermoFisher 21103049); KO-DMEM (can be commercially obtained from e.g. GIBCO®), CMRL (can be commercially obtained from e.g. GIBCO®), TCM199 (can be commercially obtained from e.g. Sigma), StemPro® (can be commercially obtained from e.g. Thermo Fisher Scientific), RPMI (can be commercially obtained from e.g. Biological Industries) or a combination thereof supplemented with the necessary additives as is further described herein.

Non-limiting examples of media than can be used with specific embodiments and specific stages of the disclosed methods are described in details in the Examples section which follows and in e.g. International Patent Application Publication No. WO2016/016894 and WO2014/174470, the contents or which are fully incorporated herein by reference.

Further, as the present inventors have identified novel culture media comprising specific factors which can be used to allow generation of trophectoderm- and extra embryonic primitive endodermal primed cells-primed cells and the development of a synthetic embryo (see the Examples section which follows), the present invention also envisages aspects related to media as described in the Examples section which follows, wherein components of the media are provided in concentrations of +20%.

According to specific embodiments, the base medium is devoid of phenol red.

According to a specific embodiment, the base medium comprises DMEM having the same components as the DMEM of GIBCO® Cat. No. 11880.

According to a specific embodiment, the base medium comprises DMEM/F12 having the same components as the DMEM/F12 of GIBCO® Cat. No. 12634-010.

According to a specific embodiment, the base medium comprises CMRL having the same components as the CMRL of GIBCOR Cat. No. 11530037.

According to a specific embodiment, the base medium comprises TCM199 having the same components as the TCM199 of Sigma Cat. No. M4530.

According to specific embodiments, the medium comprises serum or serum replacement.

According to specific embodiments, the medium comprises 10-80% 15-80%, 20-80%, 15-75%, or 20-75% [volume/volume (v/v)] serum or serum replacement.

According to specific embodiments, the medium comprises 15-60%, 15-40% or 15-30% (v/v) serum or serum replacement.

According to a specific embodiment, the medium comprises 20-40% or 20-30% (v/v) serum or serum replacement.

According to other specific embodiments, the medium comprises at least 20% (v/v) serum or serum replacement.

According to a specific embodiment, the medium comprises about 20% (v/v) serum or serum replacement.

According to a specific embodiment, the medium comprises about 30% (v/v) serum or serum replacement.

According to other specific embodiments, the medium comprises at least 30% (v/v) serum or serum replacement.

According to other specific embodiments, the medium comprises at least 35% (v/v), at least 40% (v/v), at least 45% (v/v), at least 50% (v/v), at least 55% (v/v), at least 60% (v/v), at least 65% (v/v), at least 70% (v/v) serum or serum replacement.

According to other specific embodiments, the medium comprises 40-80%, 50-80%, 60-80%, 70-80% (v/v) serum or serum replacement.

According to other specific embodiments, the medium comprises 70-80% (v/v) serum or serum replacement.

According to a specific embodiment, the medium comprises about 75% (v/v) serum or serum replacement.

According to a specific embodiment, the medium comprises increasing concentrations of serum or serum replacement throughout the culturing of the embryo.

According to a specific embodiment, the medium comprises at least 15% serum or serum replacement from the beginning until the embryo reaches an early post-implantation egg-cylinder or bilaminar disc stage and at least 30% serum or serum replacement thereafter.

The serum may be obtained from a rodent (e.g. rat, mouse) or a mammal (e.g. bovine, human).

According to specific embodiments, care should be taken that the serum (e.g. human serum) is devoid of any traces of hemolysis.

According to specific embodiments, the serum is obtained from an adult animal.

According to other specific embodiments, the serum is obtained from a fetal animal.

According to specific embodiments, the serum comprises a cord blood serum. Methods of obtaining cord blood serum (e.g. human cord blood serum) are well known in the art and are further described in the Examples section which follows.

According to specific embodiments, the serum comprises bovine serum (e.g. FCS).

According to specific embodiments, the serum comprises rat serum.

According to specific embodiments, the serum comprises human serum.

According to specific embodiments, the serum comprises human serum for at least part of the culturing.

According to specific embodiments, the human serum comprises umbilical cord serum (HCS).

According to other specific embodiments, the human serum comprises adult blood serum (HBS).

According to specific embodiments, the serum comprises rat serum and human serum.

According to specific embodiments, the ratio between the rat serum and the human serum in the medium is between 1:1-5:1 (v/v).

According to specific embodiments, the ratio between the rat serum and the human serum in the medium is between 1:1-3:1 (v/v).

According to specific embodiments, the ratio between the rat serum and the human serum in the medium is about 2:1 (v/v).

According to specific embodiments, the ratio between the rat serum and the human serum in the medium is 2:1 (v/v).

In some embodiments, optionally, the medium comprises knockout serum replacement (KSR) instead of an animal serum.

In some embodiments, optionally, the medium comprises knockout serum replacement (KSR) in addition to the animal serum e.g. the rat serum and the human serum.

In some embodiments, optionally, the KSR partially replaces one of either the human serum, the rat serum or partially replaces a quantity of both.

According to specific embodiments, the ratio between the serum and the base medium in the culture medium is between 1:0.5-10:1, 1:1-10:1 or 1:1-8:1 (v/v).

According to specific embodiments, the ratio between the serum and the base medium in the culture medium is between 1:1-5:1 (v/v).

According to specific embodiments, the ratio between the serum and the base medium in the culture medium is about 3:1 (v/v).

According to specific embodiments, the ratio between the serum and the base medium in the culture medium is 3:1 (v/v).

According to specific embodiments, the culture medium comprises 20-30% base medium, 40-60% rat serum and 20-30% human serum (v/v).

According to a specific embodiment, the culture medium comprises 25% base medium, 50% rat serum and 25% human serum (v/v).

According to specific embodiments, the serum is heat inactivated (e.g. in 55° C. 30-45 minutes).

According to specific embodiments, the serum is added to the medium just prior to use.

According to some embodiments of the invention, the medium can further include additional supplements including, but not limited to antibiotics (e.g., PEN-STREP), L-glutamine (e.g., GlutaMAX™), sodium pyruvate, non-essential amino acids (NEAA), Insulin-Transferrin-Selenium-Ethanolamine (ITS-X), β-Estradiol, progesterone, N-acetyl L-Cysteine, 3,3′,5-Triiodo-L-thyronine sodium salt (T3), sodium lactate, glucose, serum replacement and/or HEPES.

According to some embodiments of the invention, the medium comprises N2 and B27.

According to specific embodiments, the medium comprises glucose.

According to specific embodiments, the medium comprises at least 1 mg/ml, at least 2 mg/ml, at least 3 mg/ml or at least 4 mg/ml glucose.

According to specific embodiments, the medium comprises at least 4 mg/ml, at least 5 mg/ml, at least 6 mg/ml, at least 7 mg/ml or at least 8 mg/ml glucose.

According to specific embodiments, the medium comprises 2-12 mg/ml glucose, 3-12 mg/ml glucose, 4-12 mg/ml glucose or 4-8 mg/ml glucose.

According to specific embodiments, the glucose is provided in the medium in a constant or increasing concentrations throughout the culturing.

Thus, according to specific embodiments, throughout the culturing there is no decrease in the glucose concentration provided in the medium throughout the culturing (e.g. while passing from one set of conditions to a following set of conditions).

According to specific embodiments, the culture medium is devoid of MATRIGEL®.

According to specific embodiments, the conditions comprise replacement of at least half of the medium every 1-2 days of the culturing (e.g. in the static culture).

According to specific embodiments, the conditions comprise replacement of all the medium every 1-2 days of the culturing (e.g. in the dynamic culture).

According to specific embodiments, the culture is maintained under a hyperbaric pressure.

According to specific embodiments, the dynamic culture is maintained under a hyperbaric pressure.

According to specific embodiments, the roller culture is maintained under a hyperbaric pressure.

As used herein, the term “hyperbaric pressure” refers to a pressure greater than atmospheric pressure, wherein atmospheric pressure is generally regarded as 14.7 pounds per square inch (psi). Hence, wherein a specific hyperbaric pressure is indicated herein, it refers to the indicated pressure above the atmospheric pressure and not the value indicated per-se. For example, a hyperbaric pressure of 5 psi refers to a pressure of 19.7 psi, a hyperbaric pressure of 6.5 psi refers to a pressure of 21.2 psi and a hyperbaric pressure of 10.2 refers to a pressure of 24.7 psi.

According to specific embodiments, the hyperbaric pressure is more than 2.5 psi, more than 4 psi, more than 5 psi, more than 6 psi.

According to specific embodiments, the hyperbaric pressure is more than 5 psi.

According to specific embodiments, the hyperbaric pressure is less than 10.2 psi, less than 9 psi, less than 8 psi, less than 7 psi.

According to specific embodiments, the hyperbaric pressure is less than 10.2 psi.

According to specific embodiments, the hyperbaric pressure is more than 5 psi and less than 10.2 psi.

According to specific embodiments, the hyperbaric pressure is 6-7 psi.

According to specific embodiments, the hyperbaric pressure is 6.5 psi.

According to specific embodiments, the hyperbaric pressure is 0.1-0.5 psi.

According to other specific embodiments, the culture is maintained under atmospheric pressure.

According to specific embodiments, the static culture is maintained under atmospheric pressure.

According to specific embodiments, the culture is maintained in an atmosphere comprising a controlled level of O2, N₂ and/or CO₂.

According to specific embodiments, the culturing is effected in an atmosphere comprising 5% CO₂.

According to specific embodiments, the culturing is effected in an atmosphere comprising 5-40% oxygen.

According to specific embodiments, the culturing is effected in an atmosphere comprising 5-30%, 5-25% or 5-21% oxygen.

According to specific embodiments, the culturing is effected in an atmosphere comprising 10-40%, 10-30% or 15-30% oxygen.

According to specific embodiments, the culturing is effected in an atmosphere comprising 15-30% oxygen.

According to specific embodiments, the culturing is effected in an atmosphere comprising 18-23% oxygen.

According to specific embodiments, the culturing is effected in an atmosphere comprising constant or increasing oxygen concentrations throughout the culturing.

Thus, according to specific embodiments, throughout the culturing there is no decrease in the oxygen concentration throughout the culturing (e.g. while passing from one set of conditions to a following set of conditions).

According to specific embodiments, in order to control the pressure and oxygen level in the culture the following fetal incubation system comprising a gas and pressure controller and a static and/or rotating incubator is used.

### Exemplary Fetal Incubation System

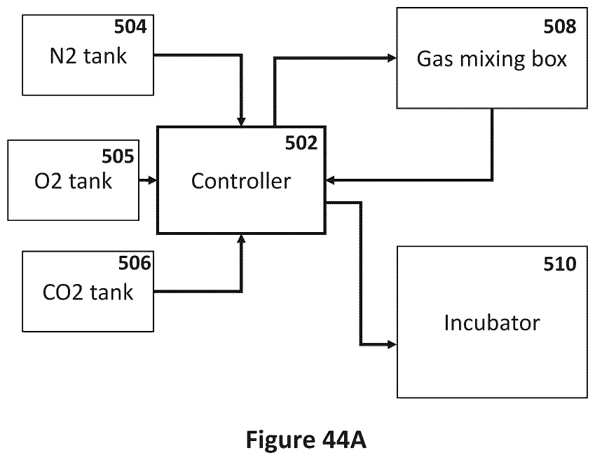

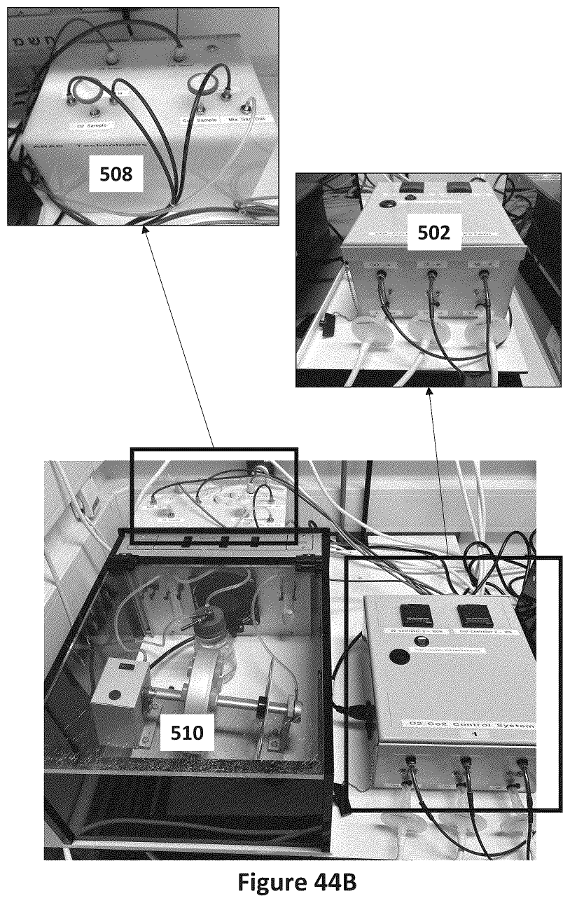

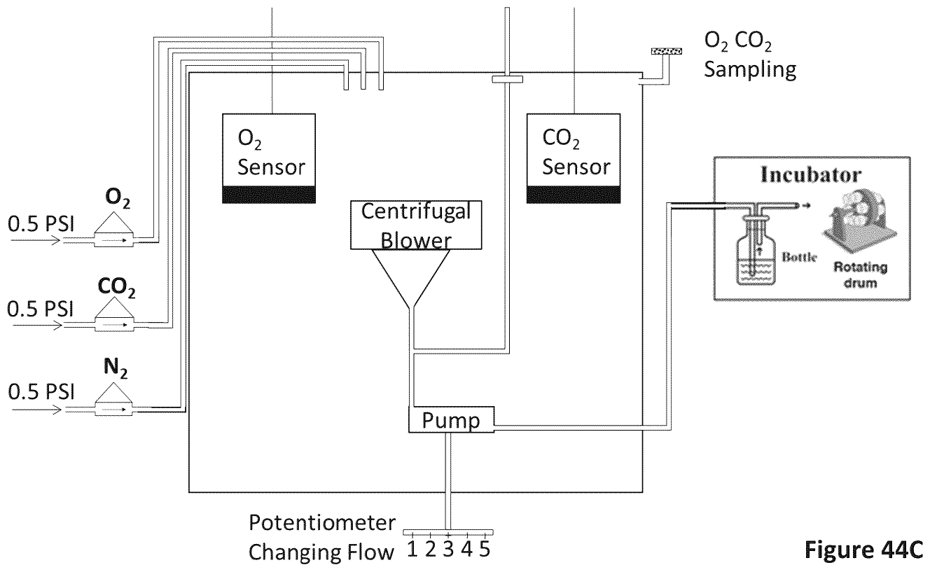

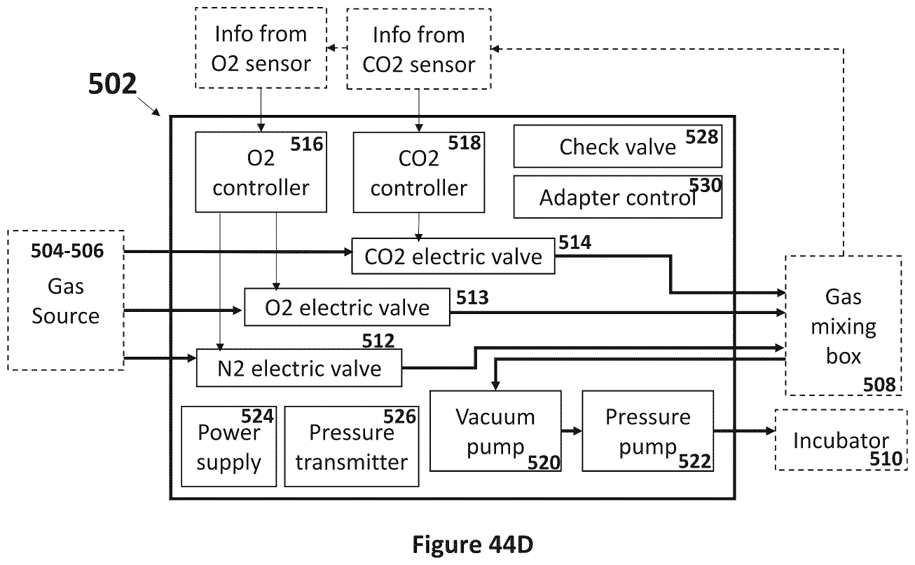

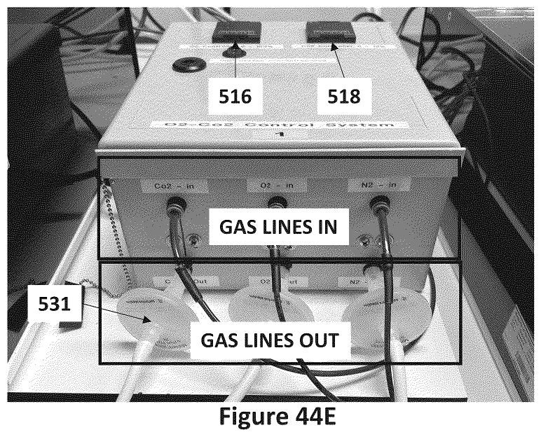

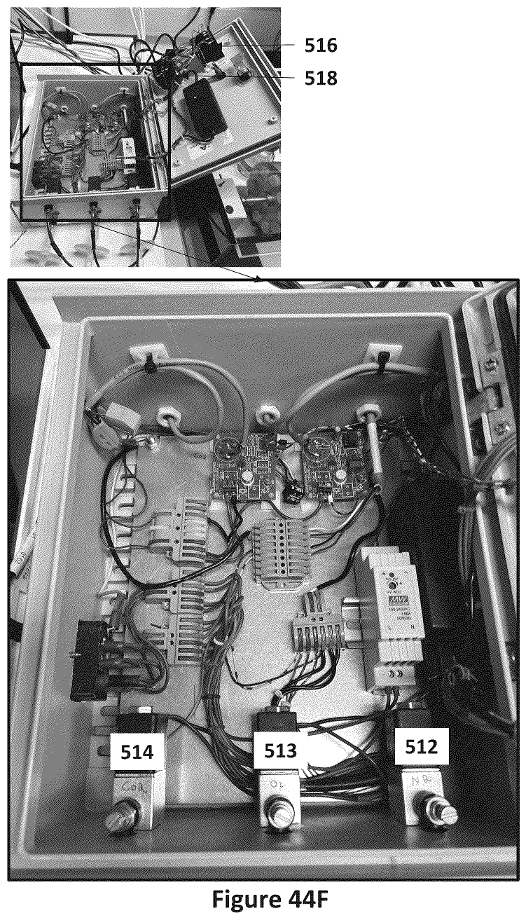

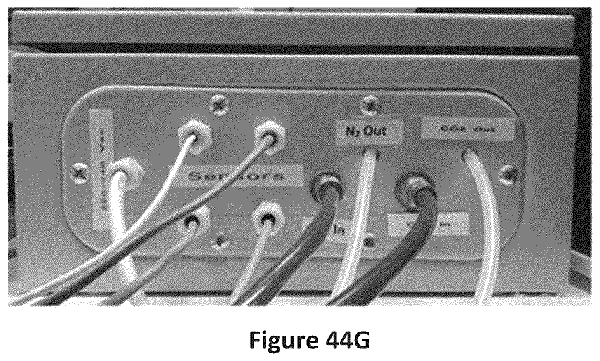

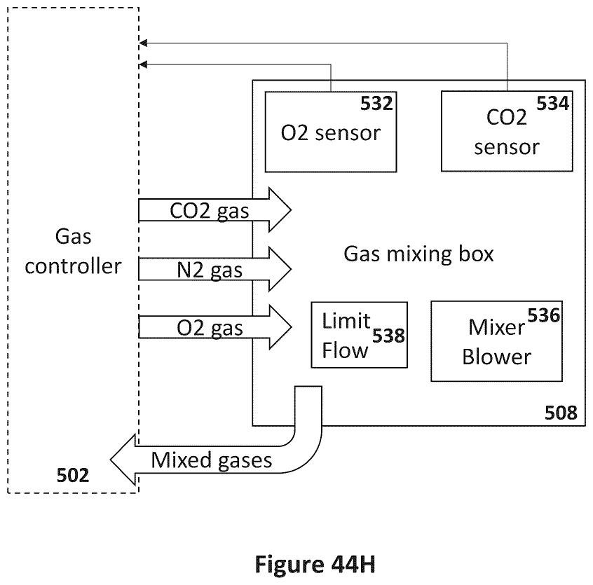

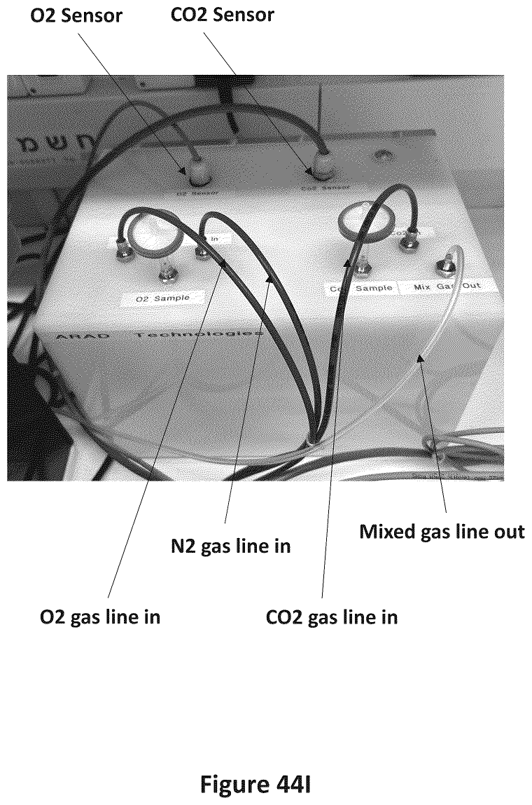

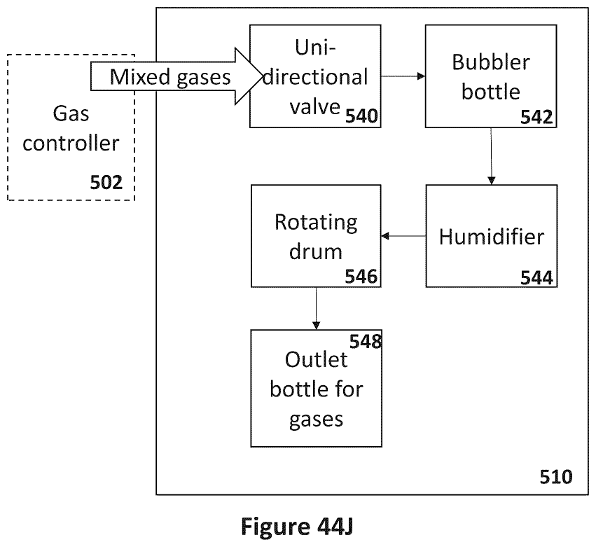

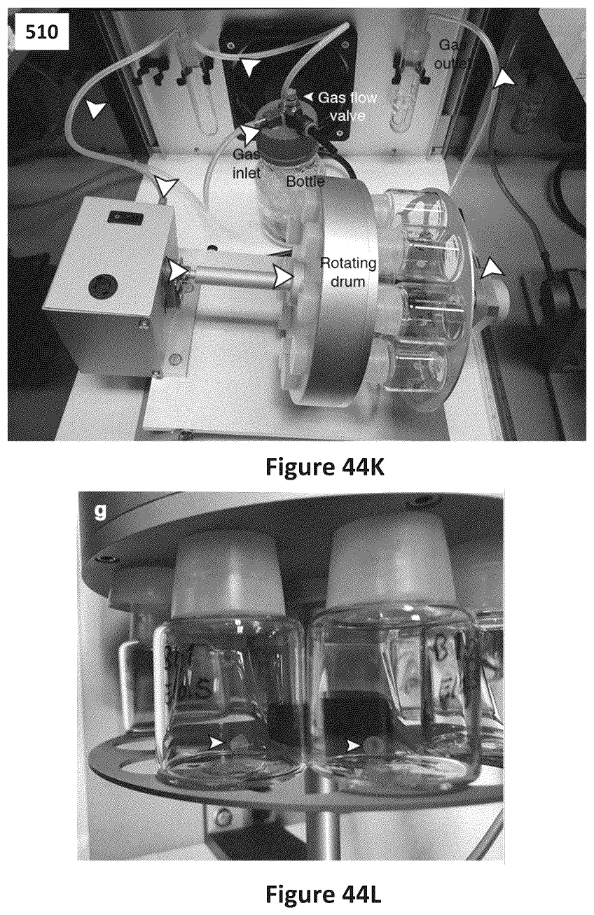

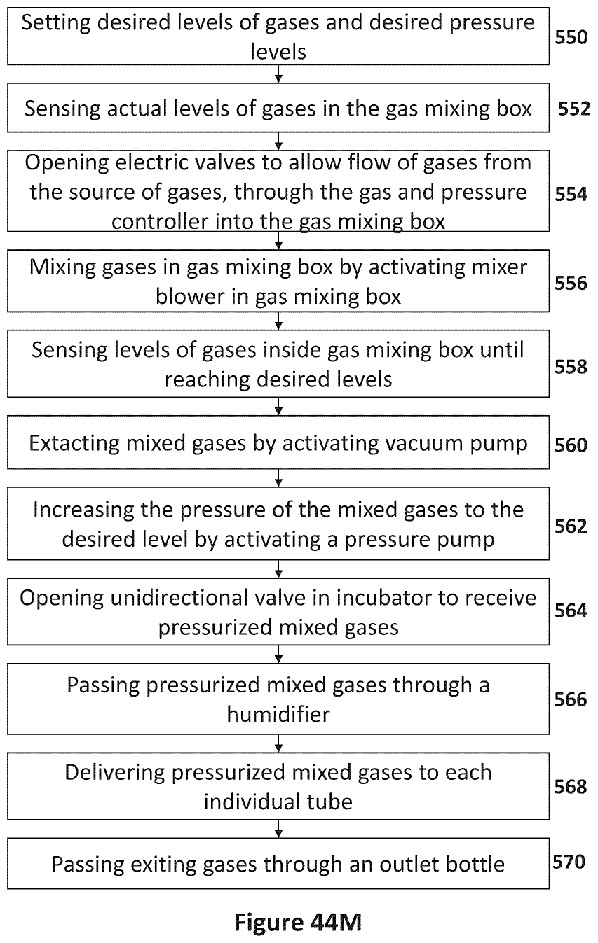

Referring now to FIG. 44A, showing a schematic representation of a fetal incubation system, according to some embodiments of the invention. In some embodiments, the system comprises a gas and pressure controller 502, one or more sources of gas 504, 505 and 506 (in FIG. 44A—Carbon dioxide (CO₂) 506, Oxygen (O₂) 505 and Nitrogen (N₂) 504 tanks are shown), a gas mixing box 508 and an incubator 510. In some embodiments, gases from the gas sources 504, 505, 506 are delivered into the gas and pressure controller 502, which delivers the gases into the gas mixing box 508. In some embodiments, once the mix of gases have reached the required concentrations, the gas is returned into the gas and pressure controller 502, which is then controlled-delivered into the incubator 510. In some embodiments, the incubator 510 optionally comprises an internal rotating incubator module configured to hold one or more vials in which the embryos are kept. In some embodiments, the mixed gases are delivered into the internal rotating incubator module, where the gases are equally delivered into each of the vials (see below for more information).

FIG. 44B shows an image of an exemplary fetal incubation system comprising the gas and pressure controller 502, the gas mixing box 508 and the incubator 510, according to some embodiments of the invention.

FIG. 44C shows a schematic general representation of an exemplary configuration of a principle of the electronic module for gas (gas and pressure controller 502 together with the gas mixing box 508) and pressure regulation. In some embodiments, N₂, O₂ and/or CO₂ enter into the system at a pressure of 0.5 psi and are mixed by a mixing centrifugal blower (see below). In some embodiments, gases are then optionally injected into a water bottle inside the incubator by a pump that allows control of the gas pressure, therefore allowing for hyperbaric conditions. In some embodiments, the system comprises one or more sampling ports (for example for O₂ and/or CO₂), which allow additional monitoring of the levels of the gases in the system.

### Exemplary Gas and Pressure Controller

Referring now to FIGS. 44D, showing a schematic representation of an exemplary gas and pressure controller 502, according to some embodiments of the invention. In some embodiments, as mentioned before one or more sources of gas 504, 505, 506 are connected to the gas and pressure controller 502. In some embodiments, each source of gas is connected to a dedicated electric valve 512, 513 and 514 in the gas and pressure controller 502. For example, gas from a source (for example a tank) of CO₂ 506 is connected to a dedicated CO₂ electric valve 514, and gas from a source (for example a tank) of N₂ 504 is connected to a dedicated N₂ electric valve 512, and gas from a source (for example a tank) of O₂ 505 is connected to a dedicated O₂ electric valve 513. In some embodiments, the gas and pressure controller 502 comprises dedicated ‘individual gas controllers 516/518′ for the manipulation and monitoring of the gases in the system (referred hereinafter as CO₂ controller 518 or O₂ controller 516—which are different from the main gas and pressure controller 502 of the fetal incubation system). For example, when the levels of CO₂ in the fetal incubation system are needed to be manipulated, a user accesses the CO₂ controller 518 to set up the required levels of CO₂ in the system, and for example when the levels of O₂ in the fetal incubation system are needed to be manipulated, a user accesses the O₂ controller 516 to set up the required levels of O₂ in the system by the addition or non-addition of N₂ gas into the system and/or by the addition or non-addition of O₂ gas into the system. It should be noted that in regular air there is about 21% oxygen, and when lower levels of oxygen are required inside the fetal incubation system, for example 5% or 10%, then nitrogen gas is inserted in order to reduce the levels of oxygen in the system. In some embodiments, when higher concentrations of oxygen are required, N₂ gases are stopped, and pure O₂ is provided, for example, to provide a 95%/5% O₂/CO₂ level inside the vials. In some embodiments, the system is configured to provide any combination of mixture of gases, for example from a mixture of gases that comprises 0% of O₂ to providing 100% of O₂; or for example any combination of O₂/CO₂ ratios, for example from about 1%/99% ratio to a 99%/1% ratio. In some embodiments, the gas and pressure controller 502, provides and ensures gases with a margin of error of about 0.2% for any of the gases provided to the fetal incubation system. In some embodiments, the margin of error is from about 0.1% to about 1% for CO₂ gases, from about 0.1% to about 2% for O₂ gases and from about 0.1% to about 2% for N₂ gases. In some embodiments, margins of error are not above 2% for CO₂. In some embodiments, margins of error are not above 5% for N₂. In some embodiments, margins of error are not above 5% for O₂. In some embodiments, each specific gas controller 516/518 controls the opening and closing of the specific electric valves 512, 513 and 514, according to the needs. In some embodiments, the needs, which are set by the user using the individual gas controllers 516/518, are monitored by dedicated sensors in the gas mixing box 508 (see below information about gas mixing box 508). Therefore, in some embodiments, information from the gas sensors in the gas mixing box 508 are delivered to the dedicated gas controllers 516/518. In some embodiments, the dedicated gas controllers 516/518 utilize the information from the gas sensors in the gas mixing box 508 to either open or close the specific electric valves 512, 513, 514, again according to the predetermined needs set by the user. In some embodiments, the gas and pressure controller 502 comprises a vacuum pump 520 and a pressure pump 522. In some embodiments, after the gases have been mixed in the gas mixing box 508 (see below information about gas mixing box 508), a vacuum pump 520 is activated to force out the mixed gases from the gas mixing box 508. In some embodiments, the gases are then accumulated in a pressure pump 522 until a predetermined level of pressure is reached before it is delivered into the incubator 510. In some embodiments, the pressure pump is configured to provide the mixed gases at a pressure of from about 1 psi to about 6 psi, optionally of from about 0.5 psi to about 8 psi, optionally from about 0.05 psi to about 12 psi. Preferably at 6.5 psi, when needed. In some embodiments, the user manually sets the pressure levels on which the pressure pump 522 will delivered the mixed gases. In some embodiments, the gas and pressure controller 502 comprises a power supply unit 524 configured to provide the dedicated power to the different parts of the gas and pressure controller 502. In some embodiments, the gas and pressure controller 502 optionally comprises a pressure transmitter 526 configured to monitor the pressure in the system/gas and pressure controller 502. In some embodiments, the gas and pressure controller 502 optionally comprises a check valve 528 configured to ensure that mixed gases exiting the gas and pressure controller 502 do not return (flow back) into the gas and pressure controller 502. In some embodiments, the gas and pressure controller 502 optionally comprises an adapter control for gases 530 configured to control the flow rate in the system. In some embodiments, the gas and pressure controller 502 comprises one or more filters-shown in FIG. 44E (531)—(for example 1μ filters) mounted on the tubes flowing gases for ensuring purity of the gases and potentially avoid contaminations.

FIGS. 44E, 44F and 44G show different images of an exemplary gas and pressure controller 502, according to some embodiments of the invention. Specifically, FIG. 44E shows a perspective view of the gas and pressure controller 502, showing the gas lines that go into the gas and pressure controller 502, and the gas lines that go out from the gas and pressure controller 502. Additionally, gas controllers 516/518 and optional filters 531 are shown. FIG. 44F shows the internal arrangement of the gas and pressure controller 502, showing exemplary electric valves 512, 513, 514, according to some embodiments of the invention. FIG. 44G shows an exemplary gas and pressure controller 502 that is configured to monitor and deliver only CO₂ and/or N₂, according to some embodiments of the invention.

### Exemplary Gas Mixing Box

Referring now to FIG. 44H, showing a schematic representation of an exemplary gas mixing box 508, according to some embodiments of the invention. In some embodiments, the gas mixing box 508 is used to ensure complete and uniform mixing of the different gases that are required to provide the necessary environment in the vials in the incubator. In some embodiments, the gas mixing box 508 comprises an internal volume of from about 250,000 cm³ to about 260,000 cm³. In some embodiments, different sizes may be used to provide different quantities of mixed gases as necessary. In some embodiments, the gas mixing box 508 is made of plastic, for example Perspex. In some embodiments, the gas mixing box 508 is made of a material other than plastic. In some embodiments, the gas mixing box 508 comprises dedicated gas sensors 532/534 configured to monitor the content percentage of those gases inside the gas mixing box 508. Following the previous description, in FIG. 44H two sensors are shown, an O₂ sensor 532 and a CO₂ sensor 534. In some embodiments, the sensors comprise a sensitivity for accuracy of from about 95% accuracy to about a 100% accuracy. In some embodiments, the gas mixing box 508 comprises a mixer blower 536 configured to thoroughly mix the gases coming from the gas and pressure controller 502. In some embodiments, once the levels of the gases detected inside the gas mixing box 508 by the sensors 532/534 arrive at the desired level, the mixed gases are sucked away by the vacuum pump 520 in the gas and pressure controller 502. In some embodiments, the gas and pressure controller 502 optionally comprises a limit flow 538 configured to maintain a uniform flow rate in the system, therefore potentially avoiding the possibility of changes in the flow rate. In some embodiments, the gas mixing box 508 comprises one or more filters—not shown—(for example 1μ filters) mounted on the tubes flowing gases for ensuring purity of the gases and potentially avoid contaminations.

FIG. 44I shows an image of an exemplary gas mixing box 508, according to some embodiments of the invention.

### Exemplary Incubator

In some embodiments, the incubator is a static incubator. In some embodiments, the incubator is a rotating incubator. In some embodiments, the incubator is a static incubator comprising a rotating module inside of it. In some embodiments, the static incubator comprises one or more temperature modulators configured to preserve the temperature inside the static incubator, including the rotating module allocated inside of it. In some embodiments, the temperatures inside the incubator are modulated to be from 4° C. to about 60° C. In some embodiments, the incubator is, for example, a “precision” incubator system (BTC01 model with gas bubbler kit—by B.T.C. Engineering—Cullum Starr Precision Engineering Ltd—UK).

Referring now to FIG. 44J, showing a schematic representation of an exemplary incubator 510, according to some embodiments of the invention. In some embodiments, mixed gases are delivered from the gas and pressure controller 502 into the incubator 510. In some embodiments, the incubator comprises a unidirectional valve 540 connected to a tube, from which the mixed gases are delivered from the gas and pressure controller 502. In some embodiments, the unidirectional valve 540 is a manual unidirectional valve, which is opened and closed manually by a user. In some embodiments, the unidirectional valve 540 is an automatic unidirectional valve controlled by a master controller (see below). In some embodiments, the mixed gases are optionally delivered into a bubbler bottle 542. In some embodiments, the bubbler bottle 542 is partially filled with a liquid. In some embodiments, the liquid is water. In some embodiments, the gases are delivered into the liquid in the bubbler bottle 542, thereby creating bubbles in the liquid. In some embodiments, the bubbler bottle 542 allows a user to see that the system is delivering mixed gases by visually assessing: 1. If there are bubbles; and 2. The rate of creation of bubbles. In some embodiments, additionally, bubbler bottle 542 works as a humidifier for the gases (see below). In some embodiments, the bubbler bottle 542 provides a safeguard from pressure coming from the delivered mixed gases into the incubator 510, for example, in case of a malfunction if mixed gases are delivered at higher pressure than the desired one, the extra pressure will be contained and dissipated in the bubbler bottle 542. In some embodiments, the mixed gases are optionally then delivered into an additional humidifier 544. In some embodiments, the inventors have discovered that in certain cases dry mixed gases could be harmful to the samples in the incubator, therefore, the addition of the humidifier 544 overcomes this issue. In some embodiments, the additional humidifier reduces excess humidity from the gases coming from bubbler bottle 542. In some embodiments, from the humidifier 544, the mixed gases are delivered into the rotating drum 546 of the rotation module, which comprises all containers (vials) comprising the samples located in the incubator. In some embodiments, the rotating drum 546 is configured to deliver mixed gases equally between the containers, optionally while rotating the samples. In some embodiments, the containers (vials) in the rotating drum 546 comprise the medium necessary for the growth and/or maintenance of the embryos. In some embodiments, the delivery of the gases is provided into the containers (vials) and absorbed/used via the medium. It should be noted that, in some embodiments, since the embryos are left in suspension in the medium, continuous delivery of new medium with already mixed gases is problematic, because these types of mechanisms require old/used medium to be extracted from the vial while inserting new medium with new mixed gases in it, which can increase the chance of losing the embryos during the exchange. Therefore, the provision of new gases is performed by delivery mixed gases into the vials without the need to change the medium for it. In some embodiments, independently of the need of providing continuous replacement of gases, medium can be changed by taking out each vial and carefully changing the medium according to known techniques. In some embodiments, the rotating drum 546 is configured to be positioned in an angle, which allows the vials to have an angle with respect to the base on which the whole rotating module is standing. In some embodiments, the angle of from about 0 degrees (no angle—vials are kept on their side as shown for example in FIG. 44K) to about 45 degrees. In some embodiments, the angle is provided so a top of a vial is always in an upper position in relation to a bottom part of the vial. In some embodiments, the rotation of the rotating drum 546 is independent from the action of delivering mixed gases into the vials. In some embodiments, the rotating drum is configured to rotate at velocities of from about 1 rpm to about 100 rpm. In some embodiments, exiting gases from the rotating drum 546 are delivered to an outlet bottle 548 for gases. In some embodiments, the outlet bottle 548 acts as a pressure buffer for the system, which helps keeping a constant pressure throughout the system. In some embodiments, the incubator 510 comprises darkened walls, which allow keeping the samples in the dark. In some embodiments, the incubator 510 optionally comprises one or more cameras for monitoring the samples, for examples regular video cameras, IR cameras, night vision cameras, etc. In some embodiments, the incubator 510 comprises one or more heaters configured to keep the samples at a certain temperature. In some embodiments, the incubator 510 comprises one or more light sources, for example, white light, IR light, UV light and/or black light.

FIG. 44K shows an image of an exemplary rotating incubator module 510, according to some embodiments of the invention. FIG. 44L shows an image of exemplary containers (vials or bottles) having samples, according to some embodiments of the invention.

### Exemplary Automated Fetal Incubation System

In some embodiments, the fetal incubation system as disclosed above, is connected to a master controller configured to perform automated actions according to predetermined protocols provided by a user. For example, a user programs the master controller to perform changes in the incubation chambers over a certain period of time. In some embodiments, the system will comprise electric valves overall the system, which will be activated/deactivated according to the programed protocols. In some embodiments, a potential advantage of utilizing automated systems is that it reduces the chances of human errors during the developments of the embryos. In some embodiments, optionally, the master controller provides periodic updates to a user to a PC or a mobile electronic device.

### Exemplary General Information Related to the System

In general, a number of culture techniques have been proposed over the years since the 1930s by culturing the embryos in conventional static conditions, in rotating bottles on a drum (referred to as “roller culture systems”) or on circulator platforms. These platforms remain highly inefficient for embryos survival and are limited to short periods of time, as the embryos begin to display developmental anomalies as early as 24 hours after culture initiation. Thus, stable and efficient protocols for extended culturing of pre-gastrulating mouse embryos all the way until advanced organogenesis stages were developed and are disclosed herein. In some embodiments, some of cell culture supplements or biomechanical principles newly established in stem cell research, were tested to assess if they could be helpful for keeping embryos alive (e.g. hyperbaric chambers, synthetic sera). In some embodiments, the “roller culture system” on a drum is used and it is integrated with a customized and in house developed electronic gas regulation module 502 that allowed precise control not only of N₂, O₂ and CO₂ levels with high sensitivity, but also allowed controlling the atmospheric pressure. In some embodiments, sequential increases in the oxygen levels every 24 hours, starting from 5% O2 at E7.5, 13% at E8.5, 18% at E9.5, and ending with 21% O₂ at E10.5 were applied and were found to be most optimal for the robust outcome reported herein. Additionally, when necessary, an increase in oxygen levels reached 95%. In addition, in some embodiments, maintaining a hyperbaric pressure of about 6.5 psi was found also critical for normal and efficient development of the embryos.

In some embodiments, the samples are kept in a static incubator. In some embodiments, the samples are kept in a dynamic incubator, for example a rotating incubator. In some embodiments, the samples are kept first in a static incubator and then moved to a dynamic incubator, or vice versa. In some embodiments, the samples are kept in a static incubator comprising, for example a rotating incubator inside of it. In some embodiments, when kept in a dynamic incubator the samples are kept in rotating bottles on a drum (referred to as “roller culture systems”) or on circulator platforms.

In some embodiments, the embryos are kept on the rotating bottles culture unit inside a “precision” incubator system (For example the BTC01 model with gas bubbler kit—by B.T.C. Engineering—Cullum Starr Precision Engineering Ltd—UK) during all the time of culture. In some embodiments, a ‘rotator’ culture method which provides continuous flow of oxygenating gas to cultures in rotating bottles was used and disclosed herein elsewhere (for example BTC Rotating Bottle Culture Unit BTC02 model by B.T.C. Engineering—Cullum Starr Precision Engineering Ltd—UK). In some embodiments, the culture bottles (Glass Bottles (Small) BTC 03 and Glass Bottles (Large) BTC 04) are plugged into the hollow rotating drum. In some embodiments, gas flows along the axis and is distributed to the culture bottles by a baffle plate within the drum. In some embodiments, the system maintains a stable pH, when compared to other systems with sealed culture bottles. In some embodiments, the rotator is supplied complete with gas filter, bubbler and leads by the manufacturer. In some embodiments, the BTC Precision Incubator uses a thyristor-controlled heater and high flow-rate fan to give a highly stable and uniform temperature throughout the easily accessible working volume. In some embodiments, the incubator has a working volume 370×350×200 mm high which is accessed through a hinged top. In some embodiments, the heater element is rated at 750 Watts. In some embodiments, Bung (Hole) BTC 06 is used to seal the bottles and Bung (Solid) BTC 07 is used to seal the drum (B.T.C. Engineering—Cullum Starr Precision Engineering Ltd—UK).

In some embodiments, in order to achieve constant O₂ and CO₂ levels in the culture medium throughout the incubation period, the incubator module is linked to the gas and pressure control unit 502 (model #-HannaLab1; assembled and sold by Arad Technologies LTD, Ashdod, Israel). In some embodiments, carbon dioxide and oxygen concentration are regulated by specific controllers located on the gas and pressure control unit 502. In some embodiments, a pressure controller allows control of the gas pressure between 5 to 10 psi (positive pressure over ambient external atmospheric pressure). In some embodiments, nitrogen, O₂ and/or CO₂ are then injected into the gas mixer box at pressure of 6.5 psi which was found as the optimal level. In some embodiments, the mixing of the gases in the gas box is homogeneous and mixed by a centrifugal mixer blower. In some embodiments, the gases are injected into the incubator by a pump that builds pressure and sufficiency according to the count of air bubbles created in a water bottle, which is under the control of a one-way flow meter. In some embodiments, the bubble rate (which indicates the speed of gas flowing into the bottles) is be adjusted as needed by the user. In some embodiments, gas flows through the inlet into the water bottle, and the speed of gas flowing into the bottle is controlled with a valve. In some embodiments, humidified gas circulates to a glass test tube and then to the inside of the bottles in the rotating drum. In some embodiments, gas flow speed is monitored by the rate of bubbles created inside an outlet water-filled test tube. In some embodiments, the bottles with the samples are placed on the rotating bottle culture system, rotating at 30 revolutions per minute at 37° C., and continuously gassed with an atmosphere of, for example 5% O₂, 5% CO₂ at 6.5 pounds per square inch (psi), or for example 5% O₂, 5% CO₂ at 0.1 pounds per square inch (psi), or for example with a gas mixture of 13% O₂, 5% CO₂, or for example in a gas atmosphere of 18% O₂ and 5% CO₂, or for example with a gas supply of 21% O₂ and 5% CO₂. In some embodiments, for media exchange, culture media is pre-heated for at least an hour by placing it inside a glass bottle on the rotating culture with an adequate gas atmosphere depending on the stage of the cultured embryos.

### Exemplary Methods Related to the Fetal Incubation System

Referring now to FIG. 44M, showing a flowchart of exemplary methods related to the fetal incubation system, according to some embodiments of the invention. In some embodiments, the user sets the desired levels of gas and pressure inside the incubator 550. For example 5% CO₂ and 10% O₂ at a pressure of 6.1 psi. In some embodiments, the gas and pressure controller activates the sensors in the gas mixing box to receive information about the actual levels of the gases in the gas mixing box 552. In some embodiments, the electric valves are opened to allow flow of gases from the gas sources, through the gas and pressure controller into the gas mixing box 554. In some embodiments, the gases are mixed inside the gas mixing box by activating a mixer blower 556. In some embodiments, the sensors in the gas mixing box are activated to monitor the levels of the gases inside until they reach the desired levels 558. In some embodiments, the mixed gases are extracted from the gas mixing box by activating a vacuum pump 560. In some embodiments, pressure of the extracted mixed gases are increased to a desired level by activating a pressure pump 562. In some embodiments, a unidirectional valve inside the incubator is opened to allow the pressurized mixed gases to flow into the incubator 564. In some embodiments, the pressurized mixed gases are passed through a humidifier 566. In some embodiments, the humidified pressurized mixed gases are delivered to the individual tubes containing the samples 568. In some embodiments, exiting gases are passed through an outlet tube before exiting the incubator 570.

According to specific embodiments, the methods further comprise isolating a cell, tissue or organ from the embryo following the culturing.

Non-limiting examples of such cells include stem cells [for example, but not limited to, embryonic stem cells, mesenchymal stem cells, neural stem cells, hematopoietic stem cells], blood cells, liver cells, insulin secreting pancreatic beta cells, muscle cells, lung epithelial cells, endothelial cells, glial cells.

These cells, tissue or organs may be used for example as a cell-based therapy, tissue replacement, organ or tissue implantation or for research purposes.

According to other specific embodiments, the methods further comprise transferring the synthetic embryo in-utero.

The established methods of generating synthetic embryos and methods and systems for culturing them ex utero as described herein may be further combined with e.g. genetic modification, chemical screens, tissue manipulation and microscopy methods and may constitute a powerful tool in basic research e.g. as a framework to investigate the emergence of cellular diversity, cell fate decisions and how tissues and organs emerge from a single totipotent cell; as well as a source of cells, tissue and organs for transplantation, generation of chimeric embryos, testing the effect of drugs on embryonic development (e.g. teratogenic effect) etc. Such methods are known in the art.

Thus, according to specific embodiments, the method comprises manipulating the synthetic embryo or the subpopulations of cells described herein.

According to specific embodiments, manipulating comprises introducing into the cells or the embryo a gene of interest.

According to specific embodiments, manipulating comprises introducing into the cells or embryo a polynucleotide of interest.

According to specific embodiments, manipulating comprises introducing into the cells or embryo a genome editing or RNA silencing agent.

According to specific embodiments, the manipulating comprises producing an embryo incompatible with life. Thus, for example, the manipulation may comprise knocking a selected gene to selectively perturb a certain organ, thus making the embryo with limited developmental potential and not being able to sustain viability, e.g. headless (e.g. deletion of Mesp1 or NKX2-5) or heartless (e.g. deletion of Lim1), as further described in the Examples section which follows.

Thus, according to specific embodiments, the manipulating comprises introducing into the embryo a polynucleotide rendering an embryo incompatible with life.

Methods of designing, expressing and introducing a polynucleotide of interest such that it will be expressed in a cell of interest are well known in the art and further described herein above and below.

According to specific embodiments manipulating comprises microinjecting cells into the embryo to thereby obtain a chimeric embryo.

As used herein, the phrase “chimeric embryo” refers to an animal comprising cells of at least two genetically distinct individuals.

It is noted that the chimeric embryo can be composed of cells of two different individuals belonging to two different species, or to the same species.

According to some embodiments of the invention, the cells are allogeneic to the embryo.

According to some embodiments of the invention, the cells are xenogeneic to the embryo.

As used herein, the term “xenogeneic” refers to at least two individuals of different species.

According to specific embodiments, the cells are stem cells [for example, but not limited to, embryonic stem cells, mesenchymal stem cells, neural stem cells, hematopoietic stem cells, induced pluripotent stem cells (iPS)].

According to some embodiments of the invention, introducing the cells is performed ex vivo via direct injection or aggregation with the developing embryo.

According to specific embodiments, the manipulating comprises introducing into the cells or the embryo a drug of interest.

According to specific embodiments, the methods further comprise determining an effect of the manipulating on development of the embryo.

According to specific embodiments, any of the genes, polynucleotides, proteins, polypeptides and/or proteinaceous moieties described herein may have a sequence of a human gene, polynucleotide, protein, polypeptide and/or proteinaceous moiety or a functional fragment or homolog thereof which exhibit the desired activity as described herein.

According to specific embodiments, the gene, polynucleotide, protein, polypeptide and/or proteinaceous moiety is of a human origin.

According to other specific embodiments, the gene, polynucleotide, protein, polypeptide and/or proteinaceous moiety is a homolog of a human gene, polynucleotide, protein, polypeptide and/or proteinaceous moiety. Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the human sequence.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion. Generally, the nomenclature used herein and the laboratory procedures utilized in the present invention include molecular, biochemical, microbiological and recombinant DNA techniques.

### Example 1

### Aggregation of Mouse Naïve PSCs, TSCs and XENs does not Generate an Organized Embryo

### Materials and Methods

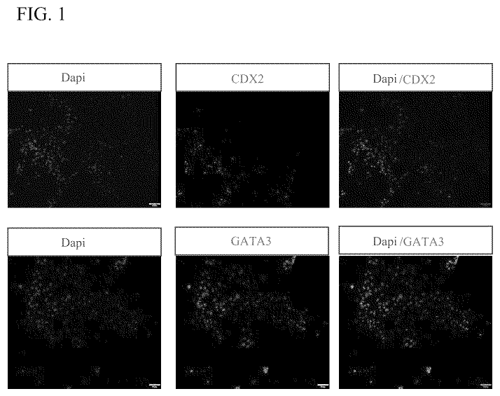

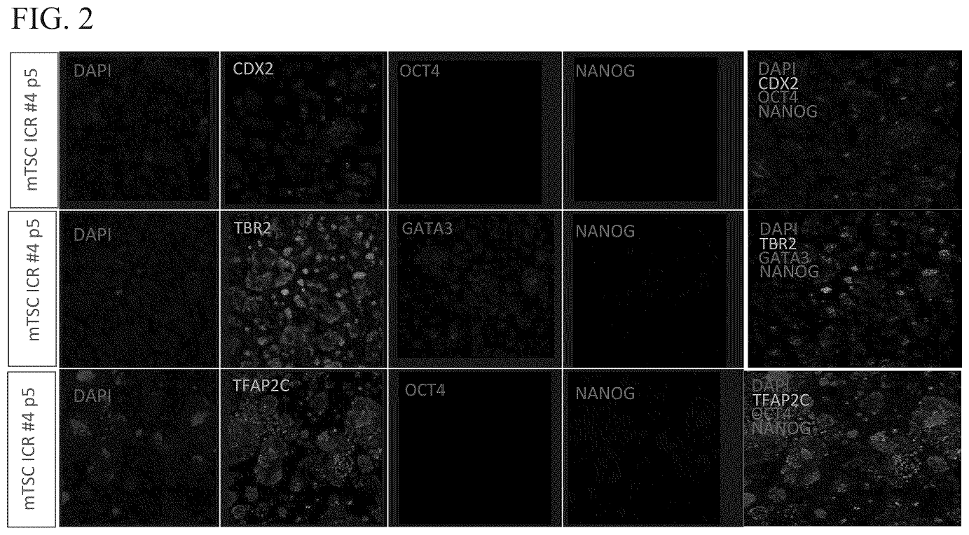

Embryo derived Mouse TSC line #1 (mTSC #1)—was established according to previously described classical protocols (doi: 10.1126/science.282.5396.2072). Briefly, TSCs were derived from E3.5 blastocysts flushed from the uterus of 5-weeks-old female CD1 mouse. The blastocysts were cultured on mitotically inactivated mouse MEF cells (feeder cells) with TSC culture medium: advanced RPMI 1640 (11875-093 Thermo Fisher Scientific or RPMI (Invitrogen) supplemented with 15-20 (v/v) FBS (Gibco, ES cell qualified; or Biological Industries), 1× GlutaMAX (35050061, Thermo Fisher Scientific; or Invitrogen), 0.1 mM β-mercaptoethanol (21985-023, Thermo Fisher Scientific; or Sigma), 1 mM sodium pyruvate (11360070, Thermo Fisher Scientific), 1% (v/v) penicillin-streptomycin (15140122, Thermo Fisher Scientific; or Biological Industries), 25 ng/ml recombinant human FGF4 (235-F4-025, R&D; or Peprotech), 1 μg/ml heparin (07980, Stem Cell; or Sigma) and 1× nonessential amino acids (Biological Industries). 25 ng/ml of FGF2 (Peprotech) was optionally added. DMEM can also be used instead of RPMI as typically practiced before in the field for making stable TSC lines. Until the outgrowth formed on day 4, the cells were subsequently disaggregated by incubated in 0.1% trypsin-EDTA for 5 minutes in the incubator. Mouse 70% embryonic fibroblast-conditioned medium containing FGF4 and heparin was added to stop the reaction and cells were returned to the incubator. Medium was replaced every other day until TS cell colonies were be observed (not before day 14-passage 3), and following the medium was changed to a TSC culture medium to maintain the cells. Immunostaining for TSC markers Cdx2, Gata3, TBR2, and TFAP2C and lack of Oct4 and Nanog pluripotency markers, confirmed TSC identity of this representative TSC line (FIGS. 1-2). The cells could be passaged for over 50 times while maintaining their morphology and markers.

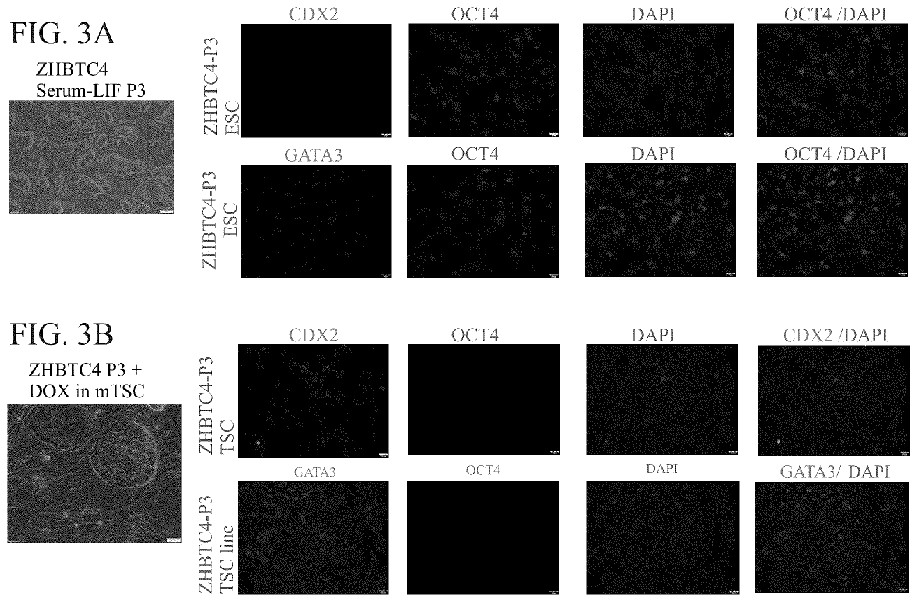

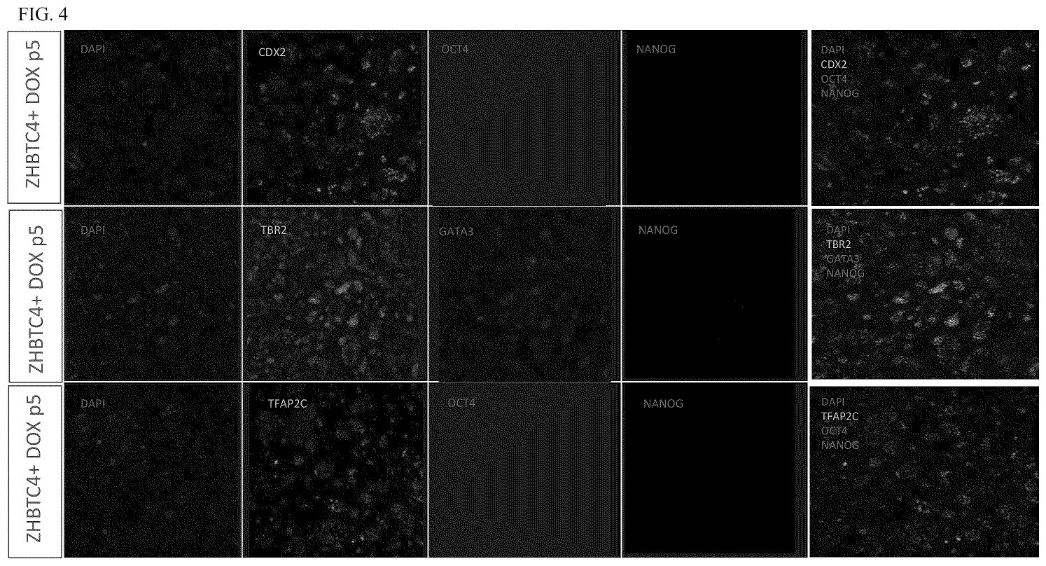

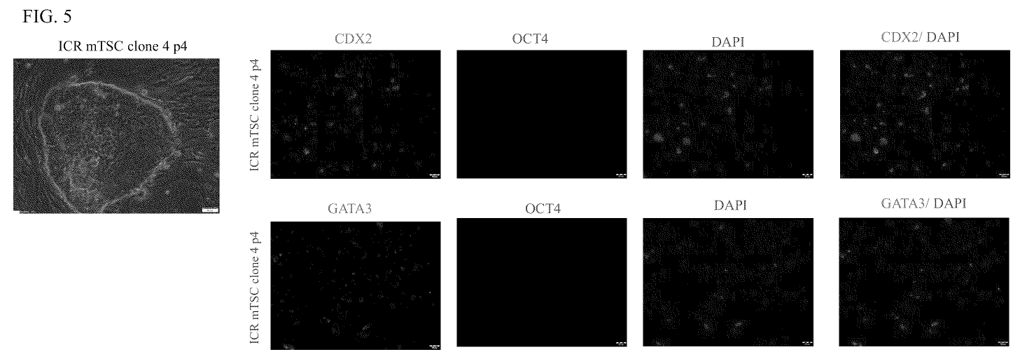

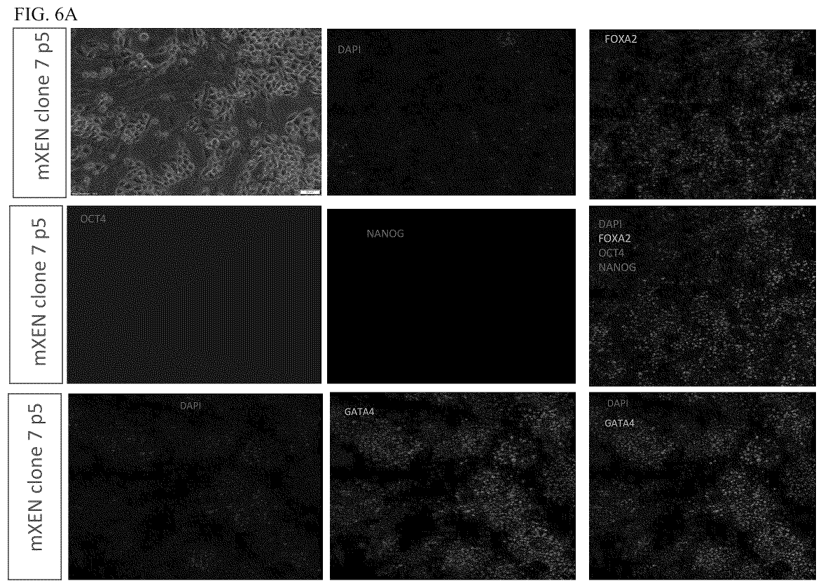

Mouse ES derived Mouse TSC line #2 (mTSC #2)—was derived as previously established (PMID: 10742100) by inhibition of Oct4 together with transferring the cells in TSC media detailed above in mTSC #1. Briefly, the ZHBTC4 mouse ES line was used in which DOX induces inhibition of Oct4 shut down (Tet-off), and was passaged in TSC media together with DOX (PMID: 10742100). Immunostaining showed that while the parental ESC line is positive for the Oct4 pluripotency marker and negative for Cdx2 and Gata3 TSC markers (FIG. 3A); the established mTSC #2 line lacks Oct4 and Nanog pluripotency markers and stably expresses the TSC markers Cdx2, TBR2, Gata3 and TFAP2C (FIGS. 3B and 4), confirming the TSC identity of this representative TSC line. Further, the mTSC #2 line was maintained on MEFs and had a classical TSC colony morphology (doi: 10.1126/science.282.5396.2072).

Mouse ES derived Mouse TSC line #3 (mTSC #3)—was derived as previously established (www(dot)doi(dot)org/10 (dot)1016/j(dot)cell(dot)2005(dot)08(dot)040) by transient overexpression of Cdx2 in mouse ESCs expanded in semi-primed FBS/LIF conditions with transferring the cells in TSC media detailed above in mTSC #1. Specifically, a previously described ICR mouse ES line (PMID: 19427283) was used in which Cdx2 was transiently transfected, and was passaged in TSC media. Following, TSC colonies were picked, passaged and validated. Immunostaining for TSC markers Cdx2 and Gata3 confirmed TSC identity of this representative TSC line (FIG. 5). Further, the mTSC #3 line was maintained on MEFs and had a classical TSC colony morphology.

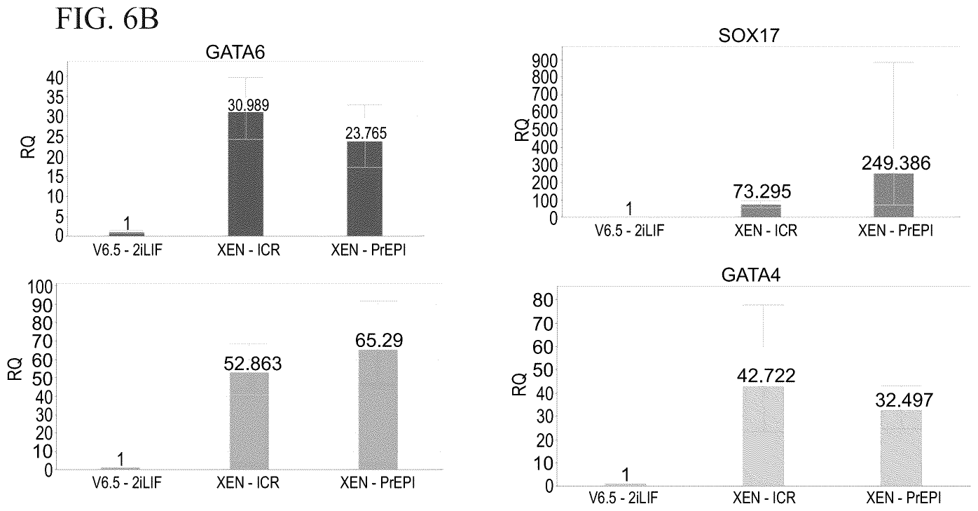

Mouse XEN line #7 (XEN-ICR clone #7)—was established according to previously described classical protocols (doi: 10.1071/rd06125). Briefly, XEN cells were derived from E3.5 blastocysts flushed from the uterus of 5-weeks-old female ICR mouse using modified XEN conditions. Mouse blastocysts were cultured on feeder cells until they formed an outgrowth on day 4 that was subsequently disaggregated by incubation in 0.1% trypsin-EDTA for 5 minutes in the incubator. Fresh mouse ES (FBS-LIF based) was added to stop the trypsin reaction, and was replaced the medium every other day until XEN cell colonies could be observed. Following, XEN cell colonies were picked and maintained as lines with a standard XEN medium: advanced RPMI 1640 (11875-093, Thermo Fisher Scientific) or RPMI1640 (Gibco 11875085) supplemented with 15% (v/v) FBS (Gibco or Biological Industries) and 0.1 mM β-mercaptoethanol (21985-023, Thermo Fisher Scientific or Sigma), 1× Glutamax (Invitrogen), 1% penicillin-streptomycin (15140122, Thermo Fisher Scientific; or Biological Industries), 1× nonessential amino acids (Biological Industries) and 1 mM Sodium-pyruvate (BI 03-042-1B). It required at least 15 days and 3 passages to be able to establish individual early passage mouse XEN lines, as previously described (doi: 10.1071/rd06125). Immunostaining of early passage mouse XEN line clone 7 at passage 5, validated loss of the pluripotency markers Oct4 and Nanog as expected, and expression of the XEN markers Foxa2 and Gata4 (FIG. 6A). In addition, real-time PCR analysis demonstrated upregulation of expression of the XEN markers Gata6, PDGFRA, Sox17 and Gata4 (FIG. 6B), further confirming their identity (10.1371/journal.pone.0009794). Mouse naïve V6.5 naïve ESCs expanded in 2i-LIF condition were used as a reference control (value set as 1).

Mouse XEN line XEN-Prep1—was established as described for XEN line #7 (side by side). Real-time PCR analysis demonstrated upregulation of expression of the XEN markers Gata6, PDGFRA, Sox17 and Gata4 (FIG. 6B), confirming their identity (10.1371/journal.pone.0009794). Mouse naïve V6.5 naïve ESCs expanded in 2i-LIF condition were used as a reference control (value set as 1).

Mouse naïve pluripotent stem cells (naïve PSCs) line-Pluripotent V6.5 GFP labeled mouse ES line (PMID: 19427283) was maintained and expanded in serum-free chemically defined N2B27-based media: 240 ml Neurobasal (ThermoFisher 21103049) and 240 ml of DMEM-F12 with HEPES (SIGMA D6421), 5 ml N2 supplement (Invitrogen; 17502048), 5-10 ml B27 supplement (Invitrogen; 17504044), 2 mM glutamine or 1 mM GLutamax (Invitrogen), 1% nonessential amino acids (Invitrogen), 0.1 mM β-mercaptoethanol (Sigma), 1% penicillin-streptomycin (Invitrogen). Optionally, the medium also included 5 mg/ml BSA (Sigma). Naïve 2i/LIF conditions for murine PSCs included 20 ng/ml recombinant human LIF (in-house made). Where indicated 2i was added: small-molecule inhibitors CHIR99021 (CH, 3 μM—Axon Medchem) and PD0325901 (PD, 1 μM—Axon Medchem 1408).

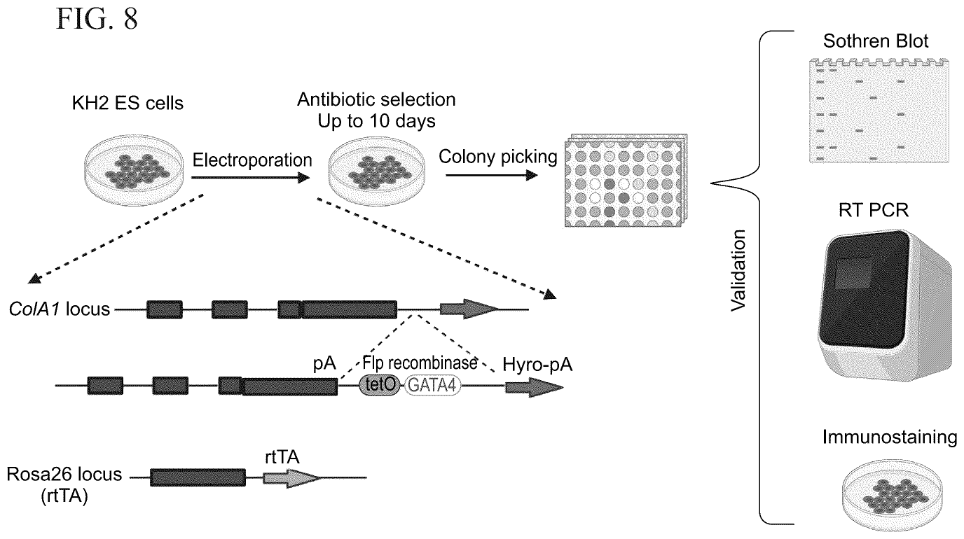

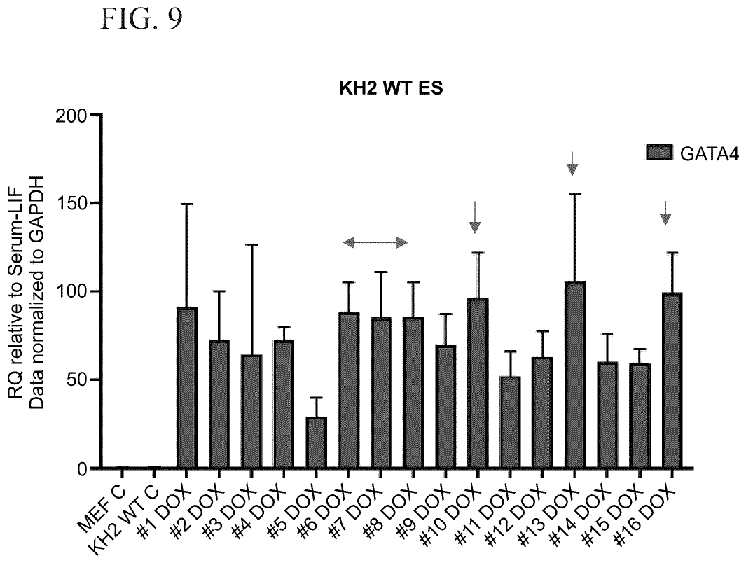

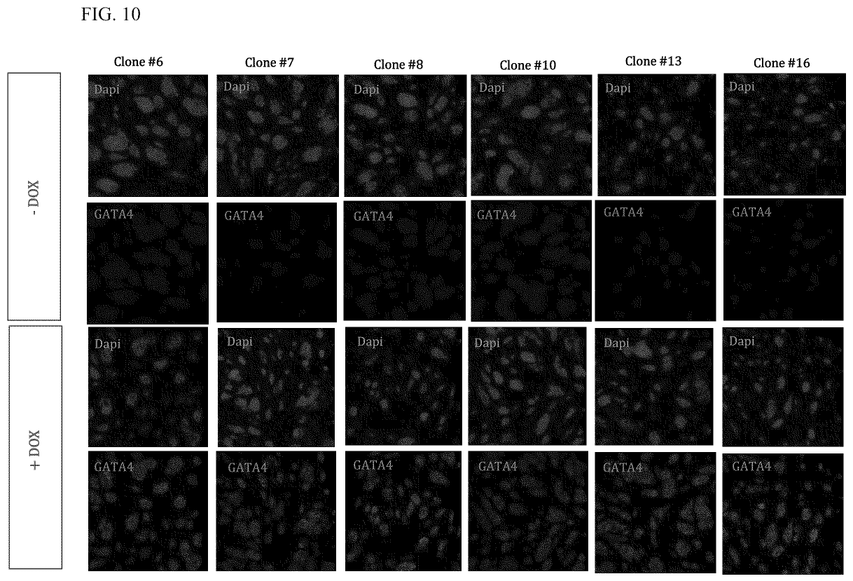

KH2-Gata4 Dox inducible cells (KH2-GATA4)-KH2-WT cell line is a v6.5 mouse ES line in which the Rosa26 locus was targeted to express M2Rtta, and the Cola1 Locus was modified to be receptive to a plasmid with TetOn promoter (PMID: 16400644) and an insert (in this case Gata4 gccctttttgggtttttgttttttttttttttttgatttttggtgacagttccgcacacccgcattctagttcttgtctgcctcgtgctcagagcttgg ggcgatgtaccaaagcctggccatggccgccaaccacggccccccgcccggcgcctacgaagcaggtggccctggcgccttcatgcac agcgcgggcgccgcgtcctcgcccgtctacgtgcccactccgcgggtgccgtcctctgtgctgggcctgtcctacctgcagggcggtggc agtgccgctgcagctggaaccacctcgggtggcagctccggggccggcccgtcgggtocagggcctgggacccagcagggtagccct ggctggagccaagctggagccgagggagccgcctacaccccgccgcccgtgtccccgcgcttctctttcccggggactactgggtccct ggcggccgctgccgccgctgccgcagcccgggaagctgcagcctacggcagtggcggcggggcggcgggcgctggtctggctggcc gagagcagtacgggcgtccgggcttcgccggctcctactccagcccctacccagcctacatggccgacgtgggagcatcctgggccgca gccgctgccgcctctgccggccccttcgacagcccagtcctgcacagcctgcctggacgggccaaccctggaagacaccccaatctcgt agatatgtttgatgacttctcagaaggcagagagtgtgtcaattgtggggccatgtccaccccactctggaggcgagatgggacgggacac tacctgtgcaatgcctgtggcctctatcacaagatgaacggcatcaaccggcccctcattaagcctcagegccgcctgtccgcttcccgccg ggtaggcctctcctgtgccaactgccagactaccaccaccacgctgtggcgtcgtaatgccgagggtgagcctgtatgtaatgcctgcggc ctctacatgaagctccatggggttcccaggcctcttgcaatgcggaaggaggggattcaaaccagaaaacggaagcccaagaacctgaat aaatctaagacgccagcaggtcctgctggtgagaccctccctccctccagtggtgcctccagcggtaactccagcaatgccactagcagca gcagcagcagtgaagagatgcgccccatcaagacagagcccgggctgtcatctcactatgggcacagcagctccatgtcccagacattca gtactgtgtccggccacgggccctccatccatccagtgctgtctgctctgaagctgtccccacaaggctatgcatctcctgtcactcagacat cgcaggccagctccaagcaggactcttggaacagcctggtcctggctgacagtcatggggacataatcaccgcgtaatcagegcccccc cttccctcttcaaattcctgctcggacttgggacgtgggggccagcaaagtaaaaggctgggaagggc; SEQ ID NO: 1) which can be flipped in the collagen locus. Targeted colonies were subjected to hygromycin selection of 10 days, and individual ES colonies were selected for further analysis to generate KH2-Gata4 that overexpress Gata4 upon DOX addition (FIG. 8). Real Time PCR analysis and immunostaining confirmed that the clones showed DOX inducible overexpression of the Gata4 insert (FIGS. 9-10).

Aggregation media (AM)—240 ml Neurobasal (ThermoFisher 21103049) and 240 ml of DMEM-F12 (Thermo 21331-020 or SIGMA D6421), 5 ml N2 supplement (Invitrogen; 17502048), 10 ml B27 supplement (Invitrogen; 17504044), 2 mM glutamine or 1× GLutamax (Invitrogen), 1× nonessential amino acids (NEAA) (Invitrogen), 0.1 mM β-mercaptoethanol (GIBCO 31350-010), 1× penicillin-streptomycin (Invitrogen), 0.45% BSA (Sigma A7979), 1% Sodium-pyruvate (BI 03-042-1B). When indicated, serum was added to aggregation media at the indicated percentage and origin.

IVC1 media—Advanced DMEM/F12 (Gibco 12634010) supplemented with Glutamax 1×, Pen/Strep 1×, ITS-X 1× (Gibco 51500056), beta-Estradiol 8 nM, Progesterone 20 ng/ml, N-acetyl-L-cysteine 25 μM and FBS or HAS as indicated.

EUCM media (also referred to as EUCM1)—25% DMEM (GIBCO 11880) or Advanced DMEM/F12 (Thermo 12634010), 25% HAS (Human Adult Serum or Human umbilical cord serum), 50% Rat Serum (RAS), Glutamax 1× (GIBCO 35050038), HEPES 11 mM (GIBCO 15630056), Pen/Strep 1×. When indicated, the media was further supplemented with D-Glucose 4-12 mg/ml, sodium pyruvate 1 mM and/or non-essential amino acids (NEAA 1×). DMEM aliquots were stored at 4° C. and used within 2 months. Rat serum was stored at −80° C. and heat inactivated at 56° C. for half an hour and filtered through a 0.22 μm PVDF filter (Millipore: SLGV033RS) prior to use. HAS should be freshly thawed and used immediately before experimentation. Rat serum and HAS can be thawed/frozen once.

Aggregation plate preparation—To prepare the 24 well AggreWell 400 plate (STEMCELL Technologies 34415) or AggreWell 800 plate (STEMCELL Technologies), 500 ml of anti-adherence rinsing solution (STEMCELL Technologies 07010) was added to each well. The plate was then centrifuged at 2,000×g for 5 minutes and was incubated for 30 minutes at room temperature. Anti-adherence Rinsing solution (Stem Cell Technologies—#07010) was then aspirated from the well and 1 ml of PBS−/− was added to wash each well, and this PBS wash was repeated twice. The 500 ml of aggregation medium (AM) was added to each well after aspirating the PBS.

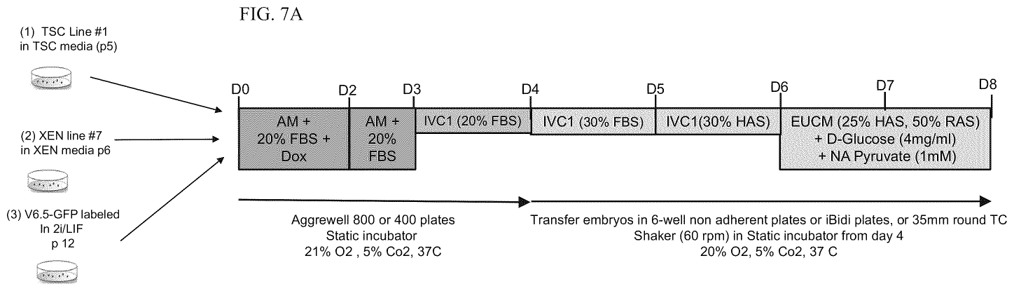

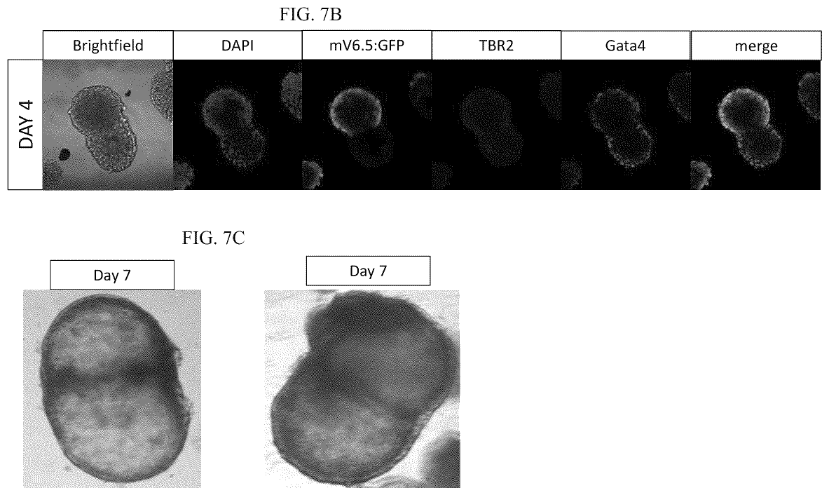

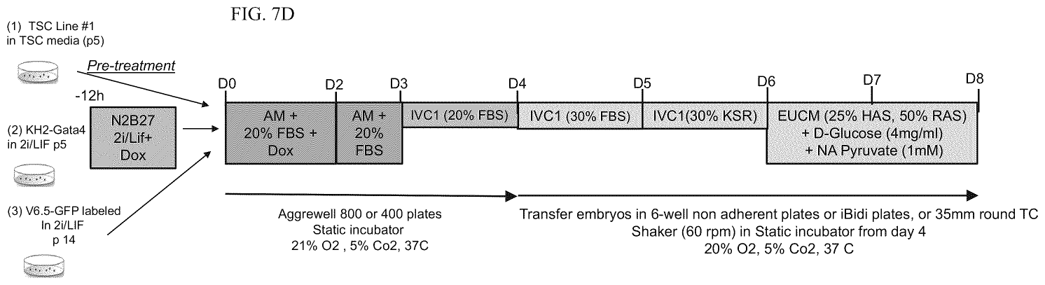

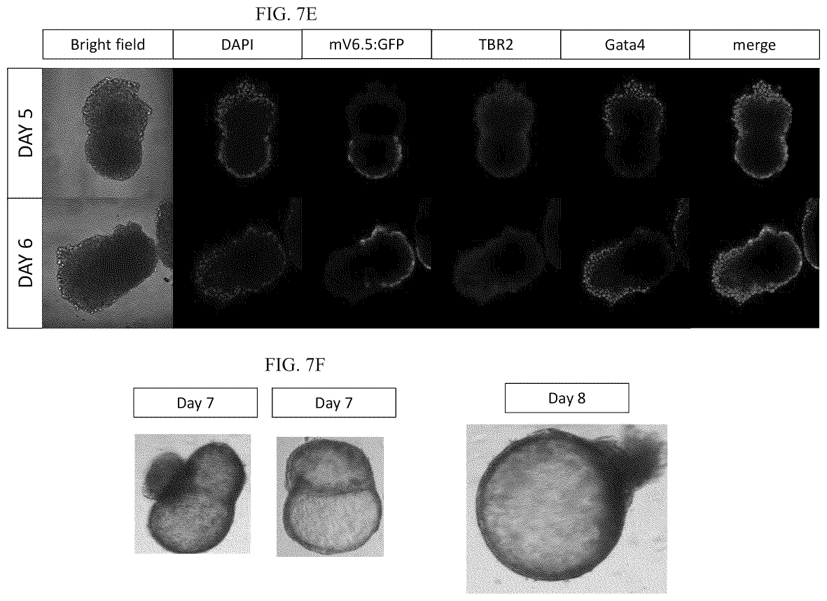

Co-aggregation and culture (FIG. 7A)—Co-aggregation was defined as time point 0 of the protocol and was made from three starting stem cell types:
1) V6.5-GFP (To give rise to embryo proper);
2) mouse XEN line #7 cells (expected to give rise to extraembryonic endoderm lineage); and
3) mouse TSC line #1 cells (expected to give rise to entire trophectoderm lineage).

Cells were trypsinised with 0.05% trypsin-EDTA for 3-5 minutes followed by feeder depletion on gelatinized plates for 20 minutes at 37° C. (when MEFs were used). Following, cells were resuspended in aggregation media (AM) supplemented with Doxycycline (2 μg/ml final concentration). Following cell count, a total number of 18000 TSCs, 6000 XEN and 6000 v6.5-GFP WT cells were added per well in an AggreWell 400 Microwell 24 well plate. In 800 Microwell plates a total number of 4500 TSCs, 1500 XEN and 1500 V6.5 GFP cells were added per well in an AggreWell 24 well. The cell mixture was added as 1 ml per well (final volume of each well of 24 well is 1.5 ml), 25 cells/well.
Day 0—The plate was centrifuged at 100×g for 3 minutes and this was marked as day 0, and then placed into static incubator at 37° C., 5% Co2 and 20% O2.
On Day 1, 1 ml of media was gently aspirated from each well and replaced with pre-warmed 1 ml of fresh AM+20% PBS+DOX as indicated in FIG. 7A.
On day 2, 1 ml of media was gently aspirated from each well and replaced with pre-warmed 1 ml of fresh AM+20% PBS as indicated in FIG. 7A.
On day 3, 1 ml of media was gently aspirated from each well and replaced with pre-warmed 1 ml of IVC1 media with 20% FBS as indicated in FIG. 7A.
On day 4, embryos were transferred with 3 ml Plastic Pasteur Pipette to a 15 ml falcon tubes, allowed to sit for 5 minutes, and most media was gently removed leaving embryos in ˜1 ml media. The embryos were then moved with the pipette gently in petri dish (35 mm Corning 430588 plates) or (6 well plates Costar 3736) or iBidi plate with 4 ml of IVC1 media with 30% FBS, and were incubated on a rotating shaker 60 rpm in a static incubator (37° C., 20% O2 and 5% Co2).
On day 5, 2-3 ml of media was gently aspirated with manual pipette and replaced with fresh IVC1 media with 30% HAS (bringing back to total volume of 4 ml) and incubated on a shaker (60 rpm) in a static incubator (37° C., 20% O2 and 5% Co2).
From at day 6 and day 7—each day 2-3 ml of media was gently aspirated with manual pipette and replaced with fresh EUCM media supplemented with D-Glucose 4 mg/ml and sodium pyruvate 1 mM as indicated in FIG. 7A (bringing back to total volume of 4 ml).
At day 8 onwards, high quality embryos were selected and transferred into a previously described ex-utero roller culture system (Alejandro-Castrjon et al. Nature 2021) in fresh EUCM media supplemented with D-Glucose 4 mg/ml and sodium pyruvate 1 mM and incubated at 20% O₂, 5% Co2 and 6.5 Psi.

Co-aggregation and culture (FIG. 7D)—KH2-Gata4 Dox inducible cells expanded in 2i/LIF were used instead of XEN cells. To this end, KH2-GATA4 cells were subjected to DOX addition in 2i/LIf 12 hours prior to aggregation. Co-aggregation was defined as time point 0 of the protocol and was made from three starting stem cell types:
1) V6.5-GFP WT naïve ESCs (To give rise to embryo proper);
2) DOX pre-treated KH2 tetO-GATA4 naïve ESC (to give rise to extraembryonic endoderm lineage); and
3) TSCs (expected to give rise to entire trophectoderm lineage).

Cells were trypsinised with 0.05% trypsin-EDTA for 3-5 minutes followed by feeder depletion on gelatinized plates for 20 minutes at 37° C. (when MEFs were used). Following, cells were re-suspended in aggregation media (AM) supplemented with Doxycycline (2 μg/ml final concentration). Following cell count, a total number of 18000 TSCs, 6000 KH2-GATA4 and 6000 V6.5-GFP naïve ES cells were added per well in AggreWell 400 Microwell 24 well plate. In 800 Microwell plates a total number of 4500 TSCs, 1500 KH2-GATA4 and 1500 V6.5 GFP WT cells were added per well in AggreWell 24 well. The cell mixture was added as 1 ml per well (final volume of each well of 24 well is 1.5 ml), 25 cells/well.
Day 0—The plate was centrifuged at 100×g for 3 minutes and this was marked as day 0, and then placed into static incubator at 37° C., 5% Co2 and 20% O2.
On Day 1, 1 ml of media was gently aspirated from each well and replaced with pre-warmed 1 ml of fresh AM+20% FBS+DOX, as indicated in FIG. 7D.
On day 2, 1 ml of media was gently aspirated from each well and replaced with pre warmed 1 ml of fresh AM+20% FBS, as indicated in FIG. 7D.
On day 3, 1 ml of media was gently aspirated from each well and replaced with pre warmer 1 ml of IVC1 media with 20% FBS, as indicated in FIG. 7D.
On day 4, embryos were transferred with a 3 ml Plastic Pasteur Pipette to 15 ml falcon tubes, allowed to sit for 5 minutes, and most media was gently removed leaving embryos in ˜1 ml media. The embryos were then moved with the pipette gently into petri dish (35 mm Corning 430588 plates) or (6 well plates Costar 3736) or iBidi plates with 4 ml of IVC1 media with 30% FBS (as indicated in FIG. 7D) and incubated on a shaker (60 rpm) in a static incubator (37° C., 20% O2 and 5% Co2).
On day 5, 2-3 ml of media was gently aspirated with manual pipette and replaced with fresh IVC1 media with 30% KSR (Knockout serum replacement—Invitrogen, as indicated in FIG. 7D, bringing back to total volume of 4 ml) and kept on the shaker rotating at 60 rpm in the static incubator (37° C., 20% O2 and 5% Co2).
From at day 6 and day 7—each day 2-3 ml of media was gently aspirated with a manual pipette and replaced with fresh EUCM media supplemented with D-Glucose 4 mg/ml and sodium pyruvate 1 mM, as indicated in FIG. 7D (bringing back to total volume of 4 ml).
At day 8 onwards, high quality embryos were selected and transferred into a previously described ex-utero roller culture system (Alejandro-Castrjon et al. Nature 2021) in fresh EUCM media supplemented with D-Glucose 4 mg/ml and sodium pyruvate 1 mM and incubated at 20% O₂, 5% Co2 and 6.5 Psi.

Whole-mount immunostaining of embryos—Embryos grown ex utero were dissected (with or without removing the yolk sac and amnion for E8.5 embryos), washed once with 1×PBS, then transferred to ibidi glass bottom 8-well slides (iBidi) and fixed with 4% PFA EM grade (Electron microscopy sciences, 15710) in PBS at 4° C. over-night. Embryos were then washed in PBS for 5 minutes 3 times, permeabilized in PBS with 0.5% Triton X-100/0.1 M glycine for 30 minutes, blocked with 10% normal donkey serum/0.1% Triton X-100 in PBS for 1 hour at room temperature (RT), and incubated over-night at 4° C. with primary antibodies, diluted in blocking solution. After, embryos were rinsed 3 times for 5 minutes each in PBS/0.2% TritonX-100, incubated for 2 hours at room temperature with secondary antibodies diluted 1:200 in blocking solution (all secondary antibodies were from Jackson ImmunoResearch), counterstained with DAPI (1 μg/ml in PBS) for 10 minutes, and washed with PBS for 5 minutes 3 times. If necessary, yolk sacs separated from the embryos were fixed and stained using this whole-mount immunostaining protocol. The following primary antibodies were used: Rabbit monoclonal anti-Brachyury (Cell Signaling, 81694) 1:100; Rabbit polyclonal anti-Cdx2 (Cell Signaling, 3977) 1:100; Mouse monoclonal anti-Cdx2 (Biogenex, AU392A-UC) 1:100; Goat polyclonal anti-Gata4 (Santa Cruz, SC-1237) 1:100; Rabbit polyclonal anti-Gata4 (Abcam, Ab84593) 1:100; Rabbit polyclonal anti-Foxa2 (Abcam, Ab40874) 1:100; Mouse monoclonal anti-Myosin Heavy Chain II (MF-20) (R&D, MAB4470) 1:100; Goat polyclonal anti-Otx2 (R&D, AF1979) 1:200; Rabbit polyclonal anti-Pax6 (Covance, PBR-278P) 1:100; Goat polyclonal anti-Sox2 (R&D, AF2018) 1:200; Rabbit polyclonal anti-Sox9 (Millipore, AB5535) 1:100; Goat polyclonal anti-Sox17 (R&D, AF1924) 1:100; Mouse monoclonal anti-Tuj1 (C-10) (Covance, MMS-435P) 1:200; Chicken polyclonal anti-GFP (Abcam, Ab13970) 1:250; Mouse monoclonal anti-Oct4 (Santa Cruz, SC-5279) 1:100; Goat polyclonal anti-Lefty 1 (R&D, AF746) 1:100, Goat polyclonal anti-mCherry/Tomato (SiCGEN, AB0040-200) 1:200.

Clearing of embryos from E9.5 to E11.5 was performed as follows: embryos were washed once with 1×PBS and fixed overnight at 4° C. with 4% PFA EM grade diluted in PBS. Samples were washed in PBS for 5 minutes three times, followed by sample pre-treatment without methanol, i.e.: Washed in PBS/0.2% TritonX-100 for 1 hour twice, then incubated in PBS/0.2% TritonX-100/20% DMSO at 37° C. overnight, then in PBS/0.1% Tween-20/0.1% TritonX-100/0.1% Deoxycholate/0.1% NP40/20% DMSO at 37° C. overnight, then washed in PBS/0.2% TritonX-100 for 1 hour twice. For immunostaining, embryos were permeabilized in PBS/0.2% TritonX-100/20% DMSO/0.3 M glycine at 37° C. overnight, and then blocked in PBS/0.2% TritonX-100/10% DMSO/6% Donkey Serum at 37° C. overnight. After blocking, embryos were incubated with primary antibodies diluted in PBS/0.2% Tween-20 with 10 μg/ml heparin (PTwH)/5% DMSO/3% Donkey Serum at 37° C. [E9.5/E10.5=24 hours; E11.5=48 hours (72 hours for Sox17 and Foxa2 antibodies)]. Afterwards, samples were washed in PTwH for 24 hours (15 minutes, 30 minutes, 1 hour, 2 hours, and overnight washes), and incubated with adequate secondary antibodies (1:200) diluted in PTwH/3% Donkey Serum at 37° C. for 48 hours.

For human cell specific NUMA staining (when used), donkey anti-rabbit Biotin and Streptavidin-Cy3 (each incubated overnight) were used for signal enhancement. After, embryos were incubated for 30 minutes with DAPI (1 μg/ml) diluted in PTwH, washed in PTwH for one day (5 minutes, 15 minutes, 30 minutes, 1 hour, 2 hours, and overnight washes) and dehydrated in methanol/H₂O series (20%, 40%, 60%, 80%, 100%) 1 hour each at room temperature, and then incubated overnight in 100% methanol. Embryos were incubated in 66.6% DCM/33.3% methanol at room temperature on shaker for 3 hours, followed by 100% DCM (Sigma; 270997) for 5 minutes, and finally cleared and stored in Benzyl Ether (Sigma: 108014). The following primary antibodies were used: Rabbit monoclonal anti-Brachyury (Cell Signaling, 81694) 1:100; Rabbit polyclonal anti-Cdx2 (Cell Signaling, 3977) 1:100; Mouse monoclonal anti-Cdx2 (Biogenex, AU392A-UC) 1:100; Goat polyclonal anti-Gata4 (Santa Cruz, SC-1237) 1:100; Rabbit polyclonal anti-Gata4 (Abcam, Ab84593) 1:100; Rabbit polyclonal anti-Foxa2 (Abcam, Ab40874) 1:50; Mouse monoclonal anti-Myosin Heavy Chain II (MF-20) (R&D, MAB4470) 1:100; Goat polyclonal anti-Otx2 (R&D, AF1979) 1:200; Rabbit polyclonal anti-Pax6 (Covance, PBR-278P) 1:100; Goat polyclonal anti-Sox2 (R&D, AF2018) 1:200; Rabbit polyclonal anti-Sox9 (Millipore, AB5535) 1:100; Goat polyclonal anti-Sox17 (R&D, AF1924) 1:50; Mouse monoclonal anti-Tuj1 (C-10) (Covance, MMS-435P) 1:200; Chicken polyclonal anti-GFP (Abcam, Ab13970) 1:250, Goat polyclonal anti-mCherry/Tomato (SiCGEN, AB0040-200) 1:200; Rabbit polyclonal anti-human TEMEM119 (Invitrogen, PA562505) 1:100; Rabbit anti-hNUMA (Abcam, ab84680) 1:100.

Confocal Microscopy—Images were acquired with a Zeiss LSM 700 inverted confocal microscope (Carl Zeiss) equipped with 405 nm, 488 nm, 555 nm and 635 nm solid state lasers, using a Plan-Apochromat 20× air objective (numerical aperture 0.8) for E5.5/E6.5 embryos, and an EC Plan Neofluar 10× air objective (numerical aperture 0.3) for E7.5 to E11.5 embryos. Images were acquired at 1024×1024 resolution. All images were acquired within the following range of parameters: Laser power: 405 nm: 10-20%; 488 nm: 5-20%; 555 nm 10-40%; 635 nm: 30-80%. Gain ranged from 350 to 600. Pixel size was 1.25 μm with a z-step of 15 μm when using the 10× objective, or 0.5 μm with z-step of 5 μm when using the 20× objective. For confocal imaging, iDISCO cleared embryos were mounted in 35 mm glass bottom dishes (In Vitro Scientific, D35201.5N), employing ethyl cinnamate (Sigma, 112372) as imaging solution. For chimeric embryos, all parameters during image acquisition were compared to stained non-injected control embryos, imaged with equal parameters as the injected embryos. Images and maximum intensity projections were processed using Zen 2 blue edition software 2011 (Carl Zeiss) and Adobe Photoshop CS4.

Immunostaining of ES, TSC and XEN cell lines-Cells were grown on glass cover slips (13 mm 1.5H; Marienfeld, 0117530) fixed with 4% paraformaldehyde/phosphate buffer for 10 minutes at room temperature, washed three times with PBS, and permeabilized in PBS/0.1% Triton for 10 minutes. Cells were blocked with blocking solution (2% normal goat serum, 0.1% BSA in PBS/0.05% Tween) for 1 hour at RT and incubated with primary antibody diluted in blocking solution overnight at 4° C. (Antibodies in this study have all been validated in the literature and by ourselves). Cells were then washed three times with PBS/0.05% Tween, incubated with secondary antibodies for 1 hour at room temperature, washed in PBS/0.05% Tween, counterstained with DAPI (1 μg/ml), washed again three times with PBS/Tween 0.05% and mounted with Shandon Immu-Mount (Thermo Scientific, 9990412), and imaged. All comparative experiments were done simultaneously. All secondary antibodies were used from Jackson ImmunoResearch.

Real-Time PCR analysis—For RT-PCR total RNA was isolated using Trizol (Ambion Life Technologies), and 1 μg of total RNA was reverse transcribed using High-Capacity Reverse Transcription Kit (Applied Biosystems). Quantitative PCR analysis was performed with the SYBR™ Green PCR Master Mix (Applied Biosystems) using 10 ng of cDNA per reaction in a Viia7 platform (Applied Biosystems). Fold change was normalized to Gapdh and/or Actin expression as indicated per figure. The following primers were used:

| | Mouse Gapdh-Forward: |
|---|---|
| (SEQ ID NO: 2) | |
| | AGTCAAGGCCGAGAATGGGAAG; |
| | Mouse Gapdh-Reverse: |
| (SEQ ID NO: 3) | |
| | AAGCAGTTGGTGGTGCAGGATG |
| | Mouse Oct4-Forward: |
| (SEQ ID NO: 4) | |
| | AGAGGATCACCTTGGGGTACA; |
| | Mouse Oct4-Reverse: |
| (SEQ ID NO: 5) | |
| | CGAAGCGACAGATGGTGGTC; |
| | Mouse Nanog-Forward: |
| (SEQ ID NO: 6) | |
| | CTCAAGTCCTGAGGCTGACA; |
| | Mouse Nanog-Reverse: |
| (SEQ ID NO: 7) | |
| | TGAAACCTGTCCTTGAGTGC; |
| | Mouse Sox2-Forward: |
| (SEQ ID NO: 8) | |
| | TAGAGCTAGACTCCGGGCGATGA; |
| | Mouse Sox2-Reverse: |
| (SEQ ID NO: 9) | |
| | TTGCCTTAAACAAGACCACGAAA; |
| | Mouse Klf4-Forward: |
| (SEQ ID NO: 10) | |
| | GCACACCTGCGAACTCACAC; |
| | Mouse Klf4-Reverse: |
| (SEQ ID NO: 11) | |
| | CCGTCCCAGTCACAGTGGTAA; |
| | Mouse Cdx2-Forward: |
| (SEQ ID NO: 12) | |
| | GCGAAACCTGTGCGAGTGGATG; |
| | Mouse Cdx2-Reverse: |
| (SEQ ID NO: 13) | |
| | CGGTATTTGTCTTTTGTCCTGGTTTTCA; |
| | Mouse Gata4-Forward: |
| (SEQ ID NO: 14) | |
| | CACAAGATGAACGGCATCAACC; |
| | Mouse Gata4-Reverse: |
| (SEQ ID NO: 15) | |
| | CAGCGTGGTGGTAGTCTG; |
| | Mouse Gata6-Forward: |
| (SEQ ID NO: 16) | |
| | CTTGCGGGCTCTATATGAAACTCCAT; |
| | Mouse Gata6-Reverse: |
| ((SEQ ID NO: 17) | |
| | TAGAAGAAGAGGAAGTAGGAGTCATAGGGACA; |
| | Mouse Brachyury(T)-Forward: |
| (SEQ ID NO: 18) | |
| | CTGTGACTGCCTACCAGAATGAGGAG; |
| | Mouse Brachyury(T)-Reverse: |
| (SEQ ID NO: 19) | |
| | GGTCGTTTCTTTCTTTGGCATCAAG; |
| | Mouse Otx2-Forward: |
| (SEQ ID NO: 20) | |
| | CTTCGGGTATGGACTTGCTG |
| | Mouse Otx2-Reverse: |
| (SEQ ID NO: 21) | |
| | CCTCATGAAGATGTCTGGGTAC; |
| | Mouse Fgf5-Forward: |
| (SEQ ID NO: 22) | |
| | CAAAGTCAATGGCTCCCACGAAG; |
| | Mouse Fgf5-Reverse: |
| (SEQ ID NO: 23) | |
| | CTACAATCCCCTGAGACACAGCAAATA; |
| | Total (endogenous + exogenous) |
| | mouse Gata4 Forward: |
| (SEQ ID NO: 24) | |
| | CCAGTGCTGTCTGCTCTGAA; |
| | Total (endogenous + exogenous) |
| | mouse Gata4 Reverse: |
| (SEQ ID NO: 25) | |
| | GCTGTTCCAAGAGTCCTGCT; |
| | Total (endogenous + exogenous) |
| | mouse Cdx2 Forward: |
| (SEQ ID NO: 26) | |
| | GCGAAACCTGTGCGAGTGGATG; |
| | Total (endogenous + exogenous) |
| | mouse Cdx2 Reverse: |
| (SEQ ID NO: 27) | |
| | CGGTATTTGTCTTTTGTCCTGGT; |
| | Exogenous mouse Gata4 Forward: |
| (SEQ ID NO: 28) | |
| | AAATTCCTGCTCGGACTTGGGAC; |
| | Exogenous mouse Gata4 Reverse: |
| (SEQ ID NO: 29) | |
| | GTGGTTTGTCCAAACTCATCAATGTATCTT; |
| | Exogenous mouse Cdx2 Forward: |
| (SEQ ID NO: 30) | |
| | GAGGGGTTTTAAACTCCACTGTCACC; |
| | Exogenous mouse Cdx2 Reverse: |
| (SEQ ID NO: 31) | |
| | GTGGTTTGTCCAAACTCATCAATGTATCTT; |
| | Endogenous mouse Gata4 Forward: |
| (SEQ ID NO: 32) | |
| | CACTGGAAAAGCCTGCGTTCTTACA; |
| | Endogenous mouse Gata4 Reverse: |
| (SEQ ID NO: 33) | |
| | AGAATAAGGAAGGAAGAAGTCTCTTGCCTC; |
| | Endogenous mouse Cdx2 Forward: |
| (SEQ ID NO: 34) | |
| | AATTATGGACCTCAGGGGAAGACATG; |
| | Endogenous mouse Cdx2 Reverse: |
| (SEQ ID NO: 35) | |
| | GCTGGCAGGAAGAGTCGGAATG. |

### Results

Co-aggregation of three starting cell types, namely a naïve mouse PSCs (V6.5-GFP), mouse XEN cells [mouse XEN line #7 cells or KH2-Gata4 Dox inducible cells) and mouse TSCs (mouse TSC line #1 cells) followed by ex-utero culturing (FIGS. 7A-F) did not lead to generation of an organized embryo. Specifically, at Day 4 of the protocol shown in FIG. 7A and days 5 and 6 of the protocol shown in FIG. 7D, trophoblast cells were aberrantly distributed in all regions of the embryos, and did not show the expected patters of being concentrated on the region opposite to the GFP+ epiblast (FIGS. 7B and 7E). At days 8 and 9, structures that lacked organized embryos in the yolk sac that did not show any sign of emergence of neural folds and cardiac regions were detected (FIGS. 7C and 7F).

### Example 2

Aggregation of Three Mouse Naïve PSCs Subpopulations Generates an Organized Embryo

### Materials and Methods

Mouse naïve pluripotent stem cells (naïve PSCs) line—As described in Example 1 hereinabove.

KH2-Gata4 Dox inducible naïve pluripotent stem cells (KH2-GATA4)—As described in Example 1 hereinabove.

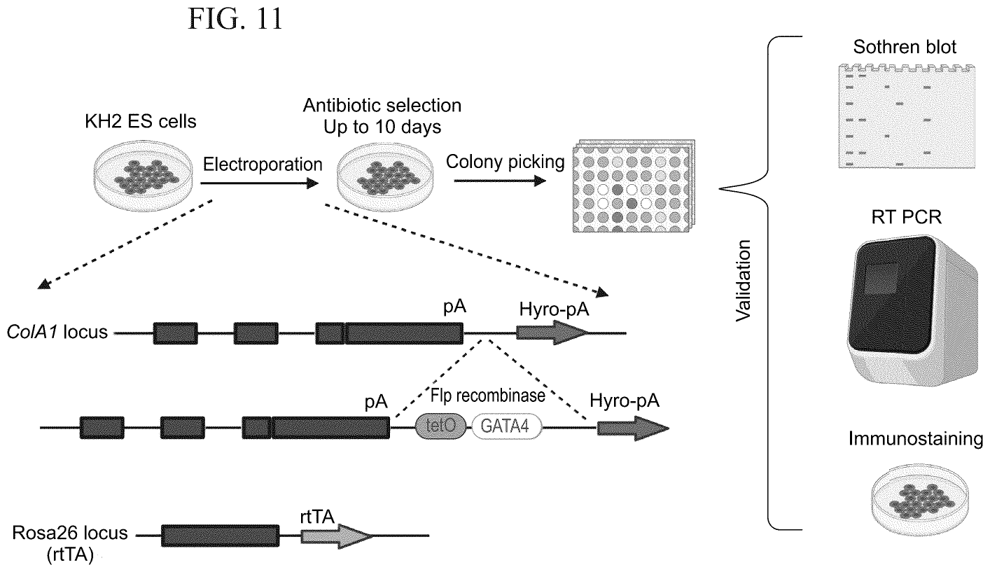

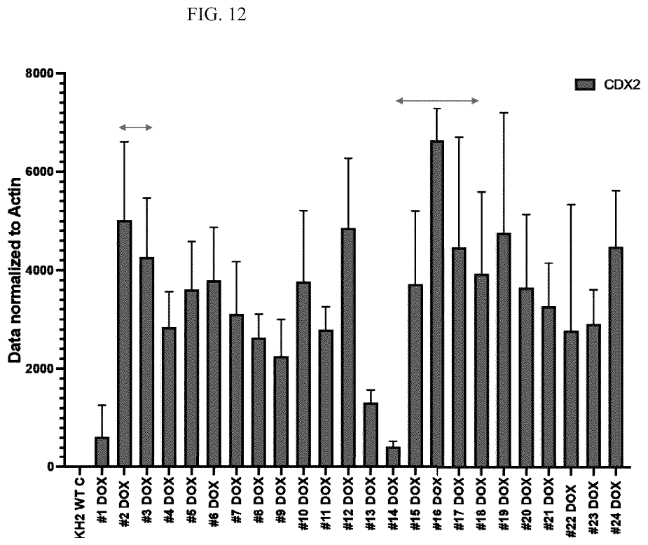

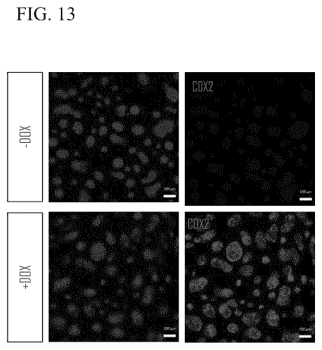

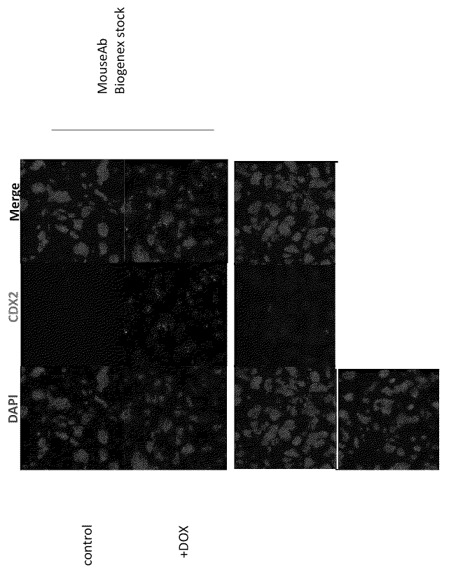

KH2-Cdx2 Dox inducible naïve pluripotent stem cells (KH2-Cdx2)—As noted hereinabove, KH2-WT cell line is a v6.5 mouse ES line in which the Rosa26 locus was targeted to express M2Rtta, and the Cola1 Locus was modified to be receptive to a plasmid with TetOn promoter (PMID: 16400644) and an insert (in this case Cdx2: atgtacgtgagctaccttctggacaaggacgtgagcatgtatcctagctccgtgcgccactccggcggcctgaacctggctccgcagaactt tgtcagtcctccgcagtacccggactacggtggttaccacgtggcggccgcggcggctgctacggcgaacttggacagcgctcagtcccc agggccatcctggcccaccgcgtacggcgcccctctccgcgaggactggaatggctacgcacccgggggcgctgcggcagccaacgc ggtagcccacggtctcaatggtggctccccggccgccgctatgggctacagcagccccgccgaataccacgcgcaccatcacccgcatc atcacccgcaccatccggccgcctcgccgtcctgcgcctccggcttgctgcagacgctcaaccteggccccccggggcccgcagccacc gccgccgccgaacagctgtcccccagcggccagcggcgaaacctgtgcgagtggatgcggaagcccgcgcagcagtccctaggaag ccaagtgaaaaccaggacaaaagacaaataccgggtggtgtacacagaccatcagcggctggagctggagaaggagtttcactttagtcg atacatcaccatcaggaggaaaagtgagctggctgccacacttgggctctccgagaggcaggttaaaatttggtttcagaaccgcagagcc aaggagaggaaaatcaagaagaagcagcagcagcaacagcagcagcagcaacaacagcctccacagccgccgccacaaccttccca gcctcagccgggtgccctgcggagcgtgcccgagcccttgagtcctgtgacctccttgcaaggctcagtgcctggttctgtccctggggttc tggggccagctggaggggttttaaactccactgtcacccagtga: SEQ ID NO: 36) which can be flipped in the collagen locus. Targeted colonies were subjected to hygromycin selection of 10 days, and individual ES colonies were selected for further analysis to generate KH2-Cdx2 that overexpress Cdx2 upon DOX addition (FIG. 11). Real Time PCR analysis and immunostaining confirmed that the clones showed DOX inducible overexpression of the Cdx2 insert (FIGS. 12-14).

Expansion and maintenance of naïve mouse PSCs—Three different mouse ES lines were separately expanded and maintained in mouse naïve N2B27 2iLIF conditions: KH2 ESC, KH2-Gata4 Dox inducible cells (KH2-GATA4) and KH2-Cdx2 Dox inducible cells (KH2-CDX2). Cells were expanded on gelatin or MEF coated plates in N2B27 2i/LIF medium for at least 3 passages (10-12 days) in order to become naïve (PMID 33915080; PMID 26860365; PMID: 26291026; PMID: 26076835; PMID 24172903; PMID 25569111) and competent to successfully execute the protocol delineated herein. Both 5% O2 and 20% O2 conditions were suitable (37° C. and 5% Co2).

N2B27 2i/LIF medium—240 ml Neurobasal (ThermoFisher 21103049) and 240 ml of DMEM-F12 (Thermo 21331-020 or SIGMA D6421), 5 ml N2 supplement (Invitrogen; 17502048), 10 ml B27 supplement (Invitrogen; 17504044), 2 mM glutamine or 1 mM GLutamax (Invitrogen), 1% nonessential amino acids (Invitrogen), 0.1 mM β-mercaptoethanol (Sigma), 1% penicillin-streptomycin (Invitrogen), 3 M CHIR99021 (GSK3i-CH, Axon Medchem), 1 μM PD0325901 (MEK/ERKi, PD, Axon Medchem 1408) and recombinant LIF 20 ng/ml.

Aggregation media (AM)—As described in Example 1 hereinabove.

EUCM media (also referred to as EUCM1)—As described in Example 1 hereinabove.

EUCM2 media—CMRL (Gibco 11530037) or a 1:1 mix of CMRL and Advanced DMEM/F12 (Thermo 12634010), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 20% FBS.

EUCM3 media—CMRL (Gibco 11530037) or a 1:1 mix of CMRL and Advanced DMEM/F12 (Thermo 12634010), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 30% FBS.

EUCM4 media—CMRL (Gibco 11530037) or a 1:1 mix of CMRL and Advanced DMEM/F12 (Thermo 12634010), Glutamax 1×, Sodium pyruvate 1 mM, Pen/Strep 1×+NEAA 1×, D-Glucose 4 mg/mL, ITS-X 1× (Gibco 51500056), beta-Estradiol 8 nM, Progesterone 20 ng/ml, 30% HAS.

EUCM4* media—4:1 mix of CMRL (Gibco 11530037) and Advanced DMEM/F12 (Thermo 12634010), Glutamax 1×, Sodium pyruvate 0.5 mM, Pen/Strep 1×+NEAA 0.5×, +extra D-Glucose 3 mg/mL, ITS-X 1× (Gibco 51500056), beta-Estradiol 8 nM, Progesterone 20 ng/ml, 20% RAS, 10% HAS+HEPES 2 mM (GIBCO 15630056). When indicated, EUCM5 also contained 10% KSR.

EUCM5 media—4:1 mix of CMRL (Gibco 11530037) and Advanced DMEM/F12 (Thermo 12634010), Glutamax 1×, Sodium pyruvate 0.5 mM, Pen/Strep 1×+NEAA 0.5×, +extra D-Glucose 3 mg/mL, ITS-X 1× (Gibco 51500056), beta-Estradiol 8 nM, Progesterone 20 ng/ml, 30% RAS, 15% HAS, HEPES 2 mM (GIBCO 15630056). When indicated, EUCM5 also contained 5% KSR.

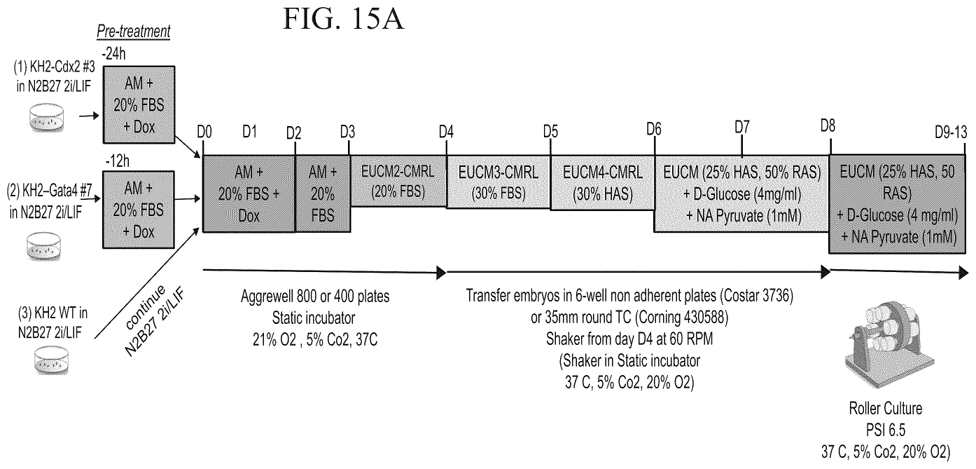

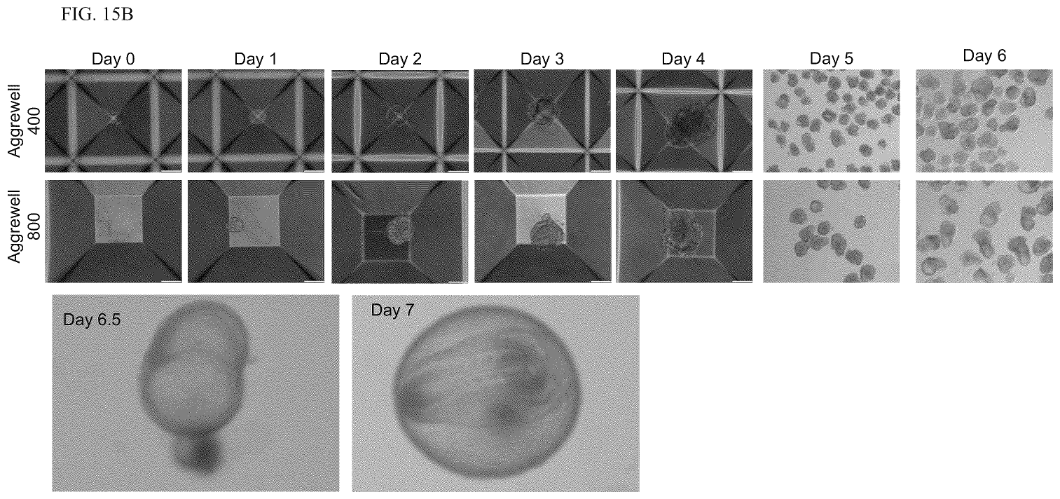

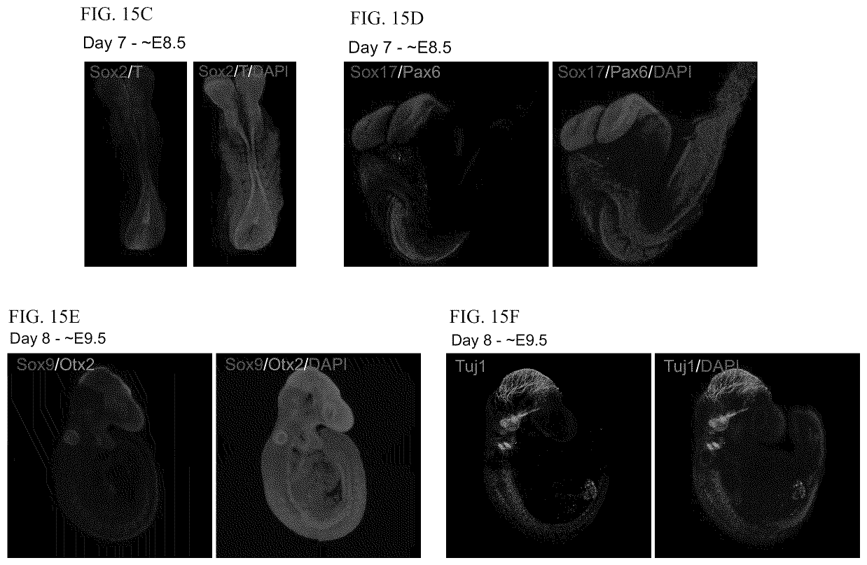

Optional short pre-aggregation Pre-Treatment—This optional step was applied where indicated for some of the donor naïve cells types already expanded in naïve N2B272i/LIF conditions for at least three passages, and were pre-treated for up to 48 hours prior to co-aggregation (defined as time 0) as indicated in the scheme. In this specific regimen (see e.g. FIGS. 15A, 17A, 19A, 20A, 21-22) naïve N2B27 2i/LIF media on KH2-Cdx2 was replaced for 48-12 hours prior to co-aggregation to AM+20% FBS+DOX; and naïve N2B27 2i/LIF media on KH2-Gata4 was replaced for 48-12 hours prior to co-aggregation to AM+20% FBS+DOX.

Aggregation plate preparation—As described in Example 1 hereinabove.

Co-aggregation and culture (FIG. 15A)-Co-aggregation was defined as time point 0 of the protocol and was made from three starting PSC cell types:
1) KH2 WT naïve ESCs (To give rise to embryo proper);
2) DOX pre-treated KH2-GATA4 naïve ES cells (to give rise to the entire extraembryonic endoderm lineage); and
3) DOX pre-treated KH2-CDX2 naïve ES cells (to give rise to entire trophectoderm lineage).
   Cells were trypsinised with 0.05% trypsin-EDTA for 3-5 minutes followed by feeder depletion on gelatinized plates for 20 minutes at 37° C. (when MEFs were used). Cells were resuspended in aggregation media (AM) supplemented with Doxycycline (2 μg/ml final concentration). Following cell count, a total number of 18000 KH2-CDX2, 6000 KH2-GATA4 and 6000 KH2-WT (empty) cells were added per well in AggreWell 400 Microwell 24 well plates. In 800 Microwell plates, a total number of 4500 KH2-CDX2, 1500 KH2-GATA4 and 1500 KH2-WT (empty) cells were added per well in AggreWell 24 well. The cell mixture was added as 1 ml per well (final volume of each well of 24 well is 1.5 ml), 25 cells/well.
Day 0—The plate was centrifuged at 100×g for 3 minutes and this was marked as day 0, and then placed into static incubator at 37° C., 5% Co2 and 20% O2.
On Day 1, 1 ml of media was gently aspirated from each well and replaced with pre-warmed 1 ml of fresh AM+20% FBS+DOX, as indicated in FIG. 15A.
On day 2, 1 ml of media was gently aspirated from each well and replaced with pre-warmed 1 ml of fresh AM+20% FBS, as indicated in FIG. 15A.
On day 3, 1 ml of media was gently aspirated from each well and replaced with pre-warmed 1 ml of EUCM2 (CMRL based) media with 20% FBS, as indicated in FIG. 15A.
On day 4, embryos were transferred with a 3 ml Plastic Pasteur Pipette to a 15 ml falcon tubes, allowed to sit for 5 minutes, and most media was gently removed leaving embryos in ˜1 ml media. The embryos were then moved with the pipette gently in petri dish (35 mm Corning 430588 plates) or (6 well plates Costar 3736) with 4 ml of EUCM3 (CMRL based) media with 30% FBS (as indicated in FIG. 15A) and incubated on a shaker at 60 rpm in a static incubator (37° C., 20% O2 and 5% Co2)].
On day 5, 2-3 ml of media was gently aspirated with a manual pipette and replaced with fresh EUCM4 (CMRL based) with 30% HAS, as indicated in FIG. 15A (bringing back to total volume of 4 ml), and kept on a shaker at 60 rpm in a static incubator (37° C., 20% O2 and 5% Co2).
On days 6 and 7—each day 2-3 ml of media was gently aspirated with a manual pipette and replaced with fresh EUCM media supplemented with D-Glucose 4 mg/ml and sodium pyruvate 1 mM, as indicated in FIG. 15A (bringing back to total volume of 4 ml).
On day 8 up to day 13—High quality embryos were selected and transferred into the previously described ex utero roller culture system (Alejandro-Castrjon et al. Nature 2021) in fresh EUCM media supplemented with D-Glucose 4 mg/ml and sodium-pyruvate 1 mM and incubated at 20% O2, 5% Co2 and 6.5 Psi, as indicated in FIG. 15A.

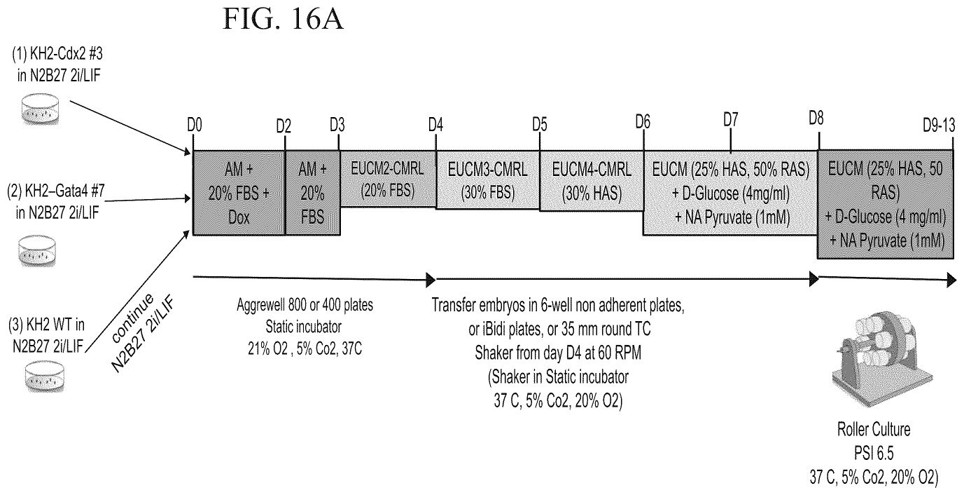

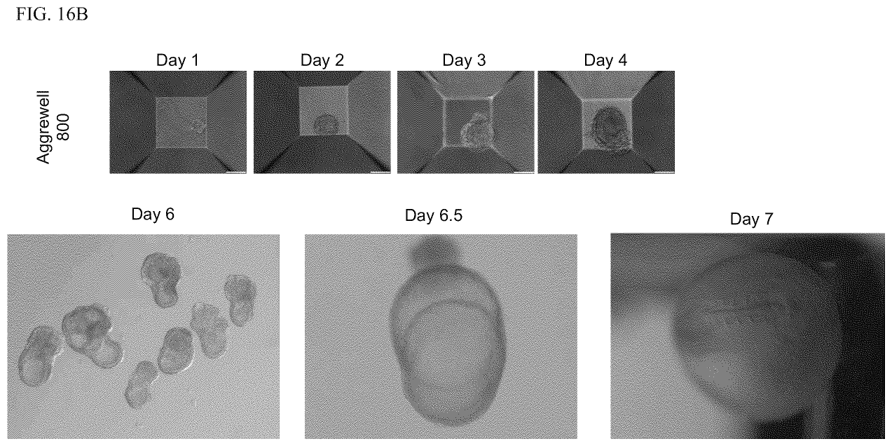

Co-aggregation and culture (FIG. 16A)—Co-aggregation was defined as time point 0 of the protocol and was made from three starting PSC cell types:
1) KH2 WT naïve ESC (To give rise to embryo proper);
2) KH2-GATA4 naïve ESCs (to give rise to the entire extraembryonic primitive endoderm (PRE) lineage); and
3) KH2-CDX2 naïve ESCs (to give rise to entire trophectoderm lineage).
   Cells were trypsinised with 0.05% trypsin-EDTA for 3-5 minutes followed by feeder depletion on gelatinized plates for 20 minutes at 37° C. (when MEFs were used). Following, cells were resuspended in aggregation media (AM) supplemented with Doxycycline (2 μg/ml final concentration). Following cell count, a total number of 18000 KH2-CDX2, 6000 KH2-GATA4 and 6000 KH2-WT (empty) cells were added per well in in 400 Microwell AggreWell 24 well plate. In 800 Microwell plates a total number of 4500 KH2-CDX2, 1500 KH2-GATA4 and 1500 KH2-WT (empty) cells were added per well in AggreWell 24 well. The cell mixture was added as 1 ml per well (final volume of each well of 24 well is 1.5 ml), 25 cells/well.
Day 0—The plate was centrifuged at 100×g for 3 minutes and this was marked as day 0, and then placed into a static incubator at 37° C., 5% Co2 and 20% O2.
On Day 1, 1 ml of media was gently aspirated from each well and replaced with pre-warmed 1 ml of fresh AM 20% FBS+DOX, as indicated in FIG. 16A.
On day 2, 1 ml of media was gently aspirated from each well and replaced with pre-warmed 1 ml of fresh AM+20% FBS+DOX, as indicated in FIG. 16A.
On day 3, 1 ml of media was gently aspirated from each well and replaced with pre-warmed 1 ml of EUCM2 (CMRL based) media with 20% FBS, as indicated in FIG. 16A.
On day 4, embryos were transferred with a 3 ml Plastic Pasteur Pipette to a 15 ml falcon tubes, allowed to sit for 5 minutes, and most media was gently removed leaving embryos in ˜1 ml media. The embryos were then moved with the pipette gently into a petri dish (35 mm Corning 430588 plates) or 6 well plates (Costar 3736) or iBidi plates with 4 ml of EUCM3 (CMRL based) media with 30% FBS (as indicated in FIG. 16A) and incubated on a shaker at 60 rpm in a static incubator (37° C., 20% O2 and 5% Co2).
On day 5, 2-3 ml of media was gently aspirated with a manual pipette and replaced with fresh EUCM4 (CMRL based) medium with 30% HAS as indicated in FIG. 16A (bringing back to total volume of 4 ml) and kept on a shaker at 60 rpm in a static incubator (37° C., 20% O2 and 5% Co2).
At days 6 and 7—each day 2-3 ml of media was gently aspirated with a manual pipette and replaced with fresh EUCM media supplemented with D-Glucose 4 mg/ml and sodium-pyruvate 1 mM as indicated in FIG. 16A (bringing back to total volume of 4 ml).
At day 8 up to day 13—High quality embryos were selected and transferred into the previously described ex utero roller culture system (Alejandro-Castrejon et al. Nature 2021) in fresh EUCM media supplemented with D-Glucose 4 mg/ml and sodium-pyruvate 1 mM and incubated at 20% O₂, 5% Co2 and 6.5 Psi, as indicated in FIG. 16A.

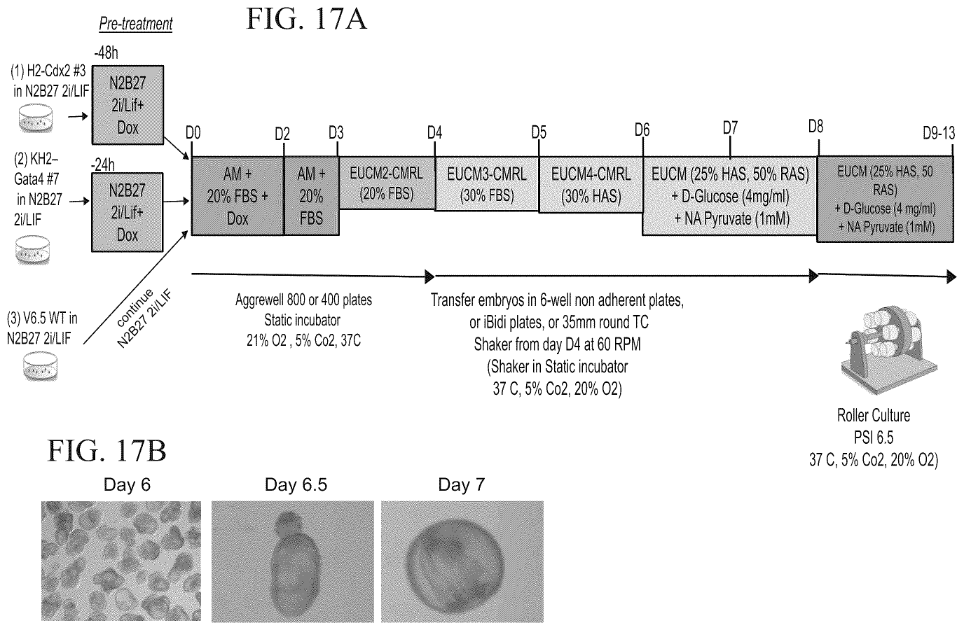

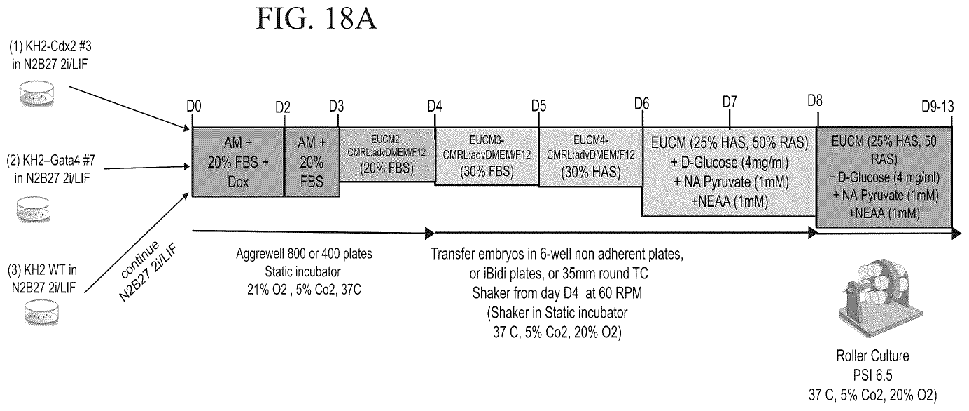

Co-aggregation and culture (FIG. 17A)—Co-aggregation was defined as time point 0 of the protocol and was made from three starting PSC cell types:
1) KH2 WT ESC (To give rise to embryo proper);
2) Dox pre-treated KH2-GATA4 (to give rise to extraembryonic primitive endoderm (PRE) lineage); and
3) Dox pre-treated KH2-CDX2 (to give rise to entire trophectoderm lineage).
   Cells were trypsinised with 0.05% trypsin-EDTA for 3-5 minutes followed by feeder depletion on gelatinized plates for 20 minutes at 37° C. (when MEFs were used). Following, cells were resuspended in aggregation media (AM) supplemented with Doxycycline (2 μg/ml final concentration). Following cell count, a total number of 18000 KH2-CDX2, 6000 KH2-GATA4 and 6000 KH2-WT (empty) cells were added per well in 400 Microwell AggreWell 24 well plates. In 800 Microwell plates a total number of 4500 KH2-CDX2, 1500 KH2-GATA4 and 1500 KH2-WT (empty) cells were added per well in AggreWell 24 well. The cell mixture was added as 1 ml per well (final volume of each well of 24 well is 1.5 ml), 25 cells/well.
Day 0—The plate was centrifuged at 100×g for 3 minutes and this was marked as day 0, and then placed into static incubator at 37% C. 5% Co2 and 20% O2.
On Day 1, 1 ml of media was gently aspirated from each well and replaced with pre-warmed 1 ml of fresh AM+20% FBS+DOX, as indicated in FIG. 17A.
On day 2, 1 ml of media was gently aspirated from each well and replaced with pre-warmed 1 ml of fresh AM+20% FBS, as indicated in FIG. 17A.
On day 3, 1 ml of media was gently aspirated from each well and replaced with pre-warmed 1 ml of EUCM2 (CMRL based) media with 20% FBS, as indicated in FIG. 17A.
On day 4, embryos were transferred with a 3 ml Plastic Pasteur Pipette to a 15 ml falcon tubes, allowed to sit for 5 minutes, and most media was gently removed leaving embryos in ˜1 ml media. The embryos were then moved with the pipette gently into a petri dish (35 mm Corning 430588 plates) or 6 well plates (Costar 3736) or iBidi plates with 4 ml of EUCM3 (CMRL based) media with 30% FBS and incubated on a shaker rotating at 60 rpm in a static incubator (37° C., 20% O2 and 5% Co2), as indicated in FIG. 17A.
On day 5, 2-3 ml of media was gently aspirated with a manual pipette and replaced with fresh EUCM4 (CMRL based) with 30% HAS as indicated in FIG. 17A (bringing back to a total volume of 4 ml) and kept on the shaker at 60 rpm in a static incubator (37° C., 20% O2 and 5% Co2).
At days 6 and 7—each day 2-3 ml of media was gently aspirated with a manual pipette and replaced with fresh EUCM media supplemented with D-Glucose 4 mg/ml and sodium pyruvate 1 mM as indicated in FIG. 17A (bringing back to total volume of 4 ml).
At day 8 up to day 13—high quality embryos were selected and transferred into previously described ex utero roller culture system (Alejandro-Castrjon et al. Nature 2021) in fresh EUCM media supplemented with D-Glucose 4 mg/ml and sodium pyruvate 1 mM and incubated at 20% O₂, 5% Co2 and 6.5 Psi, as indicated in FIG. 17A.

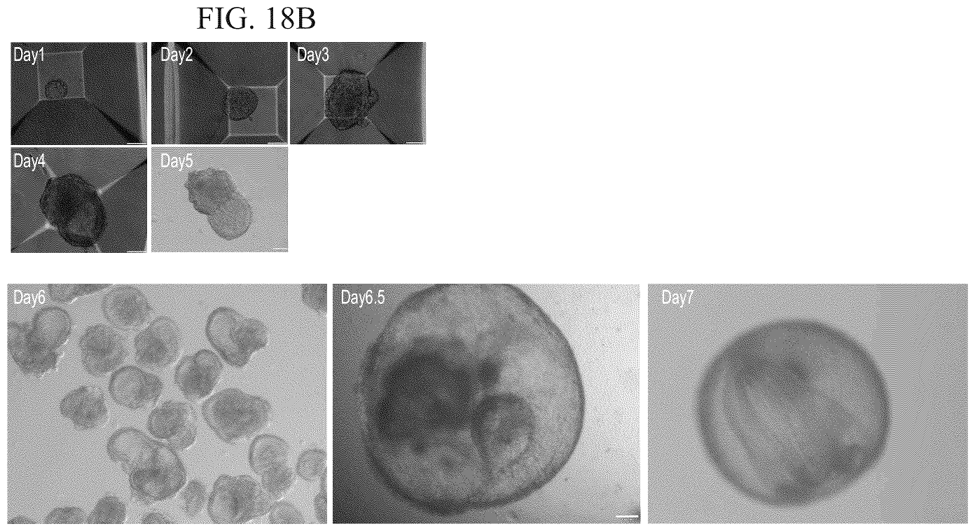

Co-aggregation and culture (FIG. 18A)—Co-aggregation was defined as time point 0 of the protocol and was made from three starting PSC cell types:
1) KH2 WT naïve ESC (To give rise to embryo proper);
2) KH2-GATA4 naïve ESC (to give rise to the entire extraembryonic primitive endoderm (PRE) lineage); and
3) KH2-CDX2 (to give rise to entire trophectoderm lineage).
   Cells were trypsinised with 0.05% trypsin-EDTA for 3-5 min followed by feeder depletion on gelatinized plates for 20 min at 37 C (when MEFs were used). Cells were resuspended in aggregation media (AM) supplemented with Doxycycline (2 μg/ml final concentration). Following cell count, a total number of 18000 KH2-CDX2, 6000 KH2-GATA4 and 6000 KH2-WT (empty) cells were added per well in in 400 Microwell AggreWell 24 well plates. In 800 Microwell plates, a total number of 4500 KH2-CDX2, 1500 KH2-GATA4 and 1500 KH2-WT (empty) cells were added per well in AggreWell 24 well. The cell mixture was added as 1 ml per well (final volume of each well of 24 well is 1.5 ml), 25 cells/well.
Day 0—The plate was centrifuged at 100×g for 3 minutes and this was marked as day 0, and then placed into static incubator at 37° C., 5% Co2 and 20% O2.
On Day 1, 1 ml of media was gently aspirated from each well and replaced with pre-warmed 1 ml of fresh AM+20% FBS+DOX, as indicated in FIG. 18A.
On day 2, 1 ml of media was gently aspirated from each well and replaced with pre-warmed 1 ml of fresh AM+20% FBS, as indicated in FIG. 18A.
On day 3, 1 ml of media was gently aspirated with from each well and replaced with pre-warmed 1 ml of EUCM2 (CMRL/advDMEM-F12 based) media with 20% FBS, as indicated in FIG. 18A.
On day 4, embryos were transferred with a 3 ml Plastic Pasteur Pipette to a 15 ml falcon tubes, allowed to sit for 5 minutes, and most media was gently removed leaving embryos in ˜1 ml media. The embryos were then moved with the pipette gently into petri dish (35 mm Corning 430588 plates) or 6 well plates (Costar 3736) or iBidi plates with 4 ml of EUCM3 (CMRL/advDMEM-F12 based) media with 30% FBS and incubated on a shaker rotating at 60 rpm in a static incubator (37° C., 20% O2 and 5% Co2), as indicated in FIG. 18A.
On day 5, 2-3 ml of media was gently aspirated with a manual pipette and replaced with fresh EUCM4 (CMRL/advDMEM-F12 based) with 30% HAS, as indicated in FIG. 18A (bringing back to a total volume of 4 ml) and kept on the shaker at 60 rpm in a static incubator (37° C., 20% O2 and 5% Co2).
At days 6 and 7—each day 2-3 ml of media was gently aspirated with manual pipette and replaced with fresh EUCM media supplemented with D-Glucose 4 mg/ml and sodium-pyruvate 1 mM and 1×NEAA, as indicated in FIG. 18A (bringing back to a total volume of 4 ml).
At day 8 up to day 13—high quality embryos were selected and transferred into previously described ex utero roller culture system (Alejandro-Castrjon et al. Nature 2021) in fresh EUCM media supplemented with D-Glucose 4 mg/ml and sodium-pyruvate 1 mM and 1×NEAA and incubated at 20% O₂, 5% Co2 and 6.5 Psi, as indicated in FIG. 18A.

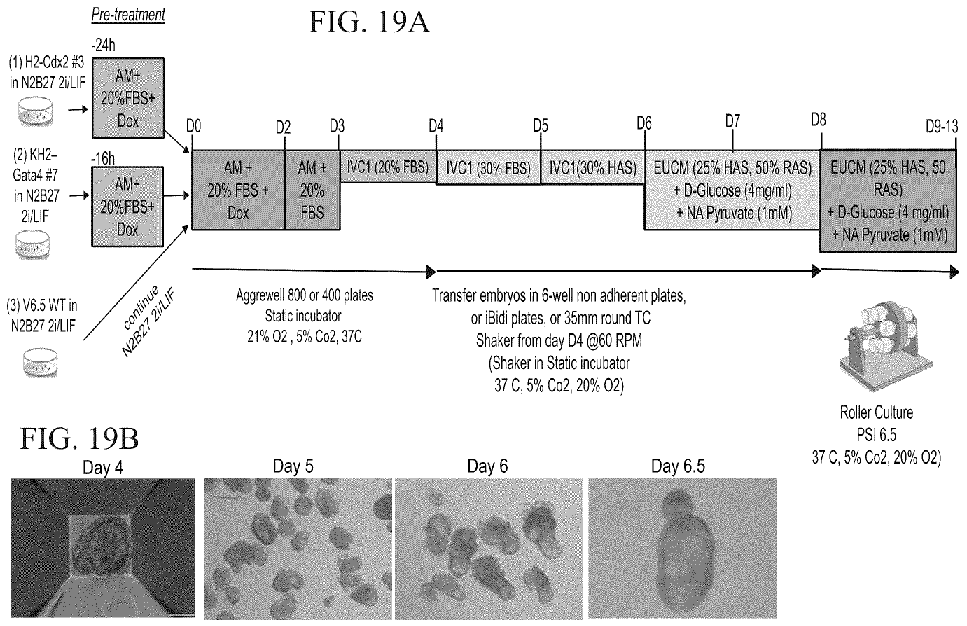

Co-aggregation and culture (FIG. 19A)—Co-aggregation was defined as time point 0 of the protocol and was made from three starting PSC cell types:
KH2 WT naïve ESC (To give rise to embryo proper);
Dox pre-treated KH2-GATA4 naïve ESCs (to give rise to the entire extraembryonic primitive endoderm (PRE) lineage); and
Dox pre-treated KH2-CDX2 (to give rise to entire extraembryonic trophectoderm lineage).

Cells were trypsinised with 0.0 5% trypsin-EDTA for 3-5 minutes followed by feeder depletion on gelatinized plates for 20 minutes at 37° C. (when MEFs were used). Following, cells were resuspended in aggregation media (AM) supplemented with Doxycycline (2 μg/ml final concentration). Following cell count, a total number of 18000 KH2-CDX2, 6000 KH2-GATA4 and 6000 KH2-WT (empty) cells were added per well in 400 Microwell AggreWell 24 well plates. In 800 Microwell plates a total number of 4500 KH2-CDX2, 1500 KH2-GATA4 and 1500 KH2-WT (empty) cells were added per well in AggreWell 24 well. The cell mixture was added as 1 ml per well (final volume of each well of 24 well is 1.5 ml), 25 cells/well.
Day 0—The plate was centrifuged at 100×g for 3 minutes and this was marked as day 0, and then placed into static incubator at 37° C., 5% Co2 and 20% O2.
On Day 1, 1 ml of media was gently aspirated from each well and replaced with pre-warmed 1 ml of fresh AM+20% FBS+DOX, as indicated in FIG. 19A.
On day 2, 1 ml of media was gently aspirated from each well and replaced with pre warmed 1 ml of fresh AM+20% FBS), as indicated in FIG. 19A.
On day 3, 1 ml of media was gently aspirated from each well and replaced with pre-warmed 1 ml of IVC1 media with 20% FBS, as indicated in FIG. 19A.
On day 4, embryos were transferred with a 3 ml Plastic Pasteur Pipette to a 15 ml falcon tubes, allowed to sit for 5 minutes, and most media was gently removed leaving embryos in ˜1 ml media. The embryos were then moved with the pipette gently in petri dish (35 mm Corning 430588 plates) or 6 well plates (Costar 3736) or iBidi plates with 4 ml of IVC1 media with 30% FBS (as indicated in FIG. 19A) and incubated on a shaker rotating at 60 rpm in a static incubator (37° C., 20% O2 and 5% Co2).
On day 5, 2-3 ml of media was gently aspirated with manual pipette and replaced with fresh IVC1 media with 30% HAS, as indicated in FIG. 19A (bringing back to a total volume of 4 ml) and kept on the shaker at 60 rpm in a static incubator (37° C., 20% O2 and 5% Co2).
At days 6 and 7—each day 2-3 ml of media was gently aspirated with a manual pipette and replaced with fresh EUCM media supplemented with D-Glucose 4 mg/ml and sodium-pyruvate 1 mM as indicated in the FIG. 19A (bringing back to a total volume of 4 ml).
At day 8 up to day 13—high quality embryos were selected and transferred into previously described ex utero roller culture system (Alejandro-Castrjon et al. Nature 2021) in fresh EUCM media supplemented with D-Glucose 4 mg/ml and sodium-pyruvate 1 mM and incubated at 20% O₂, 5% Co2 and 6.5 Psi, as indicated in FIG. 19A.

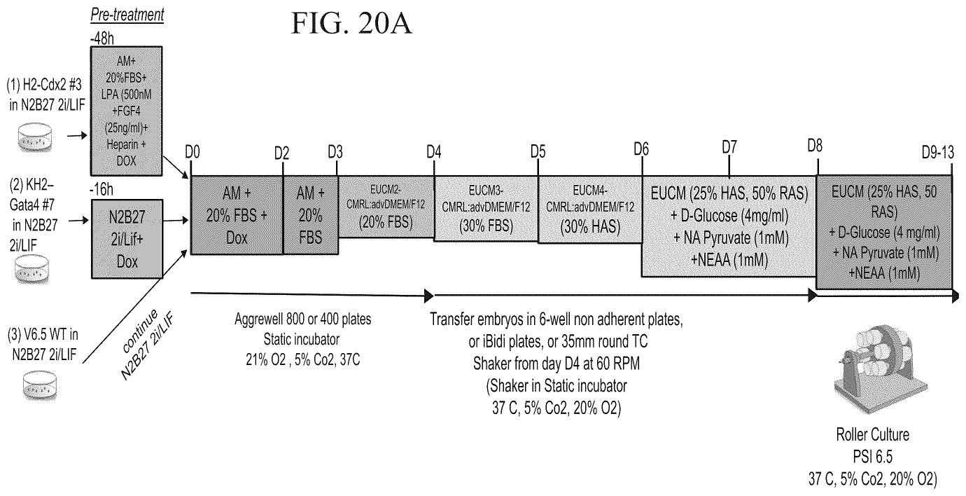

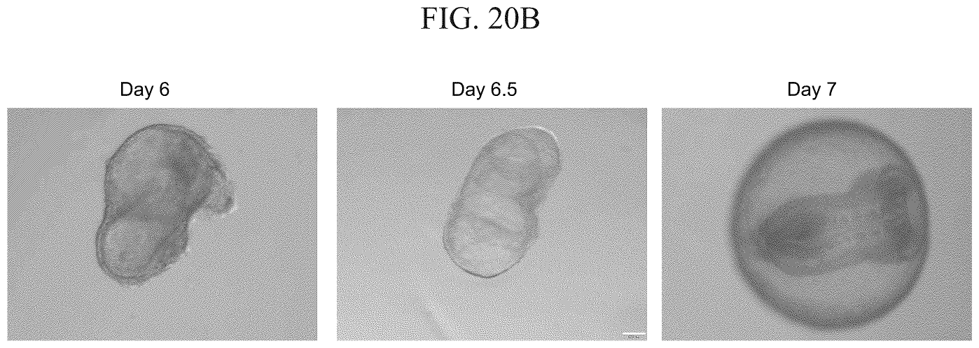

Co-aggregation and culture (FIG. 20A)—Co-aggregation was defined as time point 0 of the protocol and was made from three starting PSC cell types:
KH2 WT ESC (To give rise to embryo proper);
Dox pre-treated KH2-GATA4 (to give rise to extraembryonic primitive endoderm (Pre) lineage); and
KH2-CDX2 naïve ESCs (to give rise to entire extraembryonic trophectoderm lineage) pre-treated with DOX, LPA (500 nM, 1-Oleoyl lysophosphatidic acid sodium salt: functions as a HIPPO pathway inhibitor and YAP translocator to the nucleus), FGF4 (25 ng/ml, Peprotech) and Heparin 0.2 unit/ml (Sigma H3149).

Cells were trypsinised with 0.05% trypsin-EDTA for 3-5 minutes followed by feeder depletion on gelatinized plates for 20 minutes at 37° C. (when MEFs were used). Cells were resuspended in aggregation media (AM) supplemented with Doxycycline (2 μg/ml final concentration). Following cell count, a total number of 18000 KH2-CDX2, 6000 KH2-GATA4 and 6000 KH2-WT (empty) cells were added per well in 400 Microwell AggreWell 24 well plates. In 800 Microwell plates, a total number of 4500 KH2-CDX2, 1500 KH2-GATA4 and 1500 KH2-WT (empty) cells were added per well in AggreWell 24 well. The cell mixture was added as 1 ml per well (final volume of each well of 24 well is 1.5 ml), 25 cells/well.
Day 0—The plate was centrifuged at 100×g for 3 minutes and this was marked as day 0, and then placed into static incubator at 37° C., 5% Co2 and 20% O2.
On Day 1, 1 ml of media was gently aspirated from each well and replaced with pre-warmed 1 ml of fresh AM+20% FBS+DOX, as indicated in FIG. 20A.
On day 2, 1 ml of media was gently aspirated from each well and replaced with pre-warmed 1 ml of fresh AM+20% FBS, as indicated in FIG. 20A.
On day 3, 1 ml of media was gently aspirated from each well and replaced with pre-warmed 1 ml of EUCM2 (CMRL/advDMEM-F12 based) media with 20% FBS, as indicated in FIG. 20A.
On day 4, embryos were transferred with a 3 ml Plastic Pasteur Pipette to a 15 ml falcon tubes, allowed to sit for 5 minutes, and most media was gently removed leaving embryos in ˜1 ml media. The embryos were then moved with the pipette gently into petri dish (35 mm Corning 430588 plates) or 6 well plates (Costar 3736) or iBidi plates with 4 ml of EUCM3 (CMRL/advDMEM-F12 based) media with 30% FBS (as indicated in FIG. 20A) and incubated on a shaker rotating at 60 rpm in a static incubator (37° C., 20% O2 and 5% Co2).
On day 5, 2-3 ml of media was gently aspirated with a manual pipette and replaced with fresh EUCM4 (CMRL/advDMEM-F12 based) with 30% HAS, as indicated in FIG. 20A (bringing back to a total volume of 4 ml) and kept on the shaker at 60 rpm in a static incubator (37° C., 20% O2 and 5% Co2).
At days 6 and 7—each day 2-3 ml of media was gently aspirated with a manual pipette and replaced with fresh EUCM media supplemented with D-Glucose 4 mg/ml and sodium-pyruvate 1 mM and 1×NEAA as indicated in FIG. 20A (bringing back to a total volume of 4 ml).
At day 8 up to day 13—high quality embryos were selected and transferred into previously described ex utero roller culture system (Alejandro-Castrjon et al. Nature 2021) in fresh EUCM media supplemented with D-Glucose 4 mg/ml, sodium-pyruvate 1 mM and 1×NEAA and incubated at 20% O₂, 5% Co2 and 6.5 Psi, as indicated in FIG. 20A.

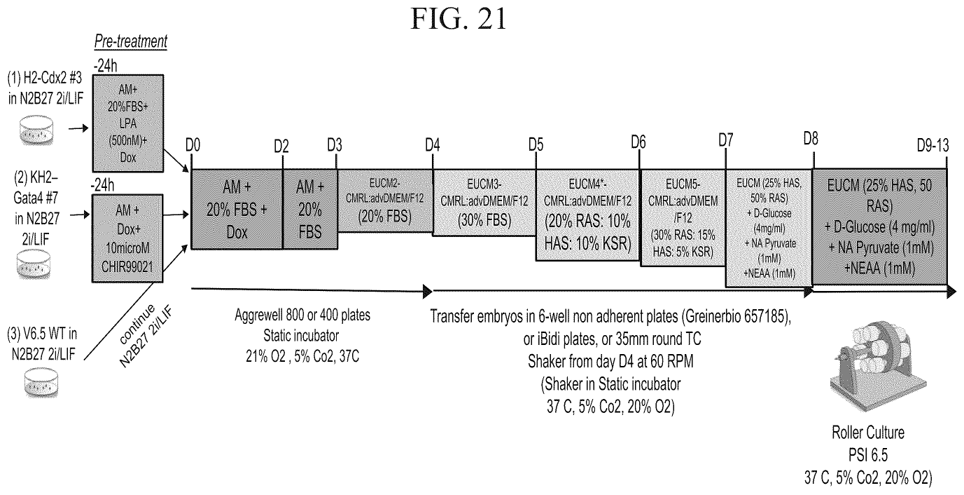

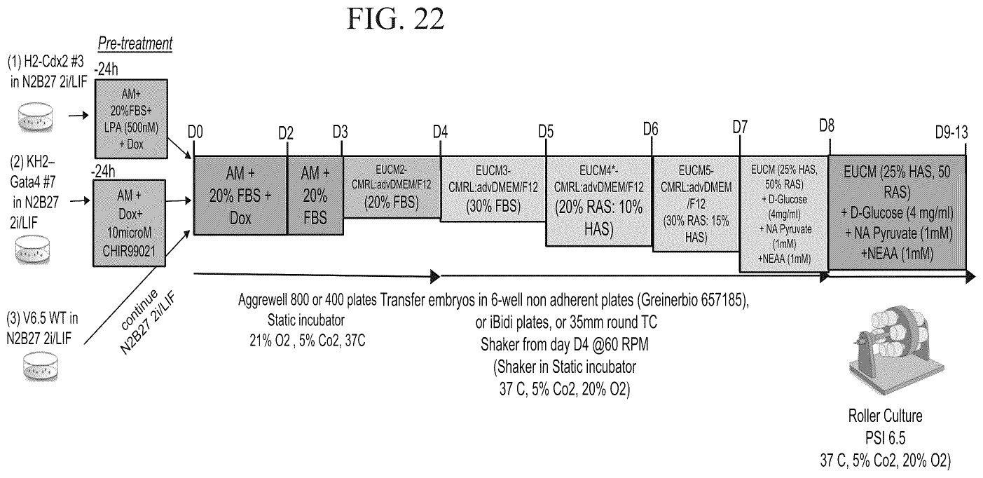

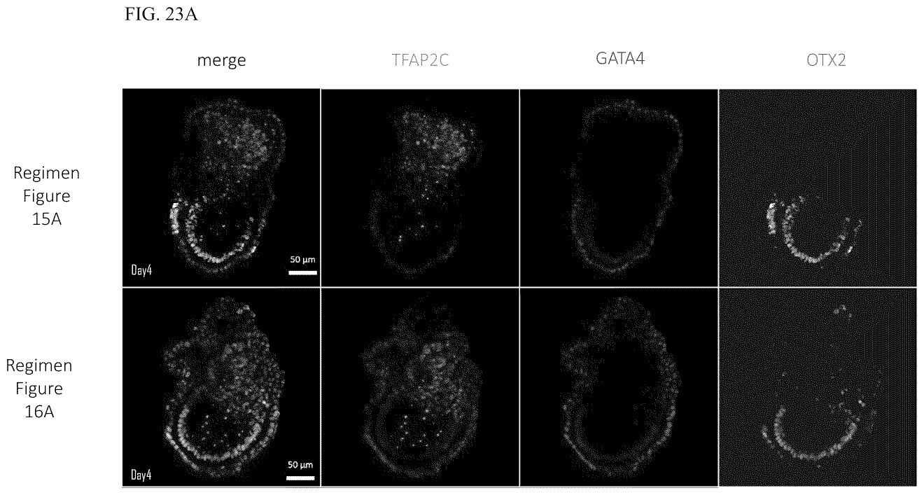

Co-aggregation and culture (FIGS. 21 and 22)—Co-aggregation was defined as time point 0 of the protocol and was made from three starting PSC cell types:
KH2 WT ESC (To give rise to embryo proper);
KH2-GATA4 (to give rise to extraembryonic endoderm lineage) pre-treated with Dox and CHIR99021; and
KH2-CDX2 (to give rise to entire trophectoderm lineage) pre-treated with DOX and LPA.
   The protocols were as described hereinabove for e.g. FIG. 20A with the modifications as indicated in FIGS. 21 and 22, respectively.

Immunostaining and Microscopy—As described in Example 1 herein above.

Real-Time PCR analysis—As described in Example 1 herein above.

### Results

The present inventors have devised a novel and inventive method to generate advanced synthetic embryos made solely from naïve ESCs or iPSCs, by transiently inducing overexpression of a factor that induces differentiation to extra embryonic primitive endodermal (Pre) cells such as Gata4 and/or Gata6 in a subpopulation of the naïve PSCs, transiently inducing overexpression of a factor that induces differentiation to trophectoderm cells such as Cdx2 and/or Gata3 and/or Gata2 and/or nuclear YAP in another subpopulation of the naïve PSCs and mixing and aggregating the two subpopulations with the same naïve PSCs that were not subjected to transient overexpression. Following, the generated aggregates may be grown ex utero to complete gastrulation and organogenesis or injected to the uterus to continue growth in-utero.

To this end, instead of using stable TSC lines made from the transgenic ESC lines described in Example 1 hereinabove and stabilized after several passages (at least 3 passages, 15 days), which might alter their characteristics, naïve mESCs that transiently overexpress Cdx2 upon addition of DOX in the early stages of embryo aggregation protocol were generated, in order to induce their rapid transition into early trophectoderm (TE). In addition, instead of using the blastocysts derived XEN cells described in Example 1 hereinabove, naïve mESCs that transiently overexpress Gata4 upon addition of DOX in the early stages of embryo aggregation protocol were generated, in order to induce their rapid transition into the extraembryonic primitive endoderm lineage.

Indeed, co-aggregation of these three stem cell populations, all originating from naïve ESCs/iPSCs as a starting material, followed by ex-utero culturing enabled generation of organized embryos, as exemplified in FIGS. 15A-23D). Interestingly, the equivalence between the culturing days and embryonic stages was as follows: Day 0-E3; Day 1-E3.5; Day 2-E4; Day 3-E4.5; Day 4-E5; Day 5-E6; Day 6-E7; Day 7-E8; Day 8-E9; Day 9-E10; Day 11-E12; Day 12-E13.

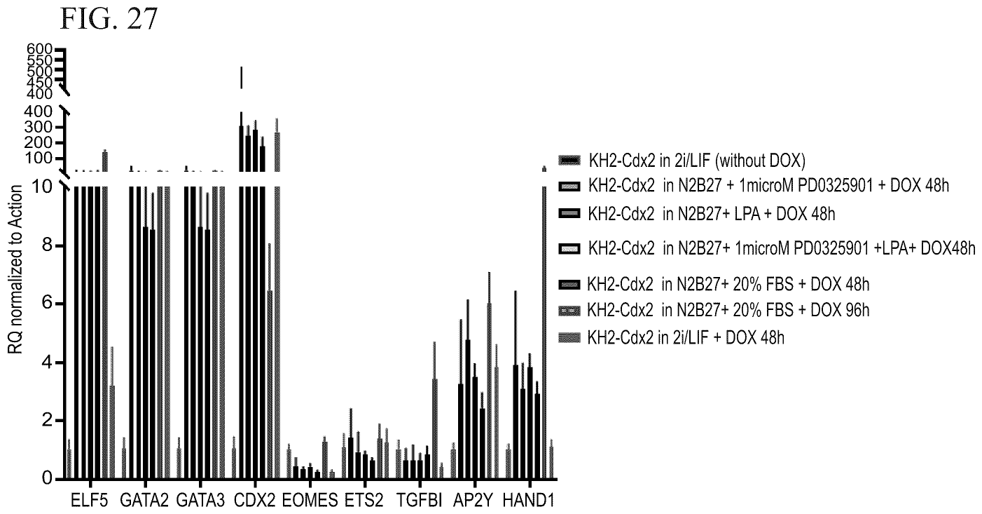

Real-time PCR analysis indicated that KH2-Gata4 cells significantly upregulate the endogenous extraembryonic ectoderm markers Gata4, Gata6 and PDGFRA following DOX induction in 2i/LIF (FIG. 24). Interestingly, while Dox treatment is stopped at day 2 of the culturing protocol (see e.g. FIG. 17A), embryos obtained at Day 4 of the protocol retain very high expression of endogenous extraembryonic ectoderm markers Gata4, Gata6 and PDGFRA (FIG. 24). In the same manner, real-time PCT analysis indicated that KH2-Cdx2 cells significantly upregulate the endogenous trophectoderm markers Elf5, Gata2, Gata3, Cdx2, Ap2gmma (also known as Tfap2c) and Hand1 (FIGS. 25-27). Interestingly, while Dox treatment is stopped at day 2 of the culturing protocol (see e.g. FIG. 17A), embryos obtained at Day 4 of the protocol retain very high expression of endogenous trophectoderm markers Elf5, Gata2, Gata3, Cdx2, Ap2gmma, EOMES, ETS2 and Hand1 (FIGS. 25-26).

### Example 3

### Aggregation of Three Mouse Naïve PSCs Subpopulations Generates an Organized Embryo

### Materials and Methods

Animals-Natural in utero developed embryos were obtained from female mice 5-8 weeks old ICR or BDF1 mated with BDF1 male studs (Harlan). Insemination was verified the next morning by the presence of a copulatory plug, and this day was defined as embryonic day 0.5 (E0.5). All animal experiments were performed according to the Animal Protection Guidelines of Weizmann Institute of Science and approved by relevant Weizmann Institute IACUC (#01390120-1, 01330120-2, 33520117-2). Mice were housed in a standard 12-hours light/12-hours dark cycle conditions in a specialized and certified animal facility.

Stem cell lines—The following mouse ES lines were used: WT V6.5 ESCs (C57B6/129sJae), BVSC ESC (Mixed BDF1 X B6 background) and KH2-WT ESC line carrying both M2RtTa allele in the Rosa26 locus and a modified Col1 locus with an FRT site to efficiently insert Tet-ON regulated alleles as previously described (Hochedlinger et al., 2005). The previously described BVSC ESC lines (Hayashi et al., 2011) carries Blimp1-mVenus and Stella-Cfp (BVSC) reporter alleles which can be useful for tracking PGC formation.

Stem cells in vitro culture conditions—Golden stocks of mouse ESCs were cultured on feeder layer of irradiated mouse embryonic fibroblast (MEFs) and maintained (and gene targeted when relevant) in conventional mouse ES medium (Serum/LIF) composed of 1×DMEM (GIBCO 41965) supplemented with 20% FBS (heat inactivated and filtered), 1 mM GlutaMAX (GIBCO, 35050061), 1% penicillin streptomycin (Biological Industries—Sartorius 03-031-1B), 1% Sodium Pyruvate (Biological Industries—Sartorius 03-042-1B), 1% nonessential amino acids (Biological Industries—Sartorius 01-340-1B), 0.1 mM β-mercaptoethanol (Thermo 31350010), 10 ng/ml recombinant human LIF (in-house prepared).

To convert the cells into ground state naïve ESCs in 2i/LIF conditions, ESCs were maintained and expanded in serum-free chemically defined N2B27-based media: 240 ml Neurobasal (Thermo 21103049) and 240 ml of DMEM-F12 with HEPES (SIGMA D6421), 5 ml N2 supplement (Invitrogen: 17502048), 5 ml B27 supplement (Invitrogen; 17504044 or in house prepared), 1 mM GlutaMAX (GIBCO, 35050061), 1% nonessential amino acids (Biological Industries—Sartorius 01-340-1B), 0.1 mM β-mercaptoethanol (Thermo 31350010), 1% penicillin-streptomycin (Biological Industries—Sartorius 03-031-1B). Naïve 2i/LIF conditions for murine PSCs included 20 ng/ml recombinant human LIF (in-house made) and small-molecule inhibitors CHIR99021 (CHIR, 3 μM—Axon Medchem 1386) and PD0325901 (PD, 1 μM—Axon Medchem 1408) (referred to as 2i). Murine naïve ESCs were expanded on feeder layer (MEFs) or on 0.2% gelatin-coated plates. At least three passages in 2i/LIF conditions were applied before initiation of experimentation. Mouse naïve ESC lines were used up to 15 passages in 2i/LIF conditions. For maintenance ESCs were passaged with 0.25% trypsinization every 3-5 days.

KH2-Gata4 Dox inducible naïve pluripotent stem cells (KH2-GATA4)—As described in Example 1 hereinabove.

KH2-Cdx2 Dox inducible naïve pluripotent stem cells (KH2-Cdx2)—As described in Example 2 hereinabove.

Generation of ELF5 EYFP-NLS reporter on KH2-Cdx2 ESCs—iCdx2 (KH2-CDX2) validated clone 3 was used for CRISPR targeting EYFP in 3′ end of mouse ELF5 gene. Cells we co-transfected with previously generated nuclear EYFP plasmid (Addgene #128833) and guide RNA plasmid (#128836). Following neomycin and ganciclovir antibiotic selection for 10 days, total clonal population was transfected with Cre and subcloned. Single cell clones were validated for correct insertion: at 3′ end using forward ATGGTCCTGCTGGAGTTCGTGAC (SEQ ID NO: 37) and reverse TGGTCCATCTGCTTGTAGGCAAGA (SEQ ID NO: 38) primer pair, and at 5′ end using forward TTCACCTTTGAAGCTAATCGTTTGAGG (SEQ ID NO: 39) and reverse AACTTGTGGCCGTTTACGTCGC (SEQ ID NO: 40) primer pair. Correctly targeted clones were further validated for off-target insertions by Southern blot analysis.

Generation of fluorescent labeled ESCs—KH2 WT ESCs, KH2-Gata4 clone 7 (iGata4) and KH2-Cdx2 clone 3 (iCdx2) were transduced with FUGW lentivirus particles constitutively expressing either fluorescent BFP, GFP or mCherry proteins, respectively. For the generation of lentivirus, HEK293T cells were plated in 10 ml DMEM, containing 10% FBS and Pen/Strep in 10 cm dishes, in aliquots of 5.5 million cells per well. On the next day, cells were transfected with the third generation Addgene lentivirus vectors [0.8 μg of pRSV-Rev (Addgene 12253), 0.8 μg of pMDLg/pRRE (Addgene 12251), 1.6 μg of pMD2.G (Addgene12259)], using jetPEI™ transfection reagent, along with 16 μg of the target plasmid of each transduced fluorescent proteins BFP, GFP and mCherry. The supernatant containing the virus was collected 48 hours and 72 hours following transfection, filtered using 0.45 μm filter and freezed in −80° C. in 1 ml aliquots. ESCs were plated in FBS/Lif condition on gelatin coated 6-well plates at low density and transduced with lentivirus in the presence of protamine sulfate (8 μg/ml). 48 hours later the infected ESCs were expanded for 1-3 passages and sorted for positive population and further expanded for experimentation.

Isolation of human umbilical cord blood serum-Collection of human cord blood serum was done as described previously (Aguilera-Castrejon et al., 2021b) following the guidelines approved by Rambam Medical Center Helsinki committee (#RMB-0452-15). Healthy women over the age of 18 and under 40 who gave their consent and were scheduled for caesarian section delivery were eligible for cord blood collection. Blood was manually drawn by the obstetrician surgeon, using a large bore 14-gauge needle and a 50 ml syringe, directly from the umbilical cord. In brief, blood was collected and quickly distributed to ⅝ mL pro-coagulant sterile test tubes (Greiner Bio-One, Z Serum Sep Clot Activator, #456005) and cooled to 4° C. for 15 minutes, followed by centrifugation at 2500G for 10 minutes at 4° C. Tubes showing signs of hemolysis were discarded. Serum was filtered through a 0.22 μM filter (Nalgene, Ref #565-0020), heat-inactivated at 55° C. in a water bath for 45 minutes and immediately aliquoted and stored at −80° C. for up to six months.

Co-aggregation and culture-AggreWell 24-well plate 400 (STEMCELL Technologies 34415), or AggreWell 24-well plate 800 (STEMCELL Technologies 34815) were used with equivalent outcome. AggreWell plate preparation was done according to manufacturer instructions. Briefly, 500 μl of anti-adherence rinsing solution (STEMCELL Technologies 07010) was added to each well. Plate was centrifuged at 2,000×g for 5 minutes and incubated 30 minutes at room temperature. After incubation, rinsing solution was removed and the plate was washed twice with PBS. 500 μl of aggregation media AM supplemented with DOX (2 μg/ml final concentration—Sigma D9891) and ROCKi Y27632 (5 nM final concentration—Axon Medchem 1683) was added to each well. The aggregation media contained: 1×DMEM (GIBCO-41965) supplemented with 20% FBS (GIBCO), 1 mM GlutaMAX (GIBCO, 35050061), 1% penicillin streptomycin (Biological Industries—Sartorius 03-031-1B), 1% Sodium Pyruvate (Biological Industries—Sartorius 03-042-1B), 1% nonessential amino acids (Biological Industries—Sartorius 01-340-1B) and 0.1 mM β-mercaptoethanol (Thermo 31350010).

To generate the synthetic embryos, referred to herein as “sEmbryo iCdx2”, three starting PSC cell types were co-aggregated:
1) naïve WT ESCs (either KH2-WT, BVSC or V6.5 ESC) in 2i/LIF;
2) naïve iGata4 ESCs in 2i/LIF pre-treated with DOX (2 μg/ml—Sigma D9891) in 2iLIF media for 24 hours prior to the co-aggregation; and
3) naïve iCdx2 ESCs in 2i/LIF pretreated with DOX (2 μg/ml—Sigma D9891) for different time points (−1 day to −14 days prior to the co-aggregation) in TSC media (25 ng/ml FGF4 (Peprotech), 1 μ/ml Heparin (Sigma)) supplemented with lysophosphatidic acid (LPA, a Hippo pathway inhibitor) 0.5-1 μM.
   Co-aggregation was defined as time point 0 of the protocol

At the day of aggregation (day 0), the three donor cell populations were trypsinized with 0.05% trypsin-EDTA solution (Biological Industries—Sartorius 03-053-1B) for 4 minutes at 37° C. Trypsin enzymatic reaction was stopped by adding aggregation media. Cells were centrifuged at 1200 rpm for 3 minutes and resuspended in aggregation medium with DOX (2 μg/ml—Sigma D9891) and ROCKi Y27632 (5 nM final concentration—Axon Medchem 1683). Following, cells were plated on gelatinized tissue culture plates for mouse embryonic fibroblast depletion for 20 minutes at 37° C. Supernatant was collected, centrifuged and cells were resuspended. The three cell fractions were counted and resuspended in aggregation media with DOX (2 μg/ml—Sigma D9891) and ROCKi Y27632 (5 nM—Axon Medchem 1683). A ratio of (1 WT-ESCs:1 iGata4 ESC:3.33 iCdx2 ESC) was maintained in aggregation experiments, as follows:

| | Number of | Number of added | # of Cells |
|---|---|---|---|
| | microwells per well | cells per each well | per single |
| | in 24 well plate | of a 24 well plate | microwell |
| AggreWell | 300 | 1-iCDX2: 5000 cells | ~27 cells |
| 800 | | 2-iGATA4: 1500 cells | |
| | | 3-WT ESC 1500 cells | |
| Aggre Well | 1200 | 1-iCDX2: 20000 cells | ~27 cells |
| 400 | | 2-iGATA4: 6000 cells | |
| | | 3-WT ESC 6000 cells | |

1 ml of cell-mix suspension was gently added drop wise to each well of the AggreWell plate followed by centrifugation at 700 rpm for 3 minutes and incubation at 37° C. (total end volume is 1.5 ml).

On the next day (day 1), 1 ml of media (out of total 1.5 ml) was gently removed from each well and replaced with 1 ml of preheated aggregation media with DOX (2 μg/ml—Sigma D9891).

On day 2, 1 ml of media was removed from each well and replaced with 1 ml of preheated aggregation media.

On day 3, 1 ml of media was removed from each well and replaced with 1 ml of preheated EUCM2 media. In this example, EUCM2 media refers to: Advanced DMEM/F12 (GIBCO 21331-020), 5 ml CMRL media (1% final concentration) as a supplement (5 mlGIBCO 11530037), extra added 1 mg/ml D (+)-Glucose Monohydrate (J.T. Baker—0113) (add 500 mg per 500 ml media), 100 nM T3 (3,3′,5-Triiodo-L-thyronine sodium salt) (Sigma-Aldrich, T6397), 1 mM Sodium pyruvate (Sigma-Aldrich, S8636), 1 mM GlutaMAX (GIBCO, 35050061), 1% penicillin streptomycin (Biological Industries—Sartorius 03-031-1B), 1× of ITS-X supplement (Thermo Fisher Scientific 51500-056), 8 nM B-estradiol (Sigma-Aldrich, E8875), 200 ng/ml progesterone (Sigma-Aldrich, P0130), 25 μM N-acetyl-L-cysteine (Sigma-Aldrich, A7250)+20% FBS.

On day 4, the synthetic embryos were gently transferred to 6-well cell suspension culture plates (Greiner, 657185) with 5 ml of preheated EUCM3 media per well and placed on shaker rotation 60 rpm/min (Thermo 88881102+88881123). In this example, EUCM3 media refers to: Advanced DMEM/F12 (GIBCO 21331-020), 5 ml CMRL media (1% final concentration) as a supplement (5mlGIBCO 11530037), extra added 1 mg/ml D (+)-Glucose Monohydrate (J.T. Baker—0113) (add 500 mg per 500 ml media), 100 nM T3 (3,3′,5-Triiodo-L-thyronine sodium salt) (Sigma-Aldrich, T6397), 1 mM Sodium pyruvate (Sigma-Aldrich, S8636), 1 mM GlutaMAX (GIBCO, 35050061), 1% penicillin streptomycin (Biological Industries—Sartorius 03-031-1B), 1× of ITS-X supplement (Thermo Fisher Scientific 51500-056), 8 nM B-estradiol (Sigma-Aldrich, E8875), 200 ng/ml progesterone (Sigma-Aldrich, P0130), 25 μM N-acetyl-L-cysteine (Sigma-Aldrich, A7250)+30% FBS.

On day 5, egg cylinder shape sEmbryos were picked and transferred to glass culture bottles (50-30 embryos per bottle) containing 2 mL of EUCM media (the contents of which are described in Example 1 hereinabove). Following, the bottles were placed on the rolling culture system previously described electronically controlled roller culture platform (Aguilera-Castrejon et al., 2021a), rotating at 30 revolutions per minute at 37° C., and continuously gassed with an atmosphere of 21% O₂, 5% CO₂ at 7-8 pounds per square inch (psi). A black non-transparent cloth was used to cover the incubator from light during unit operation.

From day 6 to day 8, 1 ml of EUCM was replaced with preheated 1 ml of EUCM and kept on rolling culture system. Culture media was pre-heated for at least an hour by placing it inside a glass bottle on the rotating culture.

Whole-mount immunostaining—synthetic embryos grown ex utero and equivalent in utero controls were fixed with 4% PFA EM grade (Electron microscopy sciences, 15710) in PBS at 4° C. over-night. Natural embryos were dissected removing the Reichert's membrane for E6.5-E7.5 embryos, or the yolk sac and amnion for E8.5 embryos and washed once with 1×PBS before fixation. Synthetic embryos were then washed in PBS for 5 minutes 3 times, permeabilized in PBS with 0.5% Triton X-100/0.1 M glycine for 30 minutes, blocked with 10% normal donkey serum/0.1% Triton X-100 in PBS for 1 hour at room temperature (RT), and incubated over-night at 4° C. with primary antibodies, diluted in blocking solution. Following, embryos were rinsed 3 times for 5 minutes each in PBS/0.2% TritonX-100, incubated for 2 hours at room temperature with secondary antibodies diluted 1:200 in blocking solution (all secondary antibodies were from Jackson ImmunoResearch), counterstained with DAPI (1 g/ml in PBS) for 10 minutes, and washed with PBS for 5 minutes 3 times. Yolk sacs isolated from natural and synthetic embryos were fixed and stained following this protocol. The primary antibodies used were the following: Rabbit polyclonal anti-Gata4 (Abcam, Ab84593) 1:200; Rabbit polyclonal anti-Foxa2 (Abcam, Ab40874) 1:100; Goat polyclonal anti-Sox2 (R&D, AF2018) 1:200; monoclonal anti-Myosin Heavy Chain II (MF-20) (R&D, MAB4470) 1:200; Goat polyclonal anti-Sox17 (R&D, AF1924) 1:100; Rabbit monoclonal anti-Brachyury (Cell Signaling, 81694) 1:100; Rabbit polyclonal anti-Cdx2 (ab76541) 1:100; Mouse monoclonal anti-Oct4 (Santa Cruz, SC-5279) 1:100; Goat polyclonal anti-Otx2 (R&D, AF1979) 1:200; Goat polyclonal anti-TFAP2C/AP2y (R&D, AF5059) 1:100; Goat polyclonal anti-Gata6 (R&D, AF1700) 1:100; Rabbit polyclonal anti-Eomes/Tbr2 (Abcam, Ab23345) 1:50; Rat Monoclonal anti-Cerberus 1 (R&D, MAB1986) 1:100; Rabbit polyclonal anti-Sox9 (Millipore, Ab5535) 1:100; Mouse monoclonal anti-Gata3 (Invitrogen, MA1-028) 1:100, Rabbit polyclonal anti-Nanog (Bethyl, A300-397A) 1:100; Goat polyclonal anti-Elf5 (Santa Cruz, sc9645) 1:100; Goat polyclonal anti-DKK1 (R&D, AF1096) 1:100 or Goat polyclonal anti-Brachyury (R&D, AF2085) 1:100.

Immunohistochemistry—For OCT-staining, on day 8 of the protocol, the synthetic embryos were fixed overnight in 4% PFA at 4° C., washed three times in PBS for 10 minutes each and submerged first in 15% Sucrose/PBS and then 30% sucrose overnight at 4° C. The day after, samples were subjected to increasing gradient of OCT concentration in Sucrose/PBS followed by embedding in OCT on dry ice and stored at −80° C. until further processing. Cryoblocks were cut with LEICA CM1950 and washed once with 1×PBS and incubated with 0.3% H₂O₂ for 20 minutes. Following permeabilization with 0.1% Triton X-100 in PBS for 10 minutes, slides were washed three times with 1×PBS for 2 minutes each and blocked in 10% normal donkey serum in PBS in humidified chamber for 20 minutes at room temperature. Slides were then incubated with a primary antibody diluted in antibody solution (1% BSA in 0.1% Triton X-100) at 4° C. overnight. Sections were then washed three times (5 minutes each) in 0.1% Triton X-100 in PBS, incubated with the appropriate secondary antibodies diluted in antibody solution at room temperature for 1 hour in the dark, counterstained with DAPI for 20 minutes and mounted with Shandon Immuno-Mount (Thermo Scientific, 9990412). The primary antibodies used were the following: Rabbit polyclonal anti-Gata4 (Abcam, Ab84593) 1:100; Rabbit polyclonal anti-Foxa2 (Abcam, Ab40874) 1:100; Goat polyclonal anti-Sox2 (R&D, AF2018) 1:100; Goat polyclonal anti-GATA6 (R&D, AF1700) 1:100; monoclonal anti-Myosin Heavy Chain II (MF-20) (R&D, MAB4470) 1:100 or Rabbit polyclonal NKX2.5 (Abcam, Ab97355) 1:100.

Confocal microscopy—Whole-mount immunofluorescence and immunohistochemistry images were acquired with a Zeiss LSM 700 inverted confocal microscope (Carl Zeiss) equipped with 405 nm, 488 nm, 555 nm and 635 nm solid state lasers, using a Plan-Apochromat 20× air objective (numerical aperture 0.8) for E5.5/E6.5 embryos, and an EC Plan Neofluar 10× air objective (numerical aperture 0.3) for synthetic embryos. Images were acquired at 1024×1024 resolution. All images were acquired within the following range of parameters: Laser power: 405 nm: 10-20%; 488 nm: 5-20%: 555 nm 10-40%; 635 nm: 30-80%. Gain ranged from 350 to 600. Pixel size was 1.25 μm with a z-step of 15 μm when using the 10× objective, or 0.5 μm with z-step of 5 μm when using the 20× objective. For confocal imaging synthetic embryos were mounted in 35 mm glass bottom dishes (In Vitro Scientific, D35201.5N). Images and maximum intensity projections were processed using Zen 2 blue edition software 2011 (Carl Zeiss) and Adobe Photoshop CS4.

Assessment of length-Morphometric measurements were performed using bright field images of synthetic embryos at the indicated time points. Length of the proximal-distal axis was measured for synthetic embryos at both day 4 and day 5. Antero-posterior axis was measured for synthetic embryos from day 8. Measurements were performed using the CellSens Entry software (Olympus). Length of in utero control natural embryos was used for comparison at matched embryonic stages as indicated.

Yolk sac erythroid progenitor staining-Synthetic embryos at day 8 of the protocol and natural E8.5 derived yolk sacs were dissected. Single cells flow cytometry staining was effected using MacsQuant VYB instrument (Miltenyi, Bergisch Gladbach, Germany). Data was analyzed with FlowJo. Staining was performed for 30 minutes at 4° C. in flow cytometry buffer (PBS, 10% fetal bovine serum and 0.02% azide). For erythroid progenitor staining, a biotinylated lineage cocktail of CD4, CD8, B22, CD11b, GR-1 and Ter119 (Miltenyi), together with anti-cKit APC (2B8), CD41 VG (MRW), CD45 PE (30-F11) (all from Biolegend) and CD34 PB (RAM34, eBioscience) was used, as previously described (Iturri et al., 2021). Streptavidin-PE-Cy7 (Biolegend) was used as a secondary conjugated antibody.

Erythroid colony forming assay-Harvested cells from day 8 or E8.5 yolk sacs were prepared as single cell suspension in Iscove's modified Dulbecco's medium supplemented with 2% FBS (GIBCO) and 1% penicillin streptomycin (Invitrogen). Isolated cells were plated in triplicate at a density of (1×10⁶) cells per 1.1 ml of MethoCult medium (Stem Cell Technologies™, SF M3436) in 35-mm dish and maintained at 37° C. with 5% CO₂ for 12 days before being scored for primitive erythroid colonies. Colonies were visualized and validated with a bright field microscope A1 microscope (Zeiss).

RNA extraction & RT-PCR analysis—Total RNA was isolated using RNeasy mini kit (Qiagen) following manufacturer instructions. 1 μg of total RNA was reverse transcribed using a High-Capacity Reverse Transcription Kit (Applied Biosystems). RT-PCR was performed in triplicate using SYBR Green PCR Master Mix (QIAGEN) and run on Viia7 platform (Applied Biosystems). Values were normalization to ACTIN and/or GAPDH across all experiments, data is presented as fold difference compared reference sample set as 1. The following primers were used:

| | mGata3-Forward: |
|---|---|
| (SEQ ID NO: 41) | |
| | CTCGGCCATTCGTACATGGAA; |
| | mGata3-Reverse: |
| (SEQ ID NO: 42) | |
| | GGATACCTCTGCACCGTAGC; |
| | mGata2 Forward: |
| | (SEQ ID NO: 43) |
| | CACCCCGCCGTATTGAATG; |
| | mGata2 Reverse: |
| (SEQ ID NO: 44) | |
| | CCTGCGAGTCGAGATGGTTG; |
| | mElf5 Forward: |
| (SEQ ID NO: 45) | |
| | ATGTTGGACTCCGTAACCCAT; |
| | mElf5-Reverse: |
| (SEQ ID NO: 46) | |
| | GCAGGGTAGTAGTCTTCATTGCT; |
| | mHand1F: |
| (SEQ ID NO: 47) | |
| | GGCAGCTACGCACATCATCA; |
| | mHand1 Reverse: |
| (SEQ ID NO: 48) | |
| | CCTGGCATCGGGACCATAG; |
| | Total mCdx2 Forward: |
| (SEQ ID NO: 12) | |
| | GCGAAACCTGTGCGAGTGGATG; |
| | Total mCdx2 Reverse: |
| (SEQ ID NO: 13) | |
| | CGGTATTTGTCTTTTGTCCTGGT; |
| | mFgfr2 Forward: |
| (SEQ ID NO: 49) | |
| | GCCTCTCGAACAGTATTCTCCT; |
| | mFgfr2 Reverse: |
| (SEQ ID NO: 50) | |
| | ACAGGGTTCATAAGGCATGGG; |
| | Endogenous mGata4 Forward: |
| (SEQ ID NO: 32) | |
| | CACTGGAAAAGCCTGCGTTCTTACA; |
| | Endogenous mGata4 Reverse: |
| (SEQ ID NO: 33) | |
| | AGAATAAGGAAGGAAGAAGTCTCTTGCCTC; |
| | Endogenous mCdx2 Forward: |
| (SEQ ID NO: 34) | |
| | AATTATGGACCTCAGGGGAAGACATG; |
| | Endogenous mCdx2 Reverse: |
| (SEQ ID NO: 35) | |
| | GCTGGCAGGAAGAGTCGGAATG; |
| | mDppa1-Forward: |
| (SEQ ID NO: 51) | |
| | ATGATGTCCCTTCAAGTCCTCA; |
| | mDppa1-Reverse: |
| (SEQ ID NO: 52) | |
| | TGTGTTGGGATCACTTCAGTGT; |
| | Total mGata4-Forward: |
| (SEQ ID NO: 14) | |
| | CACAAGATGAACGGCATCAACC; |
| | Total mGata4-Reverse: |
| (SEQ ID NO: 15) | |
| | CAGCGTGGTGGTAGTCTG; |
| | mGata6-Forward |
| (SEQ ID NO: 53) | |
| | TCAGGGGTAGGGGCATCAG; |
| | mGata6-Reverse: |
| (SEQ ID NO: 54) | |
| | GTAGAGGCCGTCTTGACCTG; |
| | mGapdh-Forward: |
| (SEQ ID NO: 2) | |
| | AGTCAAGGCCGAGAATGGGAAG; |
| | mGapdh-Reverse: |
| (SEQ ID NO: 3) | |
| | AAGCAGTTGGTGGTGCAGGATG; |
| | mSox17-Forward: |
| (SEQ ID NO: 55) | |
| | AGCAGAACCCAGATCTGCAC; |
| | mSox17-Reverse: |
| (SEQ ID NO: 56) | |
| | CGCCTTCCAAGACTTGCCTA; |
| | mActin-Forward: |
| (SEQ ID NO: 57) | |
| | GATATCGCTGCGCTGGTCG; |
| | mActin-Reverse: |
| (SEQ ID NO: 58) | |
| | CCACGATGGAGGGGAATACAG; |
| | mHnf4a-Forward: |
| (SEQ ID NO: 59) | |
| | TGTTGCTAACACGATGCCCT; |
| | mHnf4a-Forward: |
| (SEQ ID NO: 60) | |
| | GGCCACTCACACATCTGTCC; |
| | Foxa2-Forward: |
| (SEQ ID NO: 61) | |
| | GCACTCGGCTTCCAGTATGC; |
| | Foxa2-Reverse: |
| (SEQ ID NO: 62) | |
| | CGTAGTAGCTGCTCCAGTCG; |
| | mPdgfra-Forward: |
| (SEQ ID NO: 63) | |
| | AGTGGCTACATCATCCCCCT; |
| | mPdgfra-Reverse: |
| (SEQ ID NO: 64) | |
| | CCGAAGTCTGTGAGCTGTGT. |

Bulk RNA-seq (Bulk MARS-seq)—RNA-seq libraries were prepared at the crown genomics institute of the Nancy and Stephen Grand Israel National Center for Personalized Medicine, Weizmann Institute of Science. A bulk adaptation of the MARS-Seq protocol (Keren-Shaul et al.) was used to generate RNA-Seq libraries for expression profiling different samples (3 biological replicates from each). Briefly, 60 ng of input RNA from each sample was barcoded during reverse transcription and pooled. Following Agencourct Ampure XP beads cleanup (Beckman Coulter), the pooled samples underwent second strand synthesis and were linearly amplified by T7 in vitro transcription. The resulting RNA was fragmented and converted into a sequencing-ready library by tagging the samples with Illumina sequences during ligation, RT, and PCR. Libraries were quantified by Qubit and TapeStation as previously described (Keren-Shaul et al.) Sequencing was done on a NovaSeq600 using an SP 100 cycles kit (Illumina).

Bulk RNA-seq analysis—Samples were analyzed using UTAP software. Reads were trimmed using CutAdapt (Martin 2011) (parameters: -a ADAPTER1-a “A {10}”-a “T{10}”-A “A{10}”-A “T{10}”-times 2-u 3-u-3-q 20-m 25). Reads were mapped to genome mm10 using STAR v2.4.2a (parameters: -alignEndsType EndToEnd, -outFilterMismatchNoverLmax 0.05, -twopassMode Basic—alignSoftClipAtReferenceEnds No).

Sample counting was done using STAR, quantifying mm10 RefSeq annotated genes. Further analysis was done for genes having a minimum of five reads in at least one sample. Normalization of the counts and differential expression analysis was performed using DESeq2 (Love et al. 2014) with the parameters betaPrior=true, cooksCutoff=false, and independentFiltering=false. Raw P-values were adjusted for multiple testing using the procedure of Benjamini and Hochberg. Hierarchical clustering was generated in UTAP software. Expression heatmap was generated using R pheatmap package.

10× single cell RNA-seq-E8.5 natural embryos and day 8 synthetic embryos were selected for single cell RNA-sequencing. Several samples were sequenced, one samples represents sEmbryo iCdx2 short term DOX induced (from Day−1 until Day+2) and two samples represent sEmbryo iCdx2 10 day dox induced (from Day−8 until Day+2). Moreover, to obtain sampling of single embryo based single cell RNA-seq (rather than relying only on pooled samples 2 embryos), a single sEmbryo from short term induced iCdx2 was processed and sequenced. All synethetic embryos samples were processed including extraembryonic compartments without any dissection. synthetic embryos were dissociated using Trypsin-EDTA solution A 0.25% (Biological Industries: 030501B). Trypsin was neutralized with media including 10% FBS and cells were washed and resuspended in 1×PBS with 400 μg/ml BSA. Cell suspension was filtered with a 100 μm cell strainer to remove cell clumps. A percentage of cell viability higher than 90% was determined by trypan blue staining. Cells were diluted at a final concentration of 1000 cells/μL. scRNA-seq libraries were generated using the 10× Genomics Chromium v3 system (5000 cell target cell recovery) and sequenced on on Illumina NovaSeq 6000 platform according to the manufacturer's instructions.

10× Single cell RNA-seq analysis—10× Genomics data analysis was performed using Cell Ranger 7.0 software (10× Genomics) for pre-processing of raw sequencing data, and Seurat 3.6.3 for downstream analysis. The mm10-3.0.0 gene set downloaded from 10× was used for gene reference requirements. To filter out low-expressing single cells, possible doublets produced during the 10× sample processing, or single cells with extensive mitochondrial expression, we filtered out cells with under 200 expressing genes, over 4.000 expressing genes and over 15% mitochondrial gene expression. Filtering reduced the cell count from overall 61,816 to 46,428 cells across all samples. Seurat integrated analysis and anchoring of all individual samples was performed and then normalized by log-normalization using a scale-factor of 10,000. The top 2,000 variable genes were identified by the variance stabilizing transformation method, and subsequently scaled and centered. Principal components analysis was performed for dimensional examination using the ‘elbow’ method. The first 15 dimensions showed the majority of data variability. Therefore, UMAP dimensional reduction was performed on the first 15 dimensions in all samples. Clusters were detected using Seurat FindClusters function, with resolution parameter=0.6.

For cluster annotation, the area under the curve (AUC) methodology was used to identify the enrichment of each annotated gene-set to each individual single cell. The annotations were based on published gene annotations (Ibarra-Soria et al., 2018), and performed using the R package AUCELL 1.8.035, using parameters: aucMaxRank=100 (5% of the total gene count) under the AUCell_calcAUC function. Each cell was then annotated to a single tissue based on its highest AUC score prediction. Each tissue was then cross-tabulated with each cluster to assess cluster-tissue overlap, and additionally normalized by z-score and ranged to 0-1 for plotting purposes. Next, to evaluate the probability of a certain cluster being enriched in a certain tissue, the annotated AUC predictions of each cell to a tissue was used to compare the observed cluster annotation of each cell, thus producing a P value based on Mann-Whitney U statistics. This was calculated using the R package verification v1.42 roc. area function (CRAN.R-project (dot) org). Integration of both the predicted annotation overlap and its statistical enrichment to each cluster resulted in a predicted tissue per cluster. T-test was used to assess significant changes in the proportional size of each cluster between natural and synthetic embryos. Expression pattern of selected genes was shown as two parameters: normalized mean expression, and enrichment of cells that express this gene (expression>0), among the specified cluster (either in natural or in synthetic originated cells).

Statistical analysis—All statistical analysis were performed using the GraphPad Prism 8 software (La Joya, California). Data on graphs indicates means plus s.e.m. of a minimum of three independent experiments, unless otherwise stated. Kolmogorov-Smirnov test was performed to check normal distribution of data before each statistical test. Significant difference between two samples was evaluated by unpaired two-sided Student's t-test if data was normally distributed or Mann-Whitney test for non-normally distributed data. p<0.05 was considered as statistically significant.

### Results

As demonstrated in Example 2 hereinabove, co-aggregation of three stem cell populations, all originating from naïve ESCs/iPSCs as a starting material, followed by ex-utero culturing enabled generation of organized embryos.

Following, multiple rounds of testing and optimizations were conducted to determine the DOX pretreatment regimen compatible with a relatively more productive outcome, and to define optimal cell numbers and aggregation plates. The inventors also tested whether DOX continuation after co-aggregation of the three subpopulations (set as timepoint 0) could improve the experimental outcome.

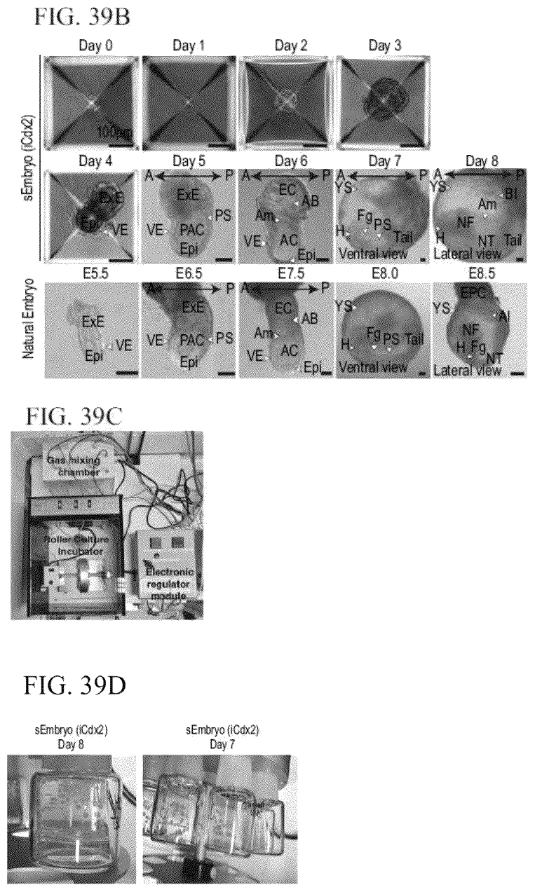

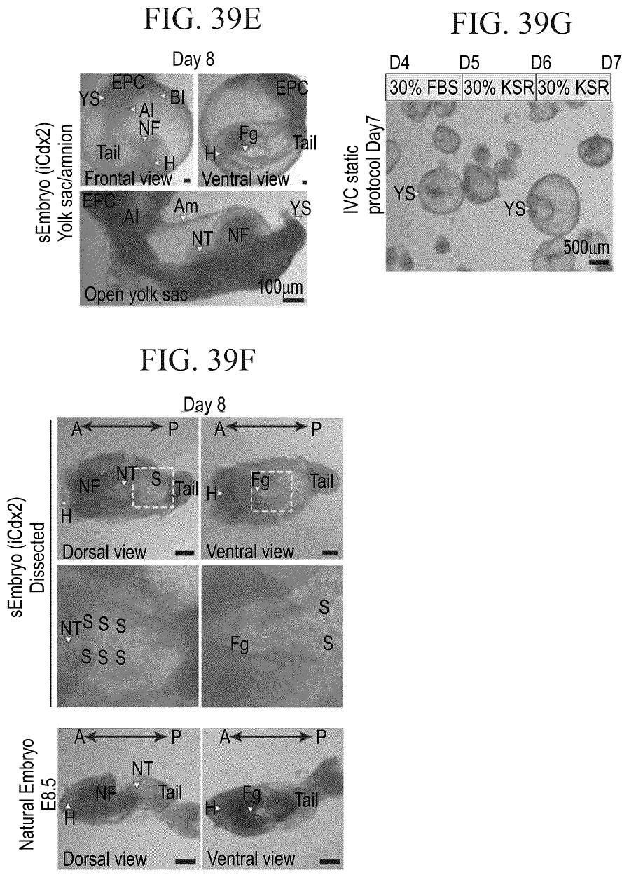

The following conditions were selected and applied throughout Example 3 (FIG. 39A):
all steps of the protocol were done in 20% O2 and 5% CO2 conditions;
pre-induction of Gata4 for 24 hours in 2i/Lif conditions prior to co-aggregation;
pre-induction of Cdx2 for between 24 hours up to 14 days in TSC-LPA medium prior to co-aggregation;
400 or 800 micron AggreWell plates;
inclusion of DOX in the aggregation medium (AM) for the first 48 hours following aggregation.
a day 3, AM media was replaced by an EUCM2 which contained 20% FBS.
due to their increase in size, the aggregates were combined on day 4 and gently transferred to non-adherent tissue culture plates on a shaker placed inside a conventional tissue culture incubator and placed in EUCM3 which contained 30% FBS.
at day 5, egg-cylinder shaped embryos (FIG. 39B) were manually picked under the stereomicroscope and transferred to the ex utero roller culture system in EUCM conditions (FIG. 39C).
from day 5 to day 8 of the protocol, synthetic embryos continued to be grown in the roller culture system (20% O₂ plus 5% CO₂ and a pressure of 6.5-8 psi), and EUCM was refreshed daily. The roller culture-EUCM platform was essential for continuation of embryo development beyond day 5-6, as continuing only with the static conditions in these days yielded a detrimental outcome.

This 8 day protocol supported the self-organization and growth of naïve ESC derived aggregates into organogenesis stage synthetic embryos (also referred to herein as “sEmbryo iCdx2”) that grow within extraembryonic membranes (FIGS. 39D-F), comparable to E8.5 in utero developed natural embryos as further described below.

Notably, synthetic embryos generated from the same iCdx2 and iGata4 ESCs but grown from day 4-7 in previous published IVC medium-static conditions based protocols (Bedzhov and Zernicka-Goetz, 2014; Harrison et al., 2017; Sozen et al., 2018), did not develop further than previously achieved (Amadei et al., 2021) and yielded empty yolk sacs that lack embryo proper structures (FIG. 39G).

Analysis of defined morphological traits during synthetic embryo growth were systematically compared to those classically well established in natural embryos at different time points in mouse embryogenesis (Dai et al., 2016; van Maele-Fabry et al., 1992; Mittenzweig et al., 2021; Ohinata et al., 2005; Parameswaran and Tam, 1995; Rossant and Tam, 2017; Schwarz and Hadjantonakis, 2020; Tam and Snow, 1980). The synthetic embryos originating from naïve ESCs starting populations faithfully resembled all stages of natural post-implantation development, going through luminogenesis, symmetry breaking and gastrulation, until early organ formation (FIG. 39B). Egg cylinder shape synthetic embryos start emerging on day 3 of the protocol, when aggregates start luminogenesis and an outer cell layer is formed, and without going through a blastocyst-like morphology in the earlier days. At day 4, the aggregates show similar morphology to an E5.5 embryo, with clear segregation into the epiblast (Epi) and extra-embryonic ectoderm (ExE) compartments surrounded by a layer of visceral endoderm (VE) cells (FIG. 39B). At day 5, the synthetic embryos closely resemble E6.5 in-utero embryos, showing a clear difference between the cup-shaped epiblast and the ExE, both enveloped by the VE. The Epi displays an expanded pro-amniotic cavity (PAC), and successfully break symmetry showing an incipient primitive streak in one side of the epiblast, adjacent to the ExE (FIG. 39B). After 6 days the synthetic embryos reach the neural plate stage. The amniotic folds have fused to form the amnion (Am) as expected, generating the amniotic (AC) and exocoelomic cavities (EC) (FIG. 39B), and an incipient allantoic bud (AB) can be observed in the ExE compartment at the opposite side of the neural plate. At day 7 there is a major expansion of the yolk sac (YS), which by then surrounds the embryo, mimicking what happens in natural in-utero embryos; while in the Epi compartment the anterior ectoderm begins to form a broad plate, the future neural groove, making evident the emergence of the head-to-tail axis (FIG. 39B). In the ventral part of the synthetic embryo at day 7, the migration of the primitive streak and the heart field are evident, and the foregut invagination starts to be seen, like E8.0 natural embryos (FIG. 39B). At day 8 the synthetic embryos closely resemble the morphology of E8.5 in utero embryos (FIGS. 39E-F). The dorso-ventral axis of the embryos is clearly seen by the neural folds facing the amnion dorsally versus the foregut facing the yolk sac ventrally (FIGS. 39E-F). The synthetic embryos continue growing completely enveloped inside the extra-embryonic membranes (yolk sac and amnion) and present an ectoplacental cone (EPC)-like structure in the opposite side of the embryo (FIG. 39E). The blood islands (BI) are clearly visible in the lateral sides of the yolk sac (FIG. 39E), and blood begins to circulate in the yolk sac vessels (vitelline circulation). The embryos display well-formed head folds, neural tube, invaginating foregut, beating heart, and between up to 4 pairs of somites, followed by the tail (FIG. 39F), demonstrating the complete establishment of the head-to-tail and dorso-ventral axis. The allantois extends from the posterior part of the embryo, connecting the tail to the EPC (FIG. 39E). Although there is a variation in size among adequately developed synthetic embryos at day 8, they were comparable to in utero developed natural E8.5 Embryos.

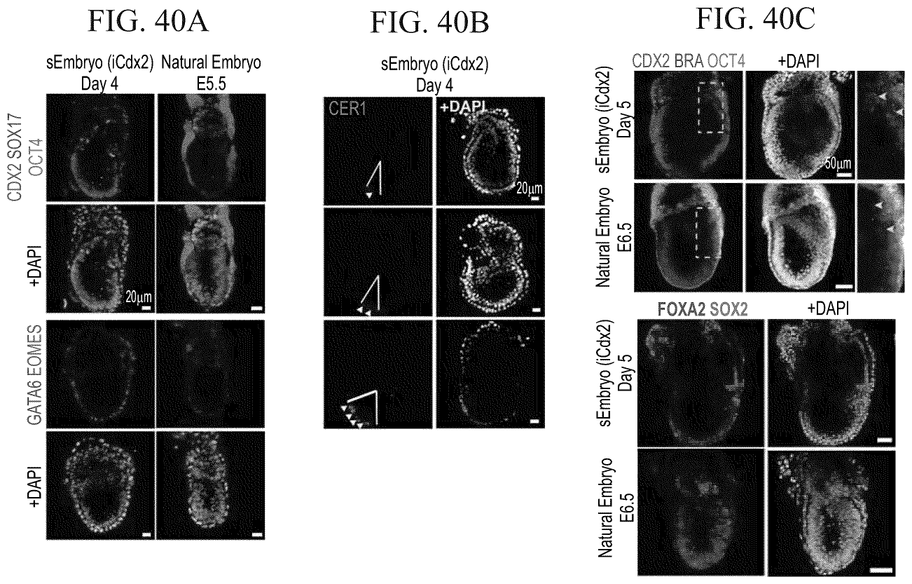

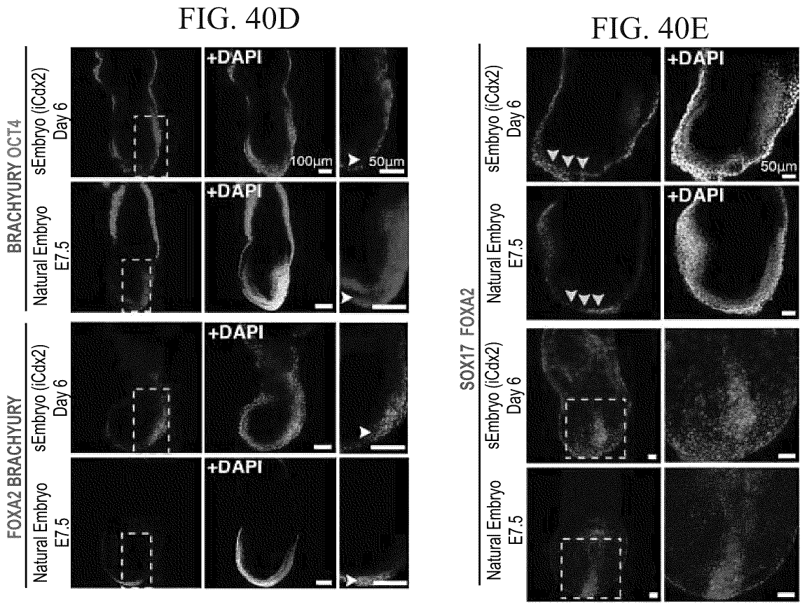

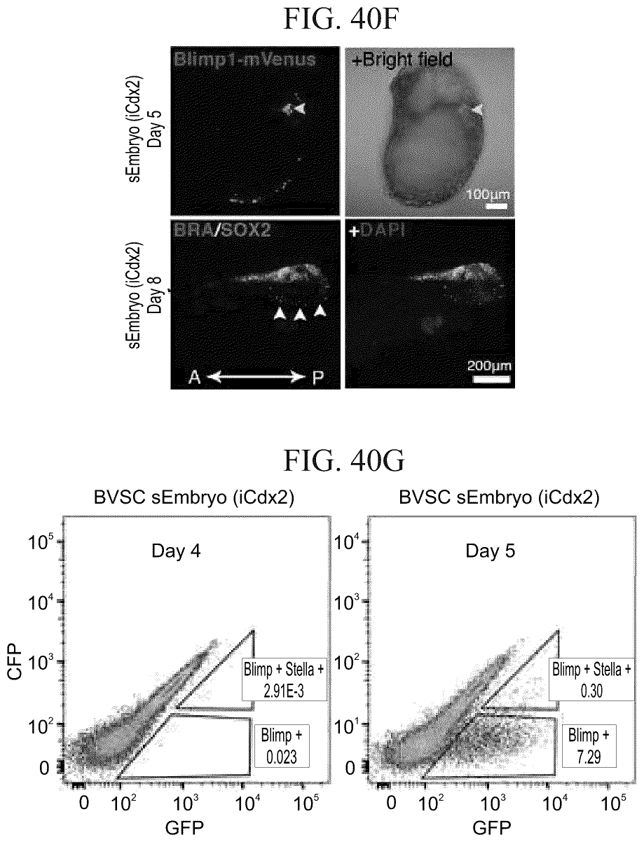

Using whole-mount immunofluorescence in natural embryos dissected at E5.5-E6.5, and in egg-cylinder synthetic embryos at day 3, 4 and 5, proper expression of canonical markers for the three cell lineages expected to be present in the mouse embryo at the egg cylinder-stage were corroborated: a cup-shaped epiblast positive for OCT4 and OTX2 (FIG. 40A); the extra-embryonic ectoderm (ExE) adjacent to the epiblast expressing CDX2, TFAP2C and EOMES (FIG. 40A); and the visceral endoderm (VE) positive for GATA4, GATA6, SOX17 and FOXA2 enveloping both compartments (FIG. 40A). OTX2 and EOMES were present also in the embryonic VE, as it occurs in natural embryos (FIG. 40A). SOX2 expression in the Epi compartment as well as the ExE was correctly recapitulated in synthetic embryos. Further, proper establishment and migration of the AVE from the distal part of the epiblast towards the future anterior part was evidenced by the staining for BMP antagonist CER1 and the WNT inhibitor DKK1 at day 4, either at the distal tip of the epiblast or asymmetrically located towards one side of the egg cylinder (FIG. 40B) similarly to as previously characterized in natural embryos and eTSC based day 4 synthetic embryos (Amadei et al., 2021). On day 5, a population of Brachyury⁺ cells appeared at the posterior side of the epiblast near the ExE boundary, opposite to Cerl and DKK1, corroborating the appearance of the primitive streak and the onset of gastrulation in day 5 synthetic embryos (FIG. 40C). At day 6, the population of Brachyury+ cells expanded and migrated towards the distal part of the Epi, between the VE and the Epiblast (FIG. 40D). Emergence of the axial mesoderm was evidenced by the presence of FOXA2/BRACHYURY+ cells at the distal tip of the primitive streak, as described for natural embryos at E7.5 (FIG. 40D). Similarly, definitive endoderm cells were identified in synthetic embryos at day 6 by co-staining of SOX17 and FOXA2, while a population of FOXA2+/SOX17− cells allocated along the distal tip of the egg-cylinder indicated the emerging midline of the embryo (FIG. 40E).

To analyze the emergence of primordial germ cells (PGCs) in the synthetic embryos the Blimp1-mVenus Stella-CFP reporter found in the BVSC ESC line (Hayashi et al., 2011) was used in the aggregation protocol alongside induction of Cdx2 and Gata4 cells. Activation of the Blimp1-mVenus fluorescent reporter was detected at day 5 by live imaging, specifically at the site of putative the primitive streak, in the boundaries of the Epi/ExE (FIG. 40F—Top row). Flow cytometry analysis confirmed the emergence of the initial PGC population in day 5 synthetic embryos which is known to originate first as Blimp1+Stella− cells in the developmentally equivalent natural E6.5 embryos (FIG. 40G) (Magnúsdóttir and Surani, 2014). Moreover, migration of PGC cells (Leitch et al., 2013) in synthetic embryos to the posterior ventral part of the embryos was observed at day 8 by SOX2 immunostaining, that were clearly separated from the neural tube SOX2+ population, indicating that PGC migration can be recapitulated as well in the synthetic embryos (FIG. 40F-Bottom row).

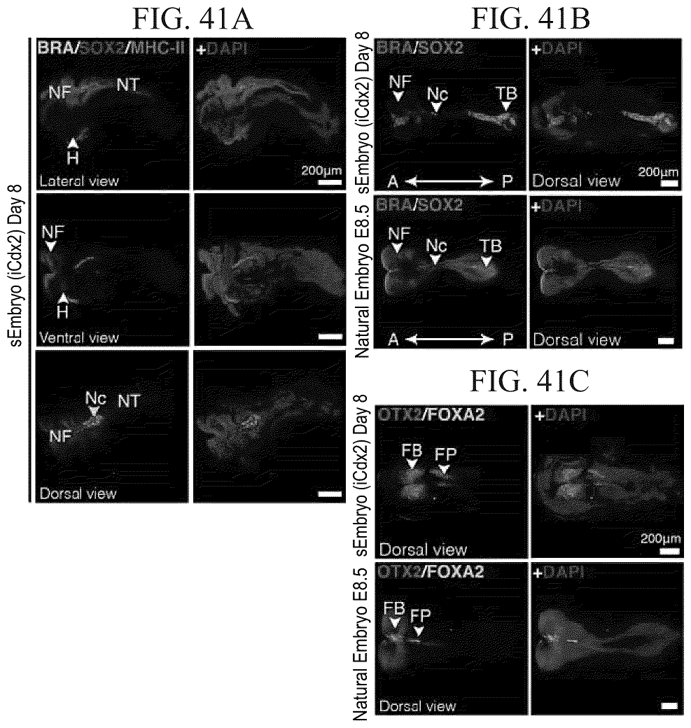

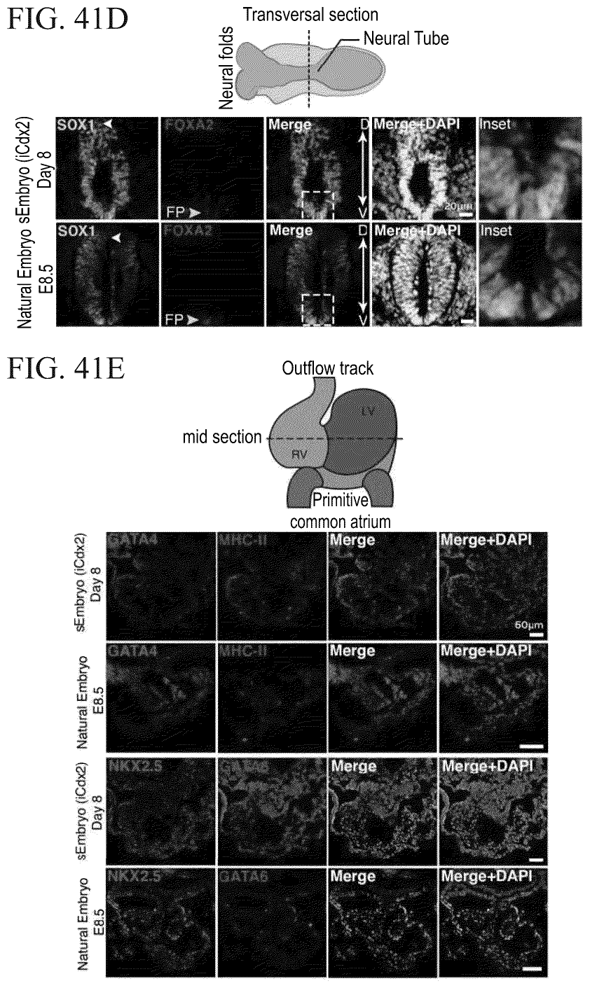

Following gastrulation, the developing embryo establishes its general body plan and displays multiple ectodermal, endodermal, and mesodermal-derived tissues. In addition to the morphological similarities observed between day 8 synthetic embryos and E8.5 natural in-utero embryos, proper differentiation and tissue morphogenesis were corroborated by assessing the expression of several lineage-specific markers by whole-mount immunofluorescence. The neural folds (NF) and neural tube (NT) derived from the epiblast ectoderm, presented strong Sox2 expression, properly allocated along the antero-posterior axis of the embryo (FIGS. 41A-B), while BRACHYURY+ cells were found along the embryonic midline, resembling the elongated notochord (Nc) and tail bud (FIG. 41B). The cardiac marker Myosin Heavy Chain II (MHC-II) was visible at the anterior ventral part of the embryo, specifically localized in the heart bud (FIG. 41A). OTX2 marks the embryonic forebrain and midbrain in the natural E8.5 embryo and was detected in the anterior part of the neural folds in the synthetic embryos (FIG. 41C). FOXA2, which is restricted to the notochord floor plate at the midline of the embryo, was also detected in day 8 synthetic embryos (FIG. 41C).

To further analyze the extent of tissue patterning, transversal plane cross-sectioning of the embryonic neural tube and heart was performed. SOX1 and FOXA2 staining indicated proper establishment of the dorso-ventral axis in the spinal cord, evidenced by the double positive cells specifically located at the ventral part, resembling the floor plate in natural embryos (FIG. 41D—yellow arrow and inset). Moreover, folding and complete closure of the proximal region of the neural tube was observed in day 8 synthetic embryos, which corresponds to what has been described in natural embryos (FIG. 41D—white arrow) (Massarwa and Niswander, 2013b; Nikolopoulou et al., 2017). Sectioning based analysis of the developing heart of the synthetic embryos at day 8 exhibited a remarkably similar morphology to the E8.5 natural embryo, suggesting proper recapitulation of heart morphogenesis. At E8.5, the primitive heart tube undergoes looping and develops into a chambered heart (Mandrycky et al., 2020). Formation of the chambers of the heart during cardiac looping (FIG. 41E), and emergence of functional heart beating that in turn propels blood circulation in the synthetic embryo (data not shown) were noted. Heart differentiation and patterning were further analyzed using four different markers of the developing heart (Gata4, Gata6, NKX2.5 and MHC-II), all of them showing proper expression in the heart tube of the synthetic embryos (FIG. 41E) in a comparable manner to equivalent E8.5 natural embryos. Altogether, these data suggest that the emergence and spatial-temporal organization of tissues derived from the three germ-layers of the mouse post-gastrulation embryo is recapitulated in the synthetic embryos described herein.

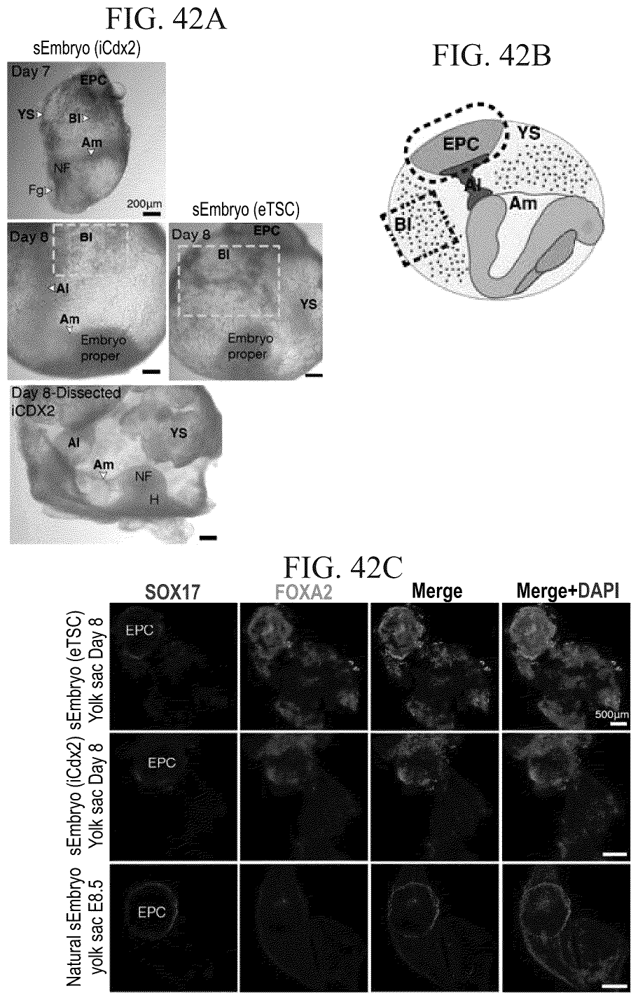

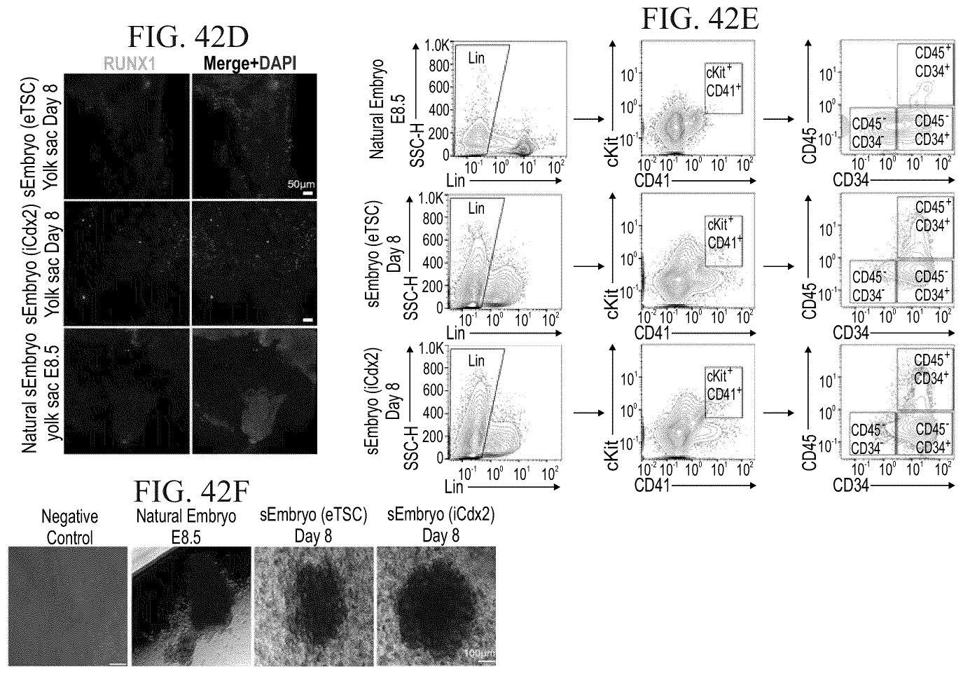

Development of the post-implantation mammalian embryo is highly dependent on the extraembryonic tissues, which are the main drivers for induction of symmetry breaking (AVE) at E5.5, and gastrulation together with formation of the primitive streak (ExE) at E6.5. As gastrulation proceeds, the mouse embryo becomes encapsulated by the rapidly enlarging extraembryonic tissues (e.g., yolk sac and amnion) (Pereira et al., 2011) (FIGS. 39B and 39E). However, currently there are no available stem cell based models that recapitulate this important process and the interactions involved in it. Remarkably, as shown in FIG. 39B, the formation and organization of extraembryonic tissues were properly recapitulated in the synethic embryos generated through the protocols described herein, based on anatomical and morphological analysis. Further, at day 7, the yolk sac starts to enlarge and engulf the embryo dorsally. Besides that, the amnion, ectoplacental cone, and yolk sac blood islands become evident (FIG. 42A). At day 8, the embryo develops completely within the yolk sac and amnion, with the amnion being the innermost membrane enveloping the embryo-proper from the dorsal part, surrounded by the vascularized yolk sac with the ectoplacental cone attached to it on the opposite side of the embryo (FIGS. 42A-B). Moreover, the presence and expression pattern of FOXA2 and SOX17 in yolk sacs and ectoplacental cones of day 8 synthetic embryos, following whole mount immunostaining, closely resembles that of natural in utero E8.5 embryonic extraembryonic compartment (FIG. 42C).

One of the important developmental events involving the extra-embryonic yolk sac, is the emergence of blood islands in the yolk sac (Yamane, 2018), which marks the first hematopoietic event during mammalian development (Nowotschin et al.; Pijuan-Sala et al., 2019). Blood islands were widely visible and abundant in developing yolk sacs (data not shown), and immunostaining of whole yolk-sacs for RUNX1 which marks these primitive hematopoietic progenitors, termed erythromyeloid progenitors (EMPs) (Iturri et al., 2021) confirmed their identity as similarly observed in natural embryos (FIG. 42D). EMPs are developmentally restricted blood progenitors the originate from the hemogenic endothelium of the yolk sac and have the potential to give rise to definitive hematopoietic cells from the erythroid and myeloid lineages. Recent studies have provided definitive surface markers that characterize the first primitive hematopoietic cells in E8-E8.75 mouse yolk sac (Iturri et al., 2021). FACS analysis confirmed authentic expression of defining surface markers delineating the different populations within the hematopoietic progenitors in the embryonic yolk sac compartment, as previously described in natural embryos, showing that LIN fraction contains double positive CD41⁺/cKit⁺ population, which can be subdivided into three subgroups based on CD34 and CD45 markers expression (FIG. 42E) (Iturri et al., 2021). Given that EMP based hematopoiesis supports embryo survival until birth through erythrocyte production, a methylcellulose based assay was used to validate erythroid functional colony forming and expansion potential from EMPs isolated from natural and synthetic yolk-sacs followed by in vitro incubation within the cytokine enriched methylcellulose culture plates (Iturri et al., 2021). Consistent with the authentic marker expression and subpopulation distribution observed (FIG. 42E), synthetic embryos derived blood progenitors robustly formed typical erythrocyte colonies with appropriate morphology in an identical manner to natural in utero derived yolk sacs derived EMPs (FIG. 42F). Of note, this latter experiment provides a proof of the ability to isolate and further in vitro expand properly differentiating cells from post-gastrulation synthetic embryos.

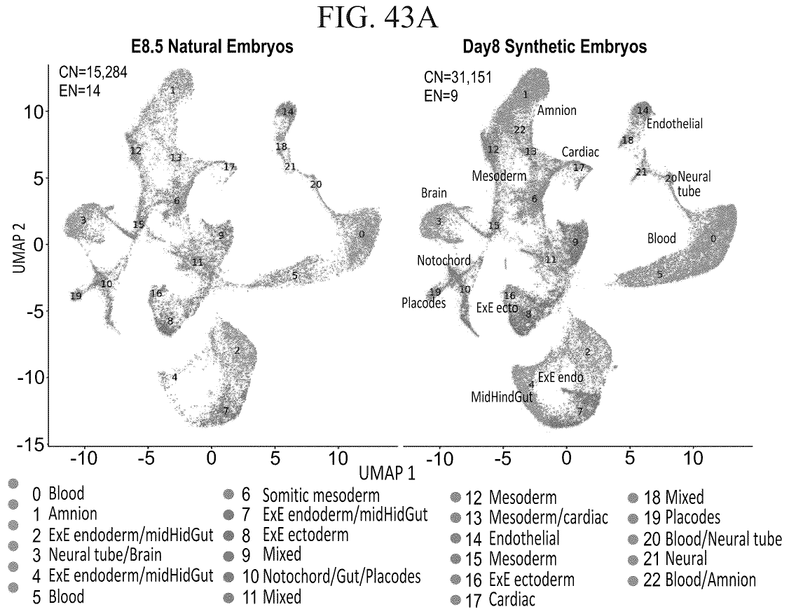

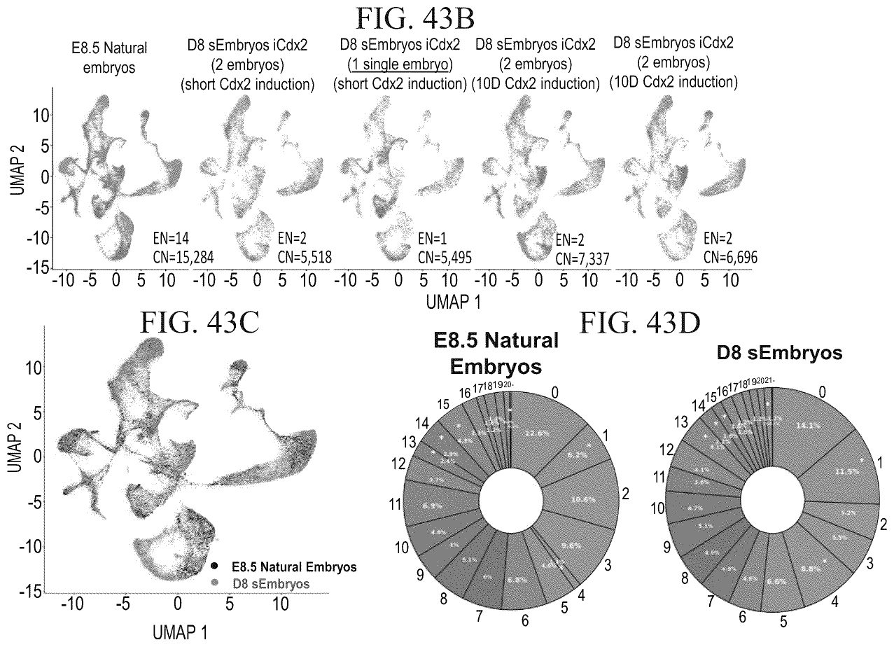

To characterize and annotate the various cell types present in the advanced synthetic embryos generated herein in a more quantitative and unbiased manner, and to identify to which extent the global transcriptional profile of lineages formed with synthetic embryos is like their natural counterparts, single cell RNA-sequencing (scRNA-seq) was performed using a 10× genomics platform. To this end, cells were collected from synthetic day 8 embryos with ˜E8.5 like morphology: 3 synthetic embryos that were developed from iCdx2 cells following brief 3 day DOX based induction (day −1 until day +2) and 4 embryos that were developed following iCdx2 cells following 10 day DOX based induction (day −7 until day +2) (FIGS. 43A-B). Clustering analysis based on differentially expressed genes revealed 22 different cell states (FIG. 43A). Subsequently, the identity of the clusters was annotated based on specific marker genes of the major cell lineages previously defined by single-cell transcriptomics of early mouse embryos (Pijuan-Sala et al., 2019). All three germ layers were represented, as well as all extraembryonic tissues, in one or more clusters, indicating the presence of different cell states within those lineages in a similar manner in both natural and synthetic embryos (FIGS. 43A-C). When examining synthetic embryo biological replicates, or when examining a single embryo based single cell RNA-seq sample (short iCdx2 induction), similar results were obtained (FIG. 43B).

Importantly, the profile of cell types found in the synthetic embryos developing ex utero was highly similar to natural embryos, demonstrating that lineage differentiation and commitment is faithfully recapitulated in the synthetic embryos at the single cell level (FIGS. 43B-C). This analysis confirmed that the composition of cell transcriptional states in the synthetic embryos developing ex utero until advanced organogenesis is equivalent to their natural counterparts. Comparison of the relative cell proportions of different cell types in synthetic and natural embryos showed no significant differences in the majority of clusters, while some moderate differences were found in only six of the cell clusters, including amnion and extra-embryonic endoderm (FIG. 43B). Transcriptionally, all cell clusters showed very high correlation (0.95-0.98) between synthetic and natural embryos (data not shown). In addition, specific markers for tissues that were validated above by morphological or immunostaining based analysis, such as somitic mesoderm, notochord, neural tube, and cardiac tissue were expressed specifically in their corresponding tissue cluster, both in synthetic and in natural embryos (data not shown). A similar conclusion could be reached when focusing on specific markers (Mittenzweig et al., 2021) for different extra-embryonic tissues (data not shown). Overall, these results prove that day 8 synthetic embryos are highly similar to their natural E8.5 counterparts.

### Example 4

### Generation of Human Naïve PSCs Transiently Expressing GATA4, CDX2 and/or GATA3

### Materials and Methods

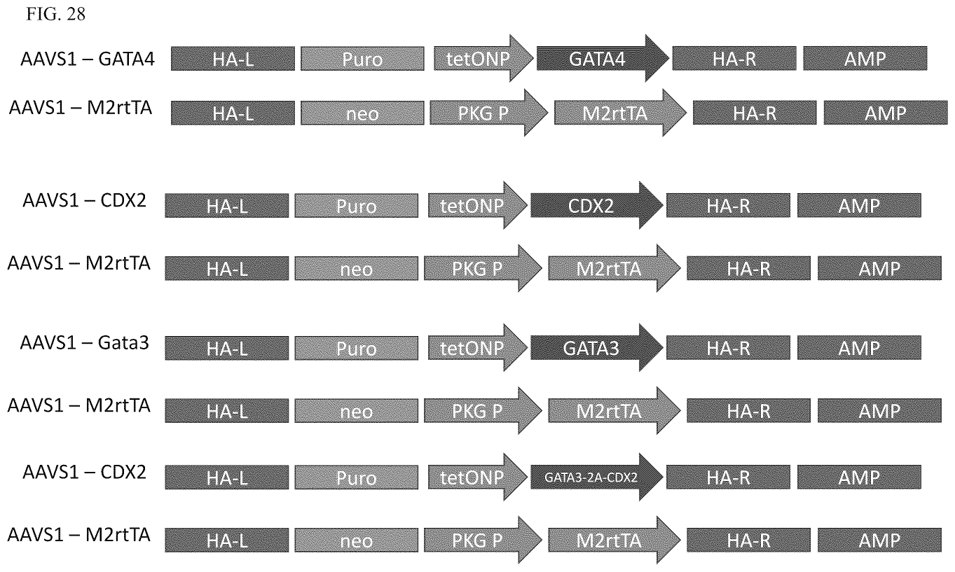

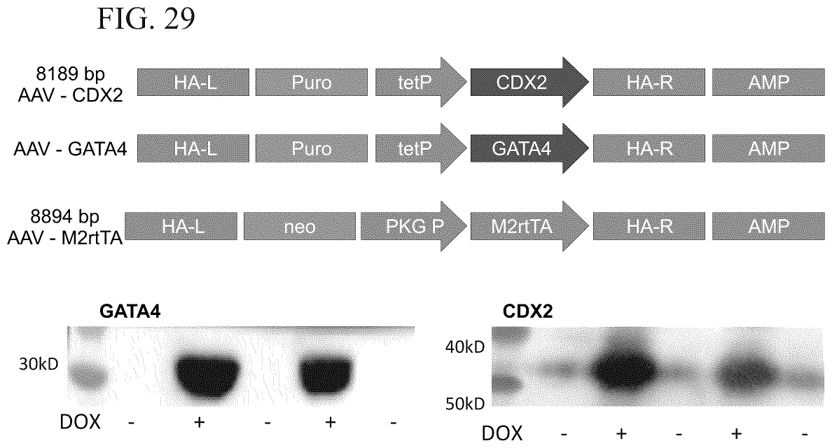

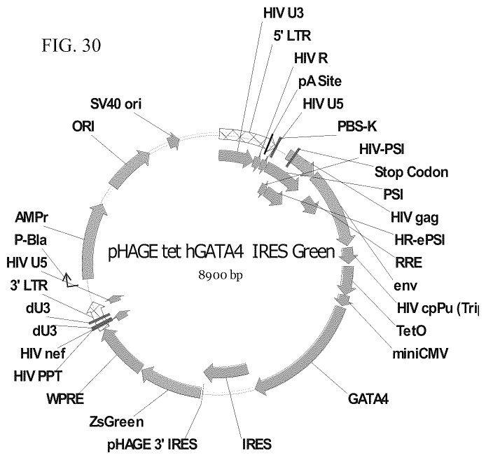

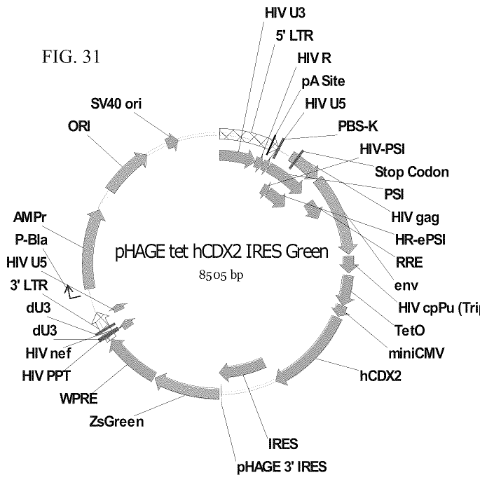

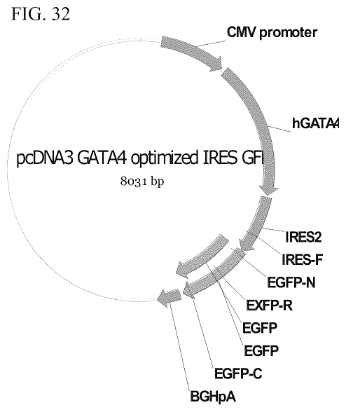

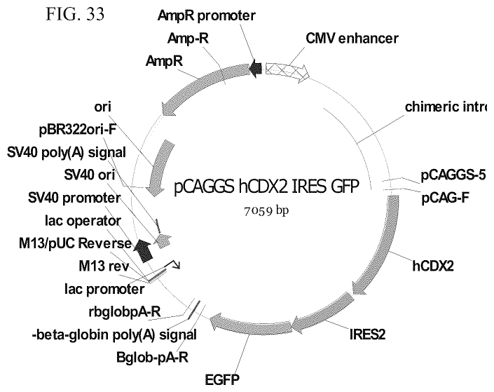

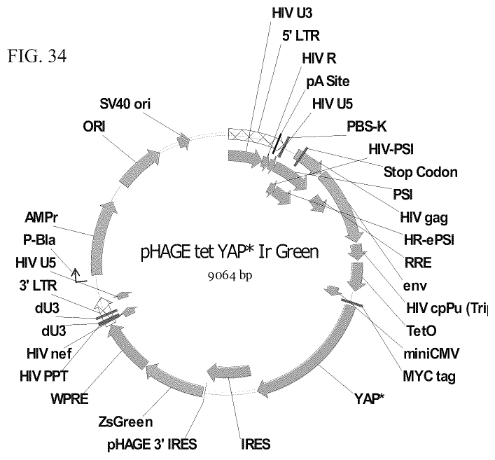

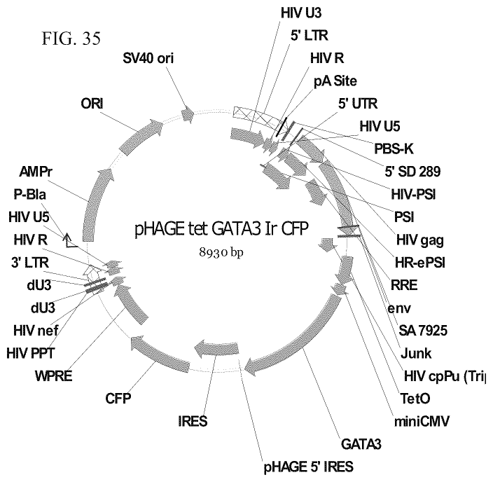

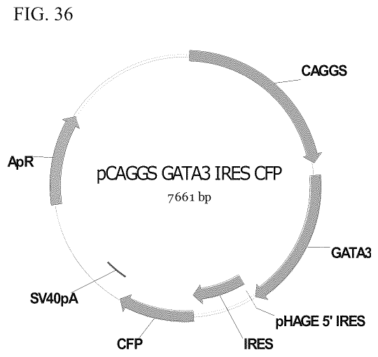

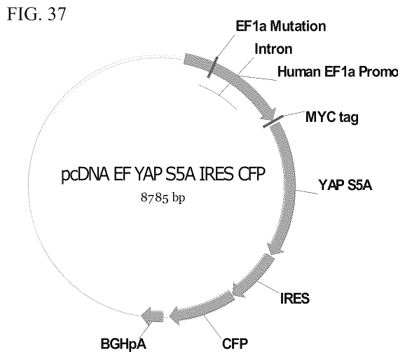

Gata4, Cdx2, Gata3 inducible naïve human pluripotent stem cells—To generate human TetON inducible Gata4, Cdx2, Gata3 of Gata3-2A-Cdx2 overexpression inserts the present inventors designed targeting vectors for the safe harbor of the AAVS1 locus in human cells (10.1038/nbt.1927). WIBR3 human ESCs and JH22 human iPSC line were targeted with CRISPR and 2 targeting vectors—one introducing the M2Rtta allele (with neo selection) and the other including the transcriptional factor insert (TF) needed with a vector carrying puromycin selection. Electroporated cells were subjected to double antibiotic selection with puromycin and neomycin for 10-14 days, and clones were picked and validated for correct targeting and overexpression. The targeting schemes, designed plasmids and validation results are presented in FIGS. 28-37.

Expansion and maintenance of human naïve pluripotent cells—WIBR3-WT, WIBR3-TetOn-CDX2 and WIBR3-TetOn-Gata 4 were expanded in human naïve pluripotency conditions termed HENSM, as described in Bayerl et al. Cell Stem Cell 2021. Briefly, HENSM medium: 240 mL Neurobasal (ThermoFisher 21103049) and 240 mL of DMEM-F12 without HEPES (ThermoFisher 21331020), 5 mL N2 supplement (ThermoFisher 17502048 or in-house prepared), 5 mL (1×) GlutaMAX (ThermoFisher 35050061), 1% nonessential amino acids (BI 01-340-1B), 1% Penicillin-Streptomycin (BI 03-031-1B), 10 mL B27 supplement (Invitrogen 17504044), Geltrex (Invitrogen A1413202/A1413302-add 1 mL rapidly in media to obtain 0.2% final conc), 50 mg/ml Vitamin C (L-Ascorbic acid 2-phosphate sesquimagnesium salt hydrate—Sigma A8950), 20 ng/ml recombinant human LIF (Peprotech 300-05) and the following small molecule inhibitors: FGFRi/MEKi/ERKi (PD0325901 1.25 μM—Axon Medchem 1408); WNTi/TNKi (XAV939 2 μM—Sigma X3004 or Axon Medchem 1527); PKCi (Go6983 2 μM—Axon Medchem 2466), SRCi (CGP77675 1.2 μM—Axon Medchem 2097). Secondary boosting components: P38i/JNKi (BIRB0796 0.8 μM—Axon Medchem 1358), ROCKi Y27632 (1.2 μM—Axon Medchem 1683), and ACTIVIN A (ACT) 10 ng/ml. Cells in HENSM were grown on 1% Growth factor reduced Matrigel (BD FAL356231) or 1% GELTREX (ThermoFisher A1413202 orA1413302) or Biolaminin-511 (Biolamina Inc; including for HENSM-XF conditions) coated plates for at least 1 hour in 37° C. After at least 4 passages in HENSM lines acquired uniform human naïve pluripotency morphology and molecular validation as described in Bayerl et al. Cell Stem Cell 2021 and Gafni et al. Nature 2013.

Aggregation medium (AM)—1:1 DMEM-F12 (Thermo 11320033) & Neurobasal (Thermo 21103049), Glutamax 1×, Sodium pyruvate 1 mM, Pen/Strep 1×, NEAA 1×, N2 supplement 1× (Thermo 17502048), B27 supplement 1× (Thermo 17504044), beta-mercaptoethanol 0.1 mM (Thermo 31350010) and 0.45% BSA (SigmaAldrich A7979) (For 500 ml Total). The aggregation media was supplemented with Doxycycline (2 μg/ml final concentration) and 5 μM of Y27632 to increase cell survival for the first 24 hours.

Co-aggregation and culture-Co-aggregation was defined as time point 0 of the protocol and was made from three starting PSC cell types:
WIBR3-WT naïve human ESCs (To give rise to embryo proper);
Dox pre-treated (16 hours) WIBR3-TetOn-Gata4 naïve human ESCs (to give rise to extraembryonic primitive endoderm (Pre) lineage); and
DOX pre-treated WIBR3-TetOn-CDX2 naïve human ESCs (to give rise to entire extraembryonic trophectoderm lineage).

Before harvesting cells were pretreated with 20 μM of ROCKi Y27632 to increase their survival after single cell passaging. Cells were dissociated into single cells with TryplE for 5 minutes at 37° C. Following, cells were resuspended in PBS, centrifuged, and rewashed with PBS and centrifuged again and PBS discarded, and resuspended in aggregation Media (AM). Following cell count, a total number of 36000 WIBR3-TetOn-Cdx2 and 12,000 WIBR3-TetOn-CDX2 human naïve ESCs and 12000 WIBR3-WT (empty) naïve human cells were added per well in 400 Microwell AggreWell 24 well plates. In 800 Microwell plates, 9000 WIBR3-TetOn-Cdx2 and 3000 WIBR3-TetOn-CDX2 human naïve ESCs and 3000 WIBR3-WT (empty) naïve human cells were added per well in AggreWell 24 well. The cell mixture was added as 1 ml per well (final volume of each well of 24 well is 1.5 ml).
Day 0—The plate was centrifuged at 100×g for 3 minutes and this was marked as day 0, and then placed into static incubator at 37° C. 5% Co2 and 5% 02.
On Day 1, 1 ml of media was gently aspirated from each well and replaced with pre-warmed 1 ml of fresh AM+DOX without ROCKi). This was repeated twice during that day to remove ROCKi.
On Day 2, 1 ml of media was gently aspirated from each well and replaced with pre-warmed 1 ml of fresh AM+DOX.
On days 3 and 4, 1 ml of media was gently aspirated from each well and replaced with pre-warmed 1 ml of fresh AM.
On days 5-8, 1 ml of media was gently aspirated from each well and replaced with pre-warmed 1 ml of fresh EUCM2 (CMRL/advDMEM-F12 based) media with 20% FBS and transfer plate into 5% Co2 and 20% O2.
On days 9-11, 1 ml of media was gently aspirated from each well and replaced with pre-warmed 1 ml of EUCM2 (CMRL/advDMEM-F12 based) media with 30% FBS.
At day 12, embryos were transferred into non-adherent 6 wells plate onto a rotating shaker in static conditions each well with 3 ml EUCM3 (CMRL/advDMEM-F12 based) media with 20% HAS and 20% KSR at 5% Co2 and 20% O2.
On days 13-15, 1.5 ml of media was gently aspirated from each well and replaced with pre-warmed 1.5 ml of EUCM3 (CMRL/advDMEM-F12 based) media with 20% HAS and 20% KSR.
On days 16-18, 1.5 ml of media was gently aspirated from each well and replaced with pre-warmed 1.5 ml of EUCM4 (CMRL/advDMEM-F12 based) media with 30% HAS and 20% KSR.
On days 19-21, 1.5 ml of media was gently aspirated from each well and replaced with pre-warmed 1.5 ml of EUCM5 (CMRL/advDMEM-F12 based) media with 40% HAS and 20% KSR.
At day 22 onwards—high quality embryos were selected and transferred into previously described ex utero roller culture system (Alejandro-Castrejon et al. Nature 2021) in fresh EUCM media supplemented with D-Glucose 4 mg/ml, sodium-pyruvate 1 mM and 1×NEAA and incubated at 20% O₂, 5% Co2 and 6.5 Psi, with the change from mouse EUCM to human embryo EUCM is that the human optimized one contains 50% HAS and 25% KSR (and no RAS).

### Results

Naïve human PSCs that transiently overexpress Cdx2 upon addition of DOX in the early stages of embryo aggregation protocol were generated, in order to induce their rapid priming and transition towards early trophectoderm (TE). In addition, naïve human PSCs that transiently overexpress Gata4 upon addition of DOX in the early stages of embryo aggregation protocol were generated, in order to induce their rapid priming and transition towards the extraembryonic primitive endoderm lineage.

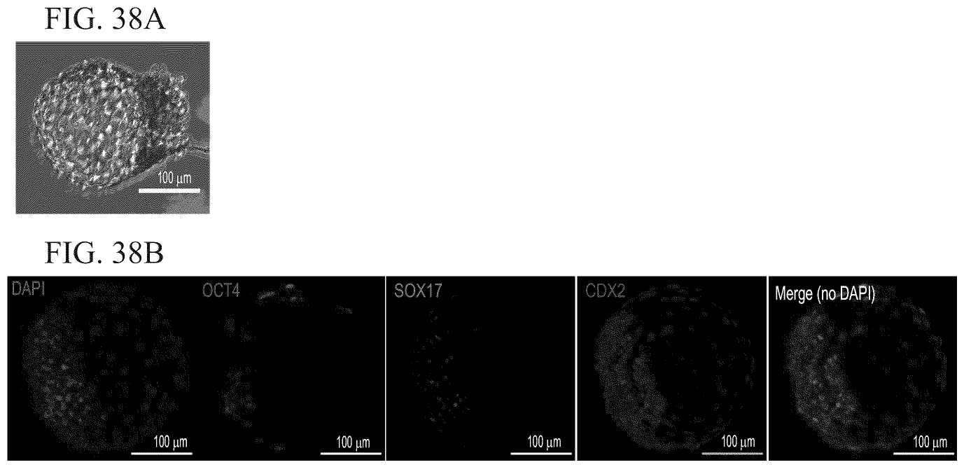

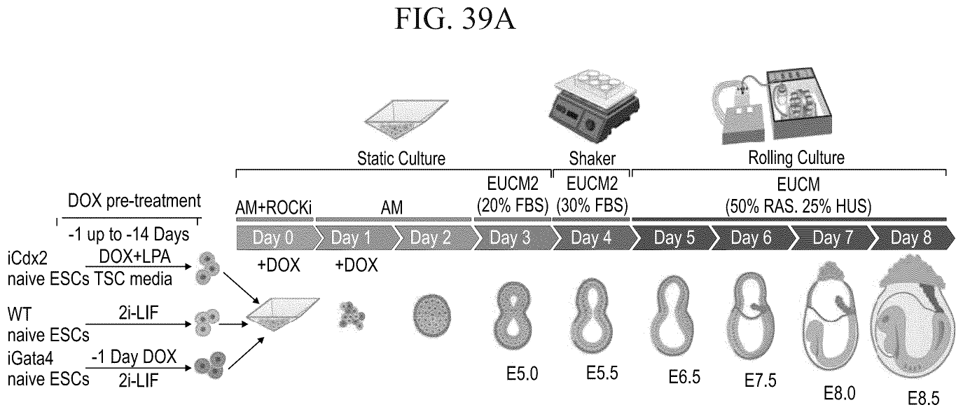

Following, co-aggregation of naïve human PSCs with these two stem cell populations followed by ex-utero culturing enabled generation of organized embryos, as exemplified in FIGS. 38A-B).

### Example 5

### Aggregation of Three Human Naïve PSCs Subpopulations Generates an Organized Embryo

### Materials and Methods

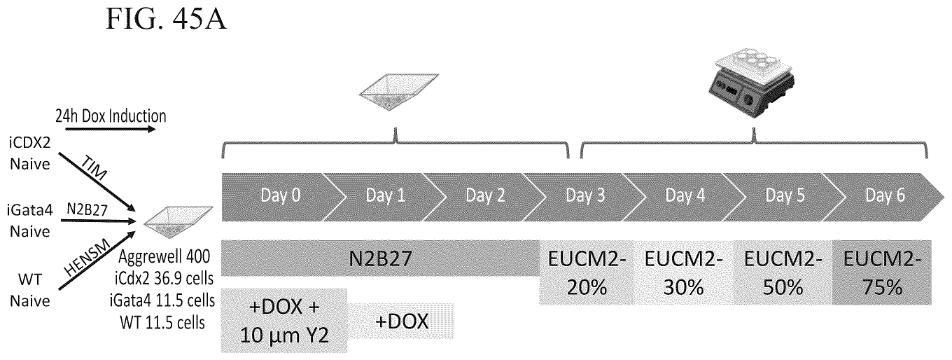

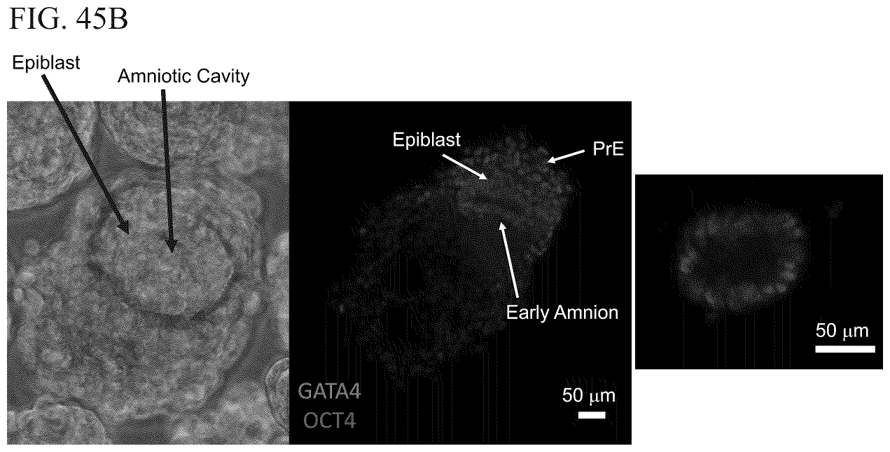

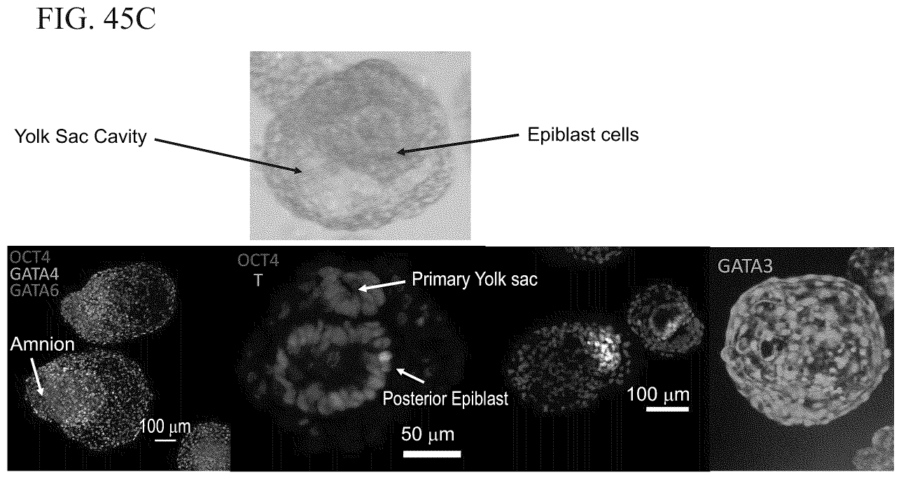

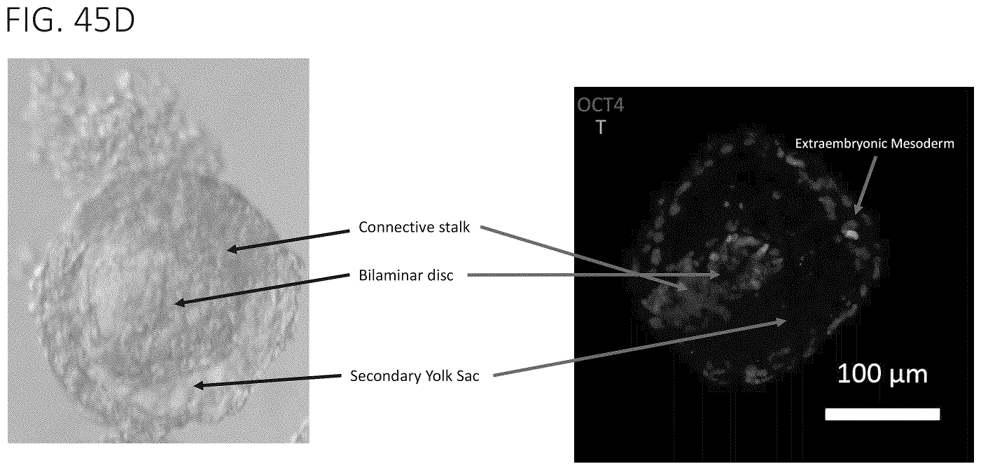

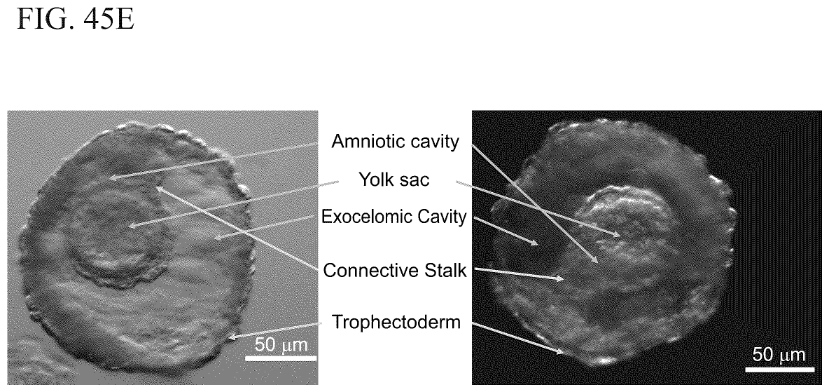

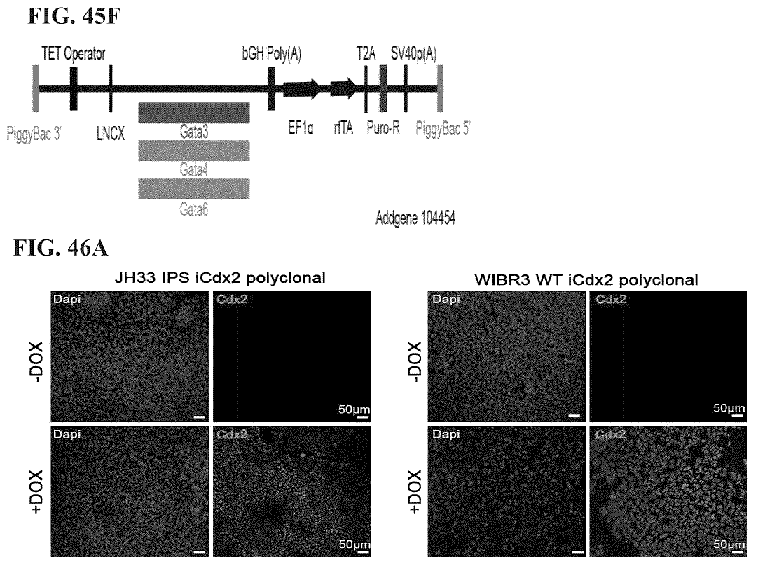

Doxycycline inducible expression lines were generated by the PiggyBac approach for transient ectopic expression of CDX2 (inducing trophectoderm lineage specification) and GATA4 (inducing primitive endoderm differentiation), as explained in Example 4 hereinabove. Following, these lines were kept in HENSM medium (5 ng/ml activin) on mouse embryonic fibroblasts. Both generated lines and the wild type line were synchronized to reach confluence on the same day. 24 hours prior to aggregation the iCDX2 line was transferred into TIM (trophoblast inducing media-N2B27 supplemented with PD0325901, A-83, LIF, LPA, Y27632) plus Doxycycline. The iGata4 line was transferred to aggregation medium (1:1 ratio neurobasal DMEM f12, sodium pyruvate, GlutaMAX, penicillin streptomycin, N2 Supplement, B27 supplement, BSA and b-mercaptoethanol) plus Y27632 and Doxycycline. Following 24 hours of induction the three cell lines were harvested using TrypLE enzyme centrifuged and re-suspended in MEF medium (DMEM, sodium pyruvate, Penicillin Streptomycin, L-Glutamine and 20% FBS), posteriorly cells were plated on gelatin coated plates for 30 minutes to deplete MEFs. Afterwards cells in suspension were collected and passed through a 70 μm strainer and re-suspended in 1 ml for counting. Cells were plated in AggreWell 400 in concentrations of 36.9 iCDX2, 11.5 iGATA4, 11.5 naïve stem cells (also referred to as WT) per 400 μm microwell, on N2B27 medium supplemented with Y27632 (10 μm final concentration) and Doxycycline, cells were centrifuged to ensure dropping on the microwells. Plate was incubated in a 5% oxygen incubator. Next day the medium was exchanged for N2B27 Medium with Doxycycline and the following day to N2B27 alone. On day +3 aggregates were transferred to a non-adherent 6-well plate (2400 aggregates per well) on EUCM2+20% FBS medium (In this Example, EUCM2 refers to Advanced DMEM f12, T3, extra D-Glucose, 1% CMRL, ITSX, Penicillin Streptomycin, GlutaMAX, progesterone, b-estradiol, n-acetyl-n Cysteine with the indicated percentage of FBS) on day +4 FBS was increased to 30%, on day +5 to 50% and on day +6 to 75%. A schematic representation of the protocol is shown in FIG. 45A.

### Results

Co-aggregation of the three stem cell populations, all originating from naïve ESCs/iPSCs as a starting material, followed by ex-utero culturing enabled generation of organized embryos, as exemplified in FIGS. 45A-E.

Specifically, during the second half of day 3 and day 4 (FIG. 45B) formation of the amniotic cavity characterized by a ring of columnar epithelium in the center of the aggregate with a cavity was identified. Immunohistochemistry demonstrated this ring comprises Oct4 positive cells and also showed a population of Gata4 positive cells predominantly in one side of the epiblast, a population forming the primitive endoderm.

On day +5 (FIG. 45C) the formation of two cavities was identified, the first surrounded by Oct4 cells (amniotic cavity) and the second one surrounded by Gata4 and Gata6 positive cells, giving rise to the yolk sac. Cells on the outer layer of the aggregates showed a strong staining for Gata3 forming the trophectoderm layer. On top of the Oct4 positive ring, a change of epithelium from a columnar to a squamous architecture losing the expression of Oct4 was evident, indicating the formation of the amnion. Also, some brachyury positive cells were also detected emerging from the posterior portion of the disc showing evidence of anterior/posterior symmetry breaking and formation of the extraembryonic mesoderm.

On Day +6 (FIG. 45D), a secondary yolk sac was evident by bright field imaging, starting to grow surrounding the bilaminar disc already formed leaving the disc hanging inside the cavity connected only by the connective stalk. Further, the extraembryonic mesoderm started to form a layer on the inner part of the secondary yolk sac characterized by brachyury expression.

On day +7 (FIG. 45E) the aggregates recapitulated a day post fertilization 14-15 human embryo. With bright field microscopy, the two cavities forming the bilaminar were identified, both of them connected by the epiblast and the formation the exocoelomic cavity was also evident.

Interestingly, the equivalence between the culturing days and embryonic stages was as follows: Day 0—PFD5; Day 1—PFD6; Day 2—PFD7; Day 3—PFD8; Day 4—PFD9; Day 5-PFD10; Day 6—PFD11; Day 7—PFD12; Day 8—PFD 13; Day 9—PFD14.

### Example 6

### Generation of Human Naïve PSCs Transiently Expressing GATA4, CDX2 and/or GATA3

### Materials and Methods

Gata4, Gata6, Cdx2, Gata3 inducible naïve human pluripotent stem cells-A PiggyBac plasmid expressing a cDNA insert of choice under the control of a doxycycline-inducible promotor was designed (FIG. 45F). The Piggybac vector used (www (dot) addgene (dot) org/104454/), carries M2Rtta and a site for cDNA insert of transcription factor of interest. This vector was used to generate 4 different plasmids: human CDX2 or Gata3 (to promote human PSC differentiation towards trophectoderm) and human GATA4 or GATA6 (to promote human primitive endoderm priming from human PSCs.

Expansion and maintenance of human naïve pluripotent cells—As described in Example 4 hereinabove.

Immunostaining—As described in Example 1 hereinabove. Antibodies used are shown in Table 1 herein below.

### Results

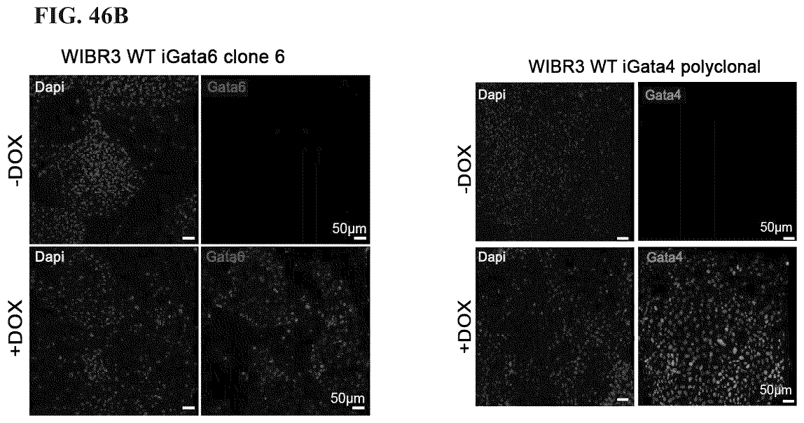

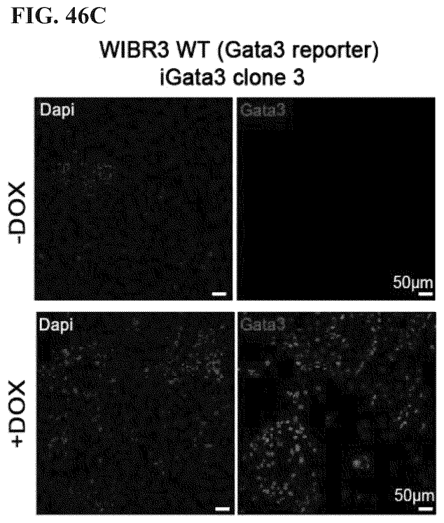

Naïve human PSCs that transiently overexpress Cdx2 or Gata3 upon addition of DOX in the early stages of embryo aggregation protocol were generated, in order to induce their rapid priming and transition towards early trophectoderm (TE). In addition, naïve human PSCs that transiently overexpress Gata4 or GATA6 upon addition of DOX in the early stages of embryo aggregation protocol were generated, in order to induce their rapid priming and transition towards the extraembryonic primitive endoderm lineage. As shown in FIGS. 46A-C, CDX2, GATA4, GATA6 or GATA3 expression was specifically detected in the transduced cells only after DOX addition.

### Example 7

### Alternative Conditions for Obtaining Mouse Naïve PSCs as the Starting Population for Generating Embryos

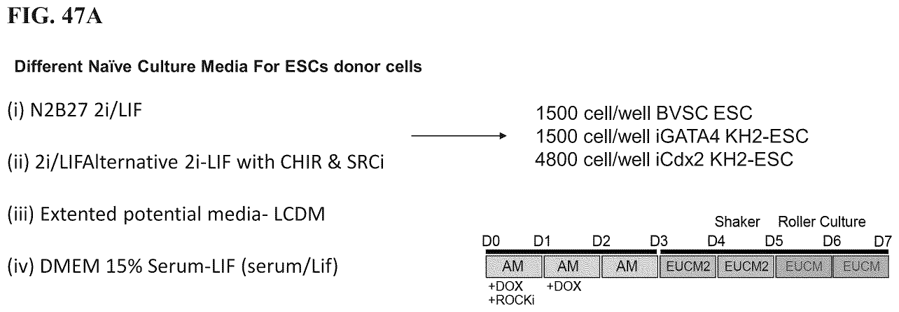

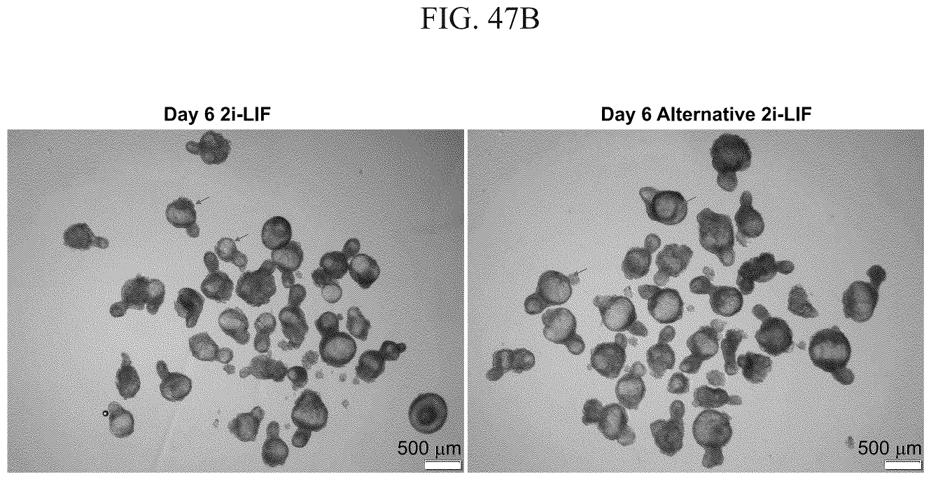

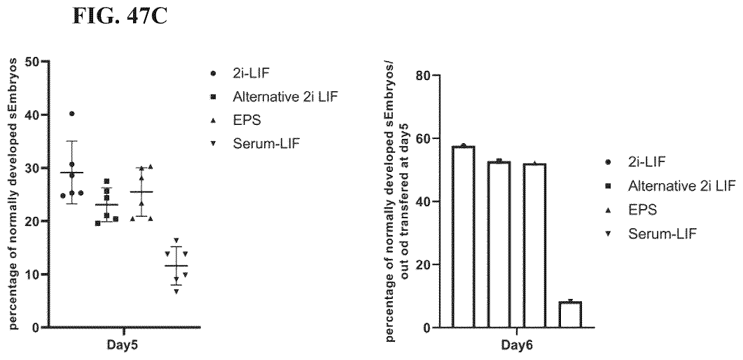

Alternative conditions to generate naïve PSCs as the starting population for generating organized embryos was were tested (FIGS. 47A-C). Under these conditions, naïve human PSCs that transiently overexpress Cdx2 upon addition of DOX and naïve human PSCs that transiently overexpress Gata4 upon addition of DOX were generated. Indeed, as explained in details hereinbelow, co-aggregation of the three stem cell populations, followed by ex-utero culturing enabled generation of organized embryos, as exemplified in FIGS. 47B-C.

FIG. 47A shows a schematic representation of the protocol.

WT ESCs were grown in one of the following media:
(i) N2B27 2i/LIF: serum-free chemically defined N2B27-based media: 240 ml Neurobasal (ThermoFisher 21103049) and 240 ml of DMEM-F12 with HEPES (SIGMA D6421), 5 ml N2 supplement (Invitrogen; 17502048), 5-10 ml B27 supplement (Invitrogen; 17504044), 2 mM glutamine or 1 mM GLutamax (Invitrogen), 1% nonessential amino acids (Invitrogen), 0.1 mM β-mercaptoethanol (Sigma), 1% penicillin-streptomycin (Invitrogen). Optionally, the medium also included 5 mg/ml BSA (Sigma). Naïve 2i/LIF conditions for murine PSCs included 20 ng/ml recombinant human LIF (in-house made). 2i was added: small-molecule inhibitors CHIR99021 (CH, 3 μM—Axon Medchem) and PD0325901 (PD, 1 μM—Axon Medchem 1408).
(ii) alternative 2i/LIF conditions (a2i or a2i-Lif conditions): serum-free chemically defined N2B27-based media: 240 ml Neurobasal (ThermoFisher 21103049) and 240 ml of DMEM-F12 with HEPES (SIGMA D6421), 5 ml N2 supplement (Invitrogen; 17502048), 5-10 ml B27 supplement (Invitrogen; 17504044), 2 mM glutamine or 1 mM GLutamax (Invitrogen), 1% nonessential amino acids (Invitrogen), 0.1 mM β-mercaptoethanol (Sigma), 1% penicillin-streptomycin (Invitrogen). Optionally, the medium also included 5 mg/ml BSA (Sigma). Naïve a2i/LIF conditions for murine PSCs included 20 ng/ml recombinant human LIF (in-house made). Small-molecule inhibitors CHIR99021 (CH, 3 μM—Axon Medchem) and SRCi CGP77675 1 microM, Axon 2097). This is an example of naïve PSCs conditions that do not involve inhibitors of the FGF/MEK/ERK pathway. Validation of this alternative condition to obtain naïve PSCs has been described in Choi, J., Huebner, A., Clement, K. et al. Nature 548, 219-223 (2017), www(dot)doi(dot)org/10.1038/nature23274.
(iii) Extended potential LCDM media [as described in Yang et al. Cell (2017) 169 (2): 243-257, www(dot)cell(dot)com/fulltext/S0092-8674% 2817%2930183-6]. Serum-free chemically defined N2B27-based media: 240 ml Neurobasal (ThermoFisher 21103049) and 240 ml of DMEM-F12 with HEPES (SIGMA D6421), 5 ml N2 supplement (Invitrogen; 17502048), 5-10 ml B27 supplement (Invitrogen; 17504044), 2 mM glutamine or 1 mM GLutamax (Invitrogen), 1% nonessential amino acids (Invitrogen), 0.1 mM β-mercaptoethanol (Sigma), 1% penicillin-streptomycin (Invitrogen). Optionally, the medium also included 5 mg/ml BSA (Sigma). LCDM (or also known as EPS) conditions for murine PSCs included 20 ng/ml recombinant human LIF (in-house made). Small-molecule inhibitors: CHIR 99021 (C, human: 1 mM, mouse: 3 mM; Tocris, 4423), (S)-(+)-Dimethindene maleate (D, 2 mM; Tocris, 1425) and Minocycline hydrochloride (M, 2 mM; Santa Cruz Biotechnology, sc-203339).
(iv) Serum/Lif (15% FBS/LIF (LIF 10 ng/ml) DMEM based conditions. 450 ml DMEM (TermmoFisher 241965-039), 2 mM glutamine or 1× GLutamax (Invitrogen), 1× nonessential amino acids (NEAA) (Invitrogen), 1× penicillin-streptomycin (Invitrogen), 1% Sodium-pyruvate (BI 03-042-1B), supplemented with 15% (v/v) FBS (Gibco or Biological Industries).
   To generate the synthetic embryos, three starting PSC cell types were co-aggregated:
1) Naïve WT BVSC in either 2i/LIF, a2i/LIF, LCDM or serum/Lif conditions as indicated;
2) Naïve iGata4 ESCs in either 2i/LIF, a2i/LIF, LCDM or serum/Lif conditions as indicated, and pre-treated with DOX (2 μg/ml—Sigma D9891) in the same media for 24 hours prior to the co-aggregation; and
3) Naïve iCdx2 ESCs in either 2i/LIF, a2i/LIF, LCDM or serum/Lif conditions as indicated, pre-treated with DOX (2 μg/ml—Sigma D9891) for 24 hours prior to the co-aggregation (from day −1) in TSC media (25 ng/ml FGF4 (Peprotech), 1 μ/ml Heparin (Sigma)) supplemented with lysophosphatidic acid (LPA, a Hippo pathway inhibitor) 01 μM.

Co-aggregation was defined as time point 0 of the protocol. At the day of aggregation (day 0), the three donor cell populations were trypsinized with 0.05% trypsin-EDTA solution (Biological Industries—Sartorius 03-053-1B) for 4 minutes at 37° C. Trypsin enzymatic reaction was stopped by adding aggregation media. Cells were centrifuged at 1200 rpm for 3 minutes and resuspended in aggregation medium with DOX (2 μg/ml—Sigma D9891) and ROCKi Y27632 (5 nM final concentration—Axon Medchem 1683). Following, cells were plated on gelatinized tissue culture plates for mouse embryonic fibroblast depletion for 20 minutes at 37° C. Supernatant was collected, centrifuged and cells were resuspended. The three cell fractions were counted and resuspended in aggregation media with DOX (2 μg/ml—Sigma D9891) and ROCKi Y27632 (5 nM—Axon Medchem 1683). A ratio of (1 WT-ESCs:1 iGata4 ESC:3.33 iCdx2 ESC) was maintained in aggregation experiments, as follows:

| | Number of | Number of added | # of Cells |
|---|---|---|---|
| | microwells per well | cells per each well | per single |
| | in 24 well plate | of a 24 well plate | microwell |
| AggreWell | 300 | 1-iCDX2: 4800 cells | ~27 cells |
| 800 | | 2-iGATA4: 1500 cells | |
| | | 3-WT ESC 1500 cells | |

1 ml of cell-mix suspension was gently added drop wise to each well of the AggreWell plate followed by centrifugation at 700 rpm for 3 minutes and incubation at 37° C. (total end volume is 1.5 ml).

On the next day (day 1), 1 ml of media (out of total 1.5 ml) was gently removed from each well and replaced with 1 ml of preheated aggregation media with DOX (2 μg/ml—Sigma D9891).

On day 2, 1 ml of media was removed from each well and replaced with 1 ml of preheated aggregation media.

On day 3, 1 ml of media was removed from each well and replaced with 1 ml of preheated EUCM2 media. In this example, EUCM2 media refers to: Advanced DMEM/F12 (GIBCO 21331-020), 5 ml CMRL media (1% final concentration) as a supplement (5mlGIBCO 11530037), extra added 1 mg/ml D (+)-Glucose Monohydrate (J.T. Baker—0113) (add 500 mg per 500 ml media), 100 nM T3 (3,3′,5-Triiodo-L-thyronine sodium salt) (Sigma-Aldrich, T6397), 1 mM Sodium pyruvate (Sigma-Aldrich, S8636), 1 mM GlutaMAX (GIBCO, 35050061), 1% penicillin streptomycin (Biological Industries—Sartorius 03-031-1B), 1× of ITS-X supplement (Thermo Fisher Scientific 51500-056), 8 nM B-estradiol (Sigma-Aldrich, E8875), 200 ng/ml progesterone (Sigma-Aldrich, P0130), 25 μM N-acetyl-L-cysteine (Sigma-Aldrich, A7250)+20% FBS.

On day 4, the synthetic embryos were gently transferred to 6-well cell suspension culture plates (Greiner, 657185) with 5 ml of preheated EUCM2 media which comprises 30% FBS per well and placed on shaker rotation 60 rpm/min (Thermo 88881102+88881123).

On day 5, egg cylinder shape sEmbryos were picked and transferred to glass culture bottles (50-30 embryos per bottle) containing 2 mL of EUCM media (the contents of which are described in Example 1 hereinabove). Following, the bottles were placed on the rolling culture system previously described electronically controlled roller culture platform (Aguilera-Castrejon et al., 2021a), rotating at 30 revolutions per minute at 37° C., and continuously gassed with an atmosphere of 21% O₂, 5% CO₂ at 7-8 pounds per square inch (psi). A black non-transparent cloth was used to cover the incubator from light during unit operation.

From day 6 to day 8, 1 ml of EUCM was replaced with preheated 1 ml of EUCM and kept on rolling culture system. Culture media was pre-heated for at least an hour by placing it inside a glass bottle on the rotating culture.

As shown in FIGS. 47B-C, egg cylinder shaped embryos were obtained following the protocols comprising the different culturing conditions.

### Example 8

### A Two Steps Aggregation Protocol Enhances Efficiency of Embryos Formation

### Materials and Methods

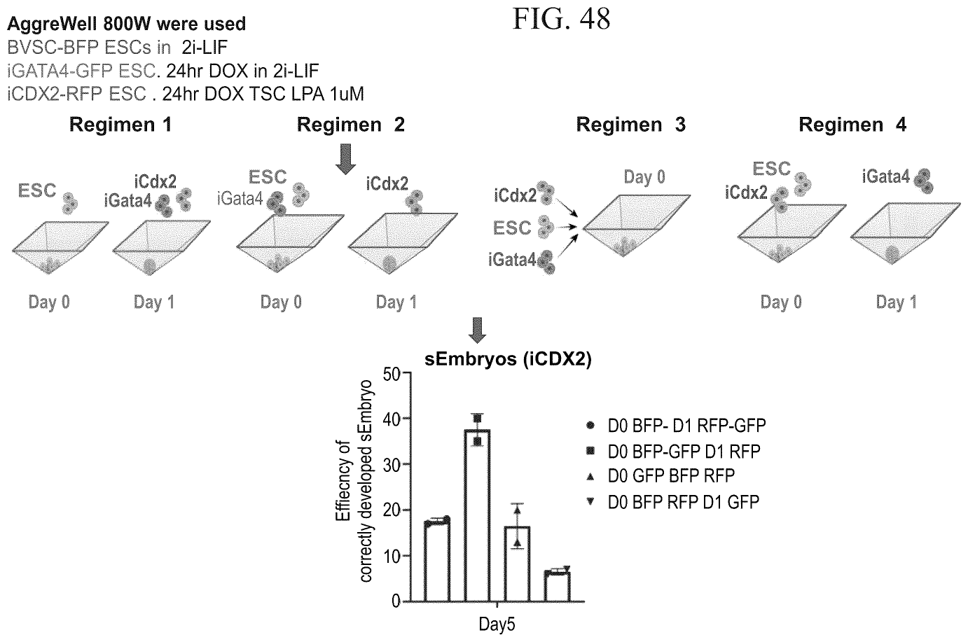

To generate the synthetic embryos, three starting PSC cell types were co-aggregated (FIG. 48):
1) Naïve WT BVSC in 2i/LIF constitutively labeled with BFP (Blue) encoding lentivirus;
2) Naïve iGata4 ESCs in 2i/LIF, labeled with GFP (Green) encoding lentivirus, pretreated with DOX (2 μg/ml—Sigma D9891) for 24 hours before aggregation (from day −1) in 2i/Lif media; and
3) Naïve iCdx2 ESCs in 2i/LIF, labeled with RFP (Red) encoding lentivirus, pretreated with DOX (2 μg/ml—Sigma D9891) for 24 hours before aggregation (from day −1) in TSC media (25 ng/ml FGF4 (Peprotech), 1 μ/ml Heparin (Sigma) supplemented with lysophosphatidic acid (LPA, a Hippo pathway inhibitor) 01 μM.
   Co-aggregation was defined as time point 0 of the protocol.

At the day of aggregation (day 0) or one day later (day 1), either one or two or all three donor cell populations were trypsinized with 0.05% trypsin-EDTA solution (Biological Industries—Sartorius 03-053-1B) for 4 minutes at 37° C. Trypsin enzymatic reaction was stopped by adding aggregation media. Cells were centrifuged at 1200 rpm for 3 minutes and resuspended in aggregation medium with DOX (2 μg/ml—Sigma D9891) and ROCKi Y27632 (5 nM final concentration—Axon Medchem 1683). Following, cells were plated on gelatinized tissue culture plates for mouse embryonic fibroblast depletion for 20 minutes at 37° C. Supernatant was collected, centrifuged and cells were resuspended. The cell fractions were counted and resuspended in aggregation media with DOX (2 μg/ml—Sigma D9891) and ROCKi Y27632 (5 nM—Axon Medchem 1683). A ratio of 1 Blue BVSc ESCs:1 Green-iGata4 ESC:3.33 Red-iCdx2 ESC was maintained in aggregation experiments, as follows:

| | Number of micro- | Number of added cells | # of Cells |
|---|---|---|---|
| | wells per well | per each well | per single |
| | in 24 well plate | of a 24 well plate | microwell |
| AggreWell | 300 | 1-Red-iCDX2: 4800 cells | ~27 cells |
| 800 | | 2-Green-iGATA4: 1500 cells | |
| | | 3-Blue-WT ESC 1500 cells | |

1 ml of cell-mix suspension was gently added drop wise to each well of the AggreWell plate followed by centrifugation at 700 rpm for 3 minutes and incubation at 37° C. (total end volume is 1.5 ml).

On the next day (day 1), 1 ml of media (out of total 1.5 ml) was gently removed from each well and replaced with 1 ml of preheated aggregation media with DOX (2 μg/ml—Sigma D9891). When one or 2 of the cell fractions was added on day 1 instead of day 0, 1 ml of cell-mix suspension was gently added drop wise to each well of the AggreWell plate followed by centrifugation at 700 rpm for 3 minutes and incubation at 37° C. (total end volume is 1.5 ml).

On day 2, 1 ml of media was removed from each well and replaced with 1 ml of preheated aggregation media.

On day 3, 1 ml of media was removed from each well and replaced with 1 ml of preheated EUCM2 media. In this example, EUCM2 media refers to: Advanced DMEM/F12 (GIBCO 21331-020), 5 ml CMRL media (1% final concentration) as a supplement (5mlGIBCO 11530037), extra added 1 mg/ml D (+)-Glucose Monohydrate (J.T. Baker—0113) (add 500 mg per 500 ml media), 100 nM T3 (3,3′,5-Triiodo-L-thyronine sodium salt) (Sigma-Aldrich, T6397), 1 mM Sodium pyruvate (Sigma-Aldrich, S8636), 1 mM GlutaMAX (GIBCO, 35050061), 1% penicillin streptomycin (Biological Industries—Sartorius 03-031-1B), 1× of ITS-X supplement (Thermo Fisher Scientific 51500-056), 8 nM B-estradiol (Sigma-Aldrich, E8875), 200 ng/ml progesterone (Sigma-Aldrich, P0130), 25 μM N-acetyl-L-cysteine (Sigma-Aldrich, A7250)+20% FBS.

On day 4, the synthetic embryos were gently transferred to 6-well cell suspension culture plates (Greiner, 657185) with 5 ml of preheated EUCM3 media per well and placed on shaker rotation 60 rpm/min (Thermo 88881102+88881123). In this example, EUCM3 media here (EUCM2 media but with 30% FBS is also known as EUCM3) refers to: Advanced DMEM/F12 (GIBCO 21331-020), 5 ml CMRL media (1% final concentration) as a supplement (5mlGIBCO 11530037), extra added 1 mg/ml D (+)-Glucose Monohydrate (J.T. Baker—0113) (add 500 mg per 500 ml media), 100 nM T3 (3,3′,5-Triiodo-L-thyronine sodium salt) (Sigma-Aldrich, T6397), 1 mM Sodium pyruvate (Sigma-Aldrich, S8636), 1 mM GlutaMAX (GIBCO, 35050061), 1% penicillin streptomycin (Biological Industries—Sartorius 03-031-1B), 1× of ITS-X supplement (Thermo Fisher Scientific 51500-056), 8 nM B-estradiol (Sigma-Aldrich, E8875), 200 ng/ml progesterone (Sigma-Aldrich, P0130), 25 μM N-acetyl-L-cysteine (Sigma-Aldrich, A7250)+30% FBS.

On day 5, egg cylinder shape sEmbryos were picked and transferred to glass culture bottles (50-30 embryos per bottle) containing 2 mL of EUCM media (the contents of which are described in Example 1 hereinabove). Following, the bottles were placed on the rolling culture system previously described electronically controlled roller culture platform (Aguilera-Castrejon et al., 2021a), rotating at 30 revolutions per minute at 37° C., and continuously gassed with an atmosphere of 21% O₂, 5% CO₂ at 6-8 pounds per square inch (psi). A black non-transparent cloth was used to cover the incubator from light during unit operation.

From day 6 to day 8, 1 ml of EUCM was replaced with preheated 1 ml of EUCM and kept on rolling culture system. Culture media was pre-heated for at least an hour by placing it inside a glass bottle on the rotating culture.

### Results

Four different regimens for co-aggregating the three mouse stem cell populations, all originating from naïve ESCs/iPSCs as a starting material, followed by ex-utero culturing were evaluated (FIG. 48), basically assessing a 1-step aggregation protocol vs. a two steps aggregation protocol. sEmbryo formation efficiency of egg cylinder was evaluated on day 5. As shown in FIG. 48, adding iCDX2 ESCs at day 1, rather than at day 0, resulted in two-four times enhancement of normal egg-cylinder sEmbryo formation efficiency (highlighted with a blue arrow).

### Example 9

### Generation of Trophectoderm Primed Cells by Inducing Endogenous Expression of Differentiation Factors in Naïve PSCs

### Materials and Methods

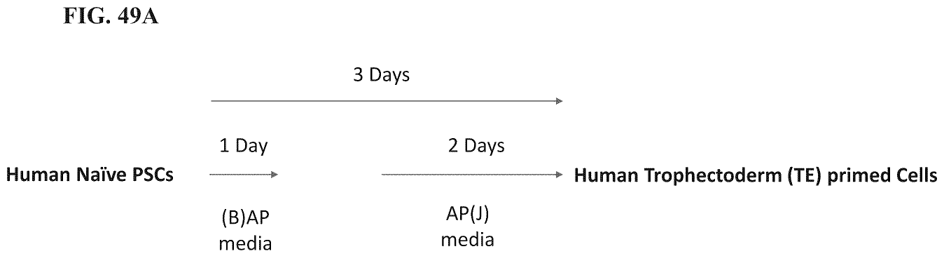

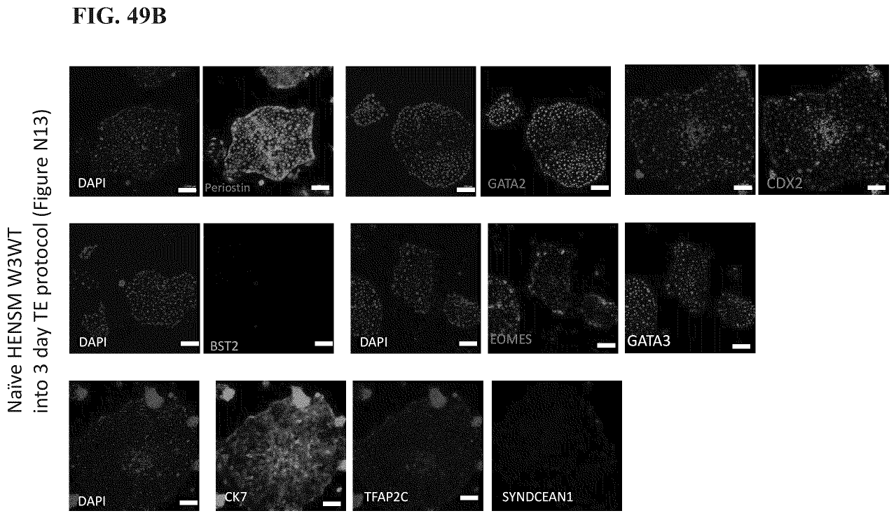

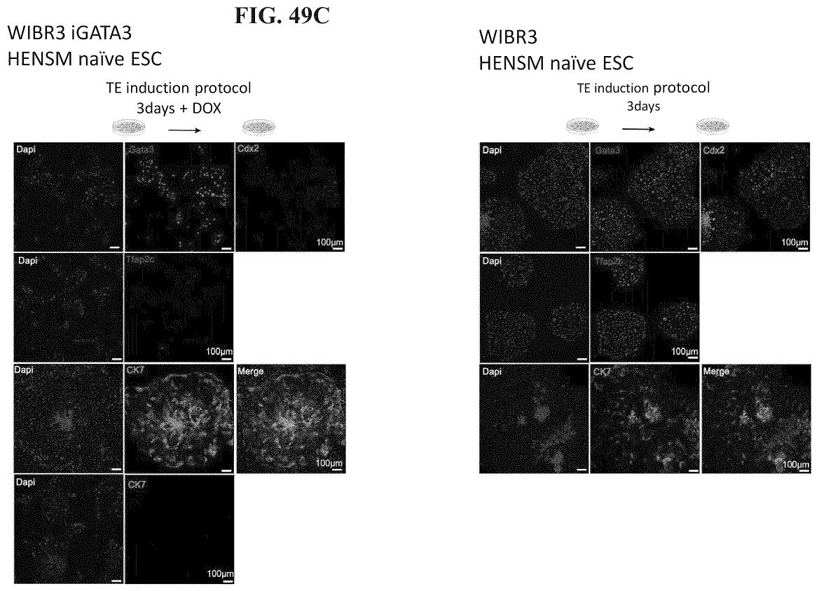

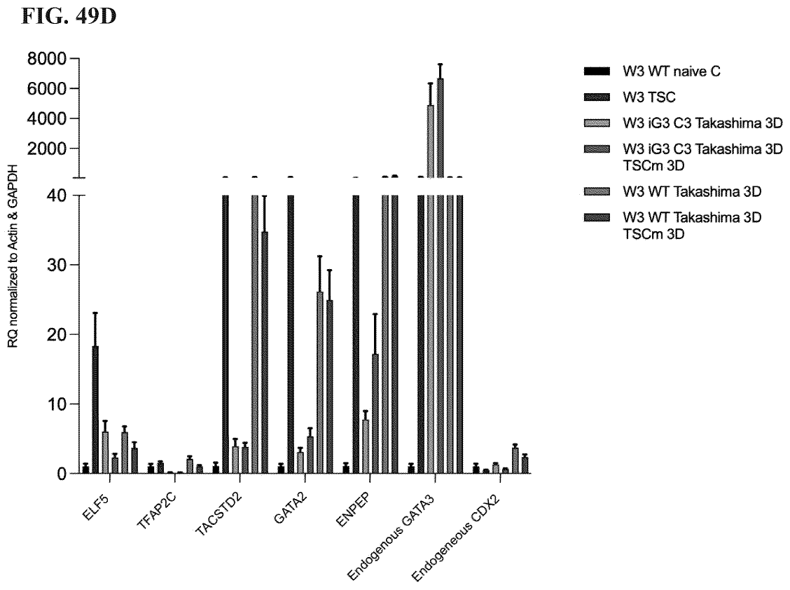

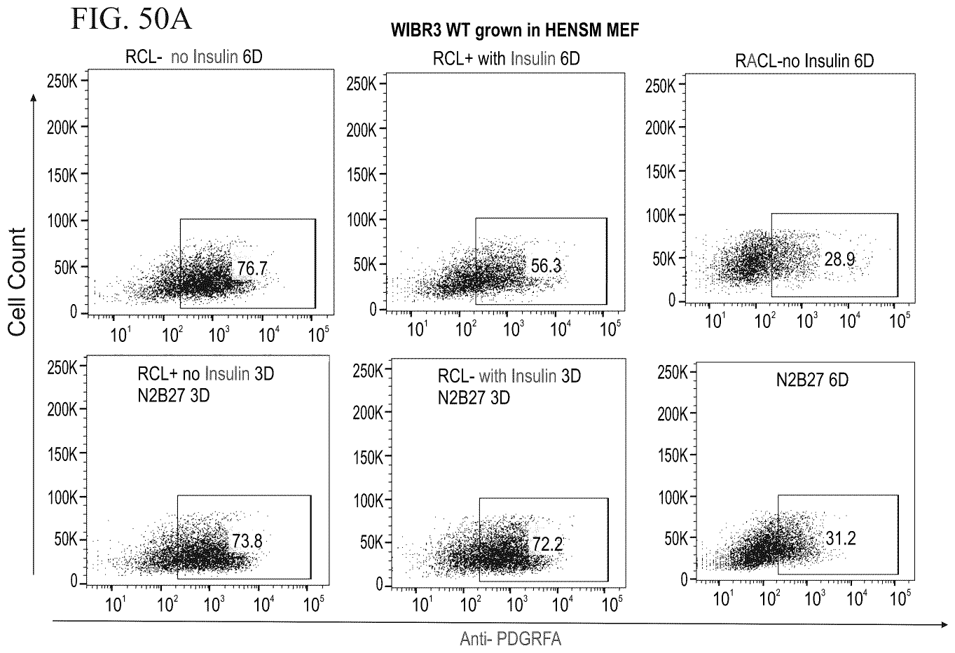

Generation of trophectoderm primed cells (FIGS. 49A-D)—Human naïve PSCs expanded in human HENSM conditions for at least 1 passage (4 days), which were plated on 1% 1% GELTREX (Thermo A1413202/A1413302) or Matrigel (BD) coated plates.

### HENSM Media:

Primary Cytokines+inhibitors:
1:1 mix of Neurobasal (Invitrogen 21103-049) and DMEM/F12 (Invitrogen 21331)—470 ml
Pen-strep 5 ml (Biological Industries 03-033-1B)
Glutamax—5 ml (Invitrogen 35050061)
NEAA—5 ml (Biological Industries 01-340-1B)
Sodium Pyruvate—5 ml (Biological Industries 03-042-1B)
10 ml B27 supplement: Invitrogen 17504-044 or Xenofree A1486701
L-ascorbic acid 2-phosphate (Sigma—A8960) (50 μg/ml final concentration) (1 vial)
Geltrex (Invitrogen A1413202/A1413302) or Matrigel—1 ml rapidly in media (0.2% final conc.)
5 ml Commercial N2 supplement (Invitrogen 17502048)
LIF (in house produced or Peprotech 300-05)—20 ng/ml final
WNTi-TNKi=XAV939 (Sigma X3004)—2 M final
PKCi Go6983 (Axon 2466)—2 μM final
FGFRi-MEK1/2i-ERK1/2i—PD0325901 (Axon 1408)—1-1.2 μM final
SRCi CGP77675 (Axon 2097)—1 μM (1 vial=50 μL).
Optionally ACTIVIN A—(Peprotech 120-14E)—5 ng/ml final.
Optionally ROCKi Y27632 (Axon 1683)—1 μM.
Optionally BIRB796 P38i (0.4 μM)
   A 3 days protocol for human trophoblast induction from human PSCs (using media adapted from Shingo Io et al. (2021) Cell Stem Cell, 28 (6): 1023-1039 and Mitsuyoshi Amita et al. (2013) PNAS E1212-1221):
   Step 1: In the first 24 hours (B)AP media was used:
1:1 mix of Neurobasal (Invitrogen 21103-049) and DMEM/F12 (Invitrogen 21331)—470 ml
Pen-strep 5 ml (Biological Industries 03-033-1B)
Glutamax—5 ml (Invitrogen 35050061)
NEAA—5 ml (Biological Industries 01-340-1B)
Sodium Pyruvate—5 ml (Biological Industries 03-042-1B)
10 ml B27 supplement: Invitrogen 17504-044 or Xenofree A1486701
L-ascorbic acid 2-phosphate (Sigma—A8960) (50 μg/ml final concentration) (1 vial)
5 ml Commercial N2 supplement (Invitrogen 17502048)
(A) TGFRi A83-01—2 μM final concentration (Axon Medchem A83-01)
(P) FGFRi/MEKi/ERK PD0325901—2 μM final concentration (Axon Medchem 1408) [FGFRi PD173074 0.5-1 μM can be used instead]
(J) human recombinant BMP4 10 ng/ml was optionally included (Peprotech 120-05ET or 120-05).
   Step 2: In the following 48 hours (from day 2-day 3 of pretreatment) AP(J) media was used:
1:1 mix of Neurobasal (Invitrogen 21103-049) and DMEM/F12 (Invitrogen 21331)—470 ml
Pen-strep 5 ml (Biological Industries 03-033-1B)
Glutamax—5 ml (Invitrogen 35050061)
NEAA—5 ml (Biological Industries 01-340-1B)
Sodium Pyruvate—5 ml (Biological Industries 03-042-1B)
10 ml B27 supplement: Invitrogen 17504-044 or Xenofree A1486701
L-ascorbic acid 2-phosphate (Sigma—A8960) (50 μg/ml final concentration) (1 vial)
5 ml Commercial N2 supplement (Invitrogen 17502048)
(A) TGFRi A83-01—2 μM final concentration (Axon Medchem A83-01)
(P) FGFRi/MEKi/ERK PD0325901—2 μM final concentration (Axon Medchem 1408) [FGFRi PD173074 0.5-1 μM can be used instead]
(J) human JAK inhibitor I—1 M final concentration was optionally included (Calbiochem 420099).
Gelatin/irradiated MEF coated plates or Merck Silk Laminin-511 (Dilute 1:200 in PBC to get working solution: 2.5 microg/ml) can be used instead of the coated Geltrex or Matrigel coated plates.

Immunostaining—As described in Example 1 hereinabove. Antibodies used are shown in Table 1 hereinbelow.

Gata3 inducible expression-A PiggyBac plasmid expressing a cDNA insert of choice under the control of a doxycycline-inducible promotor was designed (FIG. 45F). The Piggybac vector used (www (dot) addgene (dot) org/104454/), carries M2Rtta and a site for cDNA insert of transcription factor of interest. This vector was used to generate Gata3 (to promote human PSC differentiation towards trophectoderm and was introduced in human PSCs.

PCR—as described in in Example 1 hereinabove. List of primers is provided in Table 2 hereinbelow.

### Results:

Examples 2-8 hereinabove describe generation of synthetic embryos by co-aggregation of three subpopulations of stem cells all originating from naïve PSCs. The trophectoderm primed cells were obtained by transient expression of exogenous CDX2 or Gata3. In the next step, the present inventors developed culturing conditions of naive human PSCs allowing induced expression of endogenous differentiation factors leading to generation of the trophectoderm primed cells without the need for genetic modifications of the cells (FIGS. 49A-D).

Immunostaining characterization of the human trophectoderm primed cells induced according to the protocol described hereinabove demonstrated expression of known trophectoderm markers—CDX2, GATA2, Periostin, EOMES, CK7 and TFAP2C; and no expression of BST2, which marks extra-embryonic mesoderm and SYNDECAN1, which is known to emerge in later developed TSCs (FIG. 49B). Comparative analysis of 3 days trophectoderm primed cells with and without transgenic GATA3 overexpression. WT WIBR3 and iGATA3 WIBR3 naïve HPSCs were subjected side by side to the trophoblast induction protocol described hereinabove. Immunostaining characterization demonstrated that transgenic expression of GATA3 compromised the expression of the trophoblast markers TFAP2C and CK7, which was expressed only on some of the colonies of iGATA3 cells, while it was uniformly expressed on all WT cells (FIG. 49C). CDX2 expression level was much less abundant following ectopic expression of iGata3 cells compared to absence of ectopic exogenous GATA3 overexpression. RT PCR expression of different trophectoderm markers on the WT and iGATA3 WIBR3 cells isolated from experiment described in FIG. 49D. WT naïve TSCs were used as reference negative control (set as 1). WiBR3 derived TSC line was used as a reference control for some of the markers. Some samples included a regimen involved 3 d expansion in Takashima differentiation protocol (Takashima protocol=the protocol described hereinabove; and then followed by 3 more days expansion in TSC media described in Example 13 hereinbelow). Most optimal results were obtained with WT donor naïve ESCs without overexpression of exogenous GATA3 (iG3). TE markers include: CDX2, GATA3, ENPEP, GATA2, TACSTD2, TFAP2C, ELF5 (and are SIGLEC6 negative).

### Example 10

### Generation of Extra Embryonic Primitive Endodermal Primed Cells by Inducing Endogenous Expression of Differentiation Factors in Naïve PSCs

### Materials and Methods

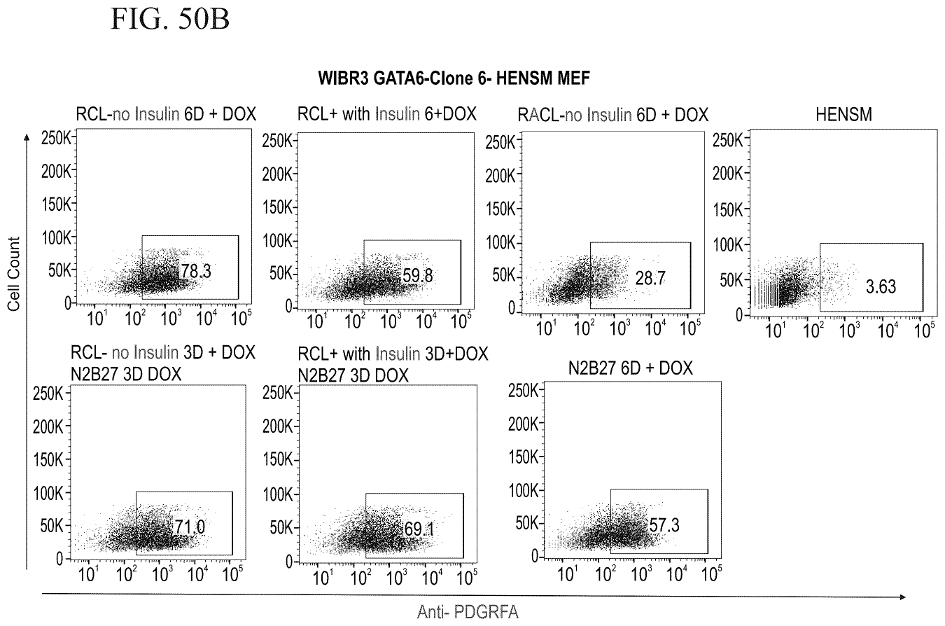

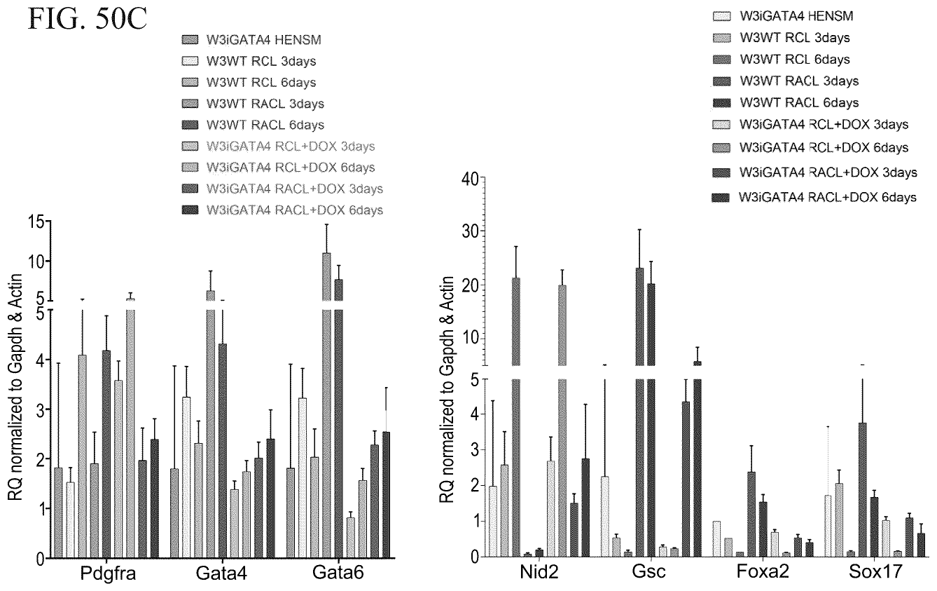

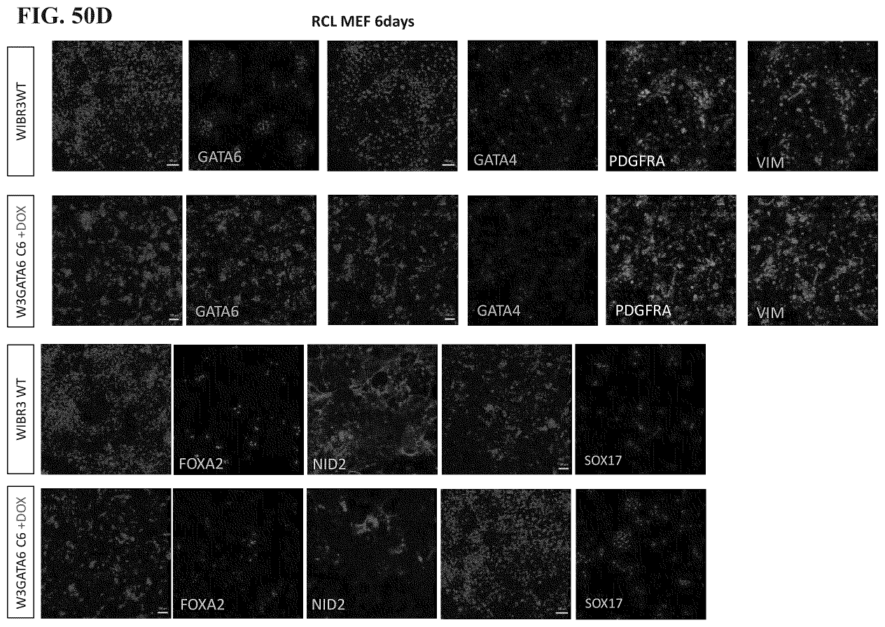

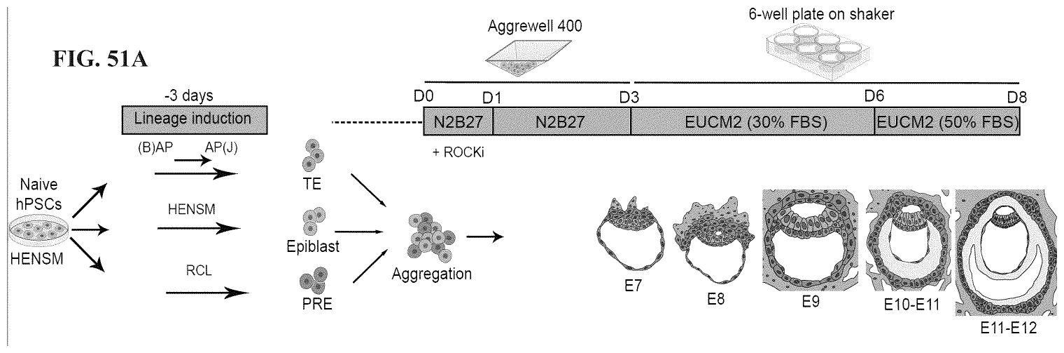

Generation of extra embryonic primitive endodermal primed cells (FIGS. 50A-D)—Previously, Josh Brickamn group has published a condition termed RACL that is able to induce primitive endoderm progenitor cells (PRE) at undefined efficiency from human naïve PSCs. FACS analysis for human PDGFRa, which marks PRE lineage, was used as an initial screen to see rapid and measure kinetics of PDGFRA induction levels (RnD systems AF-307-NA or FAB1062G). RACL media was assembled as previously published (www(dot)doi(dot)org/10.1242/dev(dot)180620) and contained: 480 ml RPMI media (21875-034 GIBCO Invitrogen), 10 ml B27 supplement (Invitrogen A18956-01 B27 without insulin), 1 mM GLutamax (Invitrogen), 1% nonessential amino acids (Invitrogen), 1% penicillin-streptomycin (Invitrogen), 100 ng/ml ActA, 3 μM CHIR99021 and 10 ng/ml LIF. An optimized RCL media which contained the same composition as RACL but without adding recombinant Activin was also used. Of note, the work of Brickman teaches away from RCL media, as it claims ACTIVIN is essential for PRE induction. Versions of RCL and RACL with B27 that was further supplemented with insulin (Invitrogen; 17504044) was also tested. Further, another version of the media tested was a N2B27 media not supplemented with CHIR99021 and LIF, making the media composed of:
1:1 mix of Neurobasal (Invitrogen 21103-049) and DMEM/F12 (Invitrogen 21331)—470 ml
Pen-strep 5 ml (Biological Industries 03-033-1B)
Glutamax—5 ml (Invitrogen 35050061)
NEAA—5 ml (Biological Industries 01-340-1B)
Sodium Pyruvate—5 ml (Biological Industries 03-042-1B)
10 ml B27 supplement: Invitrogen 17504-044 or Xenofree A1486701
5 ml Commercial N2 supplement (Invitrogen 17502048)

FACS analysis—Cell were immunostained with fluorescent labeled antibodies as indicated in Table 1 hereinbelow, washed with PBS and analyzed on FACS ARIA III and analyzed with a FACS DIVA software.

Immunostaining—As described in Example 1 hereinabove. Antibodies used are shown in Table 1 hereinbelow.

Gata6 inducible expression—As described in in Example 9 hereinabove but with Gata6 as an insert.

PCR—A described in Example 1 hereinabove. List of primed is provided in Table 2 hereinbelow.

### Results:

Examples 2-8 hereinabove describe generation of synthetic embryos by co-aggregation of three subpopulations of stem cells all originating from naïve PSCs. The extra embryonic primitive endodermal primed cells (PRE) were obtained by transient expression of GATA4 or GATA6. In the next step, the present inventors developed culturing conditions of naive human PSCs allowing induced expression of endogenous differentiation factors leading to generation of the extra embryonic primitive endodermal primed cells without the need for genetic modifications of the cells.

FACS analysis with an anti-PDGFRa antibody after 6 days of induction in the indicated media (some of which were divided into two phases of 3 d and 3 d sequential media, FIG. 50A) indicated that ACTIVIN supplementation decreases dramatically the efficiency of PRE cell induction. In addition, N2B27 media alone yielded a low fraction of PDGFRa positive cells, albeit at lower efficiency from RCL conditions. While using no insulin conditions yields better outcome over with insulin conditions, the effects were minimal. 3 days of RCL followed by 3 days of N2B27 also yielded excellent results, which were equivalent to these of 6 days RCL inductions (FIG. 50A). To test whether ectopic expression of GATA6 can yield better PRE induction results than those seen with WT WIBR3 cells, iGATA6 WIBR3 naive cells originally expanded in HENSM conditions and including DOX in the Pre induction media were used as indicated (FIG. 50B). Overall, the results show, that RACL is inferior to RCL conditions. Ectopic GATA6 does not yield higher efficiencies of induction than for WT cells placed in RCL media (FIG. 50B). RT-PCR analysis was conducted to verify expression of PRE markers (PDGFRa, GATA4, GATA6, Nid2, SOX17) and confirm lower levels of definitive endoderm markers (Gsc and Foxa2) (FIG. 50C). The results confirm that without the need for ectopic expression of GATA6 transgenes WT cells expanded in RCL media for 6 days yield wanted optimal gene expression patterns confirming PRE identity, rather than definitive endoderm identity. Cells expanded in HENSM conditions were used a negative reference control. Immunostaining analysis of naïve WT WIBR3 and iGATA6 cells originally expanded in HENSM conditions for at least 2 passages, and then placed in PRE induction in RCL condition on MEFs for 6 days demonstrate that the cells express GATA4, GATA6, PDGFRa, Vim, NID2 and SOX17 which mark PRE cells. FOXa2 which marks definitive endoderm cells, rather than PRE was expressed at very low levels in these PRE induction settings as expected. DAPI counterstaining was used for reference control.

### Example 11

### Generation of Organized Synthetic Human Embryos

### Materials and Methods

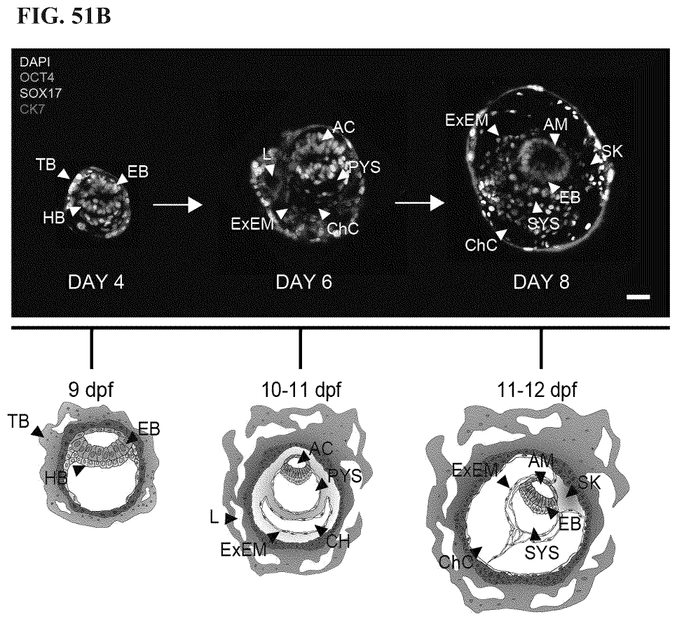

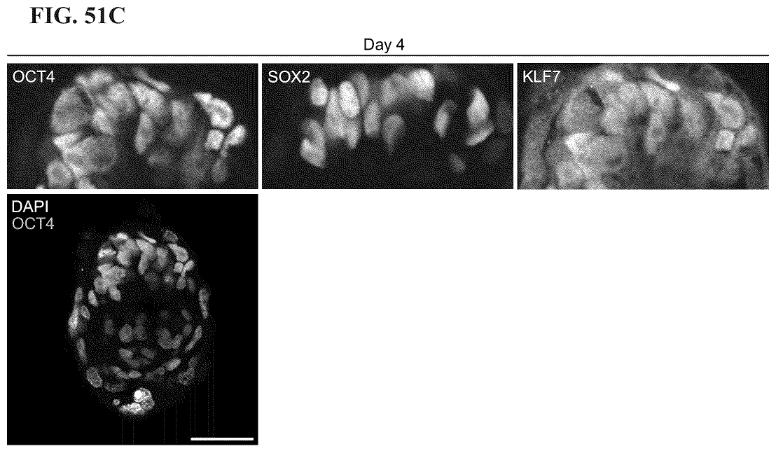

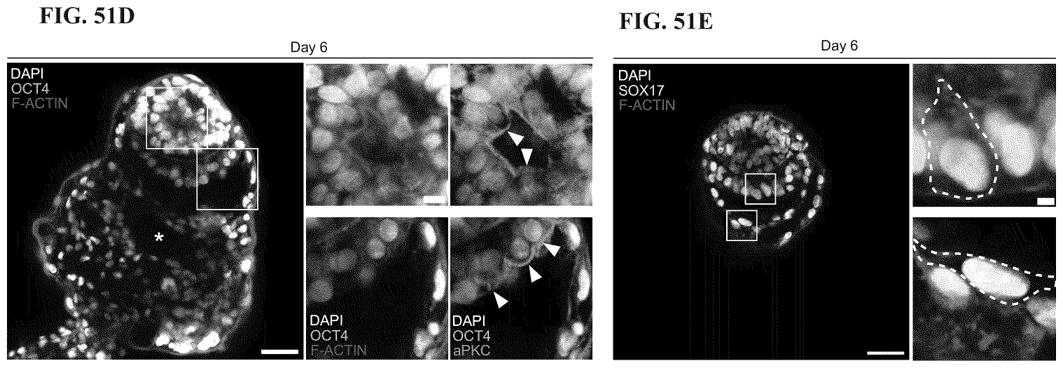

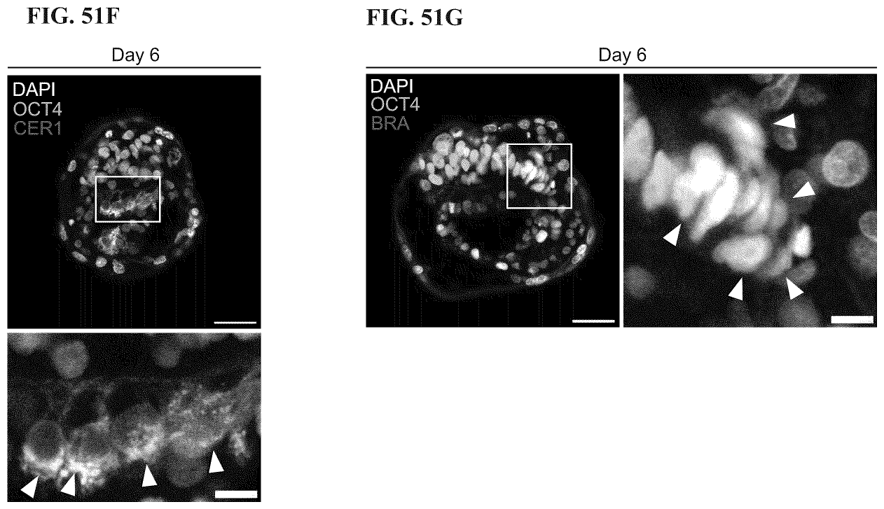

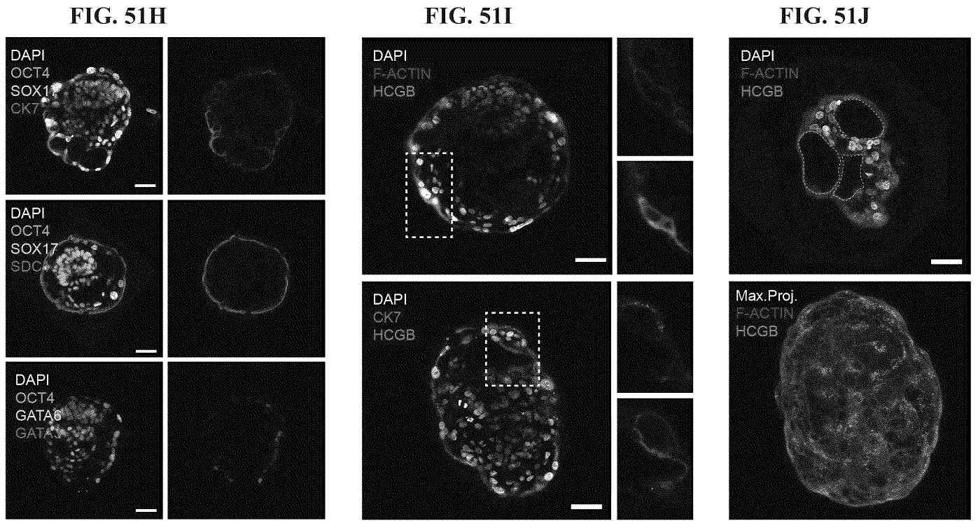

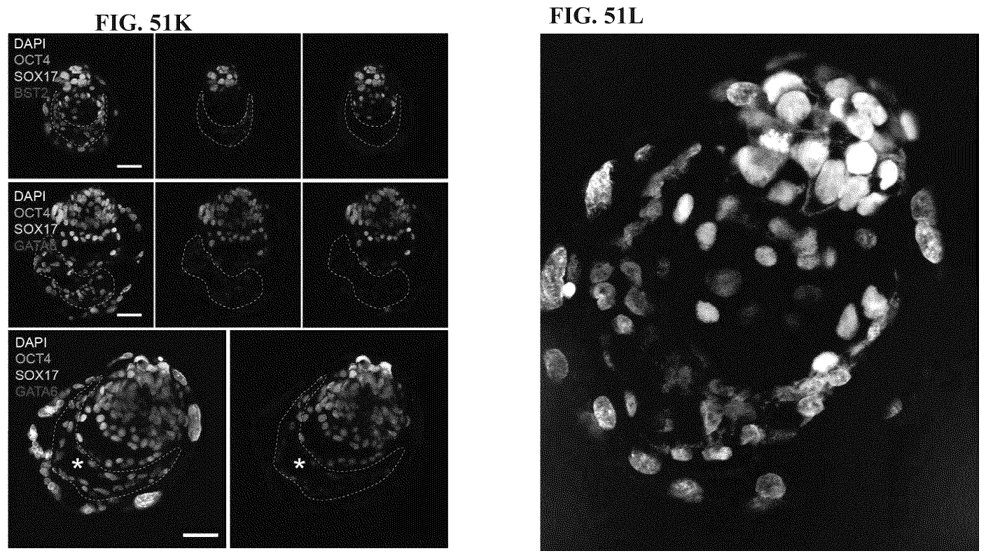

Co-aggregation and culture (FIG. 51A)—Three starting cell types were co-aggregated:
1) Naïve WT cells cultured in HENSM medium.
2) For the primitive endoderm compartment—Naïve WT cells were plated on mouse embryonic fibroblast conditions on HENSM supplemented with ROCKi 10 μM (Axon Medchem 1683), next day medium was replaced by RCL medium, RCL was kept for 72 hours with 24-hour medium exchanges.
3) For the Trophectoderm lineage—Naïve WT cells were plated on feeder free conditions (Matrigel or laminin E8) on HENSM supplemented with ROCKi 10 μM, next day medium was replaced with (B)AP medium for 24 hours followed by medium replacement to AP(J) for another 48 hours.

All three cell types were supplemented with 5 μM ROCKi Y27632 before harvesting for co-aggregation on the next day.

Co-aggregation was defined as time point 0 of the protocol.

At the day of aggregation (day 0), AggreWell plates 400, 24 wells (Stemcell technologies) were prepared according to manufacturer instructions and prefilled with 500 μL of N2B27 media and kept at 37° C. for medium equilibration. The three cell populations were disaggregated with TrypLE (Thermo Fisher 12604054) for 3 minutes for the WT and Primitive endoderm cells and 5 minutes for the Trophectoderm cells at 37° C. Following, the enzyme was removed and cells were incubated for two minutes at room temperature, afterwards cells were collected with PBS. Cells were centrifuged at 1300 rpm for 3 minutes and resuspended in N2B27 ROCKi Y27632 (10 μM final concentration). Following, primitive endoderm cells were plated on gelatinized tissue culture plates for mouse embryonic fibroblast depletion for 30 minutes at 37° C. Supernatant was collected, and all three elements were centrifuged separately and cells were resuspended on N2B27 medium. The three cell fractions were counted and resuspended in N2B27 supplemented with ROCKi Y27632 (10 μl). A ratio of 1:1:3 (WT:PRE:TE) was maintained in aggregation experiments, as follows:

| | Number of | Number of added | # of Cells |
|---|---|---|---|
| | microwells per well | cells per each well | per single |
| | in 24 well plate | of a 24 well plate | microwell |
| AggreWell | 1200 | 1-WT 27720 cells | ~120 cells |
| 400 | | 2-PrE 27720 cells | |
| | | 3-TE 88560 cells | |

0.5 ml of cell-mix suspension was gently added drop wise to each well of the AggreWell plate followed by centrifugation at 700 rpm for 3 minutes and incubation at 37° C. 5% O2 (total end volume is 1 ml).

On the next day (day 1), 900 μL of medium were gently removed from each well and replaced with 1 ml of preheated N2B27 medium. The same procedure was repeated on day 2.

On day 3 aggregates were gently transferred to 6-well cell suspension culture plates (Greiner, 657185) with 3 ml of preheated EUCM2 (20%-30% FBs) media per well and placed on shaker rotation 60 rpm/min (Thermo 88881102+88881123). Plates were transferred to 21% O2 conditions.

On day 4, 2 ml of medium were gently removed per six-well and were placed with 2 ml of preheated EUCM2 (with 30% FBS). The same procedure was repeated on day 5

On day 6, 2 ml of medium were gently removed per six-well and were placed with 2 ml of preheated EUCM2 with 50% FBS. The same procedure was repeated on day 7.

Immunostaining—As described in Example 1 hereinabove. Antibodies used are shown in Table 1 hereinbelow.

### Results:

Examples 2-8 hereinabove describe generation of synthetic embryos by co-aggregation of three subpopulations of stem cells all originating from naïve PSCs. The trophectoderm primed cells were obtained by transient expression of exogenous CDX2 or Gata3 and the extra embryonic primitive endodermal primed cells were obtained by transient expression of GATA4 or GATA6. Examples 9-10 hereinabove demonstrate culturing conditions of naive PSCs developed by the present inventors allowing induced expression of endogenous differentiation factors leading to generation of the two primed populations without the need for genetic modifications of the cells.

As exemplified in FIGS. 51A-L, co-aggregation of the naïve PSCs with the two stem cell populations described in Examples 9-10, followed by ex-utero culturing enabled generation of organized embryos. FIG. 51B demonstrates progression of human sEmbryos from Day 4 to Day 8 following aggregation. As shown in the Figure, progression of days 4, 6 and 8 sEmbryos (top) correspond to 9-12 days post fertilization (dpf) of human embryos (schematically shown at the bottom). The top panel shows human days 4-8 sEmbryos immunostained for OCT4 (Cyan), SOX17 (Yellow) and CK7 (Magenta), which are markers for epiblast, hypoblast and trophoblast, respectively. All three lineages are represented in the right location. The epiblast (Cyan) is forming a compact layer and shows expansion of amniotic cavity through time. The hypoblast (Yellow) is closing to form the primary yolk sac lining the epiblast. The trophoblast is surrounding the SEM and is forming lacunae. A population of ExEM arises, here seen as DAPI cells under the PYS, forming a chorionic cavity by day 6. The chorionic cavity expands, creating a gap separating the epiblast and yolk sac from the surrounding trophoblast by day 8. The remodelling of the ExEM will partially pinch of the primary yolk sac to form the definitive yolk sac. FIG. 51C shows characterization of epiblast pluripotency in human sEmbryos. Immunostaining of Day 4 sEmbryos demonstrates strong expression of OCT4 and SOX2, while KLf17 is expressed at low levels at this stage. FIGS. 51D-E show apical cell polarity and lumenogenesis; and FIGS. 51F-G show Anterior-posterior symmetry breaking in the human D6 sEmbryos. Trophoblast compartment characterization (FIGS. 51H-J) indicates that all three trophoblast are present in Day 6 sEmbryos and form a surrounding layer covering the structure. Also evident are the membranes and syncytiotrophoblast with presence of cyto-(CK7+ only) and syncytiotrophoblast (CK7+ and HCGB+), formation of lacuna by syncytiotrophoblast and maximum projection shows that syncytiotrophoblast surround the whole structure. Extraembryonic mesoderm evaluation (FIGS. 51K-L) indicates presence of organized extraembryonic mesoderm cells invading the space between the yolk sac (SOX17+) and the outer trophoblast layer (DAPI, SOX17−, OCT4−, BST2−). Further, the extraembryonic mesoderm cells organize and form a cavity between PYS and trophoblast cells, indicating the formation of chorionic cavity.

### Example 12

### Generation of Organized Synthetic Rhesus Macaque Embryos

### Materials and Methods

Generation of naïve Rhesus Macaque PSCs-HENSM conditions made for human naïve ESCs, were adapted for Rhesus Macaque (RM) naïve ESC and iPSC line (RM1-ESC and RM1-iPSC lines), by reducing the SRCi concentration down from 1 μM to 0.33 μM. RM1-iPSc and RM1-ESC were expanded in primed FGF/TGF conditions on mouse irradiated MEFs or in TeSR primed conditions (stem cell technologies). Monkey naïve ESC and iPSC naïve lines were expanded for at least 3 passages in HENSM conditions either on plates coated with 1% GELTREX (Thermo A1413202/A1413302) or 1% Matrigel or Biolaminin511 (5 μg/ml) or MEF/gelatin coated plates. Of note, cells were expanded in 5% O2, but it is also possible in 20% O2.

MENSM—monkey naive pluripotency media contained:
1:1 mix of Neurobasal (Invitrogen 21103-049) and DMEM/F12 (Invitrogen 21331)—470 ml
Pen-strep 5 ml (Biological Industries 03-033-1B)
Glutamax—5 ml (Invitrogen 35050061)
NEAA—5 ml (Biological Industries 01-340-1B)
Sodium Pyruvate—5 ml (Biological Industries 03-042-1B)
10 ml B27 supplement: Invitrogen 17504-044 or Xenofree A1486701
L-ascorbic acid 2-phosphate (Sigma—A8960) (50 μg/ml final concentration) (1 vial)
Optional: Geltrex (Invitrogen A1413202/A1413302) or Matrigel—1 ml rapidly in media (0.2% final conc.)
5 ml Commercial N2 supplement (Invitrogen 17502048)
LIF (in house produced or Peprotech 300-05)—20 ng/ml final
WNTi-TNKi=XAV939 (Sigma X3004)—2 μM final
PKCi Go6983 (Axon 2466)—2 μM final
FGFRi-MEK1/2i-ERK1/2i—PD0325901 (Axon 1408)—1-1.2 μM final
SRCi CGP77675 (Axon 2097)—0.33μ M
Optional: ACTIVIN A—(Peprotech 120-14E)—5-10 ng/ml final.
Optional: BIRB0796 P38i (0.4 μM)

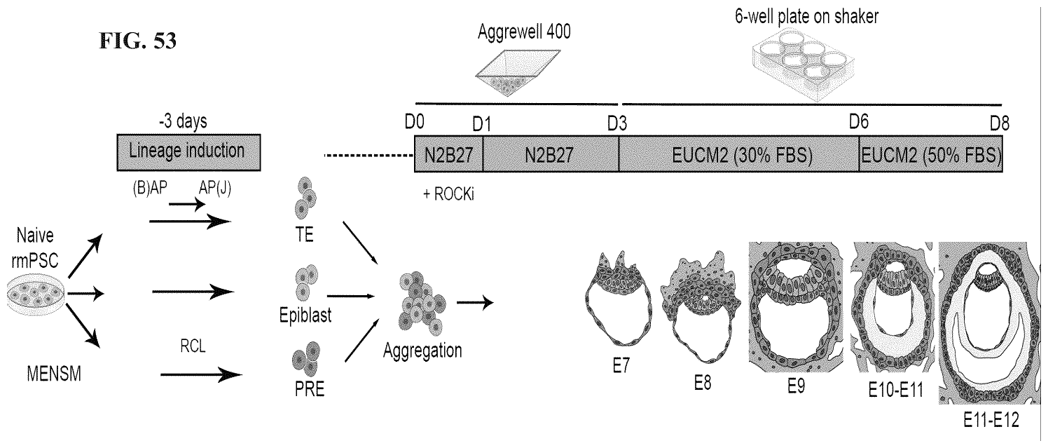

Generation of trophectoderm primed cells and extra embryonic primitive endodermal primed cells (FIG. 53)—To generate non-human primate (NHP—Rhesus Macaque sEmbryos, three starting cell types were co-aggregated:
1) Naïve WT eSC/iPSc rhesus macaque cells cultured in MENSM medium.
2) For the primitive endoderm compartment—Naïve WT RM1 ESC/iPSC were plated on mouse embryonic fibroblast conditions on MENSM supplemented with ROCKi 10 μM (Axon Medchem 1683), next day medium was replaced by RCL medium, RCL was kept for 72 hours with 24-hour medium exchanges.
3) For the Trophectoderm lineage—Naïve WT RM1 ESC or RM1 iPSCs cells were plated on feeder free conditions (Matrigel or laminin E8) on MENSM supplemented with ROCKi 10 μM, next day medium was replaced with (B)AP medium for 24 hours, followed by medium replacement with AP(J) for another 48 hours.

Co-aggregation and culture (FIG. 53)—All three cell types were supplemented with 5 μM ROCKi Y27632 before harvesting for co-aggregation on the next day. Co-aggregation was defined as time point 0 of the protocol. At the day of aggregation (day 0), AggreWell plates 400, 24 wells (Stemcell technologies) were prepared according to manufacturer instructions and prefilled with 500 μL of N2B27 media and kept at 37° C. for medium equilibration. The three cell populations were disaggregated with TrypLE (Thermo Fisher 12604054) for 3 minutes for the WT and Primitive endoderm cells and 5 minutes for the Trophectoderm cells at 37° C. Following, the enzyme was removed and cells were incubated for two minutes at room temperature, afterwards cells were collected with PBS. Cells were centrifuged at 1300 rpm for 3 minutes and resuspended in N2B27 ROCKi Y27632 (10 μM final concentration). Following, primitive endoderm cells were plated on gelatinized tissue culture plates for mouse embryonic fibroblast depletion for 30 minutes at 37° C. Supernatant was collected, and all three elements were centrifuged separately and cells were resuspended on N2B27 medium. The three cell fractions were counted and resuspended in N2B27 supplemented with ROCKi Y27632 (10 μl). A ratio of 1:1:3 (WT:PRE:TE) was maintained in aggregation experiments, as follows:

| | Number of | Number of added | # of Cells |
|---|---|---|---|
| | microwells per well | cells per each well | per single |
| | in 24 well plate | of a 24 well plate | microwell |
| AggreWell | 1200 | 1-WT 27720 cells | ~120 cells |
| 400 | | 2-PrE 27720 cells | |
| | | 3-TE 88560 cells | |

0.5 ml of cell-mix suspension was gently added drop wise to each well of the AggreWell plate followed by centrifugation at 700 rpm for 3 minutes and incubation at 37° C. 5% O2 (total end volume is 1 ml).

On the next day (day 1), 900 μL of medium were gently removed from each well and replaced with 1 ml of preheated N2B27 medium. The same procedure was repeated on day 2.

On day 3, aggregates were gently transferred to 6-well cell suspension culture plates (Greiner, 657185) with 3 ml of preheated EUCM2 (20%-30% FBS) media per well and placed on shaker rotation 60 rpm/min (Thermo 88881102+88881123). Plates were transferred to 21% 02 conditions.

On day 4, 2 ml of medium were gently removed per six-well and were placed with 2 ml of preheated EUCM 2 (with 30% FBS). The same procedure was repeated on day 5.

On day 6, 2 ml of medium were gently removed per six-well and were placed with 2 ml of preheated EUCM2 with 50% FBS. The same procedure was repeated on day 7.

### Results:

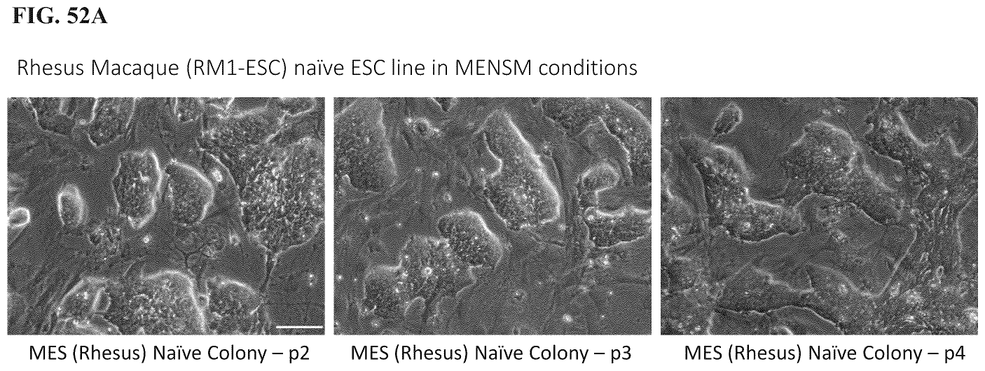

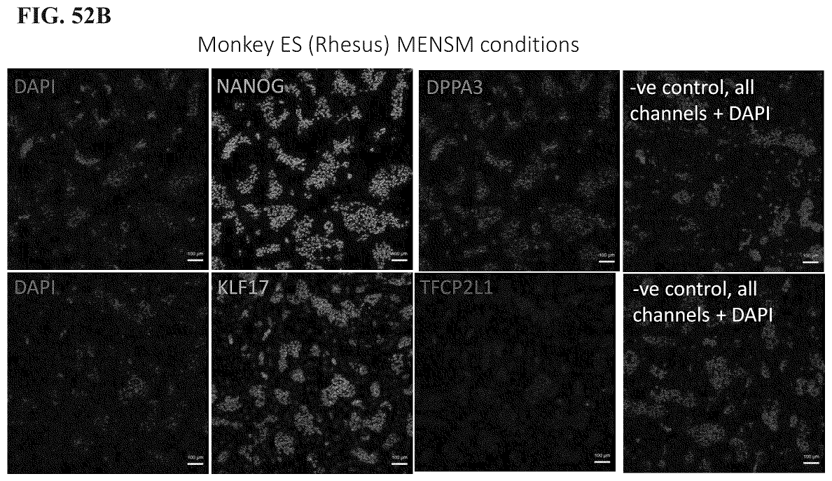

Naïve Rhesus Macaque (RM) PSCs were obtained. Phase Images show typical domed like morphology of RM1 naïve hESCs expanded in NENSM conditions at the indicated passages (FIG. 52A). Further, the cells were positive for all naïve pluripotency markers tested, namely DPPA3, TFCP2L1, KLF17 and NANOG (FIG. 52B).

Following, a subpopulation of naïve RM1 iPSCs and RM1 ESCs were cultured for 3 days in RCL condition to induce extra embryonic primitive endodermal signature, and a second subpopulation of naïve RM1 iPSCs and RM1 ESCs were cultured for 3 days in BAP-APJ protocol to induce trophectoderm signature (FIG. 53). Then, the three lineages were co-aggregated on Day 0 and the process of self-assembly of the RM sEmbryo started as indicated in the scheme including media compositions at each day.

As exemplified in FIG. 54, co-aggregation of the naïve PSCs with the two stem cell populations described, followed by ex-utero culturing enabled generation of organized embryos. The Figure demonstrates progression of RM sEmbryos from Day 6 following aggregation (on the left) corresponding to 9-12 days post fertilization (dpf) of RM embryos (schematically shown on the right). Immunostaining for OCT4, SOX17 and Gata3, markers for epiblast, hypoblast and trophoblast, respectively, indicated that all three lineages are represented in the right location. The epiblast is forming a compact layer and shows expansion of amniotic cavity through time. The hypoblast is closing to form the primary yolk sac lining the epiblast. The trophoblast is surrounding the SEM and is forming lacunae.

### Example 13

### Aggregation of Human Naïve PSCs, Pre Cells and TSCs does not Generate an Organized Embryo

### Materials and Methods

Derivation of a stable human TSC line from human naïve ESCs expanded in human naïve HENSM conditions-WIBR3 WT human female ESC line was expanded for at least 3 passages in HENSM media (as described in Example 9 hereinabove), either on plates coated with 1% GELTREX (Thermo A1413202/A1413302) or 1% Matrigel or Biolaminin511 (5 μg/ml) or MEF/gelatin coated plates. Cells were expanded in 5% O2, but it is also possible in 20% O2.

Generation of trophectoderm stem cells (TSC)—Human TSC line derived from WIBR3 naïve ESCs at (Passage 5). The latter was generated by transferring human naïve WIBR3 expanded in HENSM conditions into TSC media on MEF coated plates and serially passaged in TSC media and validated as TSC line. The TSC line was constitutively labeled with an RFP coding lentivirus (labeling it as RED). Human TSC media was previously described in Okae et al. (10.1016/j.stem.2017.11.004) with slight modifications:
DMEM/F12 (Invitrogen 21331)—470 ml
5 ml commercial N2 supplement (Invitrogen 17502048)
10 ml B27 supplement (Invitrogen 17504-044)
Sodium Pyruvate—5 ml (Biological Industries 03-042-1B)
Pen-strep 5 ml (Biological Industries 03-033-1B)
Glutamax—5 ml (Invitrogen 35050061)
NEAA—5 ml (Biological Industries 01-340-1B)
L-ascorbic acid 2-phosphate (Sigma—A8960) (50 μg/ml final concentration) (1 vial)
Human EGF 50 ng/ml final concentration (Peprotech-AF-100-15)
TGFRi A83-01—0.75-1 μM final concentration (Axon Medchem A83-01)
GSK3i CHIR99021—2 μM (Axon medchem 1386)
HDAC inhibitor—VPA 0.2 mM final concentration (WAKO #227-01071)
ROCKi Y27632 5 μM. (Axon 1683) (1 vial)

Of note, the medium described in Okae et al. comprises 1 ml of FBS to get 0.2% final FBS, and instead of N2 supplement they use ITS-X supplement and 4 times higher concertation of VPA).

Co-aggregation and culture—Three starting cell types were co-aggregated:
1) Naïve WT hESCs;
2) For the primitive endoderm compartment—Naïve WT cells were plated on mouse embryonic fibroblast conditions on HENSM supplemented with ROCKi 10 μM (Axon Medchem 1683), next day medium was replaced by RCL medium, RCL was kept for 72 hours with 24-hour medium exchanges; and
3) Human TSCs

All three cell types were supplemented with 5 μM ROCKi Y27632 before harvesting for co-aggregation on the next day. Co-aggregation was defined as time point 0 of the protocol. At the day of aggregation (day 0), Aggrewell plates 400, 24 wells (Stemcell Technologies) were prepared according to manufacturer instructions and prefilled with 500 μL of N2B27 media and kept at 37° C. for medium equilibration. The three cell populations were disaggregated with TrypLE (Thermo Fisher 12604054) for 3 minutes for the WT and Primitive endoderm cells and 5 minutes for the TSC line cells at 37° C., next the enzyme was removed and cells were incubated for two minutes at room temperature, afterwards cells were collected with PBS. Cells were centrifuged at 1300 rpm for 3 minutes and resuspended in N2B27 ROCKi Y27632 (10 μM final concentration). Following, primitive endoderm cells were plated on gelatinized tissue culture plates for mouse embryonic fibroblast depletion for 30 minutes at 37° C. The supernatant was collected, and all three elements were centrifuged separately, and cells were resuspended on N2B27 medium. The three cell fractions were counted and resuspended in N2B27 supplemented with ROCKi Y27632 (10 μl). A ratio of 1:1:3 (WT:PRE:TE) was maintained in aggregation experiments, as follows:

| | Number of | Number of added | # of Cells |
|---|---|---|---|
| | microwells per well | cells per each well | per single |
| | in 24 well plate | of a 24 well plate | microwell |
| AggreWell | 1200 | 1-WT 27720 cells | ~120 cells |
| 400 | | 2-PrE 27720 cells | |
| | | 3-TSC 88560 cells | |

0.5 ml of cell-mix suspension was gently added drop wise to each well of the AggreWell plate followed by centrifugation at 700 rpm for 3 minutes and incubation at 37° C. 5% O2 (total end volume is 1 ml).

TSCs were introduced in day 0, day 1 and day 2 of the protocol, and protocol adjustment was done accordingly.

The next day (day 1) 900 μL of medium were gently removed from each well and replaced with 1 ml of preheated N2B27 medium. Same procedure was repeated on day 2.

On day 3 aggregates were gently transferred to 6-well cell suspension culture plates (Greiner, 657185) with 3 ml of preheated EUCM2 (20%-30% FBs) media per well and placed on shaker rotation 60 rpm/min (Thermo 88881102+88881123). Plates were transferred to 21% O2 conditions.

On day 4, 2 ml of medium were gently removed per six-well and were placed with 2 ml of preheated EUCM 2 (with 30% FBS). Same procedure was repeated on day 5

On day 6, 2 ml of medium were gently removed per six-well and were placed with 2 ml of preheated EUCM2 with 50% FBS. Same procedure was repeated on day 7.

Immunostaining—As described in Example 1 hereinabove. Antibodies used are shown in Table 1 hereinbelow.

### Results:

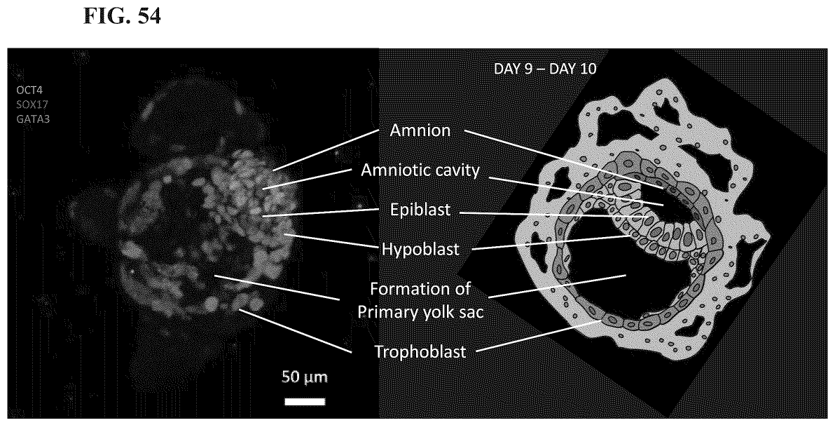

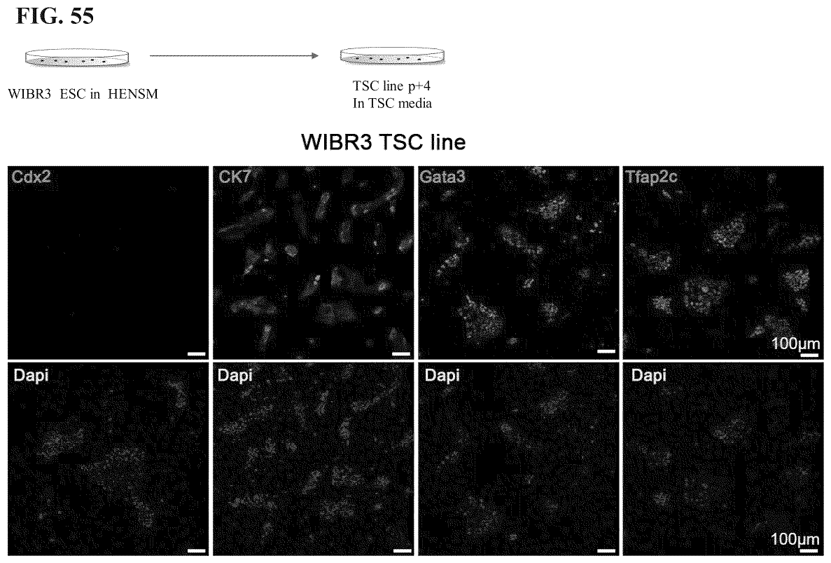

Human naïve WIBr3 ESCs expanded at least 3 passages in HENSM conditions were then transferred into TSC media, and after 3 passages a stable TSC line could be established and passaged for at least 70 more times. Cells were passaged every 5 days with TryplE. Immunostaining confirmed human TSC identity: human TSC cells were CDx2 negative, Cytokeratin7 (CK7) Positive and GATA3/TFAP2C positive consistent with previous reports. 10.1016/j.stem.2017.11.004 and 10.1016/j.stemcr.2022.09.008 (FIG. 55).

As shown in FIG. 56, co-aggregation of three starting cell types, namely naïve human ESCs PSCs, human TSCs and human PRE cells followed by ex-utero culturing did not lead to generation of an organized embryo. In all cases it could be appreciated that the cells co-localize in one side of the aggregate (as appreciated by their RED label) without surrounding the entire sEmbryo at Day 5, which does not correspond with the expected morphology and does not yield normally developed embryos that are surrounded by trophoblast lineage only on the outside periphery.

### Example 14

### Derivation of a Novel Mouse Extraembryonic Primitive Endodermal Primed Cell

Generation of extra embryonic primitive endodermal primed cells (PRE) originating from mouse naïve ESCs/iPSCs which can be permanently expanded in culture-Previously PRE cells were described to be derived from mouse E3.5 blastocysts (and not from ESCs or iPSCs) (DOI: 10.1126/science.aay3325) and the media used was a commercial base whose base is not commercially disclosed (StemFit® Basic02 (Ajinomoto) supplemented with: CHIR99021 10 μM, FGF4 50 ng/ml, PDGF 25 ng/ml and Heparin 1 μg/ml) and 10 μM REPROCELL (Reprocell).

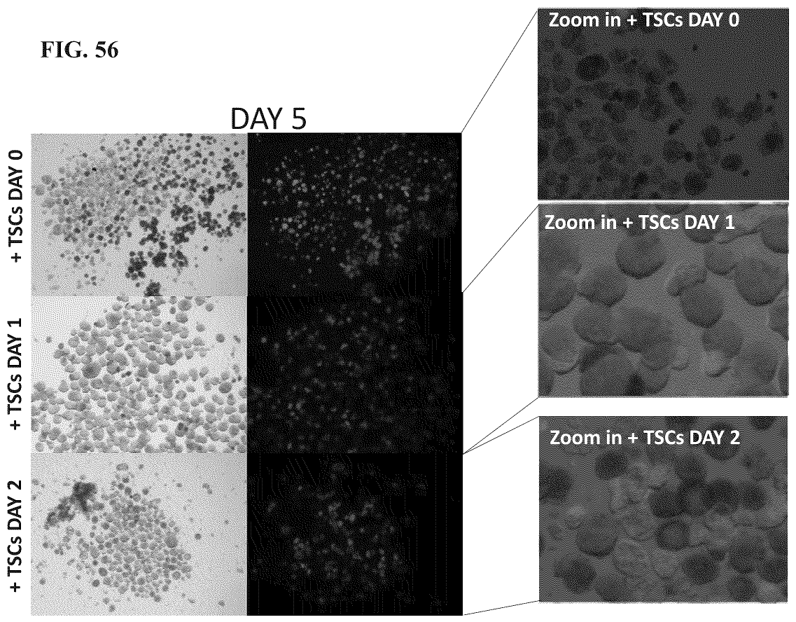

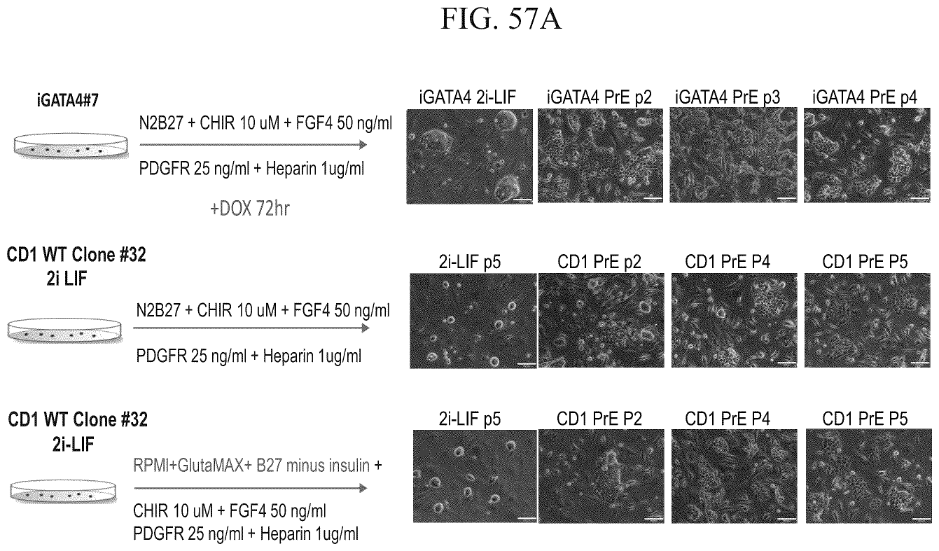

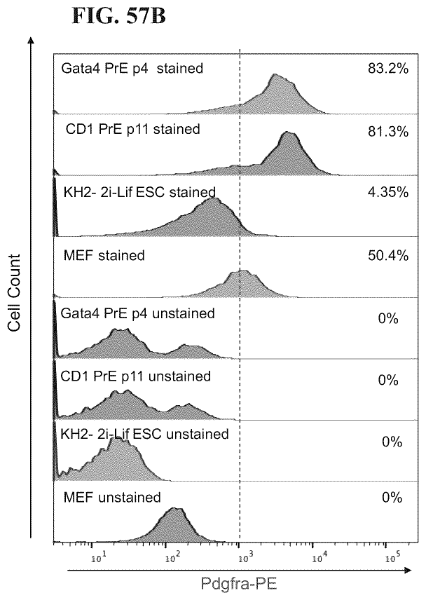

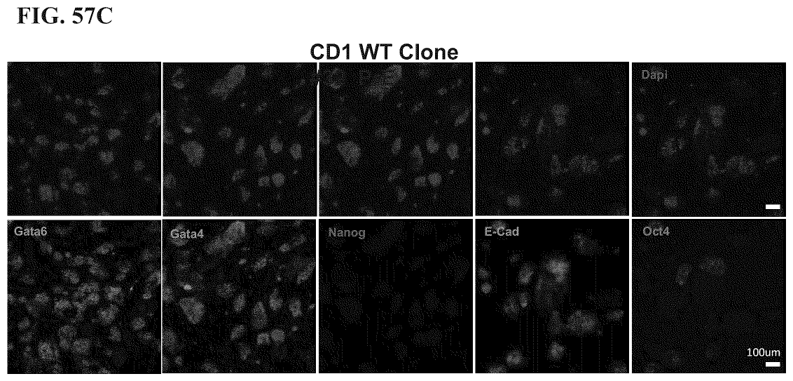

To derive PRE cells from in vitro expanded mouse ESCs and iPSCs that that can be permanently expanded in PRE media, three strategies were used (FIG. 57A).

In the top and middle panels of FIG. 57A, the PRE media used contained: serum-free chemically defined N2B27-based media: 240 ml Neurobasal (ThermoFisher 21103049) and 240 ml of DMEM-F12 with HEPES (SIGMA D6421), 5 ml N2 supplement (Invitrogen; 17502048), 5-10 ml B27 supplement (Invitrogen; 17504044 with or without insulin), 2 mM glutamine or 1 mM GLutamax (Invitrogen), 1% nonessential amino acids (Invitrogen), 0.1 mM β-mercaptoethanol (Sigma), 1% penicillin-streptomycin (Invitrogen). Optionally, the medium also included 5 mg/ml BSA (Sigma). CHIR99021 10 μM, FGF4 50 ng/ml, PDGF 25 ng/ml and Heparin 1 μg/ml). The medium did not contain REPROCELL (Reprocell).

When iGata4 cells were used (top panel of FIG. 57A), DOX was included only in the first 72 hours of the growth expansion protocol.

In both top and middle panels of FIG. 57A, the cells were maintained on MEFs in PRE media and passaged by TryplE every 4-5 days.

In the bottom panel of FIG. 57A, the RPE media used was an RPMI based media containing: 480 RPMI media (21875-034 GIBCO Invitrogen), 10 ml B27 supplement (Invitrogen; 17504044 with insulin or Invitrogen A18956-01 B27 without insulin), 1 mM GLutamax (Invitrogen), 1% nonessential amino acids (Invitrogen), 1% penicillin-streptomycin (Invitrogen), CHIR99021, 10 μM, FGF4 50 ng/ml, PDGF 25 ng/ml and Heparin 1 μg/ml). The medium was devoid of REPROCELL (Reprocell) component.

In the bottom panel of FIG. 57A, WR CD1 iPSCs were passaged in the RPMI based PRE media, and were maintained on MEFs in PRE media and passaged by TryplE every 4-5 days.

Generation of synthetic embryos—To generate the synthetic embryos, three starting cell types were co-aggregated:
1) naïve WT BVSC in 2i/LIF, as indicated.
2) CD1 iPSc derived Pre cell line expanded in PRE media as described hereinabove.
3) naïve iCdx2 ESCs in 2i/LIF, pretreated with DOX (2 μg/ml—Sigma D9891) for 24 h before aggregation (from day −1) in TSC media (25 ng/ml FGF4 (Peprotech), 1 μ/ml Heparin (Sigma)) supplemented with lysophosphatidic acid (LPA, a Hippo pathway inhibitor) 01 μM.

Co-aggregation was defined as time point 0 of the protocol

At the day of aggregation (day 0), the three donor cell populations were trypsinized with 0.05% trypsin-EDTA solution (Biological Industries—Sartorius 03-053-1B) for 4 minutes at 37° C. Trypsin enzymatic reaction was stopped by adding aggregation media. Cells were centrifuged at 1200 rpm for 3 minutes and resuspended in aggregation medium with DOX (2 μg/ml—Sigma D9891) and ROCKi Y27632 (5 nM final concentration—Axon Medchem 1683). Following, cells were plated on gelatinized tissue culture plates for mouse embryonic fibroblast depletion for 20 minutes at 37° C. Supernatant was collected, centrifuged and cells were resuspended. The three cell fractions were counted and resuspended in aggregation media with DOX (2 μg/ml—Sigma D9891) and ROCKi Y27632 (5 nM—Axon Medchem 1683). A ratio of (1 WT-ESCs:1 Pre cells: 3.33 iCdx2 ESC) was maintained in aggregation experiments, as follows:

| | Number of | Number of added | # of Cells |
|---|---|---|---|
| | microwells per well | cells per each well | per single |
| | in 24 well plate | of a 24 well plate | microwell |
| AggreWell | 300 | 1-iCDX2: 4800 cells | ~27 cells |
| 800 | | 2-Pre cells: 1500 cells | |
| | | 3-WT ESC 1500 cells | |

1 ml of cell-mix suspension was gently added drop wise to each well of the AggreWell plate followed by centrifugation at 700 rpm for 3 minutes and incubation at 37° C. (total end volume is 1.5 ml).

On the next day (day 1), 1 ml of media (out of total 1.5 ml) was gently removed from each well and replaced with 1 ml of preheated aggregation media with DOX (2 μg/ml—Sigma D9891).

On day 2, 1 ml of media was removed from each well and replaced with 1 ml of preheated aggregation media.

On day 3, 1 ml of media was removed from each well and replaced with 1 ml of preheated EUCM2 media. In this example, EUCM2 media refers to: Advanced DMEM/F12 (GIBCO 21331-020), 5 ml CMRL media (1% final concentration) as a supplement (5mlGIBCO 11530037), extra added 1 mg/ml D (+)-Glucose Monohydrate (J.T. Baker—0113) (add 500 mg per 500 ml media), 100 nM T3 (3,3′,5-Triiodo-L-thyronine sodium salt) (Sigma-Aldrich, T6397), 1 mM Sodium pyruvate (Sigma-Aldrich, S8636), 1 mM GlutaMAX (GIBCO, 35050061), 1% penicillin streptomycin (Biological Industries—Sartorius 03-031-1B), 1× of ITS-X supplement (Thermo Fisher Scientific 51500-056), 8 nM B-estradiol (Sigma-Aldrich, E8875), 200 ng/ml progesterone (Sigma-Aldrich, P0130), 25 μM N-acetyl-L-cysteine (Sigma-Aldrich, A7250)+20% FBS.

On day 4, the synthetic embryos were gently transferred to 6-well cell suspension culture plates (Greiner, 657185) with 5 ml of preheated EUCM2 media containing 30% FBS per well and placed on shaker rotation 60 rpm/min (Thermo 88881102+88881123).

On day 5, egg cylinder shape sEmbryos were picked and transferred to glass culture bottles (50-30 embryos per bottle) containing 2 mL of EUCM media (the contents of which are described in Example 1 hereinabove). Following, the bottles were placed on the rolling culture system previously described electronically controlled roller culture platform (Aguilera-Castrejon et al., 2021a), rotating at 30 revolutions per minute at 37° C., and continuously gassed with an atmosphere of 21% O₂, 5% CO₂ at 7-8 pounds per square inch (psi). A black non-transparent cloth was used to cover the incubator from light during unit operation.

From day 6 to day 8, 1 ml of EUCM was replaced with preheated 1 ml of EUCM and kept on rolling culture system. Culture media was pre-heated for at least an hour by placing it inside a glass bottle on the rotating culture.

FACS analysis—Cell were immunostained with fluorescent labeled antibodies as indicated in Table 1 hereinbelow, washed with PBS and analyzed on FACS ARIA III and analyzed with a FACS DIVA software.

Immunostaining—As described in Example 1 hereinabove.

### Results:

The present inventors obtained novel extra embryonic primitive endodermal primed cells (PRE) originating from mouse naïve ESCs/iPSCs which can be permanently expanded in culture without losing their phenotype (FIG. 57A). FACS analysis demonstrated that these cells, but not parental ESC lines, express the PRE surface marker PDGFRa (FIG. 57B). Immunostaining analysis demonstrated that these cells express the pluripotent marker Oct4 but not Nanog; the cells are also positive for Gata4 and Gata6 primitive endoderm markers and maintain E-cadherin expression (FIG. 57C). It is noted (as validated in literature DOI: 10.1126/science.aay3325) that XEN cells are negative for Oct4 which is a major difference between them and the obtained PRE cells; and mouse ESCs are positive for both Oct4 and Nanog, while the PRE cells are negative for Nanog and only positive for Oct4.

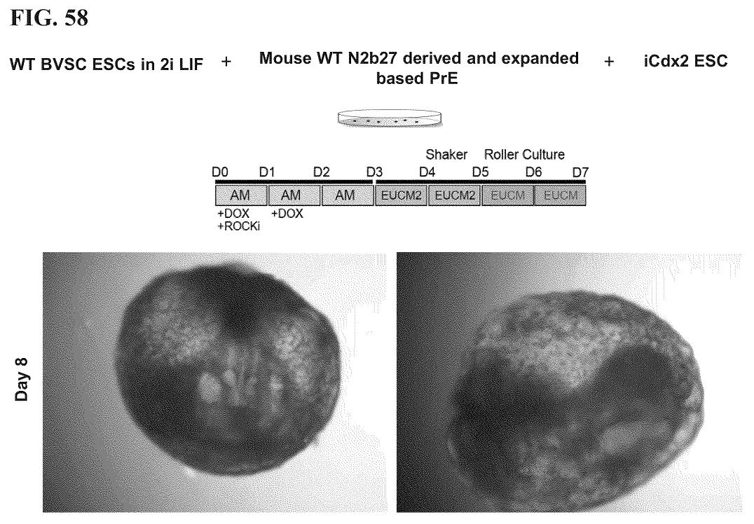

As exemplified in FIG. 58, co-aggregation of naïve mouse PSCs with this novel PRE cells and mouse iCDX2 trophectoderm primed cells, followed by ex-utero culturing enabled generation of organized embryos (FIG. 58). The Figure demonstrates generation of Day 8 mouse sEmbryos.

### Example 15

### Generation of Organized Synthetic Human Embryos

### Materials and Methods

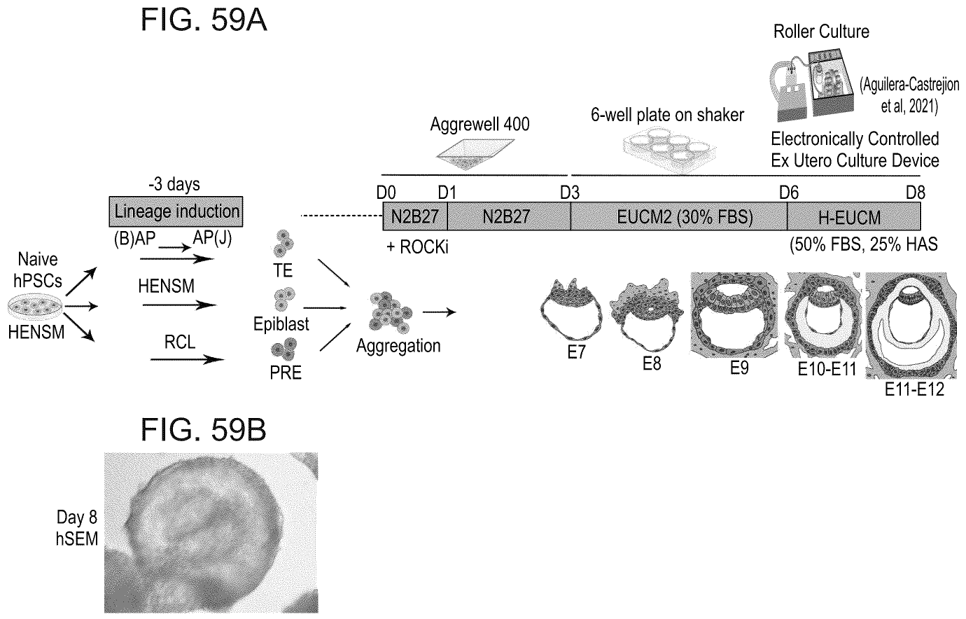

Co-aggregation and culture (FIG. 59A)—Three starting cell types were co-aggregated:
4) Naïve WT PSC cells cultured in HENSM medium.
5) For the primitive endoderm compartment—Naïve WT cells were plated on mouse embryonic fibroblast conditions on HENSM supplemented with ROCKi 10 μM (Axon Medchem 1683), next day medium was replaced by RCL medium, RCL was kept for 72 h with 24-hour medium exchanges.
6) For the Trophectoderm lineage—Naïve WT cells were plated on feeder free conditions (Matrigel or laminin E8) on HENSM supplemented with ROCKi 10 μM, next day medium was replaced with (B)AP medium for 24 hours, next medium was replaced with AP(J) for another 48 hours.

All three cell types were supplemented with 5 μM ROCKi Y27632 before harvesting for co-aggregation on the next day. Co-aggregation was defined as time point 0 of the protocol. At the day of aggregation (day 0), Aggrewell plates 400, 24 wells (Stemcell Technologies) were prepared according to manufacturer instructions and prefilled with 500 μL of N2B27 media and kept at 37° C. for medium equilibration. The three cell populations were disaggregated with TrypLE (Thermo Fisher 12604054) for 3 minutes for the WT and Primitive endoderm cells and 5 minutes for the Trophectoderm cells at 37° C., next the enzyme was removed and cells were incubated for two minutes at room temperature, afterwards cells were collected with PBS. Cells were centrifuged at 1300 rpm for 3 minutes and resuspended in N2B27 ROCKi Y27632 (10 μM final concentration). Following, primitive endoderm cells were plated on gelatinized tissue culture plates for mouse embryonic fibroblast depletion for 30 minutes at 37° C. Supernatant was collected, and all three elements were centrifuged separately and cells were resuspended on N2B27 medium. The three cell fractions were counted and resuspended in N2B27 supplemented with ROCKi Y27632 (10 μl). A ratio of 1:1:3 (WT:PRE:TE) was maintained in aggregation experiments, as follows:

| | Number of | Number of added | # of Cells |
|---|---|---|---|
| | microwells per well | cells per each well | per single |
| | in 24 well plate | of a 24 well plate | microwell |
| AggreWell | 1200 | 1-WT 27720 cells | ~120 cells |
| 400 | | 2-PrE 27720 cells | |
| | | 3-TE 88560 cells | |

0.5 ml of cell-mix suspension was gently added drop wise to each well of the AggreWell plate followed by centrifugation at 700 rpm for 3 minutes and incubation at 37° C. 5% O2 (total end volume is 1 ml).

On the next day (day 1), 900 μL of medium were gently removed from each well and replaced with 1 ml of preheated N2B27 medium. Same procedure was repeated on day 2.

On day 3 aggregates were gently transferred to 6-well cell suspension culture plates (Greiner, 657185) with 3 ml of preheated EUCM2 (20%-30% FBs) media per well and placed on shaker rotation 60 rpm/min (Thermo 88881102+88881123). Plates were transferred to 21% O2 conditions.

On day 4, 2 ml of medium were gently removed per six-well and were placed with 2 ml of preheated EUCM 2 (with 30% FBS). The same procedure was repeated on day 5

On day 6, aggregates were collected and transferred into 4 ml of H-EUCM media and placed in Ex Utero electronically controlled Ex Utero Culture Device. Every 24 hours, aggregates were transferred into a new bottle with fresh 4 ml of HEUCM media until day 8 or later.

Electronically controlled ex utero roller culture platform (FIG. 59A)—Putative human sEmbryos were kept starting from Day 6 or later in the ex utero electronically controlled roller culture platform (Aguilera-Castrejon et al., 2021). A ‘rotator’ culture method provides continuous flow of oxygenating gas to cultures in rotating bottles. sEmbryos are kept on a rotating bottles culture unit inside a “precision” incubator system during all the time of culture. Glass culture bottles are plugged into the hollowed rotating drum. Oxygenating gas flows along the axis and is distributed to the culture bottles by a baffle plate within the drum (Aguilera-Castrejon et al., 2021). The rotator is supplied complete with gas filter, bubbler and leads by the manufacturer. The Incubator uses a thyristor-controlled heater and high flow-rate fan to give a highly stable and uniform temperature as previously described (Aguilera-Castrejon et al., 2021). The incubator has a working volume 370×350×200 mm high which is accessed through the hinged Perspex top. The heater element is rated at 750 Watts. Bung (Hole) is used to seal the bottles and Bung (Solid) is used to seal the drum (Aguilera-Castrejon et al., 2021). In order to achieve constant O₂ and CO₂ levels in the culture medium throughout the incubation period, the incubator module was linked to an in-house designed and customized gas and pressure electronic control unit (models #-HannaLab1 or HannaLab1.2; designed by the Hanna lab (Aguilera-Castrejon et al., 2021). Carbon dioxide and oxygen concentration are regulated by specific controllers located inside the regulation module. A pressure transmitter allows precise and stable control of the gas pressure between 5 to 10 psi (positive pressure over ambient external atmospheric pressure), that is transmitted to the embryo bottle apparatus. Regulation of pressure generated by the pressure pump is done by setting the adequate voltage on the pressure transmitter. Oxygen and CO₂ are then injected into the gas mixer box. The mixing of the gases in the gas box is homogeneous and mixed by a centrifugal blower. The gases are injected into the incubator unit at pressure of 6.5-8 psi (as measured at the gas mixing box outlet) by a pump, which yields an effective pressure˜0.1-0.5 psi entering the rotating drum, reminiscent of pressure values measured in amniotic fluid of early embryos in vivo (Sideris and Nicolaides, 1990).

Adequate and stable control of the pressure of the gas flowing from the mixing box outlet into the water bottle inside the incubator should be measured by using a pressure gauge before each experiment. The main components of the system are the following: Oxygen and CO₂ controller, pressure pump, vacuum pump, oxygen and CO₂ sensors, power supply, check valve, mix gas box, pressure transmitter, limit flow, adapter control for gases, 1 μm filters, centrifugal blower. Gas flows from the mix box through the inlet into the water bottle, and the speed of gas flowing into the bottle can be controlled with a valve on the lid of the water bottle. The bubble rate (which indicates the speed of gas flowing into the bottles) can be adjusted as needed by the user by closing/opening the valve. Ideally, the flow of bubbles should be such to allow formation of individual bubbles at a rate of 3-4 bubbles per second in the water-filled test tube outlet, or to the first point where continuous bubbling is observed. Humidified gas circulates to a glass test tube and then to the inside of the bottles in the rotating drum. Gas flow speed can be monitored by the rate of bubbles created inside the outlet water-filled test tube. A black nontransparent cloth is used cover the incubator to provide phototoxicity protection for the ex utero cultured embryos and sEmbryos.

H-EUCM is based on EUCM was previously established in Aguilera-Castrejon (Aguilera-Castrejon et al., 2021) for mouse embryos, with adaptation to be more suitable for human sEmbryos. Specifically, the H-EUCM is composed of:
1) 25% DMEM (GIBCO 11880—DMEM, low glucose, with pyruvate, no glutamine, no phenol red (and no HEPES)) or Advanced DMEM-F12 (Invitrogen 12634010) or CMRL (Invitrogen 11530037) or TCM199 media (Gibco 11150059 or Sigma M2520);
2) 50% Fetal Bovine Serum (FBS) (GIBCO) and 25% Human Umbilical Cord Blood Serum (HUS) (or Human Adult Blood Serum (HAS)) that is prepared in-house; and
3) supplemented with a final concentration of 1× GlutaMAX (GIBCO, 35,050,061), 50 units/ml penicillin—50 mg/ml streptomycin (Biological industries, 030311B), extra added 1 mM sodium pyruvate (Biological industries, 030421B), extra added 4 mg/mL of D-glucose (J.T. Baker), and added HEPES (11 mM final concentration) (GIBCO 15630056). Alternatively, EUCM2 media composed of 50% FBS or 50% FBs and 25% HAS could also be used in the roller culture on days 6 onwards.

### Results:

Co-aggregation of the naïve PSCs with the two stem cell populations, followed by ex-utero culturing according to the protocols described hereinabove and in FIG. 59A enabled generation of organized embryos. As shown in FIG. 59B, the human sEmbryo shows bilaminar disc formation, amniotic cavity and yolk sac cavity, all surrounded by trophoblast compartment.

### Example 16

### Generation of Organized Synthetic Human Embryos

### Materials and Methods

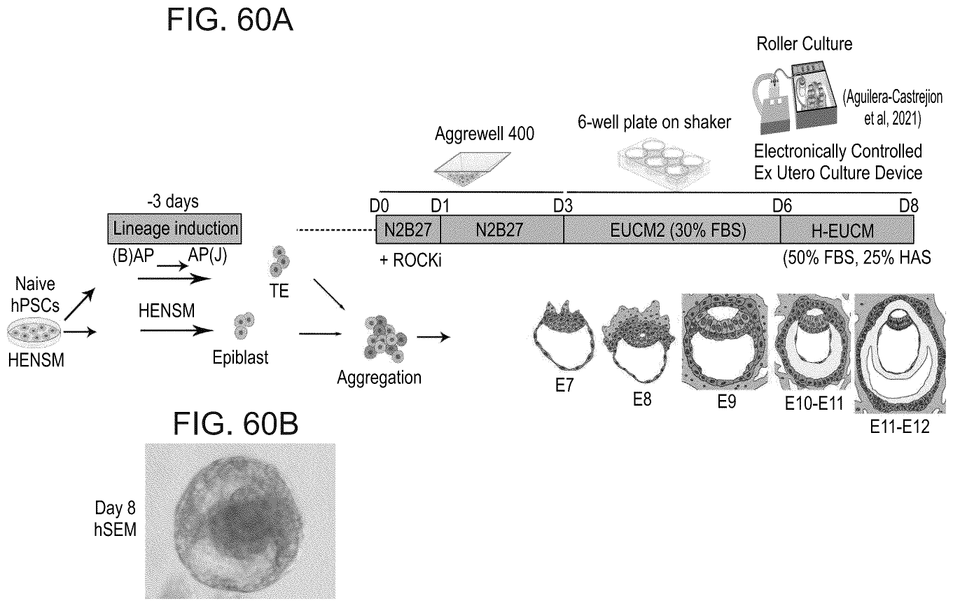

Co-aggregation and culture (FIG. 60A)—Two starting cell types were co-aggregated:
1) Naïve WT PSC cells cultured in HENSM medium, which also give rise to primitive endoderm lineage once aggregation begins in N2B27 media; and
2) For the Trophectoderm lineage—Naïve WT cells were plated on feeder free conditions (Matrigel or laminin E8) on HENSM supplemented with ROCKi 10 μM, next day medium was replaced with (B)AP medium for 24 hours, next medium was replaced with AP(J) for another 48 hours.

Both cell types were supplemented with 5 μM ROCKi Y27632 before harvesting for co-aggregation on the next day. Co-aggregation was defined as time point 0 of the protocol. At the day of aggregation (day 0), Aggrewell plates 400, 24 wells (Stemcell Technologies) were prepared according to manufacturer instructions and prefilled with 500 μL of N2B27 media and kept at 37° C. for medium equilibration. The two cell populations were disaggregated with TrypLE (Thermo Fisher 12604054) for 3 minutes for the WT PSC cells and 5 minutes for the Trophectoderm cells at 37° C., next the enzyme was removed and cells were incubated for two minutes at room temperature, afterwards cells were collected with PBS. Cells were centrifuged at 1300 rpm for 3 minutes and resuspended in N2B27 ROCKi Y27632 (10 μM final concentration). The supernatant was collected, and all elements were centrifuged separately, and cells were resuspended on N2B27 medium. The two cell fractions were counted and resuspended in N2B27 supplemented with ROCKi Y27632 (10 μl). A ratio of 2:3 (WT:TE) was maintained in aggregation experiments, as follows:

| | Number of | Number of added | # of Cells |
|---|---|---|---|
| | microwells per well | cellsper each well | per single |
| | in 24 well plate | of a 24 well plate | microwell |
| AggreWell | 1200 | 1-WT 55440 cells | ~120 cells |
| 400 | | 2-TE 88560 cells | |

0.5 ml of cell-mix suspension was gently added drop wise to each well of the AggreWell plate followed by centrifugation at 700 rpm for 3 minutes and incubation at 37° C. 5% O2 (total end volume is 1 ml).

On the next day (day 1), 900 μL of medium were gently removed from each well and replaced with 1 ml of preheated N2B27 medium. The same procedure was repeated on day 2.

On day 3 aggregates were gently transferred to 6-well cell suspension culture plates (Greiner, 657185) with 3 ml of preheated EUCM2 (20%-30% FBs) media per well and placed on shaker rotation 60 rpm/min (Thermo 88881102+88881123). Plates were transferred to 21% O2 conditions.

On day 4, 2 ml of medium were gently removed per six-well and were placed with 2 ml of preheated EUCM 2 (with 30% FBS). The same procedure was repeated on day 5.

On day 6, aggregates were collected and transferred into 4 ml of H-EUCM media and placed in Ex Utero electronically controlled Ex Utero Culture Device. Every 24 hours, aggregates were transferred into a new bottle with fresh 4 ml of HEUCM media until day 8 or later.

Electronically controlled ex utero roller culture platform (FIG. 60A)—As described in Example 15 hereinabove and according to the scheme shown in FIG. 60A.

### Results:

Co-aggregation of the naïve PSCs with the trophectoderm-primed population, followed by ex-utero culturing according to the protocols described hereinabove and in FIG. 60A enabled generation of organized embryos. As shown in FIG. 60B, the human sEmbryo shows bilaminar disc formation, amniotic cavity and yolk sac cavity, all surrounded by trophoblast compartment.

TABLE 1
Primary antibodies

| | | | | | | | | | | | Working |
|---|---|---|---|---|---|---|---|---|---|---|---|
| Ab name | Conc. | | Volume | | Aliquots | | Cat no. | Type | Host | Company | dilution |
| Oct4 (C-10 clone) | 200 | μg/ml | 100 | μl | No | | SC5279 | Monoclonal | Mouse | Santa cruz | 1:100 |
| E-cadherin [HECD-1] | 0.05 | mg/ml | 500 | μl | No | | ab1416-500 | Monoclonal | Mouse | abcam | 1:200 |
| PDGFR-A | 0.5 | mg/ml | 100 | μl | No | | 14-140-182 | Monoclonal | Rat | eBioscience | |
| Gata4 | 1 | mg/ml | 100 | μl | 5 | μl | ab84593 | Polyclonal | Rabbit | abcam | 1:100/1:200 |
| CDX2 | 0.1 | mg/ml | 100 | μl | | | #3977 | Polyclonal | Rabbit | Cell signaling | 1:100/1:200 |
| GFP | ND | | 50 | μl | 10 | μl | ab290 | Polyclonal | Rabbit | abcam | 1:250-1:500 |
| GFP | 1 | mg/ml | 100 | μl | 10 | μl | ab6673 | Polyclonal | Goat | abcam | 1:250 |
| MF 20-s (myosin) | 26 | μg/ml | 1 | ml | 100 | μl | | monoclonal | Hybridoma | DHSB | 1:250 |
| human/mouse CDX2 | 50 | mg/ml | 1 | ml | 50 | μl | AU392A-UC | monoclonal | mouse | Biogenex | 1:100 |
| mSox2 | 0.2 | mg/ml | 100 | μl | 25 | μl | AF2018 | polyclonal | Goat | R&D | 1:200 |
| Gata4 | 0.2 | mg/ml | 1 | ml | No | | sc-1237 | polyclonal | Goat | Santa Cruz | 1:200 |
| Nanog | 0.5 | mg/ml | | | 8 | μl | 14-5761 | polyclonal | Rat | eBioscience | 1:250-1:500 |
| SOX9 | 1 | mg/ml | 100 | ug | No | | ab5535 | polyclonal | Rabbit | Millipore | 1:100-1:200 |
| Anti-PKC ζ (H-1) | 0.2 | mg/ml | 1 | ml | N/A | | sc-17781 | Monoclonal | Mouse | Santa Cruz | |
| Anti-AP-2α (3B5) | 0.2 | mg/ml | 1 | ml | N/A | | sc-12726 | Monoclonal | Mouse | Santa Cruz | |
| Gata4 | 0.2 | mg/ml | 1 | ml | | | sc-25310 | Monoclonal | Mouse | Santa Cruz | 1:100 |
| Gata4 | 0.5 | mg/ml | 100 | ug | | | 14-9980-82 | Monoclonal | Rat | invitrogen | |
| Anti-PKC ζ (H-1) | 0.2 | mg/ml | 1 | ml | N/A | | sc-17781 | Monoclonal | Mouse | Santa Cruz | |
| Anti-TFAP2a | 0.2 | mg/ml | 1 | ml | N/A | | sc-12726 | Monoclonal | Mouse | Santa Cruz | |
| (AP-2α) (3B5) | | | | | | | | | | | |
| Human Otx2 | 0.2 | mg/ml | 500 | μl | 20 | μl | AF1979 | Polyclonal | Goat | R&D | 1:200 |
| Pax6 | 2 | mg/ml | 100 | μl | not | | Poly901301 | Polyclonal | Rabbit | Biolegend | 1:100 |
| | | | | | aliquoted | | | | | | |
| TUJ1 | 1 | mg/ml | 250 | ul | not | | 801202 | monoclonal | Mouse | Biolegend | 1:100 |
| | | | | | aliquoted | | | | | | |
| FOXA2 | ND | | 100 | μl | 10 | ul | ab108422 | Monoclonal | Rabbit | abcam | 1:100 |
| TBR2 (EOMES) | 1 | mg/ml | 100 | μl | 8 | μl | AB2283 | Polyclonal | Rabbit | millipore | 1:50 Low |
| | | | | | | | | | | | signal |
| Sox17 | 0.2 | mg/ml | 100 | μl | 10 | μl | AF1924 | Polyclonal | Goat | R&D | 1:100-1:50 |
| Lefty | 1 | mg/ml | 100 | μl | 10 | μl | ab22569 | Polyclonal | Rabbit | abcam | 1:100 |
| Brachyury(D2Z3J) | | | 100 | μl | | | #81694 | monoclonal | Rabbit | Cell signaling | 1:100 |
| mAb | | | | | | | | | | | |
| Human KLF17 | 0.5 | mg/ml | 100 | μl | 10 | μl | ab84196 | Polyclonal | Rabbit | abcam | |
| [N-terminus 2-51 aa | | | | | | | | | | | |
| Human KLF17 | | | 400 | μl | 20 | μl | ab174956 | Polyclonal | Rabbit | abcam | |
| [C-terminus 250-279 | | | | | | | | | | | |
| aa] (KLF17 C)] | | | | | | | | | | | |
| Human KLF17 | 0.6 | mg/ml | 100 | ul | | | HPA024629 | Polyclonal | Rabbit | sigma | |
| TFAP2c (AP2γ) | | | | | | | #2320 | Polyclonal | rabbit | cell signaling | Middle |
| | | | | | | | | | | | background |
| Myosin heavy chain | 0.2 | mg/ml | 500 | μl | 20 | μl | MAB470 | monoclonal | mouse | R&D | 1:250 |
| (clone MF20) | | | | | | | | | | | |
| CK8 Cytokeratin 8 | 1 | mg/ml | 100 | ul | | | MABT329 | monoclonal | Rat | Millipore | |
| Lefty | 0.2 | mg/ml | 500 | μl | 20 | μl | AF746 | polyclonal | Goat | R&D | 1:100 |
| mCHERRY/tdTomato | | | | | 10 | ul | AB0040200 | polyclonal | Goat | SICGEN | 1:200 |
| | | | | | | | | | | ANTIBODIES | |
| TBR2/Eomes | 1 | mg/ml | 100 | ul | 5 | ul | ab23345 | Polyclonal | Rabbit | abcam | 1:50/1:100 |
| Cdx2 | 1 | mg/ml | 100 | ul | 5 | ul | ab76541 | Monoclonal | Rabbit | abcam | 1:200/1:500 |
| DKK1 | 0.2 | mg/ml | 500 | ul | 20 | ul | AF1096 | Polyclonal | Goat | R&D | 1:100 |
| AP2gamma (Tfap2c) | 0.2 | mg/ml | 500 | ul | 10 | ul | AF5059 | monoclonal | Goat | R&D | 1:100/1:200 |
| cytokeratin7 CK7 | | | 100 | ul | 9 | ul | ab181598 | Monoclonal | Rabbit | abcam | 1:200 |
| NR2F2 | | | 100 | ul | 9 | ul | ab211776 | Monoclonal | Rabbit | abcam | |
| nanog | | | 100 | ul | 9 | ul | ab109250 | Monoclonal | Rabbit | abcam | |
| TBX3 | | | 100 | ul | 9 | ul | ab99302 | Monoclonal | Rabbit | abcam | |
| Mouse Anti-Zo-1 | 0.5 | mg/mL | 100 | ul | | | Zy-339100 | Monoclonal | Mouse | invitrogen | 1:200 |
| Brachyury | 0.2 | mg/ml | 100 | μl | 10 | μl | AF2085 | Polyclonal | Goat | R&D | 1:100 |
| Lefty | 0.2 | mg/ml | 500 | μl | 20 | μl | AF746 | polyclonal | Goat | R&D | 1:100 |
| RUNX1/AML1 | | | 100 | ul | 5 | ul | ab240639 | Monoclonal | Rabbit | Abcam | 1:100 |
| [clone EPR23044-100] | | | | | | | | | | | |
| HOXB4 | | | 100 | ul | 5 | ul | ab133521 | Monoclonal | Rabbit | Abcam | 1:100 |
| [clone EPR1917] | | | | | | | | | | | |
| FoxG1 | 0.57 | mg/ml | 100 | ul | 6 | ul | ab196868 | Monoclonal | Rabbit | abcam | 1:100 |
| (clone EPR18987) | | | | | | | | | | | |
| GATA3 | | | | | | | AF2605 | Polyclonal | Goat | R&D | 1:200 |
| GATA3 | | | | | | | MA1-028 | monoclonal | Mouse | invitrogen | 1:200 |
| Anti hCG beta | 0.2 | mg/ml | 100 | ul | 10 | ul | ab9582 | Monoclonal | Mouse | abcam | 1:200 |
| Anti-PKC ζ (H-1) | 0.2 | mg/ml | 1 | ml | N/A | | sc-17781 | Monoclonal | Mouse | Santa Cruz | |
| Anti-AP-2α (3B5) | 0.2 | mg/ml | 1 | ml | N/A | | sc-12726 | Monoclonal | Mouse | Santa Cruz | |
| Cerberus 1 | 0.2 | mg/ml | 200 | ul | 7.5 | ul | AF1075 | Polyclonal | Goat | R&D | |
| TROP-2 | 0.2 | mg/ml | 200 | ul | 7.5 | ul | MAB650 | Polyclonal | Goat | R&D | |
| [Clone 77220] | | | | | | | | | | | |
| Gata6 (clone D61E4) | | | 100 | ul | Not | | #5951 | Monoclonal | Rabbit | Cell Signaling | |
| | | | | | aliquoted | | | | | | |
| Blimp1/PDRI-BF1 | | | 100 | ul | Not | | #9115 | Monoclonal | Rabbit | Cell Signaling | |
| [Clone C14A4] | | | | | aliquoted | | | | | | |
| E-Cadherin | | | 100 | ul | Not | | #3195 | Monoclonal | Rabbit | Cell Signaling | |
| [Clone 24E10] | | | | | aliquoted | | | | | | |
| Collagen VI | | | 100 | ul | 7 | ul | ab6588 | Polyclonal | Rabbit | Abcam | |
| Wnt5a [Clone 24E10] | | | 100 | ul | 7 | ul | ab235966 | Polyclonal | Rabbit | Abcam | |
| Cytokeratin 7 | | | 100 | ul | 7 | ul | ab68459 | Monoclonal | Rabbit | Abcam | 1:200 |
| [EPR1619Y] | | | | | | | | | | | |
| FoxA1 [clone 1B1] | 1 | mg/ml | 200 | ul | 7.5 | ul | ab55178 | Monoclonal | Mouse | Abcam | |
| VEGFR2/KDR/Flk-1 | 0.2 | mg/ml | 200 | ul | 10 | ul | AF357 | Polyclonal | Goat | R&D | |
| Oct3/4 | 0.2 | mg/ml | 200 | ul | 7.5 | ul | AF1759 | Polyclonal | Goat | R&D | |
| Gata6 | 0.2 | mg/ml | 500 | ul | 10 | ul | AF1700 | Polyclonal | Goat | R&D | |
| Syndecan1 [EPR6454] | | | 100 | ul | 5.5 | ul | ab128936 | Monoclonal | Rabbit | Abcam | 1:250/1:500 |
| Islet1 [EP4182] | | | 100 | ul | 5.5 | ul | ab109517 | Monoclonal | Rabbit | Abcam | |
| PDGFR-alpha | | | 100 | ul | 5.5 | ul | ab230457 | Polyclonal | Rabbit | Abcam | |
| PDGFR-alpha | | | 100 | ul | 5.5 | ul | ab134123 | Monoclonal | Rabbit | Abcam | |
| [EPR5480] | | | | | | | | | | | |
| MIXL1 | | | 100 | ul | 5.5 | ul | HPA005662 | Polyclonal | Rabbit | SIGMA | |
| Nidogen-2 | | | 100 | ul | 5.5 | ul | AF3385 | Polyclonal | Goat | R&D | |
| GABRP | 0.5 | mg/ml | 100 | ul | 6.5 | ul | PA5-46830 | Polyclonal | Rabbit | Invitrogen | |
| Hesx1 | | | 100 | ul | 6.5 | ul | ab246949 | Polyclonal | Rabbit | Abcam | |
| Periostin | 0.5 | mg/ml | 100 | ul | 10 | ul | ab14041 | Polyclonal | Rabbit | Abcam | |
| Vimentin [RV202] | 1 | mg/ml | 100 | ul | 10 | ul | ab8978 | Monoclonal | Mouse | Abcam | |
| Tetherin/BTS2 | 0.75 | mg/ml | 100 | ul | 10 | ul | ab243230 | Monoclonal | Rabbit | Abcam | |
| [EPR20202] | | | | | | | | | | | |
| GATA2 [EPR2822] | 0.325 | mg/ml | 100 | ul | 10 | ul | ab109241 | Monoclonal | Rabbit | Abcam | |

TABLE 2
List of primers:

| Primer | Sequence | SEQ ID NO: |
|---|---|---|
| Primer: human GATA2 | ACTGACGGAGAGCATGAAGAT | 131 |
| (Forward): | | |
| Primer: human GATA2 | CCGGCACATAGGAGGGGTA | 132 |
| (Reverse): | | |
| Primer: human GATA3 | GCCCCTCATTAAGCCCAAG | 133 |
| (Forward): | | |
| Primer: human GATA3 | TTGTGGTGGTCTGACAGTTCG | 134 |
| (Reverse): | | |
| Primer: human ENPEP | TCCCAAAGAATACGGAGCACT | 135 |
| (Forward): | | |
| Primer: human ENPEP | TCATGGGGACAGACTTCTCAA | 136 |
| (Reverse): | | |
| Primer: human ELF5 | TAGGGAACAAGGAATTTTTCGGG | 137 |
| (Forward): | | |
| Primer: human ELF5 | GTACACTAACCTTCGGTCAACC | 138 |
| (Reverse): | | |
| Primer: human DAB2 | ACCACCCTTTCACAAGCAAC | 139 |
| (Forward): | | |
| Primer: human DAB2 | TGTGCCCATTTGCAATTCTA | 140 |
| (Reverse): | | |
| Primer: human TACSTD2 | ACAACGATGGCCTCTACGAC | 141 |
| (Forward): | | |
| Primer: human TACSTD2 | AGTTCACGCACCAGCACAC | 142 |
| (Reverse): | | |
| Primer: human TFAP2A | GTTACCCTGCTCACATCACTAG | 143 |
| (Forward): | | |
| Primer: human TFAP2A | TCTTGTCACTTGCTCATTGGG | 144 |
| (Reverse): | | |
| Primer: human SIGLEC6 | TCACAACCCTGGTTTTCCTC | 145 |
| (Forward): | | |
| Primer: human SIGLEC6 | CTGTCTGGAACTGGTGCTGA | 146 |
| (Reverse): | | |
| Primer: human CDX2-F | TCACCATCCGGAGGAAAGCC | 147 |
| (Forward): | | |
| Primer: human CDX2-R | CTCTCCTTTGCTCTGCGGTT | 148 |
| (Reverse): | | |
| Primer: human HAND1 | CCAAGGATGCACAGTCTGG | 149 |
| (Forward): | | |
| Primer: human HAND1 | AGGAGGAAAACCTTCGTGCTG | 150 |
| (Reverse): | | |
| Primer: human TFAP2C | ACGAAATGAGATGGCAGCTAGGAAGA | 151 |
| (Forward): | | |
| Primer: human TFAP2C | TGTCCGGTCTTGGCTGAGAAGTTC | 152 |
| (Reverse): | | |
| Primer: human ABCG2 | ACG AAC GGA TTA ACA GGG TCA | 153 |
| (Forward): | | |
| Primer: human ABCG2 | CCT CCA GAC ACA CCA CGG ATA | 154 |
| (Reverse): | | |
| Primer: human KRT19 | ACC TGG AGA TGC AGA TCG AA | 155 |
| (Forward): | | |
| Primer: human KRT19 | AAT CCA CCT CCA CAC TGA CC | 156 |
| (Reverse): | | |
| Primer: human VGLL1 | TGC CTC CCG GCT CAG TTC ACT | 157 |
| (Forward): | | |
| Primer: human VGLL1 | CCC AGT GGT TTG GTG GTG TA | 158 |
| (Reverse): | | |
| Primer: human NR2F2 | GCC ATA GTC CTG TTC ACC TCA | 159 |
| (Forward): | | |
| Primer: human NR2F2 | AAT CTC GTC GGC TGG TTG | 160 |
| (Reverse): | | |
| Primer: human SOX17 | TGG CGC AGC AGA ATC CAG AC | 161 |
| (Forward): | | |
| Primer: human SOX17 | TTG GGG TGG TCC TGC ATG TG | 162 |
| (Reverse) | | |
| Primer: human PDGFR- | TGC CTG ACA TTG ACC CTG T | 163 |
| alpha (Forward): | | |
| Primer: human PDGFR- | CCG TCT CAA TGG CAC TCT C | 164 |
| alpha (Reverse): | | |
| Primer: human CXCR4 | CCT GCC TGG TAT TGT CAT CC | 165 |
| (Forward): | | |
| Primer: human CXCR4 | GAT GGG GAT GAT TGT GGT CT | 166 |
| (Reverse): | | |
| Primer: human HHEX1 | GCG GAC GGT GAA CGA CTA | 167 |
| (Forward): | | |
| Primer: human HHEX1 | GGC CGC CTT TCC TTT TAT | 168 |
| (Reverse): | | |
| Primer: human GSC | CCT CCG CGA GGA GAA AGT | 169 |
| (Forward): | | |
| Primer: human GSC | CGT TCT CCG ACT CCT CTG AT | 170 |
| (Reverse): | | |
| Primer: human | GTG TCA GGC TTG AGG TGG AG | 171 |
| Nidogen-2 (Forward): | | |
| Primer: human | TGC CTG ACA TTG ACC CTG T | 172 |
| Nidogen-2 (Reverse): | | |
| Primer: human GATA6 | GCGGGCTCTACAGCAAGAT | 173 |
| (Forward): | | |
| Primer: human GATA6 | TGGCACAGGACAATCCAAG | 174 |
| (Reverse): | | |
| Primer: human GATA4 | GGAAGCCCAAGAACCTGAAT | 175 |
| (Forward): | | |
| Primer: human GATA4 | GTTGCTGGAGTTGCTGGAA | 176 |
| (Reverse): | | |
| Primer: human FOXA2 | TGAAGATGGAAGGGCACGAGC | 177 |
| (Forward): | | |
| Primer: human FOXA2 | CCGACATGCTCATGTACGTGTTCAT | 178 |
| (Reverse): | | |
| Primer: human VIM | AGTCCACTGAGTACCGGAGAC | 179 |
| (Forward): | | |
| Primer: human VIM | CATTTCACGCATCTGGCGTTC | 180 |
| (Reverse): | | |
| Primer: human GAPDH | AGGGCTGCTTTTAACTCTGGT | 181 |
| (Forward): | | |
| Primer: human GAPDH | CCCCACTTGATTTTGGAGGGA | 182 |
| (Reverse): | | |
| Primer: human ACTIN | CCACGAAACTACCTTCAACTCC | 183 |
| (Forward): | | |
| Primer: human ACTIN | GTGATCTCCTTCTGCATCCTGT | 184 |
| (Reverse): | | |
| Primer: human PSG3 | TCG TAA AGC GAG GTG ATG GG | 185 |
| (Forward): | | |
| Primer: human PSG3 | AAG CTC ACA GCC TCC ATG TC | 186 |
| (Reverse): | | |
| Primer: human EGFR | AAC TGT GAG GTG GTC CTT GG | 187 |
| (Forward): | | |
| Primer: human EGFR | GTT GAG GGC AAT GAG GAC AT | 188 |
| (Reverse): | | |
| Primer: human DPP4 | TGG TCT CCA AAC GGC ACT TT | 189 |
| (Forward): | | |
| Primer: human DPP4 | AAC TGT GAG GTG GTC CTT GG | 190 |
| (Reverse): | | |
| Primer: human CGA | CAC TCC ACT AAG GTC CAA GAA | 191 |
| (Forward): | GA | |
| Primer: human CGA | CCG TGT GGT TCT CCA CTT TGA | 192 |
| (Reverse): | | |
| Primer: human CDH10 | AGA TGC CGA TGA CCC TTC ATA | 193 |
| (Forward): | | |
| Primer: human CDH10 | TGT TCG GTA AAG CAG TCC TGA | 194 |
| (Reverse): | | |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of generating a synthetic embryo, the method comprising:
(a) inducing expression of a factor that induces differentiation to trophectoderm cells in a subpopulation of naïve pluripotent stem cells (PSCs) to obtain trophectoderm primed cells; and
(b) mixing said trophectoderm primed cells with naïve PSCs under conditions that allow formation of aggregated cells,
wherein when said factor that induces differentiation to trophectoderm cells is an exogenous factor said inducing said expression comprises inducing transient expression starting within 14 days-0 hours prior to said mixing and ending no later than 120 hours following said mixing; and
wherein said factor that induces differentiation to trophectoderm cells is an endogenous factor said inducing said expression comprises culturing said subpopulation of naïve PSCs under conditions enabling expression of said factor starting within 14 days-6 hours prior to said mixing,
thereby generating the synthetic embryo.

2. The method of claim 1, further comprising inducing expression of a factor that induces differentiation to extra embryonic primitive endodermal cells in a second subpopulation of naïve PSCs to obtain extra embryonic primitive endodermal primed cells in said (a); and mixing said extra embryonic primitive endodermal primed cells with said cells in said (b).

3. The method of claim 2, wherein when said factor that induces differentiation to extra embryonic primitive endodermal cells is an exogenous factor said inducing said expression comprises inducing transient expression starting within 14 days-0 hours prior to said mixing and ending no later than 120 hours following said mixing; and
wherein said factor that induces differentiation to extra embryonic primitive endodermal cells is an endogenous factor said inducing said expression comprises culturing said subpopulation of naïve PSCs under conditions enabling expression of said factor starting within 14 days-6 hours prior to said mixing.

4. The method of claim 2, wherein said mixing said naïve PSCs, said trophectoderm primed cells and said extra embryonic primitive endodermal primed cells is effected concomitantly.

5. The method of claim 2, wherein said mixing said naïve PSCs and said extra embryonic primitive endodermal primed cells is effected prior to said mixing with said trophectoderm primed cells.

6. The method of claim 1, wherein said trophectoderm cells express a pluripotency marker and a trophectoderm marker at said mixing.

7. The method of claim 2, wherein said extra embryonic endodermal primed cells express a pluripotency marker and an extra embryonic primitive endoderm marker at said mixing.

8. A mixture or aggregate, obtainable by the method of claim 1.

9. A mixture or aggregate comprising:
(i) naïve pluripotent stem cell (PSCs);
(ii) trophectoderm primed cells, wherein said trophectoderm primed cells comprise stem cells expressing an endogenous trophectoderm marker and an endogenous pluripotent marker; and
(iii) extra embryonic primitive endodermal primed cells, wherein said extra embryonic primitive endodermal primed cells comprise stem cells expressing an endogenous extra embryonic primitive endoderm marker and an endogenous pluripotency marker,
wherein said mixture or aggregate is not structurally organized as a native embryo at any developmental stage; and
wherein said mixture or aggregate is capable of forming a synthetic embryo structurally organized as a native embryo of at least a post-implantation egg cylinder or a bilaminar disc developmental stage.

10. A mixture or aggregate, comprising:
(i) trophectoderm primed cells, wherein said trophectoderm primed cells comprise stem cells expressing an exogenous factor that induces differentiation to trophectoderm cells, an endogenous trophectoderm marker and an endogenous pluripotent marker; and
(ii) naïve pluripotent stem cell (PSCs).

11. The mixture or aggregate of claim 10, further comprising:
(iii) extra embryonic primitive endodermal primed cells, wherein said extra embryonic primitive endodermal primed cells comprise stem cells expressing an exogenous factor that induces differentiation to extra embryonic primitive endodermal cells, an endogenous extra embryonic primitive endoderm marker and an endogenous pluripotency marker.

12. The method of claim 2, wherein said factor that induces differentiation to extra embryonic primitive endodermal cells comprises Gata4, Gata6 and/or SOX17.

13. The method of claim 3, wherein said inducing expression of a factor that induces differentiation to extra embryonic primitive endodermal cells comprises culturing said second subpopulation of naïve PSCs with a factor that induces differentiation to extra embryonic primitive endodermal cells starting within 14 days-6 hours prior to said mixing, wherein said factor that induces differentiation to extra embryonic primitive endodermal cells is selected from the group consisting of a GSK-3 inhibitor, WNT ligand, heparin, FGF2, FGF4, PDGF, leukemia inhibitory factor (LIF) and insulin.

14. The method of claim 1, wherein said factor that induces differentiation to trophectoderm cells is selected from the group consisting of Cdx2, Gata3 and Gata2.

15. The method of claim 1, wherein said inducing expression of a factor that induces differentiation to trophectoderm cells comprises culturing said subpopulation of naïve PSCs with a factor that induces differentiation to trophectoderm cells starting within 14 days-6 hours prior to said mixing, wherein said factor that induces differentiation to trophectoderm cells is selected from the group consisting of TGFR inhibitor, FGFR inhibitor, MEK/ERK inhibitor, BMP4, JAK inhibitor, a ROCK pathway inhibitor, FGF4, FGF2, heparin, a SUMOylation inhibitor, a Histone Deacetylase inhibitor, a HIPPO signaling pathway inhibitor and a factor that induces YAP nuclear translocation.

16. The method of claim 1, wherein said inducing said transient expression starts within 120-0 hours prior to said mixing, and/or wherein said inducing said expression of said endogenous factor starts within 10-1 days prior to said mixing.

17. The method of claim 1, wherein a ratio between said naïve PSCs and said trophectoderm primed cells is between 1:1 and 1:5.

18. The method of claim 1, further comprising ex-utero culturing said aggregated cells following said (b).

19. The method of claim 18, wherein said culturing is effected in a static culture followed by a dynamic culture.

20. The method of claim 1, wherein said cells are mammalian cells.


SEQUENCE LISTING (199 sequences)

SEQ ID NO: 1 - DNA, 1515 bp, synthetic construct
  misc_feature 1..1515: note = Gata 4 insert
  source 1..1515: mol_type = other DNA
gccctttttgggtttttgttttgttttgttttgttttgatttttggtgacagttccgcacacccgcattctagttcttgtctgcctcgtgctcagagcttggggcgatgtaccaaagcctggccatggccgccaaccacggccccccgcccggcgcctacgaagcaggtggccctggcgccttcatgcacagcgcgggcgccgcgtcctcgcccgtctacgtgcccactccgcgggtgccgtcctctgtgctgggcctgtcctacctgcagggcggtggcagtgccgctgcagctggaaccacctcgggtggcagctccggggccggcccgtcgggtgcagggcctgggacccagcagggtagccctggctggagccaagctggagccgagggagccgcctacaccccgccgcccgtgtccccgcgcttctctttcccggggactactgggtccctggcggccgctgccgccgctgccgcagcccgggaagctgcagcctacggcagtggcggcggggcggcgggcgctggtctggctggccgagagcagtacgggcgtccgggcttcgccggctcctactccagcccctacccagcctacatggccgacgtgggagcatcctgggccgcagccgctgccgcctctgccggccccttcgacagcccagtcctgcacagcctgcctggacgggccaaccctggaagacaccccaatctcgtagatatgtttgatgacttctcagaaggcagagagtgtgtcaattgtggggccatgtccaccccactctggaggcgagatgggacgggacactacctgtgcaatgcctgtggcctctatcacaagatgaacggcatcaaccggcccctcattaagcctcagcgccgcctgtccgcttcccgccgggtaggcctctcctgtgccaactgccagactaccaccaccacgctgtggcgtcgtaatgccgagggtgagcctgtatgtaatgcctgcggcctctacatgaagctccatggggttcccaggcctcttgcaatgcggaaggaggggattcaaaccagaaaacggaagcccaagaacctgaataaatctaagacgccagcaggtcctgctggtgagaccctccctccctccagtggtgcctccagcggtaactccagcaatgccactagcagcagcagcagcagtgaagagatgcgccccatcaagacagagcccgggctgtcatctcactatgggcacagcagctccatgtcccagacattcagtactgtgtccggccacgggccctccatccatccagtgctgtctgctctgaagctgtccccacaaggctatgcatctcctgtcactcagacatcgcaggccagctccaagcaggactcttggaacagcctggtcctggctgacagtcatggggacataatcaccgcgtaatcagcgcccccccttccctcttcaaattcctgctcggacttgggacgtgggggccagcaaagtaaaaggctgggaagggc

SEQ ID NO: 2 - DNA, 22 bp, synthetic construct
  misc_feature 1..22: note = Single strand DNA oligonucleotide
  source 1..22: mol_type = other DNA
agtcaaggccgagaatgggaag

SEQ ID NO: 3 - DNA, 22 bp, synthetic construct
  misc_feature 1..22: note = Single strand DNA oligonucleotide
  source 1..22: mol_type = other DNA
aagcagttggtggtgcaggatg

SEQ ID NO: 4 - DNA, 21 bp, synthetic construct
  misc_feature 1..21: note = Single strand DNA oligonucleotide
  source 1..21: mol_type = other DNA
agaggatcaccttggggtaca

SEQ ID NO: 5 - DNA, 20 bp, synthetic construct
  misc_feature 1..20: note = Single strand DNA oligonucleotide
  source 1..20: mol_type = other DNA
cgaagcgacagatggtggtc

SEQ ID NO: 6 - DNA, 20 bp, synthetic construct
  misc_feature 1..20: note = Single strand DNA oligonucleotide
  source 1..20: mol_type = other DNA
ctcaagtcctgaggctgaca

SEQ ID NO: 7 - DNA, 20 bp, synthetic construct
  misc_feature 1..20: note = Single strand DNA oligonucleotide
  source 1..20: mol_type = other DNA
tgaaacctgtccttgagtgc

SEQ ID NO: 8 - DNA, 23 bp, synthetic construct
  misc_feature 1..23: note = Single strand DNA oligonucleotide
  source 1..23: mol_type = other DNA
tagagctagactccgggcgatga

SEQ ID NO: 9 - DNA, 23 bp, synthetic construct
  misc_feature 1..23: note = Single strand DNA oligonucleotide
  source 1..23: mol_type = other DNA
ttgccttaaacaagaccacgaaa

SEQ ID NO: 10 - DNA, 20 bp, synthetic construct
  misc_feature 1..20: note = Single strand DNA oligonucleotide
  source 1..20: mol_type = other DNA
gcacacctgcgaactcacac

SEQ ID NO: 11 - DNA, 21 bp, synthetic construct
  misc_feature 1..21: note = Single strand DNA oligonucleotide
  source 1..21: mol_type = other DNA
ccgtcccagtcacagtggtaa

SEQ ID NO: 12 - DNA, 22 bp, synthetic construct
  misc_feature 1..22: note = Single strand DNA oligonucleotide
  source 1..22: mol_type = other DNA
gcgaaacctgtgcgagtggatg

SEQ ID NO: 13 - DNA, 28 bp, synthetic construct
  misc_feature 1..28: note = Single strand DNA oligonucleotide
  source 1..28: mol_type = other DNA
cggtatttgtcttttgtcctggttttca

SEQ ID NO: 14 - DNA, 22 bp, synthetic construct
  misc_feature 1..22: note = Single strand DNA oligonucleotide
  source 1..22: mol_type = other DNA
cacaagatgaacggcatcaacc

SEQ ID NO: 15 - DNA, 18 bp, synthetic construct
  misc_feature 1..18: note = Single strand DNA oligonucleotide
  source 1..18: mol_type = other DNA
cagcgtggtggtagtctg

SEQ ID NO: 16 - DNA, 26 bp, synthetic construct
  misc_feature 1..26: note = Single strand DNA oligonucleotide
  source 1..26: mol_type = other DNA
cttgcgggctctatatgaaactccat

SEQ ID NO: 17 - DNA, 32 bp, synthetic construct
  misc_feature 1..32: note = Single strand DNA oligonucleotide
  source 1..32: mol_type = other DNA
tagaagaagaggaagtaggagtcatagggaca

SEQ ID NO: 18 - DNA, 26 bp, synthetic construct
  misc_feature 1..26: note = Single strand DNA oligonucleotide
  source 1..26: mol_type = other DNA
ctgtgactgcctaccagaatgaggag

SEQ ID NO: 19 - DNA, 25 bp, synthetic construct
  misc_feature 1..25: note = Single strand DNA oligonucleotide
  source 1..25: mol_type = other DNA
ggtcgtttctttctttggcatcaag

SEQ ID NO: 20 - DNA, 20 bp, synthetic construct
  misc_feature 1..20: note = Single strand DNA oligonucleotide
  source 1..20: mol_type = other DNA
cttcgggtatggacttgctg

SEQ ID NO: 21 - DNA, 22 bp, synthetic construct
  misc_feature 1..22: note = Single strand DNA oligonucleotide
  source 1..22: mol_type = other DNA
cctcatgaagatgtctgggtac

SEQ ID NO: 22 - DNA, 23 bp, synthetic construct
  misc_feature 1..23: note = Single strand DNA oligonucleotide
  source 1..23: mol_type = other DNA
caaagtcaatggctcccacgaag

SEQ ID NO: 23 - DNA, 27 bp, synthetic construct
  misc_feature 1..27: note = Single strand DNA oligonucleotide
  source 1..27: mol_type = other DNA
ctacaatcccctgagacacagcaaata

SEQ ID NO: 24 - DNA, 20 bp, synthetic construct
  misc_feature 1..20: note = Single strand DNA oligonucleotide
  source 1..20: mol_type = other DNA
ccagtgctgtctgctctgaa

SEQ ID NO: 25 - DNA, 20 bp, synthetic construct
  misc_feature 1..20: note = Single strand DNA oligonucleotide
  source 1..20: mol_type = other DNA
gctgttccaagagtcctgct

SEQ ID NO: 26 - DNA, 22 bp, synthetic construct
  misc_feature 1..22: note = Single strand DNA oligonucleotide
  source 1..22: mol_type = other DNA
gcgaaacctgtgcgagtggatg

SEQ ID NO: 27 - DNA, 23 bp, synthetic construct
  misc_feature 1..23: note = Single strand DNA oligonucleotide
  source 1..23: mol_type = other DNA
cggtatttgtcttttgtcctggt

SEQ ID NO: 28 - DNA, 23 bp, synthetic construct
  misc_feature 1..23: note = Single strand DNA oligonucleotide
  source 1..23: mol_type = other DNA
aaattcctgctcggacttgggac

SEQ ID NO: 29 - DNA, 30 bp, synthetic construct
  misc_feature 1..30: note = Single strand DNA oligonucleotide
  source 1..30: mol_type = other DNA
gtggtttgtccaaactcatcaatgtatctt

SEQ ID NO: 30 - DNA, 26 bp, synthetic construct
  misc_feature 1..26: note = Single strand DNA oligonucleotide
  source 1..26: mol_type = other DNA
gaggggttttaaactccactgtcacc

SEQ ID NO: 31 - DNA, 30 bp, synthetic construct; identical to SEQ ID NO: 29
  misc_feature 1..30: note = Single strand DNA oligonucleotide
  source 1..30: mol_type = other DNA

SEQ ID NO: 32 - DNA, 25 bp, synthetic construct
  misc_feature 1..25: note = Single strand DNA oligonucleotide
  source 1..25: mol_type = other DNA
cactggaaaagcctgcgttcttaca

SEQ ID NO: 33 - DNA, 30 bp, synthetic construct
  misc_feature 1..30: note = Single strand DNA oligonucleotide
  source 1..30: mol_type = other DNA
agaataaggaaggaagaagtctcttgcctc

SEQ ID NO: 34 - DNA, 26 bp, synthetic construct
  misc_feature 1..26: note = Single strand DNA oligonucleotide
  source 1..26: mol_type = other DNA
aattatggacctcaggggaagacatg

SEQ ID NO: 35 - DNA, 22 bp, synthetic construct
  misc_feature 1..22: note = Single strand DNA oligonucleotide
  source 1..22: mol_type = other DNA
gctggcaggaagagtcggaatg

SEQ ID NO: 36 - DNA, 936 bp, synthetic construct
  misc_feature 1..936: note = Cdx2 insert
  source 1..936: mol_type = other DNA
atgtacgtgagctaccttctggacaaggacgtgagcatgtatcctagctccgtgcgccactccggcggcctgaacctggctccgcagaactttgtcagtcctccgcagtacccggactacggtggttaccacgtggcggccgcggcggctgctacggcgaacttggacagcgctcagtccccagggccatcctggcccaccgcgtacggcgcccctctccgcgaggactggaatggctacgcacccgggggcgctgcggcagccaacgcggtagcccacggtctcaatggtggctccccggccgccgctatgggctacagcagccccgccgaataccacgcgcaccatcacccgcatcatcacccgcaccatccggccgcctcgccgtcctgcgcctccggcttgctgcagacgctcaacctcggccccccggggcccgcagccaccgccgccgccgaacagctgtcccccagcggccagcggcgaaacctgtgcgagtggatgcggaagcccgcgcagcagtccctaggaagccaagtgaaaaccaggacaaaagacaaataccgggtggtgtacacagaccatcagcggctggagctggagaaggagtttcactttagtcgatacatcaccatcaggaggaaaagtgagctggctgccacacttgggctctccgagaggcaggttaaaatttggtttcagaaccgcagagccaaggagaggaaaatcaagaagaagcagcagcagcaacagcagcagcagcaacaacagcctccacagccgccgccacaaccttcccagcctcagccgggtgccctgcggagcgtgcccgagcccttgagtcctgtgacctccttgcaaggctcagtgcctggttctgtccctggggttctggggccagctggaggggttttaaactccactgtcacccagtga

SEQ ID NO: 37 - DNA, 23 bp, synthetic construct
  misc_feature 1..23: note = Single strand DNA oligonucleotide
  source 1..23: mol_type = other DNA
atggtcctgctggagttcgtgac

SEQ ID NO: 38 - DNA, 24 bp, synthetic construct
  misc_feature 1..24: note = Single strand DNA oligonucleotide
  source 1..24: mol_type = other DNA
tggtccatctgcttgtaggcaaga

SEQ ID NO: 39 - DNA, 27 bp, synthetic construct
  misc_feature 1..27: note = Single strand DNA oligonucleotide
  source 1..27: mol_type = other DNA
ttcacctttgaagctaatcgtttgagg

SEQ ID NO: 40 - DNA, 22 bp, synthetic construct
  misc_feature 1..22: note = Single strand DNA oligonucleotide
  source 1..22: mol_type = other DNA
aacttgtggccgtttacgtcgc

SEQ ID NO: 41 - DNA, 21 bp, synthetic construct
  misc_feature 1..21: note = Single strand DNA oligonucleotide
  source 1..21: mol_type = other DNA
ctcggccattcgtacatggaa

SEQ ID NO: 42 - DNA, 20 bp, synthetic construct
  misc_feature 1..20: note = Single strand DNA oligonucleotide
  source 1..20: mol_type = other DNA
ggatacctctgcaccgtagc

SEQ ID NO: 43 - DNA, 19 bp, synthetic construct
  misc_feature 1..19: note = Single strand DNA oligonucleotide
  source 1..19: mol_type = other DNA
caccccgccgtattgaatg

SEQ ID NO: 44 - DNA, 20 bp, synthetic construct
  misc_feature 1..20: note = Single strand DNA oligonucleotide
  source 1..20: mol_type = other DNA
cctgcgagtcgagatggttg

SEQ ID NO: 45 - DNA, 21 bp, synthetic construct
  misc_feature 1..21: note = Single strand DNA oligonucleotide
  source 1..21: mol_type = other DNA
atgttggactccgtaacccat

SEQ ID NO: 46 - DNA, 23 bp, synthetic construct
  misc_feature 1..23: note = Single strand DNA oligonucleotide
  source 1..23: mol_type = other DNA
gcagggtagtagtcttcattgct

SEQ ID NO: 47 - DNA, 20 bp, synthetic construct
  misc_feature 1..20: note = Single strand DNA oligonucleotide
  source 1..20: mol_type = other DNA
ggcagctacgcacatcatca

SEQ ID NO: 48 - DNA, 19 bp, synthetic construct
  misc_feature 1..19: note = Single strand DNA oligonucleotide
  source 1..19: mol_type = other DNA
cctggcatcgggaccatag

SEQ ID NO: 49 - DNA, 22 bp, synthetic construct
  misc_feature 1..22: note = Single strand DNA oligonucleotide
  source 1..22: mol_type = other DNA
gcctctcgaacagtattctcct

SEQ ID NO: 50 - DNA, 21 bp, synthetic construct
  misc_feature 1..21: note = Single strand DNA oligonucleotide
  source 1..21: mol_type = other DNA
acagggttcataaggcatggg

SEQ ID NO: 51 - DNA, 22 bp, synthetic construct
  misc_feature 1..22: note = Single strand DNA oligonucleotide
  source 1..22: mol_type = other DNA
atgatgtcccttcaagtcctca

SEQ ID NO: 52 - DNA, 22 bp, synthetic construct
  misc_feature 1..22: note = Single strand DNA oligonucleotide
  source 1..22: mol_type = other DNA
tgtgttgggatcacttcagtgt

SEQ ID NO: 53 - DNA, 19 bp, synthetic construct
  misc_feature 1..19: note = Single strand DNA oligonucleotide
  source 1..19: mol_type = other DNA
tcaggggtaggggcatcag

SEQ ID NO: 54 - DNA, 20 bp, synthetic construct
  misc_feature 1..20: note = Single strand DNA oligonucleotide
  source 1..20: mol_type = other DNA
gtagaggccgtcttgacctg

SEQ ID NO: 55 - DNA, 20 bp, synthetic construct
  misc_feature 1..20: note = Single strand DNA oligonucleotide
  source 1..20: mol_type = other DNA
agcagaacccagatctgcac

SEQ ID NO: 56 - DNA, 20 bp, synthetic construct
  misc_feature 1..20: note = Single strand DNA oligonucleotide
  source 1..20: mol_type = other DNA
cgccttccaagacttgccta

SEQ ID NO: 57 - DNA, 19 bp, synthetic construct
  misc_feature 1..19: note = Single strand DNA oligonucleotide
  source 1..19: mol_type = other DNA
gatatcgctgcgctggtcg

SEQ ID NO: 58 - DNA, 21 bp, synthetic construct
  misc_feature 1..21: note = Single strand DNA oligonucleotide
  source 1..21: mol_type = other DNA
ccacgatggaggggaatacag

SEQ ID NO: 59 - DNA, 20 bp, synthetic construct
  misc_feature 1..20: note = Single strand DNA oligonucleotide
  source 1..20: mol_type = other DNA
tgttgctaacacgatgccct

SEQ ID NO: 60 - DNA, 20 bp, synthetic construct
  misc_feature 1..20: note = Single strand DNA oligonucleotide
  source 1..20: mol_type = other DNA
ggccactcacacatctgtcc

SEQ ID NO: 61 - DNA, 20 bp, synthetic construct
  misc_feature 1..20: note = Single strand DNA oligonucleotide
  source 1..20: mol_type = other DNA
gcactcggcttccagtatgc

SEQ ID NO: 62 - DNA, 20 bp, synthetic construct
  misc_feature 1..20: note = Single strand DNA oligonucleotide
  source 1..20: mol_type = other DNA
cgtagtagctgctccagtcg

SEQ ID NO: 63 - DNA, 20 bp, synthetic construct
  misc_feature 1..20: note = Single strand DNA oligonucleotide
  source 1..20: mol_type = other DNA
agtggctacatcatccccct

SEQ ID NO: 64 - DNA, 20 bp, synthetic construct
  misc_feature 1..20: note = Single strand DNA oligonucleotide
  source 1..20: mol_type = other DNA
ccgaagtctgtgagctgtgt

SEQ ID NO: 65 - DNA, 203 bp, synthetic construct
  misc_feature 1..203: note = 203bp amplicon covering human XIST transcription start site
  source 1..203: mol_type = other DNA
gggtaaattttgaaccaaccaaatcacaaagatgtccggctttcaatcttctaggccacgcctcttatgctctctccgccctcagcccccccttcagttcttaaagcgctgcaattcgctgctgcagccatatttcttactctctcggggctggaagcttcctgactgaagatctgttctagaaagaaccccaagtgcagaga

SEQ ID NO: 66 - DNA, 20 bp, synthetic construct
  misc_feature 1..20: note = Single strand DNA oligonucleotide
  source 1..20: mol_type = other DNA
agggagcagtttgccctact

SEQ ID NO: 67 - DNA, 20 bp, synthetic construct
  misc_feature 1..20: note = Single strand DNA oligonucleotide
  source 1..20: mol_type = other DNA
cacatgcagcgtggtatctt

SEQ ID NO: 68 - protein, 313 aa, Homo sapiens
MYVSYLLDKDVSMYPSSVRHSGGLNLAPQNFVSPPQYPDYGGYHVAAAAAAAANLDSAQSPGPSWPAAYGAPLREDWNGYAPGGAAAAANAVAHGLNGGSPAAAMGYSSPADYHPHHHPHHHPHHPAAAPSCASGLLQTLNPGPPGPAATAAAEQLSPGGQRRNLCEWMRKPAQQSLGSQVKTRTKDKYRVVYTDHQRLELEKEFHYSRYITIRRKAELAATLGLSERQVKIWFQNRRAKERKINKKKLQQQQQQQPPQPPPPPPQPPQPQPGPLRSVPEPLSPVSSLQASVPGSVPGVLGPTGGVLNPTVTQ

SEQ ID NO: 69 - skipped

SEQ ID NO: 70 - DNA, 3448 bp, Homo sapiens
  source 1..3448: mol_type = genomic DNA
  mRNA 1..3448
aatcacggaaggccgccggcctggggctccgcacgccagcctgtggcgggtcttccccgcctctgcagcctagtgggaaggaggtgggaggaaagaaggaagaaagggagggagggaggaggcaggccagagggagggaccgcctcggaggcagaagagccgcgaggagccagcggagcaccgcgggctggggcgcagccacccgccgctcctcgagtcccctcgcccctttcccttcgtgccccccggcagcctccagcgtcggtccccaggcagcatggtgaggtctgctcccggaccctcgccaccatgtacgtgagctacctcctggacaaggacgtgagcatgtaccctagctccgtgcgccactctggcggcctcaacctggcgccgcagaacttcgtcagccccccgcagtacccggactacggcggttaccacgtggcggccgcagctgcagcggcagcgaacttggacagcgcgcagtccccggggccatcctggccggcagcgtatggcgccccactccgggaggactggaatggctacgcgcccggaggcgccgcggccgccgccaacgccgtggctcacggcctcaacggtggctccccggccgcagccatgggctacagcagccccgcagactaccatccgcaccaccacccgcatcaccacccgcaccacccggccgccgcgccttcctgcgcttctgggctgctgcaaacgctcaaccccggccctcctgggcccgccgccaccgctgccgccgagcagctgtctcccggcggccagcggcggaacctgtgcgagtggatgcggaagccggcgcagcagtccctcggcagccaagtgaaaaccaggacgaaagacaaatatcgagtggtgtacacggaccaccagcggctggagctggagaaggagtttcactacagtcgctacatcaccatccggaggaaagccgagctagccgccacgctggggctctctgagaggcaggttaaaatctggtttcagaaccgcagagcaaaggagaggaaaatcaacaagaagaagttgcagcagcaacagcagcagcagccaccacagccgcctccgccgccaccacagcctccccagcctcagccaggtcctctgagaagtgtcccagagcccttgagtccggtgtcttccctgcaagcctcagtgcctggctctgtccctggggttctggggccaactgggggggtgctaaaccccaccgtcacccagtgacccaccgggttctgcagcggcagagcaattccaggctgagccatgaggagcgtggactctgctagactcctcaggagagacccctcccctcccacccacagccatagacctacagacctggctctcagaggaaaaatgggagccaggagtaagacaagtgggatttggggcctcaagaaatatactctcccagatttttactttttcccatctggctttttctgccactgaggagacagaaagcctccgctgggcttcattccggactggcagaagcattgcctggactgaccacaccaaccaggccttcatcctcctccccagctcttctcttcctagatctgcaggctgcacctctggctagagccgaggggagagagggactcaagggaaaggcaagcttgaggccaagatggctgctgcctgctcatggccctcggaggtccagctgggcctcctgcctccgggcaggcaaggtttacactgcggaagccaaaggcagctaagatagaaagctggactgaccaaagactgcagaacccccaggtggcctgcgtcttttttctcttcccttcccagaccaggaaaggcttggctggtgtatgcacagggtgtggtatgagggggtggttattggactccaggcctgaccagggggcccgaacagggacttgtttagagagcctgtcaccagagcttctctgggctgaatgtatgtcagtgctataaatgccagagccaacctggacttcctgtcattttcacaatcttggggctgatgaagaagggggtggggggagtttgtgttgttgttgctgctgtttgggttgttggtctgtgtaacatccaagccagagtttttaaagccttctggatccatggggggagaagtgatatggtgaagggaagtggggagtatttgaacacagttgaattttttctaaaaagaaaaagagataaatgagctttccagatttcagattctgtatttatcttcagattttgtctgcaactattttttattttttaaagaaatgaaatatcttctctgcttgcaagctgatttggaaaattcagagagggagtggagattatctggacagcctcattttaccccagagcatcagaagcctgaaagagcaggttctgagaccttccctgcccccaagcctgagcccagccttggaatgcgtggctctcctgggggtgcctttgggcaaaatgcacatctgaatggccaggtgcttcccagcagtcagaaattatggggggtgggggtgagagggccccctcatagcatctcaccaaatccacatggaggctttttgcaaacagcaggcagttcccttctgcagtgactccctcctaaggacgccccacaacccagctagttaaacgtgagcattaaattttttttaaaaaaaatccccctagtttcccaagacagcatttccatgaatttagtcttctgtaaatcactgggcatttccgtgagccctttctgcctccactctcttctctgtctttgcagtttccttatccccgaccgcgcccccccttccaacccaatcaccccacaaacaagatttctagacccagtataaaatgatccttttagtgacagtttcttgttatctggccgatccactggggaccgggctgcagcctttaaaatttttgatcctggaggccgccgagctgaactttccggcaggacccgggcgaggggggcttagcccttcgtttcgatcttcccaccaacatccgagagcctaatcagcgcgcccacggaggcgccttaagggcagttggggaagatgagcagagccgggaaacagcaagaggtatagaccctctgcagcacctctccaattccgcggcccttccgggtggcgtatacagctccaggattgggaaaggggctccggtggcccggcccgcgggcgcctggcctcgaagccagagggaggagtgcggggcgtggggtcggggggtgccgggcagaggaggaagccgtgtccgggtccgctgggcgaggggtcctgggtcaggggtcctgggtcagggccggtctgagccccggtgccccgcaggtctccccgcgccccggccgcgcccacaggcccgccccctagccgccggggttgctatgcgttgccgtgaaacgcctgtcaataaaccctgtttggacagtgga

SEQ ID NO: 71 - skipped

SEQ ID NO: 72 - protein, 311 aa, Mus musculus
MYVSYLLDKDVSMYPSSVRHSGGLNLAPQNFVSPPQYPDYGGYHVAAAAAATANLDSAQSPGPSWPTAYGAPLREDWNGYAPGGAAAANAVAHGLNGGSPAAAMGYSSPAEYHAHHHPHHHPHHPAASPSCASGLLQTLNLGPPGPAATAAAEQLSPSGQRRNLCEWMRKPAQQSLGSQVKTRTKDKYRVVYTDHQRLELEKEFHFSRYITIRRKSELAATLGLSERQVKIWFQNRRAKERKIKKKQQQQQQQQQQQPPQPPPQPSQPQPGALRSVPEPLSPVTSLQGSVPGSVPGVLGPAGGVLNSTVTQ

SEQ ID NO: 73 - DNA, 2145 bp, Mus musculus
  source 1..2145: mol_type = genomic DNA
tccgcgagccaacctgcggcgggtcatccccgcctctacagcttactggcaaggaggtgggaggaaagaaggaagagaggggaggaggcaggacggagggagggactgcccgggaggcagaagctctgcaaggagccgacggagcaccgtgggctgaggtgcagccagctacctttatctctagccccctgcgcctcgcgcctctggcagccttcaacgtttgtccccagacagcatggtgaggtctgctctgggtccctcgccaccatgtacgtgagctaccttctggacaaggacgtgagcatgtatcctagctccgtgcgccactccggcggcctgaacctggctccgcagaactttgtcagtcctccgcagtacccggactacggtggttaccacgtggcggccgcggcggctgctacggcgaacttggacagcgctcagtccccagggccatcctggcccaccgcgtacggcgcccctctccgcgaggactggaatggctacgcacccgggggcgctgcggcagccaacgcggtagcccacggtctcaatggtggctccccggccgccgctatgggctacagcagccccgccgaataccacgcgcaccatcacccgcatcatcacccgcaccatccggccgcctcgccgtcctgcgcctccggcttgctgcagacgctcaacctcggccccccggggcccgcagccaccgccgccgccgaacagctgtcccccagcggccagcggcgaaacctgtgcgagtggatgcggaagcccgcgcagcagtccctaggaagccaagtgaaaaccaggacaaaagacaaataccgggtggtgtacacagaccatcagcggctggagctggagaaggagtttcactttagtcgatacatcaccatcaggaggaaaagtgagctggctgccacacttgggctctccgagaggcaggttaaaatttggtttcagaaccgcagagccaaggagaggaaaatcaagaagaagcagcagcagcaacagcagcagcagcaacaacagcctccacagccgccgccacaaccttcccagcctcagccgggtgccctgcggagcgtgcccgagcccttgagtcctgtgacctccttgcaaggctcagtgcctggttctgtccctggggttctggggccagctggaggggttttaaactccactgtcacccagtgacccctcccgtggtctgaagcggcggcggcacagcaatcccaggctgagccatgaggagtatggacgctgcgagaatcctcagaagagattcctctcctcctacccacgaacagcatctactgatggagattgaggacagaagatgagtggaattatggacctcaggggaagacatggtttagattttttttttctttttaacttttcccattccgactcttcctgccagcaacgacaaacgaagtgattcctggggcttcttcgttcatgctctttgccaggactgactaccgacatgaagctatcagcctcttttgccccagctctttgcctctctgtatttctgtgtggagctgaggagagagtgagactggatggggtgggggtagcaatacttgagccaaggtggctgtttcctgctgactgctttctgagaaccagctggccgtcctgcctccgggccagggactattcaaactacaggagccagaggcagctaagatagctggactgaccgaagtctgcagaacctcccccaccaggtggtctgggctttcttctccacaaatcaggaaggggtggtgggttcaggggctgcggtgagagggggttggttagccaacgccaggcccctgcgacaagggcttgtttagaaagcctgtcaccagagctgctgtaggcggaatgtatgtctgtgttgtaaatgccagagccaacctggacttcctgtcccttccctcgtctttggctgaagaagaccggaattgtttgctgctgttcgagtcactgatctgtgtaacgagccaaacaagccttttaaaaagccttcttgatccatgggtagagaagttgtatggtgaagggaagtcgggagggggggaaggggatccgaacacagttgacttttattttgtaaaaagacaaagataaacgaactttaac

SEQ ID NO: 74 - protein, 444 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 75 - skipped

SEQ ID NO: 76 - protein, 443 aa, Mus musculus
MEVTADQPRWVSHHHPAVLNGQHPDTHHPGLGHSYMEAQYPLTEEVDVLFNIDGQGNHVPSYYGNSVRATVQRYPPTHHGSQVCRPPLLHGSLPWLDGGKALSSHHTASPWNLSPFSKTSIHHGSPGPLSVYPPASSSSLAAGHSSPHLFTFPPTPPKDVSPDPSLSTPGSAGSARQDEKECLKYQVQLPDSMKLETSHSRGSMTTLGGASSSAHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDALEDFPKSSSFNPAALSRHMSSLSHISPFSHSSHMLTTPTPMHPPSGLSFGPHHPSSMVTAMG

SEQ ID NO: 77 - protein, 442 aa, Mus musculus
MEVTADQPRWVSHHHPAVLNGQHPDTHHPGLGHSYMEAQYPLTEEVDVLFNIDGQGNHVPSYYGNSVRATVQRYPPTHHGSQVCRPPLLHGSLPWLDGGKALSSHHTASPWNLSPFSKTSIHHGSPGPLSVYPPASSSSLAAGHSSPHLFTFPPTPPKDVSPDPSLSTPGSAGSARQDEKECLKYQVQLPDSMKLETSHSRGSMTTLGGASSSAHHPITTYPPYVPEYSSGLFPPSSLLGGSPTGFGCKSRPKARSSTGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDALEDFPKSSSFNPAALSRHMSSLSHISPFSHSSHMLTTPTPMHPPSGLSFGPHHPSSMVTAMG

SEQ ID NO: 78 - protein, 366 aa, Mus musculus
MEVTADQPRWVSHHHPAVLNGQHPDTHHPGLGHSYMEAQYPLTEEVDVLFNIDGQGNHVPSYYGNSVRATVQRYPPTHHGSQVCRPPLLHGSLPWLDGGKALSSHHTASPWNLSPFSKTSIHHGSPGPLSVYPPASSSSLAAGHSSPHLFTFPPTPPKDVSPDPSLSTPGSAGSARQDEKECLKYQVQLPDSMKLETSHSRGSMTTLGGASSSAHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNTPDYEERRHPDPKPEDV

SEQ ID NO: 79 - protein, 365 aa, Mus musculus
MEVTADQPRWVSHHHPAVLNGQHPDTHHPGLGHSYMEAQYPLTEEVDVLFNIDGQGNHVPSYYGNSVRATVQRYPPTHHGSQVCRPPLLHGSLPWLDGGKALSSHHTASPWNLSPFSKTSIHHGSPGPLSVYPPASSSSLAAGHSSPHLFTFPPTPPKDVSPDPSLSTPGSAGSARQDEKECLKYQVQLPDSMKLETSHSRGSMTTLGGASSSAHHPITTYPPYVPEYSSGLFPPSSLLGGSPTGFGCKSRPKARSSTGRECVNCGATSTPLWRRDGTGHYLCNACGLYHKMNGQNRPLIKPKRRLSAARRAGTSCANCQTTTTTLWRRNANGDPVCNACGLYYKLHNTPDYEERRHPDPKPEDV

SEQ ID NO: 80 - DNA, 3257 bp, Mus musculus
  source 1..3257: mol_type = genomic DNA
agctgtctgcgaacactgagctgcctggcgccgtcttgatagtttcagaaagaatgcattccctgtaaaaaaaaaatactgagagagggagaggagaaagagagagagactgagagagcgagacatagagagctacgcaatctgaccgggcaggtcacacgcctcctcctcctcctctacgctccttgctactcaggtgatcggaagagcaaccgtctctgagcgccaaggaatcagtgtgcagtgtggtcacactcggattcctctctccctcctttttttttttttttttttgacccctttattcctccgtgtctgctttttttttggggggggggatcgccctcattcttttctttttcttctttcccttcctttcttttgctaaactatcccgcaaagatttttctttcctccctaaaccctcctttttgctctccttttctatacccttaactgcaaacaaaccattaaacgacccctctcctgggcctccgacggcaggagtccgcggacctcccaggccgacagccctccctctacccgcgagggttccgggccgggcgagagggcgcgagcacagccgaggacatggaggtgactgcggaccagccgcgctgggtgagccaccatcaccccgcggtcctcaacggtcagcacccagacacgcaccacccgggcctcggccattcgtacatggaagctcagtatccgctgacggaagaggtggacgtactttttaacatcgatggtcaaggcaaccacgtcccgtcctactacggaaactccgtcagggctacggtgcagaggtatcctccgacccaccacgggagccaggtatgccgcccgcctctgctgcacggatctctgccctggctggatggcggcaaagccctgagcagccaccacaccgcctcgccctggaacctcagccccttctccaagacgtccatccaccacggctctccggggcctctgtccgtttaccctccggcttcatcctcttctctggcggccggccactccagtcctcatctcttcaccttcccgcccaccccgccgaaagacgtctccccagacccgtcgctgtccaccccgggatccgccgggtcggccaggcaagatgagaaagagtgcctcaagtatcaggtgcagctgccagatagcatgaagctggagacgtctcactctcgaggcagcatgaccaccctgggtggggcctcatcctcagcccaccaccccattaccacctatccgccctatgtgcccgagtacagctctggactcttcccacccagcagcctgctgggaggatcccctaccgggttcggatgtaagtcgaggcccaaggcacgatccagcacagaaggcagggagtgtgtgaactgcggggcaacctctaccccactgtggcggcgagatggtaccgggcactacctttgcaatgcctgcggactctaccataaaatgaatgggcagaaccggccccttatcaagcccaagcgaaggctgtcggcagcaaggagagcagggacatcctgcgcgaactgtcagaccaccaccaccaccctctggaggaggaacgctaatggggacccggtctgcaatgcctgtgggctgtactacaagcttcataatattaacagacccctgactatgaagaaagaaggcatccagacccgaaaccggaagatgtctagcaaatcgaaaaagtgcaaaaaggtgcatgacgcgctggaggacttccccaagagcagctccttcaacccggccgctctctccagacacatgtcatccctgagccacatctctcccttcagccactccagccacatgctgaccacaccgacgcccatgcatccgccctccggcctctccttcggacctcaccacccttccagcatggtcaccgccatgggttagagaggcagagccctgctccacatgcgtgaggagtctccaagtgtgcgaagagttcctccgaccccttctacttgcgtttttcgcaggagcagtatcatgaagcccgaaagcgacagatctgtgtttttgaaggcagaaagcaaaatgtttgcttcttttttcaaaggagctcgaggtggtgtctgcattccaaccactgaatccggatcccatttgtgaataagccattcagactcatattccctatttaacagggtctctagtgctgtgaaaaaaatattgctgaacattgcatataacttatattgtaagaaatactgtacatttgaggaagactttattgtacctggatagctgtaagaaaggcatgaaggacgccaagagttttaaggaatatagggggattaaagtatggagatacagaagaaaccactaagtctgatgtccaaatgggcacactgtcagttttgtttcccttcagttgtttgatgcatttaaaaaaaaaaaaaagaaagaaaaagaaaaaaaggggggggggggagaaaaaaataaattaaaaaaaaaaaaaaaaaaagaaaagaaagaaaaatctaagaaaaaaaaaaaaaaggttgtaggcaaatcatttgttccaggctgtgagcctgtgcaaaagagatttcagatctgggcaatgggtgtgtgatctcacccactgaagatctgagaatgtcatggctaggcctacatgctctgtgaatcagtccctgtaattgttgtttgtatgtataattcagaagcaccaaaataagaaaagatgtagatttatttcatcatattatacagactgaattgttgtataaatttatttactgctagtgttaggaactgcttttttttttttttttggttttaatgttttttttttttttgttttttgtttttttttttctttctctctggatttttggttgaataaactagattgctttcagttgacttaaggtggatgtactctggagggtttatttttccttttattattatttttgatggtatttattaaatagcttctatgggcccggcggtacctgtctttttcgtcacttttcttgcagcctaaactatgaaggtagcagcgtaccagctaccaacatgcatgtcagagacccggccactcacaggcctggtcctgagagccacctggctgactgttagcccctgtgtgttctgtattagtgatcactgcctttaaacagtctgttggaataatactataaaaataataataaagttaaaatattttaaaacaaaaaaaaaaaaaaaaaaaaaaaaaaaaaaaaaaaaaaaaaaaa

SEQ ID NO: 81 - DNA, 3212 bp, Mus musculus
  source 1..3212: mol_type = genomic DNA
agctgtctgcgaacactgagctgcctggcgccgtcttgatagtttcagaaagaatgcattccctgtaaaaaaaaaatactgagagagggagaggagaaagagagagagactgagagagcgagacatagagagctacgcaatctgaccgggcaggtcacacgcctcctcctcctcctctacgctccttgctactcaggtgatcggaagagcaaccgtctctgagcgccaaggaatcagtgtgcagtgtggtcacactcggattcctctctccctcctttttttttttttttttttgacccctttattcctccgtgtctgctttttttttggggggggggatcgccctcattcttttctttttcttctttcccttcctttcttttgctaaactatcccgcaaagatttttctttcctccctaaaccctcctttttgctctccttttctatacccttaactgcaaacaaaccattaaacgacccctctcctgggcctccgacggcaggagtccgcggacctcccaggccgacagccctccctctacccgcgagggttccgggccgggcgagagggcgcgagcacagccgaggacatggaggtgactgcggaccagccgcgctgggtgagccaccatcaccccgcggtcctcaacggtcagcacccagacacgcaccacccgggcctcggccattcgtacatggaagctcagtatccgctgacggaagaggtggacgtactttttaacatcgatggtcaaggcaaccacgtcccgtcctactacggaaactccgtcagggctacggtgcagaggtatcctccgacccaccacgggagccaggtatgccgcccgcctctgctgcacggatctctgccctggctggatggcggcaaagccctgagcagccaccacaccgcctcgccctggaacctcagccccttctccaagacgtccatccaccacggctctccggggcctctgtccgtttaccctccggcttcatcctcttctctggcggccggccactccagtcctcatctcttcaccttcccgcccaccccgccgaaagacgtctccccagacccgtcgctgtccaccccgggatccgccgggtcggccaggcaagatgagaaagagtgcctcaagtatcaggtgcagctgccagatagcatgaagctggagacgtctcactctcgaggcagcatgaccaccctgggtggggcctcatcctcagcccaccaccccattaccacctatccgccctatgtgcccgagtacagctctggactcttcccacccagcagcctgctgggaggatcccctaccgggttcggatgtaagtcgaggcccaaggcacgatccagcacaggcagggagtgtgtgaactgcggggcaacctctaccccactgtggcggcgagatggtaccgggcactacctttgcaatgcctgcggactctaccataaaatgaatgggcagaaccggccccttatcaagcccaagcgaaggctgtcggcagcaaggagagcagggacatcctgcgcgaactgtcagaccaccaccaccaccctctggaggaggaacgctaatggggacccggtctgcaatgcctgtgggctgtactacaagcttcataatattaacagacccctgactatgaagaaagaaggcatccagacccgaaaccggaagatgtctagcaaatcgaaaaagtgcaaaaaggtgcatgacgcgctggaggacttccccaagagcagctccttcaacccggccgctctctccagacacatgtcatccctgagccacatctctcccttcagccactccagccacatgctgaccacaccgacgcccatgcatccgccctccggcctctccttcggacctcaccacccttccagcatggtcaccgccatgggttagagaggcagagccctgctccacatgcgtgaggagtctccaagtgtgcgaagagttcctccgaccccttctacttgcgtttttcgcaggagcagtatcatgaagcccgaaagcgacagatctgtgtttttgaaggcagaaagcaaaatgtttgcttcttttttcaaaggagctcgaggtggtgtctgcattccaaccactgaatccggatcccatttgtgaataagccattcagactcatattccctatttaacagggtctctagtgctgtgaaaaaaatattgctgaacattgcatataacttatattgtaagaaatactgtacatttgaggaagactttattgtacctggatagctgtaagaaaggcatgaaggacgccaagagttttaaggaatatagggggattaaagtatggagatacagaagaaaccactaagtctgatgtccaaatgggcacactgtcagttttgtttcccttcagttgtttgatgcatttaaaaaaaaaaaaaagaaagaaaaagaaaaaaaggggggggggggagaaaaaaataaattaaaaaaaaaaaaaaaaaaagaaaagaaagaaaaatctaagaaaaaaaaaaaaaaggttgtaggcaaatcatttgttccaggctgtgagcctgtgcaaaagagatttcagatctgggcaatgggtgtgtgatctcacccactgaagatctgagaatgtcatggctaggcctacatgctctgtgaatcagtccctgtaattgttgtttgtatgtataattcagaagcaccaaaataagaaaagatgtagatttatttcatcatattatacagactgaattgttgtataaatttatttactgctagtgttaggaactgcttttttttttttttttggttttaatgttttttttttttttgttttttgtttttttttttctttctctctggatttttggttgaataaactagattgctttcagttgacttaaggtggatgtactctggagggtttatttttccttttattattatttttgatggtatttattaaatagcttctatgggcccggcggtacctgtctttttcgtcacttttcttgcagcctaaactatgaaggtagcagcgtaccagctaccaacatgcatgtcagagacccggccactcacaggcctggtcctgagagccacctggctgactgttagcccctgtgtgttctgtattagtgatcactgcctttaaacagtctgttggaataatactataaaaataataataaagttaaaatattttaaaacaa

SEQ ID NO: 82 - DNA, 3207 bp, Mus musculus
  source 1..3207: mol_type = genomic DNA
agctgtctgcgaacactgagctgcctggcgccgtcttgatagtttcagaaagaatgcattccctgtaaaaaaaaaatactgagagagggagaggagaaagagagagagactgagagagcgagacatagagagctacgcaatctgaccgggcaggtcacacgcctcctcctcctcctctacgctccttgctactcaggtgatcggaagagcaaccgtctctgagcgccaaggaatcagtgtgcagtgtggtcacactcggattcctctctccctcctttttttttttttttttttgacccctttattcctccgtgtctgctttttttttggggggggggatcgccctcattcttttctttttcttctttcccttcctttcttttgctaaactatcccgcaaagatttttctttcctccctaaaccctcctttttgctctccttttctatacccttaactgcaaacaaaccattaaacgacccctctcctgggcctccgacggcaggagtccgcggacctcccaggccgacagccctccctctacccgcgagggttccgggccgggcgagagggcgcgagcacagccgaggacatggaggtgactgcggaccagccgcgctgggtgagccaccatcaccccgcggtcctcaacggtcagcacccagacacgcaccacccgggcctcggccattcgtacatggaagctcagtatccgctgacggaagaggtggacgtactttttaacatcgatggtcaaggcaaccacgtcccgtcctactacggaaactccgtcagggctacggtgcagaggtatcctccgacccaccacgggagccaggtatgccgcccgcctctgctgcacggatctctgccctggctggatggcggcaaagccctgagcagccaccacaccgcctcgccctggaacctcagccccttctccaagacgtccatccaccacggctctccggggcctctgtccgtttaccctccggcttcatcctcttctctggcggccggccactccagtcctcatctcttcaccttcccgcccaccccgccgaaagacgtctccccagacccgtcgctgtccaccccgggatccgccgggtcggccaggcaagatgagaaagagtgcctcaagtatcaggtgcagctgccagatagcatgaagctggagacgtctcactctcgaggcagcatgaccaccctgggtggggcctcatcctcagcccaccaccccattaccacctatccgccctatgtgcccgagtacagctctggactcttcccacccagcagcctgctgggaggatcccctaccgggttcggatgtaagtcgaggcccaaggcacgatccagcacagaaggcagggagtgtgtgaactgcggggcaacctctaccccactgtggcggcgagatggtaccgggcactacctttgcaatgcctgcggactctaccataaaatgaatgggcagaaccggccccttatcaagcccaagcgaaggctgtcggcagcaaggagagcagggacatcctgcgcgaactgtcagaccaccaccaccaccctctggaggaggaacgctaatggggacccggtctgcaatgcctgtgggctgtactacaagcttcataatacccctgactatgaagaaagaaggcatccagacccgaaaccggaagatgtctagcaaatcgaaaaagtgcaaaaaggtgcatgacgcgctggaggacttccccaagagcagctccttcaacccggccgctctctccagacacatgtcatccctgagccacatctctcccttcagccactccagccacatgctgaccacaccgacgcccatgcatccgccctccggcctctccttcggacctcaccacccttccagcatggtcaccgccatgggttagagaggcagagccctgctccacatgcgtgaggagtctccaagtgtgcgaagagttcctccgaccccttctacttgcgtttttcgcaggagcagtatcatgaagcccgaaagcgacagatctgtgtttttgaaggcagaaagcaaaatgtttgcttcttttttcaaaggagctcgaggtggtgtctgcattccaaccactgaatccggatcccatttgtgaataagccattcagactcatattccctatttaacagggtctctagtgctgtgaaaaaaatattgctgaacattgcatataacttatattgtaagaaatactgtacatttgaggaagactttattgtacctggatagctgtaagaaaggcatgaaggacgccaagagttttaaggaatatagggggattaaagtatggagatacagaagaaaccactaagtctgatgtccaaatgggcacactgtcagttttgtttcccttcagttgtttgatgcatttaaaaaaaaaaaaaagaaagaaaaagaaaaaaaggggggggggggagaaaaaaataaattaaaaaaaaaaaaaaaaaaagaaaagaaagaaaaatctaagaaaaaaaaaaaaaaggttgtaggcaaatcatttgttccaggctgtgagcctgtgcaaaagagatttcagatctgggcaatgggtgtgtgatctcacccactgaagatctgagaatgtcatggctaggcctacatgctctgtgaatcagtccctgtaattgttgtttgtatgtataattcagaagcaccaaaataagaaaagatgtagatttatttcatcatattatacagactgaattgttgtataaatttatttactgctagtgttaggaactgcttttttttttttttttggttttaatgttttttttttttttgttttttgtttttttttttctttctctctggatttttggttgaataaactagattgctttcagttgacttaaggtggatgtactctggagggtttatttttccttttattattatttttgatggtatttattaaatagcttctatgggcccggcggtacctgtctttttcgtcacttttcttgcagcctaaactatgaaggtagcagcgtaccagctaccaacatgcatgtcagagacccggccactcacaggcctggtcctgagagccacctggctgactgttagcccctgtgtgttctgtattagtgatcactgcctttaaacagtctgttggaataatactataaaaataataataaagttaaaatattttaaaacaa

SEQ ID NO: 83 - DNA, 3204 bp, Mus musculus
  source 1..3204: mol_type = genomic DNA
agctgtctgcgaacactgagctgcctggcgccgtcttgatagtttcagaaagaatgcattccctgtaaaaaaaaaatactgagagagggagaggagaaagagagagagactgagagagcgagacatagagagctacgcaatctgaccgggcaggtcacacgcctcctcctcctcctctacgctccttgctactcaggtgatcggaagagcaaccgtctctgagcgccaaggaatcagtgtgcagtgtggtcacactcggattcctctctccctcctttttttttttttttttttgacccctttattcctccgtgtctgctttttttttggggggggggatcgccctcattcttttctttttcttctttcccttcctttcttttgctaaactatcccgcaaagatttttctttcctccctaaaccctcctttttgctctccttttctatacccttaactgcaaacaaaccattaaacgacccctctcctgggcctccgacggcaggagtccgcggacctcccaggccgacagccctccctctacccgcgagggttccgggccgggcgagagggcgcgagcacagccgaggacatggaggtgactgcggaccagccgcgctgggtgagccaccatcaccccgcggtcctcaacggtcagcacccagacacgcaccacccgggcctcggccattcgtacatggaagctcagtatccgctgacggaagaggtggacgtactttttaacatcgatggtcaaggcaaccacgtcccgtcctactacggaaactccgtcagggctacggtgcagaggtatcctccgacccaccacgggagccaggtatgccgcccgcctctgctgcacggatctctgccctggctggatggcggcaaagccctgagcagccaccacaccgcctcgccctggaacctcagccccttctccaagacgtccatccaccacggctctccggggcctctgtccgtttaccctccggcttcatcctcttctctggcggccggccactccagtcctcatctcttcaccttcccgcccaccccgccgaaagacgtctccccagacccgtcgctgtccaccccgggatccgccgggtcggccaggcaagatgagaaagagtgcctcaagtatcaggtgcagctgccagatagcatgaagctggagacgtctcactctcgaggcagcatgaccaccctgggtggggcctcatcctcagcccaccaccccattaccacctatccgccctatgtgcccgagtacagctctggactcttcccacccagcagcctgctgggaggatcccctaccgggttcggatgtaagtcgaggcccaaggcacgatccagcacaggcagggagtgtgtgaactgcggggcaacctctaccccactgtggcggcgagatggtaccgggcactacctttgcaatgcctgcggactctaccataaaatgaatgggcagaaccggccccttatcaagcccaagcgaaggctgtcggcagcaaggagagcagggacatcctgcgcgaactgtcagaccaccaccaccaccctctggaggaggaacgctaatggggacccggtctgcaatgcctgtgggctgtactacaagcttcataatacccctgactatgaagaaagaaggcatccagacccgaaaccggaagatgtctagcaaatcgaaaaagtgcaaaaaggtgcatgacgcgctggaggacttccccaagagcagctccttcaacccggccgctctctccagacacatgtcatccctgagccacatctctcccttcagccactccagccacatgctgaccacaccgacgcccatgcatccgccctccggcctctccttcggacctcaccacccttccagcatggtcaccgccatgggttagagaggcagagccctgctccacatgcgtgaggagtctccaagtgtgcgaagagttcctccgaccccttctacttgcgtttttcgcaggagcagtatcatgaagcccgaaagcgacagatctgtgtttttgaaggcagaaagcaaaatgtttgcttcttttttcaaaggagctcgaggtggtgtctgcattccaaccactgaatccggatcccatttgtgaataagccattcagactcatattccctatttaacagggtctctagtgctgtgaaaaaaatattgctgaacattgcatataacttatattgtaagaaatactgtacatttgaggaagactttattgtacctggatagctgtaagaaaggcatgaaggacgccaagagttttaaggaatatagggggattaaagtatggagatacagaagaaaccactaagtctgatgtccaaatgggcacactgtcagttttgtttcccttcagttgtttgatgcatttaaaaaaaaaaaaaagaaagaaaaagaaaaaaaggggggggggggagaaaaaaataaattaaaaaaaaaaaaaaaaaaagaaaagaaagaaaaatctaagaaaaaaaaaaaaaaggttgtaggcaaatcatttgttccaggctgtgagcctgtgcaaaagagatttcagatctgggcaatgggtgtgtgatctcacccactgaagatctgagaatgtcatggctaggcctacatgctctgtgaatcagtccctgtaattgttgtttgtatgtataattcagaagcaccaaaataagaaaagatgtagatttatttcatcatattatacagactgaattgttgtataaatttatttactgctagtgttaggaactgcttttttttttttttttggttttaatgttttttttttttttgttttttgtttttttttttctttctctctggatttttggttgaataaactagattgctttcagttgacttaaggtggatgtactctggagggtttatttttccttttattattatttttgatggtatttattaaatagcttctatgggcccggcggtacctgtctttttcgtcacttttcttgcagcctaaactatgaaggtagcagcgtaccagctaccaacatgcatgtcagagacccggccactcacaggcctggtcctgagagccacctggctgactgttagcccctgtgtgttctgtattagtgatcactgcctttaaacagtctgttggaataatactataaaaataataataaagttaaaatattttaaaacaa

SEQ ID NO: 84 - protein, 480 aa, Homo sapiens
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG

SEQ ID NO: 85 - protein, 466 aa, Homo sapiens
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG

SEQ ID NO: 86 - protein, 480 aa, Homo sapiens; identical to SEQ ID NO: 84

SEQ ID NO: 87 - DNA, 3383 bp, Homo sapiens
  source 1..3383: mol_type = genomic DNA
gagagagaggcagcgtgagcgccaggaaggtagcgaggccagcgtcgccccgggactcgctgctcaagtctgtctattgcctgccgccacatccatcctagcagggccccgtcgcccaccaggcggacaaaagcggtccgctgaacaccatgcggccgctcggcgtgccgcccaggctctgctggtgagcgccgccaccccgcgcccaggtcccgcgagcccgcctgccgcgcacctcgccctgctcccagctctactccaggccccgtccgcccgggggcgccgcccaccgcgcctcgctcgggccgttgccgtctgcacccagaccctgagccgccgccgccggccatggaggtggcgcccgagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctagggaacagatggacgtcgaggaccgggcactcccgggatgggtggaccaaacccttagcagcccagcatttcccgaaggccgacaccactcctgccagcccggctcggcccagcaccccctctcctggagggcgcccagcagcctgccagcagttactgtgaatgttccccaccgctgagaggctgcctccgcacctgaccgctgcccaggtggggtttcctgcatggacagttgtttggagaacaacaaggacaactttatgtagagaaaaggaggggacgggacagacgaaggcaaccatttttagaaggaaaaaggattaggcaaaaataatttattttgctcttgtttctaacaaggacttggagacttggtggtctgagctgtcccaagtcctccggttcttcctcgggattggcgggtccacttgccagggctctgggggcagatttgtggggacctcagcctgcaccctcttctcctctggcttccctctctgaaatagccgaactccaggctgggctgagccaaagccagagtggccacggcccagggagggtgagctggtgcctgctttgacgggccaggccctggagggcagagacaatcacgggcggtcctgcacagattcccaggccagggctgggtcacaggaaggaaacaacattttcttgaaaggggaaacgtctcccagatcgctcccttggctttgaggccgaagctgctgtgactgtgtccccttactgagcgcaagccacagcctgtcttgtcaggtggaccctgtaaatacatcctttttctgctaacccttcaaccccctcgcctcctactctgagacaaaagaaaaaatattaaaaaaatgcataggcttaactcgctgatgagttaattgttttatttttaaactctttttgggtccagttgattgtacgtagccacaggagccctgctatgaaaggaataaaacctacacacaaggttggagctttgcaattctttttggaaaagagctgggatcccacagccctagtatgaaagctgggggtggggaggggcctttgctgcccttggtttctgggggctggttggcatttgctggcctggcagggggtgaaggcaggagttgggggcaggtcaggaccaggacccagggagaggctgtgtccctgctggggtctcaggtccagctttactgtggctgtctggatccttcccaaggtacagctgtatataaacgtgtcccgagcttagattctgtatgcggtgacggcggggtgtggtggcctgtgaggggcccctggcccaggaggaggattgtgctgatgtagtgaccaagtgcaatatgggcgggcagtcgctgcagggagcaccacggccagaagtaacttattttgtactagtgtccgcataagaaaaagaatcggcagtattttctgtttttatgttttatttggcttgttttattttggattagtgaactaagttattgttaattatgtacaacatttatatattgtctgtaaaaaatgtatgctatcctcttattcctttaaagtgagtactgttaagaataataaaatactttttgtgaa

SEQ ID NO: 88 - DNA, 3470 bp, Homo sapiens
  source 1..3470: mol_type = genomic DNA
actgggtcaagcacagccctgagcggccgcgtgtccgaggcccaggtgccctctagagccctgtagttcctgcccctctctgcccctctcggctcctgctgttccgccgctgtcgtccgaaccatcccaacccccagtccacccagacagcgcccgagctaggggagggaacggtctgggagtcggcagctggcgccagggcggccggaggatgccgaggggccggagccgggagggcccgaggccgaggcgcactctacccccagctcctaccctgtaagccccgccagcctccggacgtgctgtccctgggcccgtcgccctcggggctcccgccggaactccttcactctcagaggccgagtccctcccctccccacggctgcgtgtggccgttgccgtctgcacccagaccctgagccgccgccgccggccatggaggtggcgcccgagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctagggaacagatggacgtcgaggaccgggcactcccgggatgggtggaccaaacccttagcagcccagcatttcccgaaggccgacaccactcctgccagcccggctcggcccagcaccccctctcctggagggcgcccagcagcctgccagcagttactgtgaatgttccccaccgctgagaggctgcctccgcacctgaccgctgcccaggtggggtttcctgcatggacagttgtttggagaacaacaaggacaactttatgtagagaaaaggaggggacgggacagacgaaggcaaccatttttagaaggaaaaaggattaggcaaaaataatttattttgctcttgtttctaacaaggacttggagacttggtggtctgagctgtcccaagtcctccggttcttcctcgggattggcgggtccacttgccagggctctgggggcagatttgtggggacctcagcctgcaccctcttctcctctggcttccctctctgaaatagccgaactccaggctgggctgagccaaagccagagtggccacggcccagggagggtgagctggtgcctgctttgacgggccaggccctggagggcagagacaatcacgggcggtcctgcacagattcccaggccagggctgggtcacaggaaggaaacaacattttcttgaaaggggaaacgtctcccagatcgctcccttggctttgaggccgaagctgctgtgactgtgtccccttactgagcgcaagccacagcctgtcttgtcaggtggaccctgtaaatacatcctttttctgctaacccttcaaccccctcgcctcctactctgagacaaaagaaaaaatattaaaaaaatgcataggcttaactcgctgatgagttaattgttttatttttaaactctttttgggtccagttgattgtacgtagccacaggagccctgctatgaaaggaataaaacctacacacaaggttggagctttgcaattctttttggaaaagagctgggatcccacagccctagtatgaaagctgggggtggggaggggcctttgctgcccttggtttctgggggctggttggcatttgctggcctggcagggggtgaaggcaggagttgggggcaggtcaggaccaggacccagggagaggctgtgtccctgctggggtctcaggtccagctttactgtggctgtctggatccttcccaaggtacagctgtatataaacgtgtcccgagcttagattctgtatgcggtgacggcggggtgtggtggcctgtgaggggcccctggcccaggaggaggattgtgctgatgtagtgaccaagtgcaatatgggcgggcagtcgctgcagggagcaccacggccagaagtaacttattttgtactagtgtccgcataagaaaaagaatcggcagtattttctgtttttatgttttatttggcttgttttattttggattagtgaactaagttattgttaattatgtacaacatttatatattgtctgtaaaaaatgtatgctatcctcttattcctttaaagtgagtactgttaagaataataaaatactttttgtgaa

SEQ ID NO: 89 - DNA, 3263 bp, Homo sapiens
  source 1..3263: mol_type = genomic DNA
gagtcggcagctggcgccagggcggccggaggatgccgaggggccggagccgggagggcccgaggccgaggcgcactctacccccagctcctaccctgtaagccccgccagcctccggacgtgctgtccctgggcccgtcgccctcggggctcccgccggaactccttcactctcagaggccgagtccctcccctccccacggctgcgtgtggccgttgccgtctgcacccagaccctgagccgccgccgccggccatggaggtggcgcccgagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctagggaacagatggacgtcgaggaccgggcactcccgggatgggtggaccaaacccttagcagcccagcatttcccgaaggccgacaccactcctgccagcccggctcggcccagcaccccctctcctggagggcgcccagcagcctgccagcagttactgtgaatgttccccaccgctgagaggctgcctccgcacctgaccgctgcccaggtggggtttcctgcatggacagttgtttggagaacaacaaggacaactttatgtagagaaaaggaggggacgggacagacgaaggcaaccatttttagaaggaaaaaggattaggcaaaaataatttattttgctcttgtttctaacaaggacttggagacttggtggtctgagctgtcccaagtcctccggttcttcctcgggattggcgggtccacttgccagggctctgggggcagatttgtggggacctcagcctgcaccctcttctcctctggcttccctctctgaaatagccgaactccaggctgggctgagccaaagccagagtggccacggcccagggagggtgagctggtgcctgctttgacgggccaggccctggagggcagagacaatcacgggcggtcctgcacagattcccaggccagggctgggtcacaggaaggaaacaacattttcttgaaaggggaaacgtctcccagatcgctcccttggctttgaggccgaagctgctgtgactgtgtccccttactgagcgcaagccacagcctgtcttgtcaggtggaccctgtaaatacatcctttttctgctaacccttcaaccccctcgcctcctactctgagacaaaagaaaaaatattaaaaaaatgcataggcttaactcgctgatgagttaattgttttatttttaaactctttttgggtccagttgattgtacgtagccacaggagccctgctatgaaaggaataaaacctacacacaaggttggagctttgcaattctttttggaaaagagctgggatcccacagccctagtatgaaagctgggggtggggaggggcctttgctgcccttggtttctgggggctggttggcatttgctggcctggcagggggtgaaggcaggagttgggggcaggtcaggaccaggacccagggagaggctgtgtccctgctggggtctcaggtccagctttactgtggctgtctggatccttcccaaggtacagctgtatataaacgtgtcccgagcttagattctgtatgcggtgacggcggggtgtggtggcctgtgaggggcccctggcccaggaggaggattgtgctgatgtagtgaccaagtgcaatatgggcgggcagtcgctgcagggagcaccacggccagaagtaacttattttgtactagtgtccgcataagaaaaagaatcggcagtattttctgtttttatgttttatttggcttgttttattttggattagtgaactaagttattgttaattatgtacaacatttatatattgtctgtaaaaaatgtatgctatcctcttattcctttaaagtgagtactgttaagaataataaaatactttttgtgaatgcccaaaaaaaaa

SEQ ID NO: 90 - protein, 480 aa, Mus musculus
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHSPWTVSPFSKTPLHPSAAGAPGGPLSVYPGAAGGSGGGSGSSVASLTPTAAHSGSHLFGFPPTPPKEVSPDPSTTGAASPASSSAGGSVARGEDKDGVKYQVSLSESMKMEGGSPLRPGLATMGTQPATHHPIPTYPSYVPAAAHDYGSSLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSSKSKKSKKGAECFEELSKCMQEKSPPFSAAALAGHMAPVGHLPPFSHSGHILPTPTPIHPSSSLSFGHPHPSSMVTAMG

SEQ ID NO: 91 - protein, 480 aa, Mus musculus; identical to SEQ ID NO: 90

SEQ ID NO: 92 - DNA, 3258 bp, Mus musculus
  source 1..3258: mol_type = genomic DNA
gtctgtgcaggagtcggcagctggcgccagggcggccggaggatgcagaggggccggagccgggcgggccggaggccgagacgcgcgctgtcccccacccctatcccgtgaatccgccggccctggaacgcgctgtcgctgggcccgccgtacccgggctctcctggtgtctcttactctctactgctgagccctccccttcccgcgccgctgcgagtggccgccccaccttcgcctggttcccaagacacagtagtggaccatggaggtggcgcctgagcagcctcgctggatggcgcaccccgccgtattgaatgcgcagcaccccgactcgcaccatccgggcctggcgcataactacatggagccagcacagctgctgcctcccgacgaggtggatgtcttcttcaaccatctcgactcgcagggcaacccttactacgccaacccggcccacgcgcgcgcgcgcgtttcctacagcccggcgcatgcccgtctcaccggaggccagatgtgccgaccacacttgttgcacagcccaggcttgccgtggctggacgggggcaaagcagctctctctgccgccgctgcccatcaccacagtccctggaccgtcagcccgttctccaagaccccgctgcacccctcagctgctggagcacccggagggcctctgtctgtttacccaggggctgcgggtgggagcgggggaggcagtgggagctccgtggcctccctcacccccactgcagcccactcgggctcccatctcttcggcttcccacccacgccacccaaagaagtgtctccagaccccagcacaacaggagctgcttccccggcctcttcttctgcagggggtagtgtagcccggggtgaggacaaggatggcgtcaagtaccaagtgtcactctccgagagcatgaagatggaaggcggcagtcccctgcgcccgggcctagctaccatgggcacccagcctgcaacacaccacccgatacccacctatccctcctatgtgcccgccgcagctcatgactatggcagcagtctcttccatccaggaggcttcctgggtggccccgcctccagcttcacccctaagcagagaagcaaggctcgctcctgctcagaaggccgggagtgtgtcaactgtggtgccacagccacccctctctggcgacgagatggcacgggccactacctgtgcaatgcctgtgggctctaccacaagatgaatggacagaaccggccgctcatcaagcccaagcggaggctgtctgctgccagaagagcgggcacctgttgtgcaaattgtcagacgacaaccaccaccttatggcgccggaacgccaacggggaccctgtgtgcaacgcctgtggcctctactacaagctgcacaatgttaacaggccactgaccatgaagaaggaagggatccagacccggaatcggaagatgtccagcaaatccaagaagagcaagaaaggggctgaatgtttcgaggagctctccaagtgcatgcaagagaagtcaccgcccttcagtgcggctgccctggctggacacatggcacctgtgggacacctcccaccttttagtcactctggacacatcctacccacgcccacgcctatccacccttcctccagtctctcttttggccacccccacccgtccagcatggtgactgccatgggctaggcaagcctcccactggacagacatggacatcaagggtggtttggcagaaccagagcgaggctgggcactcccaggatgggtggaacatactcttggctcccgcccatcccaagagacccacttcctcctgccagcctagcctggccgaagccacctctccttggaggactcccagccttgtgccgccattactgtgaatatttctaactgggctgcagctcgcgtgtgcccggggtgctgcccagaaaagtgttttcacggagagtgtttgtttggagagcaaaatggacaggtttacagatttatagcaagaagagactggggatagaaaaatgaaacctttttttttctttttctttttttcttcttctgttttatttttttgatggagaaaggagtaggcaagaagaaaaataatttattttgctcttatttcttacaagaacgtgaagacatggaggcgtgtgctatttgtgttcttggggtccttctttgggacctcctgccaccagtcagggctctcgggggcagacttagaggtcctcagcctgagcctccttcaccccagcctgcctgcagggtagcccctgccctgacgcagccctagagggcagagacaattgcaggcggtcctgcgcagattcccaggccagggctgggtcacaggaaggaaacattctctggaaaggggaaacgtctcccagatcattcccctggcttccagaggccaaagctggtgtgacccaaatgggccagagctgcagcctgtgctctaggccagtcggacccctgtaaatacaaccttcttttctgctaaaccctcggccccctccccctctaagataaataagaaaatactcaaagcgaaaaccaaactgcataagcttaacccgctgatgagtggttttattttgaaactcgttttttgggtccagtcaattgtacgttgccacagaagccccgctatggaaaaaaataaataaaacctacaaaccaggcctgagcttcacagtcctttgagtggttcttgggtcccacagccctggcagggggctcgggacaagggggaatcttatgctcttggtttctgggagacagggggcaggcaggcagtggccctgtgatcccaggcttctgttctgctgtggctggctgaatccttcaaggtacagttgtacataaaaagtgtcccaagcttcgattctgtgtgtggtggtggcagtggtgcagcagccagcaagggggccccgagtgagcccagggagacgattgtgctgagtcaaccaagtgcaatatcggtgtccagttgctgcagagcaccctaaccggaagtaacttattttgtgctagtacccgcataagagaagaatcggcagtattttctgtttttatgttttgggcttgttttattttgaattagtgacctaagttattgttaactgtgtacaacatttaaatattgtctgtaaaaattgtatgctaccctcttattcctttaaagtgaatactgttaaaaataataaaatactttttgtgaaaaaaaaaaaaaaaaa

SEQ ID NO: 93 - DNA, 3150 bp, Mus musculus
  source 1..3150: mol_type = genomic DNA
gcggccgcgctgactcccgcgggccgaccagccacgccgctcacaccgctgcctccctgctgagccctggccgtggccgcccgagggcgccgcccaccgcgccgcagtcgggccgccccaccttcgcctggttcccaagacacagtagtggaccatggaggtggcgcctgagcagcctcgctggatggcgcaccccgccgtattgaatgcgcagcaccccgactcgcaccatccgggcctggcgcataactacatggagccagcacagctgctgcctcccgacgaggtggatgtcttcttcaaccatctcgactcgcagggcaacccttactacgccaacccggcccacgcgcgcgcgcgcgtttcctacagcccggcgcatgcccgtctcaccggaggccagatgtgccgaccacacttgttgcacagcccaggcttgccgtggctggacgggggcaaagcagctctctctgccgccgctgcccatcaccacagtccctggaccgtcagcccgttctccaagaccccgctgcacccctcagctgctggagcacccggagggcctctgtctgtttacccaggggctgcgggtgggagcgggggaggcagtgggagctccgtggcctccctcacccccactgcagcccactcgggctcccatctcttcggcttcccacccacgccacccaaagaagtgtctccagaccccagcacaacaggagctgcttccccggcctcttcttctgcagggggtagtgtagcccggggtgaggacaaggatggcgtcaagtaccaagtgtcactctccgagagcatgaagatggaaggcggcagtcccctgcgcccgggcctagctaccatgggcacccagcctgcaacacaccacccgatacccacctatccctcctatgtgcccgccgcagctcatgactatggcagcagtctcttccatccaggaggcttcctgggtggccccgcctccagcttcacccctaagcagagaagcaaggctcgctcctgctcagaaggccgggagtgtgtcaactgtggtgccacagccacccctctctggcgacgagatggcacgggccactacctgtgcaatgcctgtgggctctaccacaagatgaatggacagaaccggccgctcatcaagcccaagcggaggctgtctgctgccagaagagcgggcacctgttgtgcaaattgtcagacgacaaccaccaccttatggcgccggaacgccaacggggaccctgtgtgcaacgcctgtggcctctactacaagctgcacaatgttaacaggccactgaccatgaagaaggaagggatccagacccggaatcggaagatgtccagcaaatccaagaagagcaagaaaggggctgaatgtttcgaggagctctccaagtgcatgcaagagaagtcaccgcccttcagtgcggctgccctggctggacacatggcacctgtgggacacctcccaccttttagtcactctggacacatcctacccacgcccacgcctatccacccttcctccagtctctcttttggccacccccacccgtccagcatggtgactgccatgggctaggcaagcctcccactggacagacatggacatcaagggtggtttggcagaaccagagcgaggctgggcactcccaggatgggtggaacatactcttggctcccgcccatcccaagagacccacttcctcctgccagcctagcctggccgaagccacctctccttggaggactcccagccttgtgccgccattactgtgaatatttctaactgggctgcagctcgcgtgtgcccggggtgctgcccagaaaagtgttttcacggagagtgtttgtttggagagcaaaatggacaggtttacagatttatagcaagaagagactggggatagaaaaatgaaacctttttttttctttttctttttttcttcttctgttttatttttttgatggagaaaggagtaggcaagaagaaaaataatttattttgctcttatttcttacaagaacgtgaagacatggaggcgtgtgctatttgtgttcttggggtccttctttgggacctcctgccaccagtcagggctctcgggggcagacttagaggtcctcagcctgagcctccttcaccccagcctgcctgcagggtagcccctgccctgacgcagccctagagggcagagacaattgcaggcggtcctgcgcagattcccaggccagggctgggtcacaggaaggaaacattctctggaaaggggaaacgtctcccagatcattcccctggcttccagaggccaaagctggtgtgacccaaatgggccagagctgcagcctgtgctctaggccagtcggacccctgtaaatacaaccttcttttctgctaaaccctcggccccctccccctctaagataaataagaaaatactcaaagcgaaaaccaaactgcataagcttaacccgctgatgagtggttttattttgaaactcgttttttgggtccagtcaattgtacgttgccacagaagccccgctatggaaaaaaataaataaaacctacaaaccaggcctgagcttcacagtcctttgagtggttcttgggtcccacagccctggcagggggctcgggacaagggggaatcttatgctcttggtttctgggagacagggggcaggcaggcagtggccctgtgatcccaggcttctgttctgctgtggctggctgaatccttcaaggtacagttgtacataaaaagtgtcccaagcttcgattctgtgtgtggtggtggcagtggtgcagcagccagcaagggggccccgagtgagcccagggagacgattgtgctgagtcaaccaagtgcaatatcggtgtccagttgctgcagagcaccctaaccggaagtaacttattttgtgctagtacccgcataagagaagaatcggcagtattttctgtttttatgttttgggcttgttttattttgaattagtgacctaagttattgttaactgtgtacaacatttaaatattgtctgtaaaaattgtatgctaccctcttattcctttaaagtgaatactgttaaaaataataaaatactttttgtgaaaaaaaaaaaaaaaaa

SEQ ID NO: 94 - protein, 206 aa, Homo sapiens
MSGPGTAAVALLPAVLLALLAPWAGRGGAAAPTAPNGTLEAELERRWESLVALSLARLPVAAQPKEAAVQSGAGDYLLGIKRLRRLYCNVGIGFHLQALPDGRIGGAHADTRDSLLELSPVERGVVSIFGVASRFFVAMSSKGKLYGSPFFTDECTFKEILLPNNYNAYESYKYPGMFIALSKNGKTKKGNRVSPTMKVTHFLPRL

SEQ ID NO: 95 - DNA, 3165 bp, Homo sapiens
  source 1..3165: mol_type = genomic DNA
ctggcgctcagggaggcgcgcactgctcctcagagtcccagctccagccgcgcgctttccgcccggctcgccgctccatgcagccggggtagagcccggcgcccgggggccccgtcgcttgcctcccgcacctcctcggttgcgcactcctgcccgaggtcggccgtgcgctcccgcgggacgccacaggcgcagctctgccccccagcttcccgggcgcactgaccgcctgaccgacgcacggccctcgggccgggatgtcggggcccgggacggccgcggtagcgctgctcccggcggtcctgctggccttgctggcgccctgggcgggccgagggggcgccgccgcacccactgcacccaacggcacgctggaggccgagctggagcgccgctgggagagcctggtggcgctctcgttggcgcgcctgccggtggcagcgcagcccaaggaggcggccgtccagagcggcgccggcgactacctgctgggcatcaagcggctgcggcggctctactgcaacgtgggcatcggcttccacctccaggcgctccccgacggccgcatcggcggcgcgcacgcggacacccgcgacagcctgctggagctctcgcccgtggagcggggcgtggtgagcatcttcggcgtggccagccggttcttcgtggccatgagcagcaagggcaagctctatggctcgcccttcttcaccgatgagtgcacgttcaaggagattctccttcccaacaactacaacgcctacgagtcctacaagtaccccggcatgttcatcgccctgagcaagaatgggaagaccaagaaggggaaccgagtgtcgcccaccatgaaggtcacccacttcctccccaggctgtgaccctccagaggacccttgcctcagcctcgggaagcccctgggagggcagtgccgagggtcaccttggtgcactttcttcggatgaagagtttaatgcaagagtaggtgtaagatatttaaattaattatttaaatgtgtatatattgccaccaaattatttatagttctgcgggtgtgttttttaattttctggggggaaaaaaagacaaaacaaaaaaccaactctgacttttctggtgcaacagtggagaatcttaccattggatttctttaacttgtcaaaagttgtcacgagtgtgctgctattctgtgttttaaaaaaaggtgacattggattccgatgtcatcccctgtagtatggcgtggagcatctctgtctggaaaggcccgcctgaggcttgggcagccagttcagggagctcccaggcttggctctcggctagcatcctcagaggcccactccctttgtgccctgttgctattaatcgggacatatcggtttacttcgggtacagaaagtgcggtgttgaagtcctcgctgccactctgtttttagatctgccaagactgacctttgaactttcctgtagtcaatcttcctcgatctaccagatgggagagacccttggacaactttataaactcctgtttgccttttttggatcagcgacagcccccatcgctgtgactattggggaaaagacgaagctctttcataaattccatggagaggaatcaatatcccactggaaggctagaaatggacaagatagtgtatttgcaatcacaaacaaaaccctagtgatgaaaaataatttgtgatggcagatgcttctgatggtgtgatagaatatgtttttgaaaacaaaccatcgaaccccccgccccacccccaaaacgggcttccctgtgtttagggagctttgggctagaactagctacgatttttaggtgaaatgtccttgtaattgtacaaagcacttggtgcagtgtttgcgtggagcagcctgctgctttctgatgcattccctgtttaagtgcgtttaacatctacctcacaagccctgaaaccccaggcaaaacccacagaaagctcatacccggtgcaggagtttgccatcccaagtggctttttttccatatgtagccaaaaaggattgcagatagcgtcggtgcgtcccattcgaaccttgtcacgtttgagctatctttaccctgtgatttacttttagtaagggtgatcatggtgaaaatatttgcagacagctgttacagtacactatatggtcaccaagtaaccttatatttttctttatatattttacaaatgtaacccctgtcattgaagcaaccgtggaagaggcagggtcggtgatgtttaaaaaaagttccgaggtgatggcaaacatttaattttaatgaatgactttttagagtttatacaaaatgaccttagcttgctaccagaaatgctccgaatgtttcgtcaagactttaatactctcctaggatgtttctgaactgtctcccgaattaactttatgggagtctacagacagcaagactggaaaatctgattggagtttttgtctttcacattccttttgaaaactctttgttcgaatgcaaatcatcgacttaaaatactattcttaaccaaggcctggaagaaagaagacacttgcaaagccgctaagacaggaccacacatcttaaactgctgttcctaccatgcactaaactgtttttaagttttaaaccacaccctaggctccaggagtgttcaggaaagatggtgtttgtaggtctccatgctgtttggcgttggggggtgtggagggatcatccgtcgactttctgaattttaatgtattcacttagtaacaaaccatgattgtcttaaatgccttaaattattatgagatttcttgtctcagagcccaatcagattgtcaggaattaacatgtgttaggtttgatcacccttgaccacttcttatagatatttcttcaacaaatcatgtgtgatgcctgtaggaacacaactgtacctttaaaatattgttttcatattgctgtgatggggattcgaggttcctgtatgtgccactgttttcagaatctgtagttttatacaggtgccgaccctcgttgtgatgtatgtgctgtgcacattgacatgctgaccgacaatgataagcgtttatcgtgtataaaaagacaccactggactggatgtacacaactgggaaaggaattaaaagctattaaaattgtgccttgaaa

SEQ ID NO: 96 - protein, 202 aa, Mus musculus
MAKRGPTTGTLLPRVLLALVVALADRGTAAPNGTRHAELGHGWDGLVARSLARLPVAAQPPQAAVRSGAGDYLLGLKRLRRLYCNVGIGFHLQVLPDGRIGGVHADTRDSLLELSPVQRGVVSIFGVASRFFVAMSSRGKLFGVPFFTDECKFKEILLPNNYNAYESYAYPGMFMALSKNGRTKKGNRVSPTMKVTHFLPRL

SEQ ID NO: 97 - DNA, 3032 bp, Mus musculus
  source 1..3032: mol_type = genomic DNA
gcgcggggactatcccgccaccgttgcgtccctatttgctctcgctacttaggtctgtgcgcagcactcaccgaactcacggcccgcagctcgaactcacgcacggcccgcgggccgggatggcgaaacgcgggccgaccacagggacgctgctgcccagggtcctgctggccctggtggtggccctggcggaccgagggaccgccgcacccaacggcacgcggcacgcagaattggggcacggctgggacggcttggtggcccgctcgctggcacgcctgccggtggccgcgcagcccccgcaggcggcggtccgcagcggcgcaggggactacctgctgggcctcaaaaggcttcggcggctctactgcaacgtgggcatcggattccacctgcaggtgctgcccgacggccgcatcggtggtgtgcacgcagacacgagggacagtcttctggagctctctccggtgcagcgaggcgtggtgagcatcttcggagtggccagccggttcttcgtggccatgagcagcaggggcaagctcttcggtgtgcctttctttaccgacgagtgtaaattcaaagaaatacttctgcccaacaactacaacgcctacgaatcctacgcgtaccccggtatgttcatggccctcagtaagaacgggcggaccaagaaggggaaccgagtgtcgcctaccatgaaggtaacccacttccttcctagactgtgaccctccggagccctgcctcagcctcggaagcacaccctacccctcaggaggagcactttctctcgatggagaattgtttgcaaaaaaactggtctaagatatttaaattatttaaatatgtatatatggacgcccaattatttataattttatgtattctcattttcggggggggggatatgaccaaaagaacaaacaaattgcagctcagacctctttggaatagcggaacagacttctttcttcactatcaaagatatgggtgtgatgctgtttcatgtgtgcctctaaaacttggtgacatcagtttccaagggtgcctggcctgtaactggaaaggcctgcctgggacctctgaggcagtgagaaggaccctgagcgttcccgatcgggagcattctgccgtcgccgctccctctgcttcctttggtatgaaccctgtcggatcggtttactccagggacagaagtcctcccgcctctgtttttagatctccaagactgatctttgaactctcttgcagtcaatcatcttcttggacctaccggataggagacccttagacaactttataaactcctgtttgccatttttggactggccaacagggcacgttgcttgtagccactggaactttgcttttctggagaggaactaggaatggacaagagggtgtgtgcgctcgccacaactcaaaactctggggatgaaattctgttttgtgatagaggatgatttgttgggatataacaatgtattttgcaagaatcaaactgagaaaaacaggcttccctgaatatgggaagctttgtgctggaactccataatttaagggtgagctgccctttcccaccccaggcagcctttatgcaggcagactgcaggagtccgaattccggtgctgccccaaggtgaaggaattctatccccatctacctcagagttcctgagaccctggtgactaagctggaggctctgctgggtgcatctgcctgcttctcttccacatgtagtatctggacccttgtcacgttccagctatctttacccaatgatttacttttagtaaggggtattcatggtgaaaatatgcacgaccagctgtcggagtgcactatatggtcaccaagtcaagtaacccttatatttttctttatatattttacgaatgtaacccctgtcactgagaccaaaatggaagagcagggtcggtggcatttaaaaaaaagggctgaggtgagggagaacaattaattttaatgaatgactttggagtttatacaaaatgaccttagctcgcttcagggaatgcttctccccaagatgttaagactctgctgggagacttctgagcaacctcccgaattaactttatgggaggctacagacagcaagactggaaaatctcattggcattttttttttttgtctttcacattcctttagaaaactctttgtttggatgctaatgggatacttaaaatactattctgtaccacagcccaagatggaagaagccacaccccaaagctgaggtgggagctcctcccaaacttcctttctgtctggtggctcacaggacaataagattttgtgttttttaaatccaggccctaggctccaagagtgttggggagaagacagttgaaatgtcttttcttcttgttattttctaaaagacggtctcatagcccaggctgtacctgaacttgctatgtcactaaagatgaccttgaatttctgatcctcttgccccatttcctagtgctaaggttacaggcattgcccaccacgcccactccttaggtgctgggaattgaaatcaagtgcctgccaggccactccacagagataggcctccctcttttttgtgggggaggaaatgtggggtcttactctgtagccaaggctggcctactgcttgagcccaagtgttgagactacaggtagaaggcacctgccctgttctgatgggattctctgcagctatgttgcttcaggcaggctgtggtcctacctgacacctattacctgtttcttcagccagctagctccctttcctgcgcccagggacaagactgtatctttggaattttgttaccagaatgggtttgatgtttctgctctgtatctgccactgttaccgagtcggtctgtagttactacaggtgtcgacccagagatagtatgtgctatgcacactggatgctccatccaaagagaagcattcaatcatgtatagacagccccatggactgatgggaatgattgggagagatattaaaatgacaaacgtgtctgcaa

SEQ ID NO: 98 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 99 - protein, 155 aa, Homo sapiens [sequence omitted]

SEQ ID NO: 100 - DNA, 6801 bp, Homo sapiens
  source 1..6801: mol_type = genomic DNA
gtggccgaaccgccgaactcagaggccggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccacatctaatctcatttcacatgaaagaagaagtatattttagaaatttgttaatgagagtaaaagaaaataaatgtgtatagctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaacttctcgaaccgctgtgtctcctacgtaaaaaaagagatgtacaaatcaataataattacacttttagaaactgtatcatcaaagattttcagttaaagtagcattatgtaaaggctcaaaacattaccctaacaaagtaaagttttcaatacaaattctttgccttgtggatatcaagaaatcccaaaatattttcttaccactgtaaattcaagaagcttttgaaatgctgaatatttctttggctgctacttggaggcttatctacctgtacatttttggggtcagctctttttaacttcttgctgctctttttcccaaaaggtaaaaatatagattgaaaagttaaaacattttgcatggctgcagttcctttgtttcttgagataagattccaaagaacttagattcatttcttcaacaccgaaatgctggaggtgtttgatcagttttcaagaaacttggaatataaataattttataattcaacaaaggttttcacattttataaggttgatttttcaattaaatgcaaatttgtgtggcaggatttttattgccattaacatatttttgtggctgctttttctacacatccagatggtccctctaactgggctttctctaattttgtgatgttctgtcattgtctcccaaagtatttaggagaagccctttaaaaagctgccttcctctaccactttgctggaaagcttcacaattgtcacagacaaagatttttgttccaatactcgttttgcctctatttttcttgtttgtcaaatagtaaatgatatttgcccttgcagtaattctactggtgaaaaacatgcaaagaagaggaagtcacagaaacatgtctcaattcccatgtgctgtgactgtagactgtcttaccatagactgtcttacccatcccctggatatgctcttgttttttccctctaatagctatggaaagatgcatagaaagagtataatgttttaaaacataaggcatttgtctgccatttttcaattacatgctgacttcccttacaattgagatttgcccataggttaaacatggttagaaacaactgaaagcataaaagaaaaatctaggccgggtgcagtggctcatgcctatattccctgcactttgggaggccaaagcaggaggatcgcttgagcccaggagttcaagaccaacctggtgaaaccccgtctctacaaaaaaacacaaaaaatagccaggcatggtggcgtgtacatgtggtctcagatacttgggaggctgaggtgggagggttgatcacttgaggctgagaggtcaaggttgcagtgagccataatcgtgccactgcagtccagcctaggcaacagagtgagactttgtctcaaaaaaagagaaattttccttaataagaaaagtaatttttactctgatgtgcaatacatttgttattaaatttattatttaagatggtagcactagtcttaaattgtataaaatatcccctaacatgtttaaatgtccatttttattcattatgctttgaaaaataattatggggaaatacatgtttgttattaaatttattattaaagatagtagcactagtcttaaatttgatataacatctcctaacttgtttaaatgtccatttttattctttatgtttgaaaataaattatggggatcctatttagctcttagtaccactaatcaaaagttcggcatgtagctcatgatctatgctgtttctatgtcgtggaagcactggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttgtattttttattattttaatatactgtaagccaaactgaaataacatttgctgttttataggtttgaagaacataggaaaaactaagaggttttgtttttatttttgctgatgaagagatatgtttaaatatgttgtattgttttgtttagttacaggacaataatgaaatggagtttatatttgttatttctattttgttatatttaataatagaattagattgaaataaaatataatgggaaataatctgcagaatgtgggttttcctggtgtttccctctgactctagtgcactgatgatctctgataaggctcagctgctttatagttctctggctaatgcagcagatactcttcctgccagtggtaatacgattttttaagaaggcagtttgtcaattttaatcttgtggatacctttatactcttagggtattattttatacaaaagccttgaggattgcattctattttctatatgaccctcttgatatttaaaaaacactatggataacaattcttcatttacctagtattatgaaagaatgaaggagttcaaacaaatgtgtttcccagttaactagggtttactgtttgagccaatataaatgtttaactgtttgtgatggcagtattcctaaagtacattgcatgttttcctaaatacagagtttaaataatttcagtaattcttagatgattcagcttcatcattaagaatatcttttgttttatgttgagttagaaatgccttcatatagacatagtctttcagacctctactgtcagttttcatttctagctgctttcagggttttatgaattttcaggcaaagctttaatttacactaagcttaggaagtatggctaatgccaacggcagtttttttcttcttaattccacatgactgaggcatatatgatctctgggtaggtgagttgttgtgacaaccacaagcactttttttttttttaaagaaaaaaaggtagtgaatttttaatcatctggactttaagaaggattctggagtatacttaggcctgaaattatatatatttggcttggaaatgtgtttttcttcaattacatctacaagtaagtacagctgaaattcagaggacccataagagttcacatgaaaaaaatcaatttatttgaaaaggcaagatgcaggagagaggaagccttgcaaacctgcagactgctttttgcccaatatagattgggtaaggctgcaaaacataagcttaattagctcacatgctctgctctcacgtggcaccagtggatagtgtgagagaattaggctgtagaacaaatggccttctctttcagcattcacaccactacaaaatcatcttttatatcaacagaagaataagcataaactaagcaaaaggtcaataagtacctgaaaccaagattggctagagatatatcttaatgcaatccattttctgatggattgttacgagttggctatataatgtatgtatggtattttgatttgtgtaaaagttttaaaaatcaagctttaagtacatggacatttttaaataaaatatttaaagacaatttagaaaattgccttaatatcattgttggctaaatagaataggggacatgcatattaaggaaaaggtcatggagaaataatattggtatcaaacaaatacattgatttgtcatgatacacattgaatttgatccaatagtttaaggaataggtaggaaaatttggtttctatttttcgatttcctgtaaatcagtgacataaataattcttagcttattttatatttccttgtcttaaatactgagctcagtaagttgtgttaggggattatttctcagttgagactttcttatatgacattttactatgttttgactacctgactattaaaaataaatagtagatacaattttcataaagtgaagaattatataatcactgctttataactgactttattatatttatttcaaagttcatttaaaggctactattcatcctctgtgatggaatggtcaggaatttgttttctcatagtttaattccaacaacaatattagtcgtatccaaaataacctttaatgctaaactttactgatgtatatccaaagcttctcattttcagacagattaatccagaagcagtcataaacagaagaataggtggtatgttcctaatgatattatttctactaatggaataaactgtaatattagaaattatgctgctaattatatcagctctgaggtaatttctgaaatgttcagactcagtcggaacaaattggaaaatttaaatttttattcttagctataaagcaagaaagtaaacacattaatttcctcaacatttttaagccaattaaaaatataaaagatacacaccaatatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctattgtatgtgttaatgattttatgtaaaaatgtaattgcttttcatgagtagtatgaataaaattgattagtttgtgttttcttgtctccca

SEQ ID NO: 101 - DNA, 6652 bp, Homo sapiens
  source 1..6652: mol_type = genomic DNA
gccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccacatctaatctcatttcacatgaaagaagaagtatattttagaaatttgttaatgagagtaaaagaaaataaatgtgtatagctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaacttctcgaaccgctgtgtctcctacgtaaaaaaagagatgtacaaatcaataataattacacttttagaaactgtatcatcaaagattttcagttaaagtagcattatgtaaaggctcaaaacattaccctaacaaagtaaagttttcaatacaaattctttgccttgtggatatcaagaaatcccaaaatattttcttaccactgtaaattcaagaagcttttgaaatgctgaatatttctttggctgctacttggaggcttatctacctgtacatttttggggtcagctctttttaacttcttgctgctctttttcccaaaaggtaaaaatatagattgaaaagttaaaacattttgcatggctgcagttcctttgtttcttgagataagattccaaagaacttagattcatttcttcaacaccgaaatgctggaggtgtttgatcagttttcaagaaacttggaatataaataattttataattcaacaaaggttttcacattttataaggttgatttttcaattaaatgcaaatttgtgtggcaggatttttattgccattaacatatttttgtggctgctttttctacacatccagatggtccctctaactgggctttctctaattttgtgatgttctgtcattgtctcccaaagtatttaggagaagccctttaaaaagctgccttcctctaccactttgctggaaagcttcacaattgtcacagacaaagatttttgttccaatactcgttttgcctctatttttcttgtttgtcaaatagtaaatgatatttgcccttgcagtaattctactggtgaaaaacatgcaaagaagaggaagtcacagaaacatgtctcaattcccatgtgctgtgactgtagactgtcttaccatagactgtcttacccatcccctggatatgctcttgttttttccctctaatagctatggaaagatgcatagaaagagtataatgttttaaaacataaggcatttgtctgccatttttcaattacatgctgacttcccttacaattgagatttgcccataggttaaacatggttagaaacaactgaaagcataaaagaaaaatctaggccgggtgcagtggctcatgcctatattccctgcactttgggaggccaaagcaggaggatcgcttgagcccaggagttcaagaccaacctggtgaaaccccgtctctacaaaaaaacacaaaaaatagccaggcatggtggcgtgtacatgtggtctcagatacttgggaggctgaggtgggagggttgatcacttgaggctgagaggtcaaggttgcagtgagccataatcgtgccactgcagtccagcctaggcaacagagtgagactttgtctcaaaaaaagagaaattttccttaataagaaaagtaatttttactctgatgtgcaatacatttgttattaaatttattatttaagatggtagcactagtcttaaattgtataaaatatcccctaacatgtttaaatgtccatttttattcattatgctttgaaaaataattatggggaaatacatgtttgttattaaatttattattaaagatagtagcactagtcttaaatttgatataacatctcctaacttgtttaaatgtccatttttattctttatgtttgaaaataaattatggggatcctatttagctcttagtaccactaatcaaaagttcggcatgtagctcatgatctatgctgtttctatgtcgtggaagcactggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttgtattttttattattttaatatactgtaagccaaactgaaataacatttgctgttttataggtttgaagaacataggaaaaactaagaggttttgtttttatttttgctgatgaagagatatgtttaaatatgttgtattgttttgtttagttacaggacaataatgaaatggagtttatatttgttatttctattttgttatatttaataatagaattagattgaaataaaatataatgggaaataatctgcagaatgtgggttttcctggtgtttccctctgactctagtgcactgatgatctctgataaggctcagctgctttatagttctctggctaatgcagcagatactcttcctgccagtggtaatacgattttttaagaaggcagtttgtcaattttaatcttgtggatacctttatactcttagggtattattttatacaaaagccttgaggattgcattctattttctatatgaccctcttgatatttaaaaaacactatggataacaattcttcatttacctagtattatgaaagaatgaaggagttcaaacaaatgtgtttcccagttaactagggtttactgtttgagccaatataaatgtttaactgtttgtgatggcagtattcctaaagtacattgcatgttttcctaaatacagagtttaaataatttcagtaattcttagatgattcagcttcatcattaagaatatcttttgttttatgttgagttagaaatgccttcatatagacatagtctttcagacctctactgtcagttttcatttctagctgctttcagggttttatgaattttcaggcaaagctttaatttacactaagcttaggaagtatggctaatgccaacggcagtttttttcttcttaattccacatgactgaggcatatatgatctctgggtaggtgagttgttgtgacaaccacaagcactttttttttttttaaagaaaaaaaggtagtgaatttttaatcatctggactttaagaaggattctggagtatacttaggcctgaaattatatatatttggcttggaaatgtgtttttcttcaattacatctacaagtaagtacagctgaaattcagaggacccataagagttcacatgaaaaaaatcaatttatttgaaaaggcaagatgcaggagagaggaagccttgcaaacctgcagactgctttttgcccaatatagattgggtaaggctgcaaaacataagcttaattagctcacatgctctgctctcacgtggcaccagtggatagtgtgagagaattaggctgtagaacaaatggccttctctttcagcattcacaccactacaaaatcatcttttatatcaacagaagaataagcataaactaagcaaaaggtcaataagtacctgaaaccaagattggctagagatatatcttaatgcaatccattttctgatggattgttacgagttggctatataatgtatgtatggtattttgatttgtgtaaaagttttaaaaatcaagctttaagtacatggacatttttaaataaaatatttaaagacaatttagaaaattgccttaatatcattgttggctaaatagaataggggacatgcatattaaggaaaaggtcatggagaaataatattggtatcaaacaaatacattgatttgtcatgatacacattgaatttgatccaatagtttaaggaataggtaggaaaatttggtttctatttttcgatttcctgtaaatcagtgacataaataattcttagcttattttatatttccttgtcttaaatactgagctcagtaagttgtgttaggggattatttctcagttgagactttcttatatgacattttactatgttttgactacctgactattaaaaataaatagtagatacaattttcataaagtgaagaattatataatcactgctttataactgactttattatatttatttcaaagttcatttaaaggctactattcatcctctgtgatggaatggtcaggaatttgttttctcatagtttaattccaacaacaatattagtcgtatccaaaataacctttaatgctaaactttactgatgtatatccaaagcttctcattttcagacagattaatccagaagcagtcataaacagaagaataggtggtatgttcctaatgatattatttctactaatggaataaactgtaatattagaaattatgctgctaattatatcagctctgaggtaatttctgaaatgttcagactcagtcggaacaaattggaaaatttaaatttttattcttagctataaagcaagaaagtaaacacattaatttcctcaacatttttaagccaattaaaaatataaaagatacacaccaatatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctattgtatgtgttaatgattttatgtaaaaatgtaattgcttttcatgagtagtatgaataaaattgattagtttgtgttttcttgtctccca

SEQ ID NO: 102 - protein, 154 aa, Mus musculus
MAASGITSLPALPEDGGAAFPPGHFKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 103 - DNA, 695 bp, Mus musculus
  source 1..695: mol_type = genomic DNA
ggccccgggccgttgtacactcaaggggctctctcggcttcaggaagagtccggctgcactgggctgggagcccggcgggacacggactgggaggctggcagcccgcgggcgagccgcgctggggggccgaggccggggtcggggccggggagccccaagagctgccacagcggggtcccggggccgcggaagggccatggctgccagcggcatcacctcgcttcccgcactgccggaggacggcggcgccgccttcccaccaggccacttcaaggaccccaagcggctctactgcaagaacggcggcttcttcctgcgcatccatcccgacggccgcgtggatggcgtccgcgagaagagcgacccacacgtcaaactacaactccaagcagaagagagaggagttgtgtctatcaagggagtgtgtgccaaccggtaccttgctatgaaggaagatggacggctgctggcttctaagtgtgttacagaagagtgtttcttctttgaacgactggaatctaataactacaatacttaccggtcacggaaatactccagttggtatgtggcactgaaacgaactgggcagtataaactcggatccaaaacgggacctggacagaaggccatactgtttcttccaatgtctgctaagagctgactcacttttgacactgtcactgagacactgtca

SEQ ID NO: 104 - protein, 443 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLVDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 105 - protein, 236 aa, Homo sapiens
MFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 106 - protein, 442 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 107 - protein, 236 aa, Homo sapiens; identical to SEQ ID NO: 105

SEQ ID NO: 108 - protein, 194 aa, Homo sapiens
MFDDFSEGRECVNCGAMSTPLWRRDGTGHYLCNACGLYHKMNGINRPLIKPQRRLVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 109 - DNA, 3419 bp, Homo sapiens
  source 1..3419: mol_type = genomic DNA
ggggacttggaggcggccggcgcaggggccgcgagaggcttcgtcgccgctgcagctccgggggctcccaggggagcgtgcgcggaacctccaggcccagcaggaccccggctgcggcgaggaggaaggagccagcctagcagcttctgcgcctgtggccgcgggtgtcctggaggcctctcggtgtgacgagtgggggacccgaaggctcgtgcgccacctccaggcctggacgctgccctccgtcttctgcccccaataggtgcgccggaccttcaggccctggggtgaattcagctgctcctacatcagcttccggaaccaccaaaaattcaaattgggattttccggagtaaacaagagcctagagccctttgctcaatgctggatttaatacgtatatatttttaagcgagttggttttttcccctttgatttttgatcttcgcgacagttcctcccacgcatattatcgttgttgccgtcgttttctctccccgcgtggctccttgacctgcgagggagagagaggacaccgaagccgggagctcgcagggaccatgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaatcttccctcttccctcctcaaattcctgcacggacctgggacttggaggatagcaaagaaggaggccctgggctcccaggggccggcctcctctgcctggtaatgactccagaacaacaactgggaagaaacttgaagtcgacaatctggttaggggaagcgggtgttggattttctcagatgcctttacacgctgatgggactggagggagcccacccttcagcacgagcacactgcatctctcctgtgagttggagacttctttcccaagatgtccttgtcccctgcgttccccactgtggcctagaccgtgggttttgcattgtgtttctagcaccgaggatctgagaacaagcggagggccgggccctgggacccctgctccagcccgaatgacggcatctgtttgccatgtacctggatgcgacgggcccctggggacaggcccttgccccatccatccgcttgaggcatggcaccgccctgcatccctaataccaaatctgactccaaaattgtggggtgtgacatacaagtgactgaacacttcctggggagctacaggggcacttaacccaccacagcacagcctcatcaaaatgcagctggcaacttctcccccaggtgccttccccctgctgccggcctttgctccttcacttccaacatctctcaaaataaaaatccctcttcccgctctgagcgattcagctctgcccgcagcttgtacatgtctctcccctggcaaaacaagagctgggtagtttagccaaacggcaccccctcgagttcactgcagacccttcgttcaccgtgtcacacatagaggggttctgagtaagaacaaaacgttctgctgctcaagccagtctggcaagcactcagcccagcctcgaggtccttctggggagagtgtaagtggacagagtcctggtcagggggcaggagtgtcccaagggctggcccacctgctgtctgtctgctcctcctagcccttggtcagatggcagccagagtccctcaggacctgcagcctcgccccggcagaagtcttttgtccaggaggcaaaaagccagagattctgcaacacgaattcgaagcaaacaaacacaacacaacagaattcctggaaagaagacgactgctaagacacggcaggggggcctggagggagcctccgactctgagctgctccgggatctgccgcgttctcctctgcacattgctgtttctgcccctgatgctggagctcaaggagactccttcctctttctcagcagagctgtagctgactgtggcattactacgcctccccacacgcccagacccctcactccaaaatcctactggctgtagcagagaatacctttgaaccaagattctgttttaatcatcatttacattgttttcttccaaaggccccctcgtataccctccctaacccacaaacctgttaacattgtcttaaggtgaaatggctggaaaatcagtatttaactaataaatttatctgtattcctctttc

SEQ ID NO: 110 - DNA, 2640 bp, Homo sapiens
  source 1..2640: mol_type = genomic DNA
agagggagctgggagacacggctcacaacgtctccctcccacccggctgagaacagcctggaatccctgtgcagagtttgcctcacaccaggccttgagcagccgcagggacgccgctccccaccgcggggacgcatcctgctctgcaccctggttctcggcgctgcgcccgcggaggctcgtgcagggcaggctgcccgtgcgggtgaggactgagtgccgcgcagggaaggagtatcgcagaccggcgcccaggcccagcgggggaatccaagggccgtgttgcaggactcggcattcgttctgcgcgggtcaccttgaatgtctgtccggatccctcgcggcagggccgcagaggcgcgtccatatcttggaggaattcgttccatagaatgagtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaatcttccctcttccctcctcaaattcctgcacggacctgggacttggaggatagcaaagaaggaggccctgggctcccaggggccggcctcctctgcctggtaatgactccagaacaacaactgggaagaaacttgaagtcgacaatctggttaggggaagcgggtgttggattttctcagatgcctttacacgctgatgggactggagggagcccacccttcagcacgagcacactgcatctctcctgtgagttggagacttctttcccaagatgtccttgtcccctgcgttccccactgtggcctagaccgtgggttttgcattgtgtttctagcaccgaggatctgagaacaagcggagggccgggccctgggacccctgctccagcccgaatgacggcatctgtttgccatgtacctggatgcgacgggcccctggggacaggcccttgccccatccatccgcttgaggcatggcaccgccctgcatccctaataccaaatctgactccaaaattgtggggtgtgacatacaagtgactgaacacttcctggggagctacaggggcacttaacccaccacagcacagcctcatcaaaatgcagctggcaacttctcccccaggtgccttccccctgctgccggcctttgctccttcacttccaacatctctcaaaataaaaatccctcttcccgctctgagcgattcagctctgcccgcagcttgtacatgtctctcccctggcaaaacaagagctgggtagtttagccaaacggcaccccctcgagttcactgcagacccttcgttcaccgtgtcacacatagaggggttctgagtaagaacaaaacgttctgctgctcaagccagtctggcaagcactcagcccagcctcgaggtccttctggggagagtgtaagtggacagagtcctggtcagggggcaggagtgtcccaagggctggcccacctgctgtctgtctgctcctcctagcccttggtcagatggcagccagagtccctcaggacctgcagcctcgccccggcagaagtcttttgtccaggaggcaaaaagccagagattctgcaacacgaattcgaagcaaacaaacacaacacaacagaattcctggaaagaagacgactgctaagacacggcaggggggcctggagggagcctccgactctgagctgctccgggatctgccgcgttctcctctgcacattgctgtttctgcccctgatgctggagctcaaggagactccttcctctttctcagcagagctgtagctgactgtggcattactacgcctccccacacgcccagacccctcactccaaaatcctactggctgtagcagagaatacctttgaaccaagattctgttttaatcatcatttacattgttttcttccaaaggccccctcgtataccctccctaacccacaaacctgttaacattgtcttaaggtgaaatggctggaaaatcagtatttaactaataaatttatctgtattcctctttc

SEQ ID NO: 111 - DNA, 3416 bp, Homo sapiens
  source 1..3416: mol_type = genomic DNA
ggggacttggaggcggccggcgcaggggccgcgagaggcttcgtcgccgctgcagctccgggggctcccaggggagcgtgcgcggaacctccaggcccagcaggaccccggctgcggcgaggaggaaggagccagcctagcagcttctgcgcctgtggccgcgggtgtcctggaggcctctcggtgtgacgagtgggggacccgaaggctcgtgcgccacctccaggcctggacgctgccctccgtcttctgcccccaataggtgcgccggaccttcaggccctggggtgaattcagctgctcctacatcagcttccggaaccaccaaaaattcaaattgggattttccggagtaaacaagagcctagagccctttgctcaatgctggatttaatacgtatatatttttaagcgagttggttttttcccctttgatttttgatcttcgcgacagttcctcccacgcatattatcgttgttgccgtcgttttctctccccgcgtggctccttgacctgcgagggagagagaggacaccgaagccgggagctcgcagggaccatgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaatcttccctcttccctcctcaaattcctgcacggacctgggacttggaggatagcaaagaaggaggccctgggctcccaggggccggcctcctctgcctggtaatgactccagaacaacaactgggaagaaacttgaagtcgacaatctggttaggggaagcgggtgttggattttctcagatgcctttacacgctgatgggactggagggagcccacccttcagcacgagcacactgcatctctcctgtgagttggagacttctttcccaagatgtccttgtcccctgcgttccccactgtggcctagaccgtgggttttgcattgtgtttctagcaccgaggatctgagaacaagcggagggccgggccctgggacccctgctccagcccgaatgacggcatctgtttgccatgtacctggatgcgacgggcccctggggacaggcccttgccccatccatccgcttgaggcatggcaccgccctgcatccctaataccaaatctgactccaaaattgtggggtgtgacatacaagtgactgaacacttcctggggagctacaggggcacttaacccaccacagcacagcctcatcaaaatgcagctggcaacttctcccccaggtgccttccccctgctgccggcctttgctccttcacttccaacatctctcaaaataaaaatccctcttcccgctctgagcgattcagctctgcccgcagcttgtacatgtctctcccctggcaaaacaagagctgggtagtttagccaaacggcaccccctcgagttcactgcagacccttcgttcaccgtgtcacacatagaggggttctgagtaagaacaaaacgttctgctgctcaagccagtctggcaagcactcagcccagcctcgaggtccttctggggagagtgtaagtggacagagtcctggtcagggggcaggagtgtcccaagggctggcccacctgctgtctgtctgctcctcctagcccttggtcagatggcagccagagtccctcaggacctgcagcctcgccccggcagaagtcttttgtccaggaggcaaaaagccagagattctgcaacacgaattcgaagcaaacaaacacaacacaacagaattcctggaaagaagacgactgctaagacacggcaggggggcctggagggagcctccgactctgagctgctccgggatctgccgcgttctcctctgcacattgctgtttctgcccctgatgctggagctcaaggagactccttcctctttctcagcagagctgtagctgactgtggcattactacgcctccccacacgcccagacccctcactccaaaatcctactggctgtagcagagaatacctttgaaccaagattctgttttaatcatcatttacattgttttcttccaaaggccccctcgtataccctccctaacccacaaacctgttaacattgtcttaaggtgaaatggctggaaaatcagtatttaactaataaatttatctgtattcctctttc

SEQ ID NO: 112 - DNA, 2343 bp, Homo sapiens
  source 1..2343: mol_type = genomic DNA
ggggacttggaggcggccggcgcaggggccgcgagaggcttcgtcgccgctgcagctccgggggctcccaggggagcgtgcgcggaacctccaggcccagcagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaatcttccctcttccctcctcaaattcctgcacggacctgggacttggaggatagcaaagaaggaggccctgggctcccaggggccggcctcctctgcctggtaatgactccagaacaacaactgggaagaaacttgaagtcgacaatctggttaggggaagcgggtgttggattttctcagatgcctttacacgctgatgggactggagggagcccacccttcagcacgagcacactgcatctctcctgtgagttggagacttctttcccaagatgtccttgtcccctgcgttccccactgtggcctagaccgtgggttttgcattgtgtttctagcaccgaggatctgagaacaagcggagggccgggccctgggacccctgctccagcccgaatgacggcatctgtttgccatgtacctggatgcgacgggcccctggggacaggcccttgccccatccatccgcttgaggcatggcaccgccctgcatccctaataccaaatctgactccaaaattgtggggtgtgacatacaagtgactgaacacttcctggggagctacaggggcacttaacccaccacagcacagcctcatcaaaatgcagctggcaacttctcccccaggtgccttccccctgctgccggcctttgctccttcacttccaacatctctcaaaataaaaatccctcttcccgctctgagcgattcagctctgcccgcagcttgtacatgtctctcccctggcaaaacaagagctgggtagtttagccaaacggcaccccctcgagttcactgcagacccttcgttcaccgtgtcacacatagaggggttctgagtaagaacaaaacgttctgctgctcaagccagtctggcaagcactcagcccagcctcgaggtccttctggggagagtgtaagtggacagagtcctggtcagggggcaggagtgtcccaagggctggcccacctgctgtctgtctgctcctcctagcccttggtcagatggcagccagagtccctcaggacctgcagcctcgccccggcagaagtcttttgtccaggaggcaaaaagccagagattctgcaacacgaattcgaagcaaacaaacacaacacaacagaattcctggaaagaagacgactgctaagacacggcaggggggcctggagggagcctccgactctgagctgctccgggatctgccgcgttctcctctgcacattgctgtttctgcccctgatgctggagctcaaggagactccttcctctttctcagcagagctgtagctgactgtggcattactacgcctccccacacgcccagacccctcactccaaaatcctactggctgtagcagagaatacctttgaaccaagattctgttttaatcatcatttacattgttttcttccaaaggccccctcgtataccctccctaacccacaaacctgttaacattgtcttaaggtgaaatggctggaaaatcagtatttaactaataaatttatctgtattcctctttc

SEQ ID NO: 113 - DNA, 2376 bp, Homo sapiens
  source 1..2376: mol_type = genomic DNA
ggggacttggaggcggccggcgcaggggccgcgagaggcttcgtcgccgctgcagctccgggggctcccaggggagcgtgcgcggaacctccaggcccagcaggaccccggctgcggcgaggaggaaggagccagcctagcagcttctgcgcctgtggccgcgggtgtcctggaggcctctcggtgtgacgagtgggggacccgaaggctcgtgcgccacctccaggcctggacgctgccctccgtcttctgcccccaatagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaatcttccctcttccctcctcaaattcctgcacggacctgggacttggaggatagcaaagaaggaggccctgggctcccaggggccggcctcctctgcctggtaatgactccagaacaacaactgggaagaaacttgaagtcgacaatctggttaggggaagcgggtgttggattttctcagatgcctttacacgctgatgggactggagggagcccacccttcagcacgagcacactgcatctctcctgtgagttggagacttctttcccaagatgtccttgtcccctgcgttccccactgtggcctagaccgtgggttttgcattgtgtttctagcaccgaggatctgagaacaagcggagggccgggccctgggacccctgctccagcccgaatgacggcatctgtttgccatgtacctggatgcgacgggcccctggggacaggcccttgccccatccatccgcttgaggcatggcaccgccctgcatccctaataccaaatctgactccaaaattgtggggtgtgacatacaagtgactgaacacttcctggggagctacaggggcacttaacccaccacagcacagcctcatcaaaatgcagctggcaacttctcccccaggtgccttccccctgctgccggcctttgctccttcacttccaacatctctcaaaataaaaatccctcttcccgctctgagcgattcagctctgcccgcagcttgtacatgtctctcccctggcaaaacaagagctgggtagtttagccaaacggcaccccctcgagttcactgcagacccttcgttcaccgtgtcacacatagaggggttctgagtaagaacaaaacgttctgctgctcaagccagtctggcaagcactcagcccagcctcgaggtccttctggggagagtgtaagtggacagagtcctggtcagggggcaggagtgtcccaagggctggcccacctgctgtctgtctgctcctcctagcccttggtcagatggcagccagagtccctcaggacctgcagcctcgccccggcagaagtcttttgtccaggaggcaaaaagccagagattctgcaacacgaattcgaagcaaacaaacacaacacaacagaattcctggaaagaagacgactgctaagacacggcaggggggcctggagggagcctccgactctgagctgctccgggatctgccgcgttctcctctgcacattgctgtttctgcccctgatgctggagctcaaggagactccttcctctttctcagcagagctgtagctgactgtggcattactacgcctccccacacgcccagacccctcactccaaaatcctactggctgtagcagagaatacctttgaaccaagattctgttttaatcatcatttacattgttttcttccaaaggccccctcgtataccctccctaacccacaaacctgttaacattgtcttaaggtgaaatggctggaaaatcagtatttaactaataaatttatctgtattcctctttc

SEQ ID NO: 114 - protein, 442 aa, Mus musculus
MYQSLAMAANHGPPPGAYEAGGPGAFMHSAGAASSPVYVPTPRVPSSVLGLSYLQGGGSAAAAGTTSGGSSGAGPSGAGPGTQQGSPGWSQAGAEGAAYTPPPVSPRFSFPGTTGSLAAAAAAAAAREAAAYGSGGGAAGAGLAGREQYGRPGFAGSYSSPYPAYMADVGASWAAAAAASAGPFDSPVLHSLPGRANPGRHPNLVDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAGPAGETLPPSSGASSGNSSNATSSSSSSEEMRPIKTEPGLSSHYGHSSSMSQTFSTVSGHGPSIHPVLSALKLSPQGYASPVTQTSQASSKQDSWNSLVLADSHGDIITA

SEQ ID NO: 115 - protein, 441 aa, Mus musculus
MYQSLAMAANHGPPPGAYEAGGPGAFMHSAGAASSPVYVPTPRVPSSVLGLSYLQGGGSAAAAGTTSGGSSGAGPSGAGPGTQQGSPGWSQAGAEGAAYTPPPVSPRFSFPGTTGSLAAAAAAAAAREAAAYGSGGGAAGAGLAGREQYGRPGFAGSYSSPYPAYMADVGASWAAAAAASAGPFDSPVLHSLPGRANPGRHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAGPAGETLPPSSGASSGNSSNATSSSSSSEEMRPIKTEPGLSSHYGHSSSMSQTFSTVSGHGPSIHPVLSALKLSPQGYASPVTQTSQASSKQDSWNSLVLADSHGDIITA

SEQ ID NO: 116 - DNA, 3405 bp, Mus musculus
  source 1..3405: mol_type = genomic DNA
gcgccgggagcaggggacaagccggaggcccgcagagtggccgcccgaggctcagccgcagttgcagctccgcggactcacggagatcgcgccggttttctgggaaactggagctggccaggactgccgcttcgcttcgaagggaccgggccctctttgtcattcttcgctggagccgctctggagctagcagctgcgcctgggtgtgtagcaggcagaaagcaaggactaggcttctttagccggtgggtgatccgaaggcctgctcagggtgttcgagaccagcctggactgcgtctgggcacctccagcctctgggccctggaatagagtccgccctcccgcacgatttctggagcaaccgcaaatccaatttgggattttctttttcctgagcaaaccagagcctagaggtttctgctttgatgctggatttaattcgtatatattttgagcgagttgggcctctcctcgttttttgatctccggttgttttttttttggggggggggttagtttttgggtttttgttttgttttgttttgttttgatttttggtgacagttccgcacacccgcattctagttcttgtctgcctcgtgctcagagcttggggcgatgtaccaaagcctggccatggccgccaaccacggccccccgcccggcgcctacgaagcaggtggccctggcgccttcatgcacagcgcgggcgccgcgtcctcgcccgtctacgtgcccactccgcgggtgccgtcctctgtgctgggcctgtcctacctgcagggcggtggcagtgccgctgcagctggaaccacctcgggtggcagctccggggccggcccgtcgggtgcagggcctgggacccagcagggtagccctggctggagccaagctggagccgagggagccgcctacaccccgccgcccgtgtccccgcgcttctctttcccggggactactgggtccctggcggccgctgccgccgctgccgcagcccgggaagctgcagcctacggcagtggcggcggggcggcgggcgctggtctggctggccgagagcagtacgggcgtccgggcttcgccggctcctactccagcccctacccagcctacatggccgacgtgggagcatcctgggccgcagccgctgccgcctctgccggccccttcgacagcccagtcctgcacagcctgcctggacgggccaaccctggaagacaccccaatctcgatatgtttgatgacttctcagaaggcagagagtgtgtcaattgtggggccatgtccaccccactctggaggcgagatgggacgggacactacctgtgcaatgcctgtggcctctatcacaagatgaacggcatcaaccggcccctcattaagcctcagcgccgcctgtccgcttcccgccgggtaggcctctcctgtgccaactgccagactaccaccaccacgctgtggcgtcgtaatgccgagggtgagcctgtatgtaatgcctgcggcctctacatgaagctccatggggttcccaggcctcttgcaatgcggaaggaggggattcaaaccagaaaacggaagcccaagaacctgaataaatctaagacgccagcaggtcctgctggtgagaccctccctccctccagtggtgcctccagcggtaactccagcaatgccactagcagcagcagcagcagtgaagagatgcgccccatcaagacagagcccgggctgtcatctcactatgggcacagcagctccatgtcccagacattcagtactgtgtccggccacgggccctccatccatccagtgctgtctgctctgaagctgtccccacaaggctatgcatctcctgtcactcagacatcgcaggccagctccaagcaggactcttggaacagcctggtcctggctgacagtcatggggacataatcaccgcgtaatcagcgcccccccttccctcttcaaattcctgctcggacttgggacgtgggggccagcaaagtaaaaggctggggcacccttggccagcccctttgtctgggaacaactcctgaagaacaactgggtagaacttgaagttgttgacaatcacttagggatatgggtgttccgggttgttcaaacacctttccaggtggagcactggaaaagcctgcgttcttacagagaagcccaccttggctgcaagcacagcacagtgaggcaagagacttcttccttccttattctccacctgcctgtccaggacagacacataatctccttcaccccagctccccacccagttgtggtggtgggtttttctttgtgatcctagagtggctgtaggggcggaggcttcaagacaccatctacagtctgagcagggtgtctacttgttgtagactagacatagaagccctgcccttgtccaacactccccttgcttgaggcatggcacatctctgcatgtcccataccagatctgactccaaagtgctgggttcaatgcagatgttactgaatgcttcctggggagattaggtgaggggaaggcacatcacccatcacacagaatagcttcatcaaatcgcagcctggccatggtgccttcccttcctctcccaggaacatcaaaccccttgctctccagcctgaacatctaccctctgcaaaagtagagcccagttgtgcagctaatgccactaggtgctatatcccagcatccttttcaccccttcacacacaggggttccaaggaggaacaaaacctgctaccaaagcagccttggtgactatggctcatctgcacctcagggggtgggggagggccctctggaggttgtgtctacagcacaatactgttcccaggactctagcttgcttgccccgagcctgccaagccaagccctcttaagtcagacagttacctggctctgggactttctccagcacagatcctttgtctagaaaatacagactgtttgcaaaataaattcaaagcagaaacaactaaaggaaatttgtgaaaggacaaaggtgatagacgggagaagatgtccccagggctggcgggacagtcatgatagcagctgtcctaggattggcctccctcccatctcccaccattactggggctcccagagattcttccttgtcctcatcacccacagagctgtagccaactgtggcattactttattttacccaaaattcccagccccacccctaaaccttactggccgtagcagagaatagcttcgaaccaagattctgttgtaatcattttcgctgtttctccctcaaggccgccttccccatgcctgcccctcctccacaacccgttaacattgtcttaaggtgaaatggctgtaaaatcagtatttaactaataaatttatctgtattcctgtttcctccga

SEQ ID NO: 117 - DNA, 3408 bp, Mus musculus
  source 1..3408: mol_type = genomic DNA
gcgccgggagcaggggacaagccggaggcccgcagagtggccgcccgaggctcagccgcagttgcagctccgcggactcacggagatcgcgccggttttctgggaaactggagctggccaggactgccgcttcgcttcgaagggaccgggccctctttgtcattcttcgctggagccgctctggagctagcagctgcgcctgggtgtgtagcaggcagaaagcaaggactaggcttctttagccggtgggtgatccgaaggcctgctcagggtgttcgagaccagcctggactgcgtctgggcacctccagcctctgggccctggaatagagtccgccctcccgcacgatttctggagcaaccgcaaatccaatttgggattttctttttcctgagcaaaccagagcctagaggtttctgctttgatgctggatttaattcgtatatattttgagcgagttgggcctctcctcgttttttgatctccggttgttttttttttggggggggggttagtttttgggtttttgttttgttttgttttgttttgatttttggtgacagttccgcacacccgcattctagttcttgtctgcctcgtgctcagagcttggggcgatgtaccaaagcctggccatggccgccaaccacggccccccgcccggcgcctacgaagcaggtggccctggcgccttcatgcacagcgcgggcgccgcgtcctcgcccgtctacgtgcccactccgcgggtgccgtcctctgtgctgggcctgtcctacctgcagggcggtggcagtgccgctgcagctggaaccacctcgggtggcagctccggggccggcccgtcgggtgcagggcctgggacccagcagggtagccctggctggagccaagctggagccgagggagccgcctacaccccgccgcccgtgtccccgcgcttctctttcccggggactactgggtccctggcggccgctgccgccgctgccgcagcccgggaagctgcagcctacggcagtggcggcggggcggcgggcgctggtctggctggccgagagcagtacgggcgtccgggcttcgccggctcctactccagcccctacccagcctacatggccgacgtgggagcatcctgggccgcagccgctgccgcctctgccggccccttcgacagcccagtcctgcacagcctgcctggacgggccaaccctggaagacaccccaatctcgtagatatgtttgatgacttctcagaaggcagagagtgtgtcaattgtggggccatgtccaccccactctggaggcgagatgggacgggacactacctgtgcaatgcctgtggcctctatcacaagatgaacggcatcaaccggcccctcattaagcctcagcgccgcctgtccgcttcccgccgggtaggcctctcctgtgccaactgccagactaccaccaccacgctgtggcgtcgtaatgccgagggtgagcctgtatgtaatgcctgcggcctctacatgaagctccatggggttcccaggcctcttgcaatgcggaaggaggggattcaaaccagaaaacggaagcccaagaacctgaataaatctaagacgccagcaggtcctgctggtgagaccctccctccctccagtggtgcctccagcggtaactccagcaatgccactagcagcagcagcagcagtgaagagatgcgccccatcaagacagagcccgggctgtcatctcactatgggcacagcagctccatgtcccagacattcagtactgtgtccggccacgggccctccatccatccagtgctgtctgctctgaagctgtccccacaaggctatgcatctcctgtcactcagacatcgcaggccagctccaagcaggactcttggaacagcctggtcctggctgacagtcatggggacataatcaccgcgtaatcagcgcccccccttccctcttcaaattcctgctcggacttgggacgtgggggccagcaaagtaaaaggctggggcacccttggccagcccctttgtctgggaacaactcctgaagaacaactgggtagaacttgaagttgttgacaatcacttagggatatgggtgttccgggttgttcaaacacctttccaggtggagcactggaaaagcctgcgttcttacagagaagcccaccttggctgcaagcacagcacagtgaggcaagagacttcttccttccttattctccacctgcctgtccaggacagacacataatctccttcaccccagctccccacccagttgtggtggtgggtttttctttgtgatcctagagtggctgtaggggcggaggcttcaagacaccatctacagtctgagcagggtgtctacttgttgtagactagacatagaagccctgcccttgtccaacactccccttgcttgaggcatggcacatctctgcatgtcccataccagatctgactccaaagtgctgggttcaatgcagatgttactgaatgcttcctggggagattaggtgaggggaaggcacatcacccatcacacagaatagcttcatcaaatcgcagcctggccatggtgccttcccttcctctcccaggaacatcaaaccccttgctctccagcctgaacatctaccctctgcaaaagtagagcccagttgtgcagctaatgccactaggtgctatatcccagcatccttttcaccccttcacacacaggggttccaaggaggaacaaaacctgctaccaaagcagccttggtgactatggctcatctgcacctcagggggtgggggagggccctctggaggttgtgtctacagcacaatactgttcccaggactctagcttgcttgccccgagcctgccaagccaagccctcttaagtcagacagttacctggctctgggactttctccagcacagatcctttgtctagaaaatacagactgtttgcaaaataaattcaaagcagaaacaactaaaggaaatttgtgaaaggacaaaggtgatagacgggagaagatgtccccagggctggcgggacagtcatgatagcagctgtcctaggattggcctccctcccatctcccaccattactggggctcccagagattcttccttgtcctcatcacccacagagctgtagccaactgtggcattactttattttacccaaaattcccagccccacccctaaaccttactggccgtagcagagaatagcttcgaaccaagattctgttgtaatcattttcgctgtttctccctcaaggccgccttccccatgcctgcccctcctccacaacccgttaacattgtcttaaggtgaaatggctgtaaaatcagtatttaactaataaatttatctgtattcctgtttcctccga

SEQ ID NO: 118 - protein, 595 aa, Homo sapiens
MALTDGGWCLPKRFGAAGADASDSRAFPAREPSTPPSPISSSSSSCSRGGERGPGGASNCGTPQLDTEAAAGPPARSLLLSSYASHPFGAPHGPSAPGVAGPGGNLSSWEDLLLFTDLDQAATASKLLWSSRGAKLSPFAPEQPEEMYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA

SEQ ID NO: 119 - protein, 358 aa, Homo sapiens
MGDWSFLGNFLEEVHKHSTVVGKVWLTVLFIFRMLVLGTAAESSWGDEQADFRCDTIQPGCQNVCYDQAFPISHIRYWVLQIIFVSTPSLVYMGHAMHTVRMQEKRKLREAERAKEVRGSGSYEYPVAEKAELSCWEEGNGRIALQGTLLNTYVCSILIRTTMEVGFIVGQYFIYGIFLTTLHVCRRSPCPHPVNCYVSRPTEKNVFIVFMLAVAALSLLLSLAELYHLGWKKIRQRFVKPRQHMAKCQLSGPSVGIVQSCTPPPDFNQCLENGPGGKFFNPFSNNMASQQNTDNLVTEQVRGQEQTPGEGFIQVRYGQKPEVPNGVSPGHRLPHGYHSDKRRLSKASSKARSDDLSV

SEQ ID NO: 120 - protein, 589 aa, Mus musculus
MALTDGGWCLPKRFGAAAADAGDSGPFPAREPSSPLSPISSSSSSCSRGGDRGPCGASNCRTPQLDAEAVAGPPGRSLLLSPYASHPFAAAHGAAAPGVAGPGSALSTWEDLLLFTDLDQAATASKLLWSSRGAKLSPFAAEQPEEMYQTLAALSSQGPAAYDGAPGGFVHSAAAAAAAAAAASSPVYVPTTRVGSMLSGLPYLQGAGSGPSNHAGGAGAHPGWSQASADSPPYGGGGAAGGGAAGPGGAGSATAHASARFPYSPSPPMANGAARDPGGYVAAGGTGAGSVSGGGGSLAAMGGREHQYSSLSAARPLNGTYHHHHHHHPTYSPYMAAPLTPAWPAGPFETPVLHSLQGRAGAPLPVPRGPSTDLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKACSGNSSGSVPMTPTSSSSNSDDCTKNTSPSTQATTSGVGASVMSAVGENANPENSDLKYSGQDGLYIGVSLSSPAEVTSSVRQDSWCALALA

SEQ ID NO: 121 - DNA, 3194 bp, Mus musculus
  source 1..3194: mol_type = genomic DNA
cccacagcctggcacccttcggctagcgctgtttgtttagggctcggtgagtccaatcaggagcgcaggctgcagttttccggcagagcagtaagaggcgcctcctctctcctttttattcaccagcagcgactagcagaccccggactctcgctctcccgccggcgccctccgcctctctccgcgccccggagcaccctcggtcgcggccgttcttctcgcacatcgctcgaggaatcaaaagtcaggttggagtagcgccggacagtggatggccttgactgacggcggctggtgcctgccaaagcgtttcggggctgctgctgcggacgccggcgactccgggccctttccagcgcgggagccctcctcgccgctttcccccatctcgtcttcgtcctcctcctgctcccggggcggggatcgcggtccctgcggcgccagcaactgcaggacgccgcagctcgacgccgaggcggtggcgggacctccgggccgctcgctcttgctcagcccctacgcctcgcatcccttcgccgctgcccacggagccgcggcgcccggggtcgcaggccccgggagcgccctgtcgacttgggaggacctgttgctcttcactgacctcgatcaggccgcgaccgccagcaagctgttgtggtccagccggggcgccaaactgagccccttcgcggccgagcagccggaggaaatgtaccagaccctcgccgccctgtccagccaggggcccgccgcttacgacggcgcgcccggcggcttcgtgcactccgcagcggcggcggccgctgccgccgcggcagccagctccccggtctacgtgcccaccacgcgcgtgggctccatgctgtccggcctgccctaccttcaaggggcgggcagcgggcccagcaatcacgcgggcggagcgggtgcccacccaggctggtcccaggcctccgccgacagccccccgtatggcgggggtggcgcagccggcggcggcgcggccggacctggaggtgcgggatcggctacggcccacgcctctgcacgctttccctactcgcccagcccgcccatggccaacggcgccgcgcgagaccccgggggctacgtggctgcgggcggcacgggcgcaggcagtgtgagtggaggtggcggcagcctggcggccatgggtggccgggagcaccagtacagctcgctgtccgcagctcggccgctgaacggaacgtaccaccaccaccatcaccatcacccgacctactcgccctacatggccgcaccgctgactcctgcctggccagcaggacccttcgaaacgccggtgctccacagcttacagggccgcgcgggagctccactcccggtgccacggggccccagcacagacctgttggaggacctgtcggagagccgcgagtgcgtgaactgcggctccatccagacgccactgtggagacgagacggcaccggtcattacctgtgcaatgcatgcggtctctacagcaagatgaatggcctcagcaggcccctcatcaagccacagaagcgcgtgccttcatcacggcggcttggactgtcctgtgccaactgtcacaccacaaccactaccttatggcgtagaaatgctgagggtgagcctgtgtgcaatgcttgcgggctctatatgaaactccatggggtgcctcgaccacttgctatgaaaaaagaaggaattcaaaccaggaaacgaaaacctaaaaatataaataagtcaaaagcttgctccggtaacagcagtggctctgtccctatgactcctacttcctcttcttctaattcagatgactgcaccaaaaatacttctccttctacacaagcgaccacctcaggggtaggggcatcagtgatgtctgcagtgggagaaaacgccaaccccgagaacagtgacctcaagtattcaggtcaagacggcctctacataggtgtcagtctgtcctcccctgccgaagtcacatcctccgtgcgacaggattcttggtgtgctctggccctggcctgagctggtgctaccaagaggcaaggagggctctgaaggcctcataccacttgtgtctgatattgtccagcagtccagatggcagcaaaaatgcagacataacattccttcgatgcgtgatttctgtgcctttgttttgaaagagatatatttctcaagaagcttactgaagtaagaagagatgggcttttgcaggaagggccagcaccgtgggcatgtggcctgctcctgccagcctgggctgcttcctgcctctgactctgccccataccagtgggagaaactgtgacaatgaccggggccttgtctgctaaggaagattgagagatttaagagaaaatgtttgtgtattgctccaaatcatgtgcttcttgtgatcaacctcggttatcccagaacccattcatccccgaccaccgtgcacatttcacaagcgttcgtggagaggagcactgggagccatttggtctatcctggaggcggagtgcattcctggggtctcaacaagaatattaatttgcaagattgcatcatgacagacactgactgacttatctcaacgttcatcgtaacgtggctgatctgaggtcacttggaatttgtaaacagggtagcaaacaagatatttttcttccatgtacacaataatttttttaagtgcaatttgcgttgcagcaatcagtgttaaatcatttgcataagatttaacagcatttttataatgaatgtaaacattttaacttaatggtacttaaaataatttaaaaaaaagttaactttagacatatgcttcttacactcacagcccacttctgtgttcccaattgtttaaaagaaaaaaaaaaagatttcaagaacaaatcttctctcaggaaattgccttttctccatttatgaatttttatacaagaacaccaacacagtccccgttcttttactgaggaaaaagtgctggaaattgcaacaaacctttactacctagagaatagcatttgtaaatattctaagtatctgtaacactcttgacgcctgtaccacgtgaccaacccacaggttggtttacattattattttttttaatgggatatcatatggaaacctatttcaccagagttttaaaaaataaaaagggtattgttttgtgttctgtacagtg

SEQ ID NO: 122 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 123 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 124 - protein, 88 aa, Homo sapiens
MKVLAAVHSPGGAVPQQPGQAMWPQRDGLPALPRQRHGEGQAGGAVPHSRVPWHLPGQHHPGPEDPQPQCPQPPQQAQRHRRHPARPP

SEQ ID NO: 125 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 126 - protein, 357 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEVGQSPAAVGLGAGEQGGT

SEQ ID NO: 127 - protein, 335 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILALDLLDRMLTFNPNKRITVEEALAHPYLEQYYDPTDEPVAEEPFTFAMELDDLPKERLKELIFQETARFQPGVLEAP

SEQ ID NO: 128 - DNA, 1745 bp, Homo sapiens
  source 1..1745: mol_type = genomic DNA
ccccgtagaaccgagggggtgggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggcttctcctccccacccgccctccccacgggcctcgggacctcaggtgggcccagttcaatctcccgctgctgctgctgcgcccttaccttccccagcgtcccagtctctggcagttttggaatggaagggttctggctgccccaacctgctgaagggcagaggtggagggtggggggcgctgagtagggactcacggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggctgcccctgccacctccctgacccgtctaatatataaatatagagatgtgtctatggctgaaaaaaaaaa

SEQ ID NO: 129 - protein, 360 aa, Homo sapiens [sequence omitted]

SEQ ID NO: 130 - protein, 360 aa, Homo sapiens [sequence omitted]

SEQ ID NO: 131 - DNA, 21 bp, synthetic construct
  misc_feature 1..21: note = Single strand DNA oligonucleotide
  source 1..21: mol_type = other DNA
actgacggagagcatgaagat

SEQ ID NO: 132 - DNA, 19 bp, synthetic construct
  misc_feature 1..19: note = Single strand DNA oligonucleotide
  source 1..19: mol_type = other DNA
ccggcacataggaggggta

SEQ ID NO: 133 - DNA, 19 bp, synthetic construct
  misc_feature 1..19: note = Single strand DNA oligonucleotide
  source 1..19: mol_type = other DNA
gcccctcattaagcccaag

SEQ ID NO: 134 - DNA, 21 bp, synthetic construct
  misc_feature 1..21: note = Single strand DNA oligonucleotide
  source 1..21: mol_type = other DNA
ttgtggtggtctgacagttcg

SEQ ID NO: 135 - DNA, 21 bp, synthetic construct
  misc_feature 1..21: note = Single strand DNA oligonucleotide
  source 1..21: mol_type = other DNA
tcccaaagaatacggagcact

SEQ ID NO: 136 - DNA, 21 bp, synthetic construct
  misc_feature 1..21: note = Single strand DNA oligonucleotide
  source 1..21: mol_type = other DNA
tcatggggacagacttctcaa

SEQ ID NO: 137 - DNA, 23 bp, synthetic construct
  misc_feature 1..23: note = Single strand DNA oligonucleotide
  source 1..23: mol_type = other DNA
tagggaacaaggaatttttcggg

SEQ ID NO: 138 - DNA, 22 bp, synthetic construct
  misc_feature 1..22: note = Single strand DNA oligonucleotide
  source 1..22: mol_type = other DNA
gtacactaaccttcggtcaacc

SEQ ID NO: 139 - DNA, 20 bp, synthetic construct
  misc_feature 1..20: note = Single strand DNA oligonucleotide
  source 1..20: mol_type = other DNA
accaccctttcacaagcaac

SEQ ID NO: 140 - DNA, 20 bp, synthetic construct
  misc_feature 1..20: note = Single strand DNA oligonucleotide
  source 1..20: mol_type = other DNA
tgtgcccatttgcaattcta

SEQ ID NO: 141 - DNA, 20 bp, synthetic construct
  misc_feature 1..20: note = Single strand DNA oligonucleotide
  source 1..20: mol_type = other DNA
acaacgatggcctctacgac

SEQ ID NO: 142 - DNA, 19 bp, synthetic construct
  misc_feature 1..19: note = Single strand DNA oligonucleotide
  source 1..19: mol_type = other DNA
agttcacgcaccagcacac

SEQ ID NO: 143 - DNA, 22 bp, synthetic construct
  misc_feature 1..22: note = Single strand DNA oligonucleotide
  source 1..22: mol_type = other DNA
gttaccctgctcacatcactag

SEQ ID NO: 144 - DNA, 21 bp, synthetic construct
  misc_feature 1..21: note = Single strand DNA oligonucleotide
  source 1..21: mol_type = other DNA
tcttgtcacttgctcattggg

SEQ ID NO: 145 - DNA, 20 bp, synthetic construct
  misc_feature 1..20: note = Single strand DNA oligonucleotide
  source 1..20: mol_type = other DNA
tcacaaccctggttttcctc

SEQ ID NO: 146 - DNA, 20 bp, synthetic construct
  misc_feature 1..20: note = Single strand DNA oligonucleotide
  source 1..20: mol_type = other DNA
ctgtctggaactggtgctga

SEQ ID NO: 147 - DNA, 20 bp, synthetic construct
  misc_feature 1..20: note = Single strand DNA oligonucleotide
  source 1..20: mol_type = other DNA
tcaccatccggaggaaagcc

SEQ ID NO: 148 - DNA, 20 bp, synthetic construct
  misc_feature 1..20: note = Single strand DNA oligonucleotide
  source 1..20: mol_type = other DNA
ctctcctttgctctgcggtt

SEQ ID NO: 149 - DNA, 19 bp, synthetic construct
  misc_feature 1..19: note = Single strand DNA oligonucleotide
  source 1..19: mol_type = other DNA
ccaaggatgcacagtctgg

SEQ ID NO: 150 - DNA, 21 bp, synthetic construct
  misc_feature 1..21: note = Single strand DNA oligonucleotide
  source 1..21: mol_type = other DNA
aggaggaaaaccttcgtgctg

SEQ ID NO: 151 - DNA, 26 bp, synthetic construct
  misc_feature 1..26: note = Single strand DNA oligonucleotide
  source 1..26: mol_type = other DNA
acgaaatgagatggcagctaggaaga

SEQ ID NO: 152 - DNA, 24 bp, synthetic construct
  misc_feature 1..24: note = Single strand DNA oligonucleotide
  source 1..24: mol_type = other DNA
tgtccggtcttggctgagaagttc

SEQ ID NO: 153 - DNA, 21 bp, synthetic construct
  misc_feature 1..21: note = Single strand DNA oligonucleotide
  source 1..21: mol_type = other DNA
acgaacggattaacagggtca

SEQ ID NO: 154 - DNA, 21 bp, synthetic construct
  misc_feature 1..21: note = Single strand DNA oligonucleotide
  source 1..21: mol_type = other DNA
cctccagacacaccacggata

SEQ ID NO: 155 - DNA, 20 bp, synthetic construct
  misc_feature 1..20: note = Single strand DNA oligonucleotide
  source 1..20: mol_type = other DNA
acctggagatgcagatcgaa

SEQ ID NO: 156 - DNA, 20 bp, synthetic construct
  misc_feature 1..20: note = Single strand DNA oligonucleotide
  source 1..20: mol_type = other DNA
aatccacctccacactgacc

SEQ ID NO: 157 - DNA, 21 bp, synthetic construct
  misc_feature 1..21: note = Single strand DNA oligonucleotide
  source 1..21: mol_type = other DNA
tgcctcccggctcagttcact

SEQ ID NO: 158 - DNA, 20 bp, synthetic construct
  misc_feature 1..20: note = Single strand DNA oligonucleotide
  source 1..20: mol_type = other DNA
cccagtggtttggtggtgta

SEQ ID NO: 159 - DNA, 21 bp, synthetic construct
  misc_feature 1..21: note = Single strand DNA oligonucleotide
  source 1..21: mol_type = other DNA
gccatagtcctgttcacctca

SEQ ID NO: 160 - DNA, 18 bp, synthetic construct
  misc_feature 1..18: note = Single strand DNA oligonucleotide
  source 1..18: mol_type = other DNA
aatctcgtcggctggttg

SEQ ID NO: 161 - DNA, 20 bp, synthetic construct
  misc_feature 1..20: note = Single strand DNA oligonucleotide
  source 1..20: mol_type = other DNA
tggcgcagcagaatccagac

SEQ ID NO: 162 - DNA, 20 bp, synthetic construct
  misc_feature 1..20: note = Single strand DNA oligonucleotide
  source 1..20: mol_type = other DNA
ttggggtggtcctgcatgtg

SEQ ID NO: 163 - DNA, 19 bp, synthetic construct
  misc_feature 1..19: note = Single strand DNA oligonucleotide
  source 1..19: mol_type = other DNA
tgcctgacattgaccctgt

SEQ ID NO: 164 - DNA, 19 bp, synthetic construct
  misc_feature 1..19: note = Single strand DNA oligonucleotide
  source 1..19: mol_type = other DNA
ccgtctcaatggcactctc

SEQ ID NO: 165 - DNA, 20 bp, synthetic construct
  misc_feature 1..20: note = Single strand DNA oligonucleotide
  source 1..20: mol_type = other DNA
cctgcctggtattgtcatcc

SEQ ID NO: 166 - DNA, 20 bp, synthetic construct
  misc_feature 1..20: note = Single strand DNA oligonucleotide
  source 1..20: mol_type = other DNA
gatggggatgattgtggtct

SEQ ID NO: 167 - DNA, 18 bp, synthetic construct
  misc_feature 1..18: note = Single strand DNA oligonucleotide
  source 1..18: mol_type = other DNA
gcggacggtgaacgacta

SEQ ID NO: 168 - DNA, 18 bp, synthetic construct
  misc_feature 1..18: note = Single strand DNA oligonucleotide
  source 1..18: mol_type = other DNA
ggccgcctttccttttat

SEQ ID NO: 169 - DNA, 18 bp, synthetic construct
  misc_feature 1..18: note = Single strand DNA oligonucleotide
  source 1..18: mol_type = other DNA
cctccgcgaggagaaagt

SEQ ID NO: 170 - DNA, 20 bp, synthetic construct
  misc_feature 1..20: note = Single strand DNA oligonucleotide
  source 1..20: mol_type = other DNA
cgttctccgactcctctgat

SEQ ID NO: 171 - DNA, 20 bp, synthetic construct
  misc_feature 1..20: note = Single strand DNA oligonucleotide
  source 1..20: mol_type = other DNA
gtgtcaggcttgaggtggag

SEQ ID NO: 172 - DNA, 19 bp, synthetic construct
  misc_feature 1..19: note = Single strand DNA oligonucleotide
  source 1..19: mol_type = other DNA
tgcctgacattgaccctgt

SEQ ID NO: 173 - DNA, 19 bp, synthetic construct
  misc_feature 1..19: note = Single strand DNA oligonucleotide
  source 1..19: mol_type = other DNA
gcgggctctacagcaagat

SEQ ID NO: 174 - DNA, 19 bp, synthetic construct
  misc_feature 1..19: note = Single strand DNA oligonucleotide
  source 1..19: mol_type = other DNA
tggcacaggacaatccaag

SEQ ID NO: 175 - DNA, 20 bp, synthetic construct
  misc_feature 1..20: note = Single strand DNA oligonucleotide
  source 1..20: mol_type = other DNA
ggaagcccaagaacctgaat

SEQ ID NO: 176 - DNA, 19 bp, synthetic construct
  misc_feature 1..19: note = Single strand DNA oligonucleotide
  source 1..19: mol_type = other DNA
gttgctggagttgctggaa

SEQ ID NO: 177 - DNA, 21 bp, synthetic construct
  misc_feature 1..21: note = Single strand DNA oligonucleotide
  source 1..21: mol_type = other DNA
tgaagatggaagggcacgagc

SEQ ID NO: 178 - DNA, 25 bp, synthetic construct
  misc_feature 1..25: note = Single strand DNA oligonucleotide
  source 1..25: mol_type = other DNA
ccgacatgctcatgtacgtgttcat

SEQ ID NO: 179 - DNA, 21 bp, synthetic construct
  misc_feature 1..21: note = Single strand DNA oligonucleotide
  source 1..21: mol_type = other DNA
agtccactgagtaccggagac

SEQ ID NO: 180 - DNA, 21 bp, synthetic construct
  misc_feature 1..21: note = Single strand DNA oligonucleotide
  source 1..21: mol_type = other DNA
catttcacgcatctggcgttc

SEQ ID NO: 181 - DNA, 21 bp, synthetic construct
  misc_feature 1..21: note = Single strand DNA oligonucleotide
  source 1..21: mol_type = other DNA
agggctgcttttaactctggt

SEQ ID NO: 182 - DNA, 21 bp, synthetic construct
  misc_feature 1..21: note = Single strand DNA oligonucleotide
  source 1..21: mol_type = other DNA
ccccacttgattttggaggga

SEQ ID NO: 183 - DNA, 22 bp, synthetic construct
  misc_feature 1..22: note = Single strand DNA oligonucleotide
  source 1..22: mol_type = other DNA
ccacgaaactaccttcaactcc

SEQ ID NO: 184 - DNA, 22 bp, synthetic construct
  misc_feature 1..22: note = Single strand DNA oligonucleotide
  source 1..22: mol_type = other DNA
gtgatctccttctgcatcctgt

SEQ ID NO: 185 - DNA, 20 bp, synthetic construct
  misc_feature 1..20: note = Single strand DNA oligonucleotide
  source 1..20: mol_type = other DNA
tcgtaaagcgaggtgatggg

SEQ ID NO: 186 - DNA, 20 bp, synthetic construct
  misc_feature 1..20: note = Single strand DNA oligonucleotide
  source 1..20: mol_type = other DNA
aagctcacagcctccatgtc

SEQ ID NO: 187 - DNA, 20 bp, synthetic construct
  misc_feature 1..20: note = Single strand DNA oligonucleotide
  source 1..20: mol_type = other DNA
aactgtgaggtggtccttgg

SEQ ID NO: 188 - DNA, 20 bp, synthetic construct
  misc_feature 1..20: note = Single strand DNA oligonucleotide
  source 1..20: mol_type = other DNA
gttgagggcaatgaggacat

SEQ ID NO: 189 - DNA, 20 bp, synthetic construct
  misc_feature 1..20: note = Single strand DNA oligonucleotide
  source 1..20: mol_type = other DNA
tggtctccaaacggcacttt

SEQ ID NO: 190 - DNA, 20 bp, synthetic construct
  misc_feature 1..20: note = Single strand DNA oligonucleotide
  source 1..20: mol_type = other DNA
aactgtgaggtggtccttgg

SEQ ID NO: 191 - DNA, 23 bp, synthetic construct
  misc_feature 1..23: note = Single strand DNA oligonucleotide
  source 1..23: mol_type = other DNA
cactccactaaggtccaagaaga

SEQ ID NO: 192 - DNA, 21 bp, synthetic construct
  misc_feature 1..21: note = Single strand DNA oligonucleotide
  source 1..21: mol_type = other DNA
ccgtgtggttctccactttga

SEQ ID NO: 193 - DNA, 21 bp, synthetic construct
  misc_feature 1..21: note = Single strand DNA oligonucleotide
  source 1..21: mol_type = other DNA
agatgccgatgacccttcata

SEQ ID NO: 194 - DNA, 21 bp, synthetic construct
  misc_feature 1..21: note = Single strand DNA oligonucleotide
  source 1..21: mol_type = other DNA
tgttcggtaaagcagtcctga

SEQ ID NO: 195 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 196 - protein, 408 aa, Mus musculus
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEEQSQGTGLEYPERPASRANTVRSFHHEEHLENIPGTSESSAFRFLFNLSSIPENEVISSAELRLFREQVDQGPDWEQGFHRINIYEVMKPPAEMVPGHLITRLLDTRLVHHNVTRWETFDVSPAVLRWTREKQPNYGLAIEVTHLHQTRTHQGQHVRISRSLPQGSGDWAQLRPLLVTFGHDGRGHTLTRRRAKRSPKHHPQRSRKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 197 - protein, 414 aa, Homo sapiens [sequence omitted]

SEQ ID NO: 198 - DNA, 2346 bp, Homo sapiens
  source 1..2346: mol_type = genomic DNA
gcagtgtcactaggccggctgggggccctgggtacgctgtagaccagaccgcgacaggccagaacacgggcggcggcttcgggccgggagacccgcgcagccctcggggcatctcagtgcctcactccccaccccctcccccgggtcgggggaggcggcgcgtccggcggagggttgaggggagcggggcaggcctggagcgccatgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtgtgacaggtccctgatccgccccagcctgcaggccagaagcagtgttacacacttcctggaggagctaaggaaatcctcagactcctgggtttttgttgttgctgttgttgttttttaaaaggtgtgttggcatataatttatggtaatttattttgtctgccacttgaacagtttgggggggtgaggtttcatttaaaatttgttcagagatttgtttcccatagttggattgtcaaaaccctatttccaagttcaagttaactagctttgaatgtgtcccaaaacagcttcctccatttcctgaaagtttattgatcaaagaaatgttgtcctgggtgtgttttttcaatcttctaaaaaataaaatctggaatcctgcttttttgctctactagtacctctgtcacactagtcttatcaaaaaccagttcttaagatcaatgttaagtttattagttaatgtaaatttctcatcctcgaaaagggtgaacataaatgcctttaaggagtatatctaaaaataaacattaggatatctaagtttgatgtaattgtttcaggaaggaaaaaagaaaagcattctggaatgagcctacttcaagtaatcttagtttctaaaactaacagttaatattttcaattccagtatatcactttaagtagaaggggatgtccaagtaattttggttttctaactgttgaatcataagcttgacctgcccccagaggctttttggatgtttttatctgtgttttgccatctctttacactcctcgacattcagtttaccttaatcttcacatttttacaccttgggaagtggcaagcatcgctgggtttaagataaaggagtcacaaaaactaatcaaaataaaatttgcattatgacaacttttaa

SEQ ID NO: 199 - protein, 419 aa, Mus musculus
MSSPDAGYASDDQSQPRSAQPAVMAGLGPCPWAESLSPLGDVKVKGEVVASSGAPAGTSGRAKAESRIRRPMNAFMVWAKDERKRLAQQNPDLHNAELSKMLGKSWKALTLAEKRPFVEEAERLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAPPPPPQQQHPAHGPGQPSPPPEALPCRDGTESNQPTELLGEVDRTEFEQYLPFVYKPEMGLPYQGHDCGVNLSDSHGAISSVVSDASSAVYYCNYPDI